(19) United States
(12) Patent Application Publication
(10) Pub. No.: US 2026/0131017 A1
(43) Pub. Date: May 14, 2026

(54) CD22 ANTIBODY AND DRUG CONJUGATES AND USES THEREOF

(71) Applicant: MEDIMMUNE LLC, Gaithersburg, MD (US)

(72) Inventors: Marcello MARELLI, Gaithersburg, MD (US); Huiying HAN, Wilmington, DE (US); Qun DU, Wilmington, DE (US)

(21) Appl. No.: 19/354,251

(22) Filed: Oct. 9, 2025

Related U.S. Application Data
(60) Provisional application No. 63/706,134, filed on Oct. 11, 2024.
(60) Provisional application No. 63/718,026, filed on Nov. 8, 2024.

(51) Int. Cl. A61K 47/68; A61P 35/00; C07K 16/28; G01N 33/575

(52) U.S. Cl. CPC A61K 47/68037; A61K 47/6849; A61K 47/6889; A61P 35/00; C07K 16/2803; G01N 33/5759; C07K 2317/21; C07K 2317/52; C07K 2317/565; C07K 2317/71; C07K 2317/732; G01N 2333/70503

(57) ABSTRACT

The present disclosure relates to CD22 binding molecules, such as anti-CD22 antibodies, for the treatment of cancer, and related antibody-drug conjugates.

### CROSS REFERENCE TO RELATED APPLICATIONS

This application claims priority to U.S. Provisional Application No. 63/706,134, filed Oct. 11, 2024, and U.S. Provisional Application No. 63/718,026, filed Nov. 8, 2024, the disclosures of each of which are incorporated by reference in its entirety.

### REFERENCE TO SEQUENCE LISTING SUBMITTED ELECTRONICALLY

This application contains a sequence listing which is submitted electronically and is hereby incorporated by reference in its entirety. The sequence listing submitted herewith is contained in the XML filed created Oct. 8, 2025, entitled “24-1168-US_Sequence-Listing.xml” and is 20,599 bytes in size.

### FIELD OF THE DISCLOSURE

The present disclosure relates to CD22 binding molecules, such as anti-CD22 antibodies, for the treatment of cancer, and related anti-CD22 antibody-drug conjugates.

### BACKGROUND

Despite years of research into and development of potential anti-cancer drugs, cancer remains one of the leading diseases globally, with one in three individuals developing some form of cancer in their lifetime.

The principal therapies for cancer remain chemotherapy and radiotherapy. However, these therapies are associated with various undesirable side effects, from fatigue through to sickness and hair loss. These issues are exacerbated by the often-lengthy courses of chemotherapy used.

Over the last couple of decades, a number of antibody therapies for cancer have been developed and marketed, leading to a reduction in the need for harsh forms of therapy (e.g., surgery and chemotherapy) for a number of cancer types. Although the availability of methodology for producing antibodies (e.g., monoclonal antibodies) has greatly improved over this time period, there are relatively few clinically available anti-cancer antibodies, and even fewer that may be used to target a broad spectrum of cancer types. Furthermore, there is a need to increase the potency of therapeutic antibodies, which is generally limited by the target antigen and subsequent effects on the cancer cell following antibody binding.

CD22, also known as Siglec-2 (sialic acid-binding immunoglobulin-like lectin 2), is a cell surface protein that is primarily expressed on B-cells. It plays a role in regulating B-cell signaling and survival. CD22 is overexpressed on the surface of malignant B-cells in various B-cell malignancies, including non-Hodgkin lymphoma, acute lymphoblastic leukemia, and chronic lymphocytic leukemia. Inotuzumab ozogamicin (BESPONSA®) is a CD22 antibody drug conjugate approved for the treatment of relapsed or refractory B-cell precursor acute lymphoblastic leukemia (ALL). BESPONSA® has the cytotoxic payload, calicheamicin, which can lead to hepatotoxicity (liver damage) and potentially severe liver-related adverse events.

There is a need to provide alternative therapies to treat B-cell malignancies which may have improved safety/toxicity profiles and/or improved efficacy.

### SUMMARY OF THE DISCLOSURE

The present disclosure is directed to an anti-CD22 antibody or antigen binding fragment thereof, comprising a heavy chain CDR1 (HCDR1) comprising the amino acid sequence of SEQ ID NO: 1, a heavy chain CDR2 (HCDR2) comprising the amino acid sequence of SEQ ID NO: 2, a heavy chain CDR3 (HCDR3) comprising the amino acid sequence of SEQ ID NO: 3, a light chain CDR1 (LCDR1) comprising the amino acid sequence of SEQ ID NO: 4, a light chain CDR2 (LCDR2) comprising the amino acid sequence of SEQ ID NO: 5, and a light chain CDR3 (LCDR3) comprising the amino acid sequence of SEQ ID NO: 6.

In some aspects, the anti-CD22 antibody or antigen binding fragment thereof comprises a variable heavy (VH) chain at least 90%, at least 91% at least 92%, at least 93%, at least 94%, at least 95%, at least 96%, at least 97%, at least 98%, or at least 99% identical to the amino acid sequence of SEQ ID NO: 7, and a variable light (VL) chain at least 90%, at least 91% at least 92%, at least 93%, at least 94%, at least 95%, at least 96%, at least 97%, at least 98%, or at least 99% identical to the amino acid sequence of SEQ ID NO: 8.

In some aspects, the anti-CD22 antibody or antigen binding fragment thereof comprises a VH chain comprising the amino acid sequence of SEQ ID NO: 7 and a VL chain comprising the amino acid sequence of SEQ ID NO: 8.

In some aspects, the anti-CD22 antibody or binding fragment thereof comprises an Fc region.

In some aspects, the anti-CD22 antibody or antigen binding fragment thereof comprises an Fc region comprising one or more mutations, which has reduced antibody dependent cellular cytotoxicity (ADCC) compared to an antibody having a wild-type Fc region.

In some aspects, the Fc region comprises a L234F/L235E/P331S (EU Index numbering) triple mutation (TM).

In some aspects, the anti-CD22 antibody or antigen binding fragment thereof comprises a constant heavy chain comprising the amino acid sequence of SEQ ID NO: 11 and a constant light chain comprising the amino acid sequence of SEQ ID NO: 12.

In some aspects, the anti-CD22 antibody or antigen binding fragment thereof comprises a heavy chain comprising the amino acid sequence of SEQ ID NO: 9.

In some aspects, the anti-CD22 antibody or antigen binding fragment thereof comprises a light chain comprising the amino acid sequence of SEQ ID NO: 10.

In some aspects, the anti-CD22 antibody or antigen binding fragment thereof comprises a heavy chain comprising the amino acid sequence of SEQ ID NO: 9; and a light chain comprising the amino acid sequence of SEQ ID NO: 10.

In some aspects, the anti-CD22 antibody or antigen binding fragment thereof comprises a heavy chain comprising the amino acid sequence of SEQ ID NO: 13; and a light chain comprising the amino acid sequence of SEQ ID NO: 10.

In some aspects, the anti-CD22 antigen binding fragment is a single chain Fv, Fab fragment, a Fab′ fragment, or a F(ab′)2 fragment.

In some aspects, the anti-CD22 antibody or antigen-binding fragment thereof is humanized, chimeric, or fully human.

In some aspects, the anti-CD22 antibody or antigen-binding fragment thereof is fully human.

In some aspects, the anti-CD22 antibody or antigen-binding fragment thereof is a monoclonal antibody.

In some aspects, the anti-CD22 antibody or antigen-binding fragment thereof is an IgG1.

In some aspects, the anti-CD22 antibody or antigen binding fragment thereof binds a B-cell derived cell line.

The present disclosure is also directed to pharmaceutical compositions comprising the anti-CD22 antibody or antigen binding fragment thereof as disclosed herein.

The present disclosure is also directed to a polynucleotide encoding the anti-CD22 antibody or antigen binding fragment thereof as disclosed herein.

In some aspects, the polynucleotide of comprises a heavy chain nucleic acid sequence of SEQ ID NO: 16 and a light chain nucleic acid sequence of SEQ ID NO: 15.

The present disclosure is also directed to host cells comprising the polynucleotides encoding the anti-CD22 antibody or antigen binding fragment thereof as disclosed herein.

The present disclosure is also directed to a method for producing the anti-CD22 antibody or antigen binding fragment thereof as disclosed herein in a host cell.

The present disclosure is also directed to an anti-CD22 antibody or antigen binding fragment thereof obtainable by the methods disclosed herein.

In another aspect, the disclosure provides the anti-CD22 antibody or antigen binding fragment thereof as disclosed herein, wherein the antibody or antigen binding fragment thereof is conjugated to one or more drugs.

In some aspects, the anti-CD22 antibody or antigen binding fragment thereof is conjugated to one or more drugs selected from the group consisting of a cytotoxin, a tubulysin derivative, an antimicrobial agent, a therapeutic agent, a prodrug, a peptide, a protein, an enzyme, a lipid, a biological response modifier, a pharmaceutical agent, a lymphokine, a heterologous antibody, a fragment of a heterologous antibody, a detectable label, a polyethylene glycol (PEG), a radioisotope, and a combination thereof.

In some aspects, the anti-CD22 antibody or antigen binding fragment thereof to one or more drugs that is a cytotoxin selected from the group consisting of a DNA scission agent, a DNA binding agent, a DNA intercalating agent, a topoisomerase I inhibitor, a topoisomerase II inhibitor, a tubulysin derivative, and an antimitotic agent.

In some aspects, the one or more drugs is a cytotoxin selected from the group consisting of calicheamicins, pyrrolobenzodiazepines, camptothecins, daunorubicins, doxorubicins, auristatins, and maytansinoids.

In some aspects, the one or more drugs is a cytotoxin which is a topoisomerase I inhibitor.

In some aspects, the topoisomerase I inhibitor is exatecan:

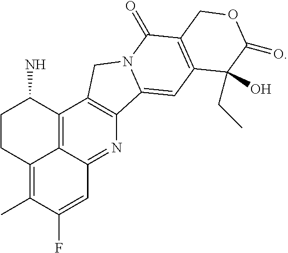 [SMILES: CC[C@@]1(O)C(=O)OCc2c1cc1n(c2=O)Cc2c-1nc1cc(F)c(C)c3c1c2[C@@H](N)CC3]

In some aspects, the topoisomerase I inhibitor is:

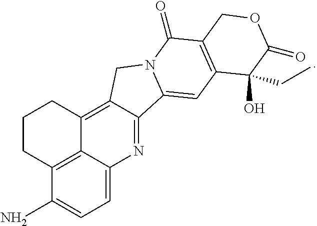 [SMILES: CC[C@@]1(O)C(=O)OCc2c1cc1n(c2=O)Cc2c-1nc1ccc(N)c3c1c2CCC3]

In some aspects, the anti-CD22 antibody or antigen binding fragment thereof is conjugated to a drug at a drug to antibody ratio (DAR) of about 4 to about 8.

In some aspects, the anti-CD22 antibody or antigen binding fragment thereof is conjugated to a drug at a drug to antibody ratio (DAR) of about 8.

In some aspects, the anti-CD22 antibody or antigen binding fragment thereof is conjugated to a drug at a drug to antibody ratio (DAR) of 4.

In another aspect, this disclosure provides an antibody-drug conjugate (ADC) of Formula (I):

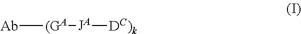

wherein
Ab is an anti-CD22 antibody or antigen-binding fragment thereof as disclosed herein, k is an integer from 1 to 10;
each G^{A} is independently a conjugation group conjugated to the antibody or antigen-binding fragment thereof;
each D^{C} is selected from the group consisting of a cytotoxin, a tubulysin derivative, an antimicrobial agent, a therapeutic agent, a prodrug, a peptide, a protein, an enzyme, a lipid, a biological response modifier, a pharmaceutical agent, a lymphokine, a heterologous antibody, a fragment of a heterologous antibody, a detectable label, a polyethylene glycol (PEG) and a radioisotope; and
each J^{A} is independently a linker.

In some aspects of the antibody-drug conjugate each J^{A} is independently a group of Formula (ICA):

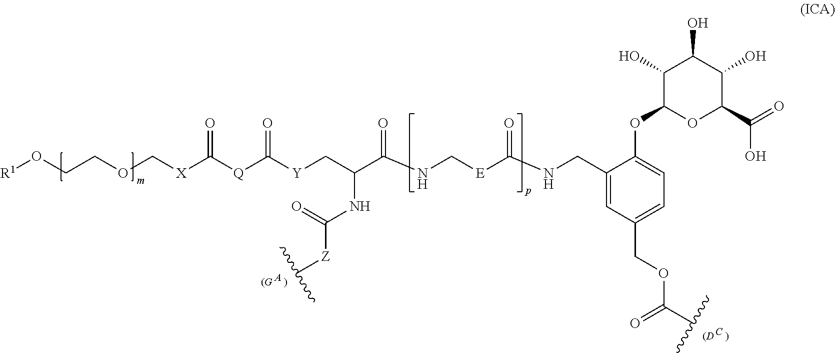

E is (CH₂)ₙ₁, wherein n1 is 0, 1, 2, or 3;
Q is

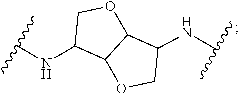 [SMILES: *NC1COC2C(N*)COC12]

R¹ is C₁₋₄ alkyl;
X is (CH₂)ₙ₂, wherein n2 is 0, 1, 2, or 3;
Y is (CH₂)ₙ₃, wherein n3 is 0, 1, 2, 3, or 4;
Z is (CH₂)ₙ₄, wherein n4 is 1, 2, 3, 4, or 5;
m is an integer from 5 to 17;
p is 1 or 0;
(G^{A}) indicates the point of attachment to G^{A}; and
(D^{C}) indicates the point of attachment to D^{C}.

In some aspects of the antibody-drug conjugate Q is:

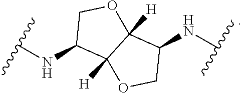 [SMILES: *N[C@H]1CO[C@H]2[C@@H]1OC[C@@H]2N*]

In some aspects of the antibody-drug conjugate m is 9, 10, 11, 12, or 13.

In some aspects of the antibody-drug conjugate R₁ is CH₃.

In some aspects of the antibody-drug conjugate E is CH₂.

In some aspects of the antibody-drug conjugate X is CH₂.

In some aspects of the antibody-drug conjugate Y is (CH₂)₂.

In some aspects of the antibody-drug conjugate Z is (CH₂)₂.

In some aspects of the antibody-drug conjugate Z is CH₂.

In some aspects of the antibody-drug conjugate p is 1.

In some aspects of the antibody-drug conjugate each J_{A} is a group of Formula (ICB):

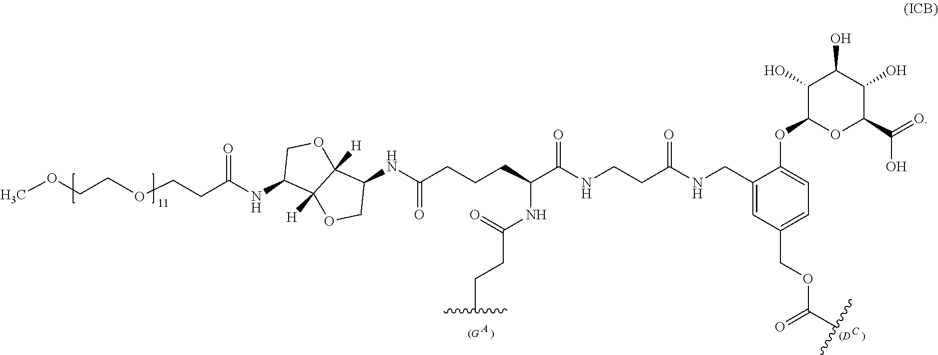

In some aspects of the antibody-drug conjugate G^{A} is selected from the group consisting of:

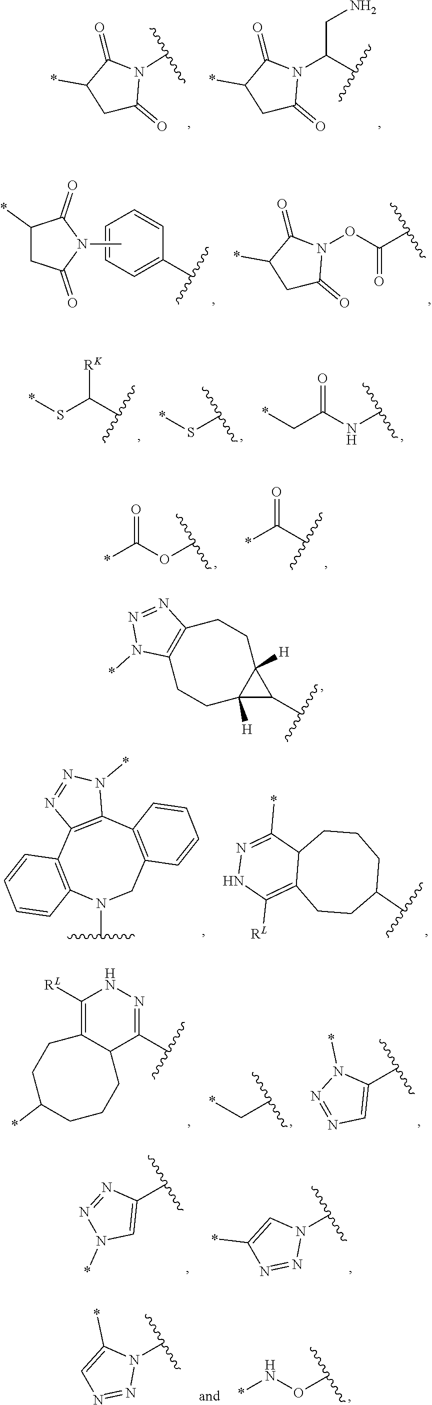

wherein R^{K} is H or CH₃, R^{L} is C₁₋₆ alkyl, and [symbol] indicates the point of attachment to the antibody or antigen-binding fragment thereof.

In some aspects of the antibody-drug conjugate G^{A} is:

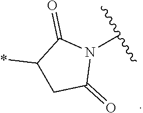 [SMILES: *C1CC(=O)N(*)C1=O]

In some aspects of the antibody-drug conjugate G^{A} is:

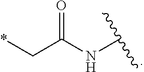 [SMILES: *CC(=O)N*]

In some aspects of the antibody-drug conjugate k is an integer from about 4 to about 8.

In some aspects of the antibody-drug conjugate k is about 4.

In some aspects of the antibody-drug conjugate k is about 8.

In some aspects of the antibody-drug conjugate each D^{C} is:

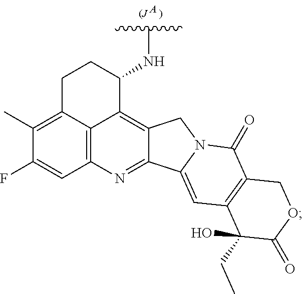

and (J^{A}) indicates the point of attachment to J^{A}.

In some aspects of the antibody-drug conjugate each D^{C} is:

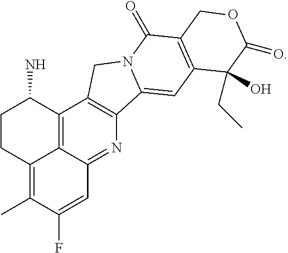

In some aspects of the antibody-drug conjugate G^{A}-J^{A}-D^{C} is:

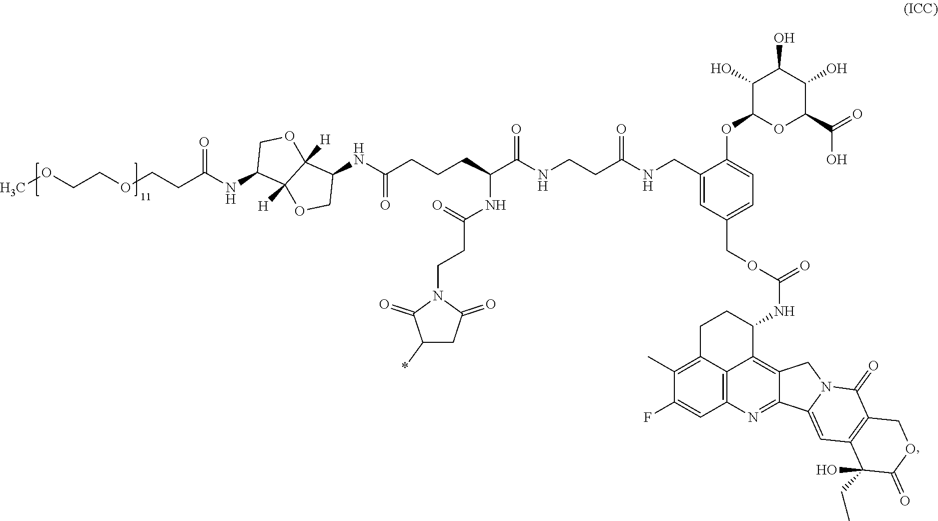

wherein [symbol] indicates the point of attachment to the antibody or antigen-binding fragment thereof.

In some aspects of the antibody-drug conjugate G^{A}-J^{A}-D^{C} is:

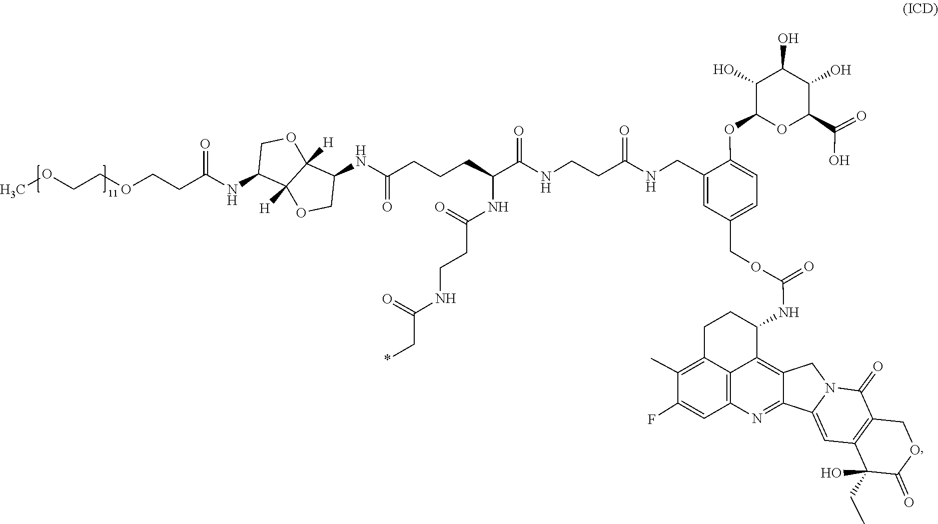

wherein [symbol] indicates the point of attachment to the antibody or antigen-binding fragment thereof.

In some aspects of the antibody-drug conjugate each J^{A} is a group of Formula (ICE):

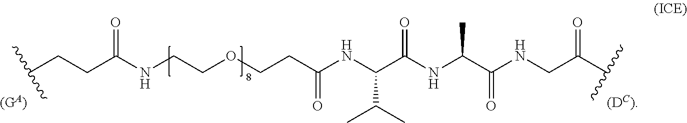

In some aspects of the antibody-drug conjugate G^{A} is selected from the group consisting of:

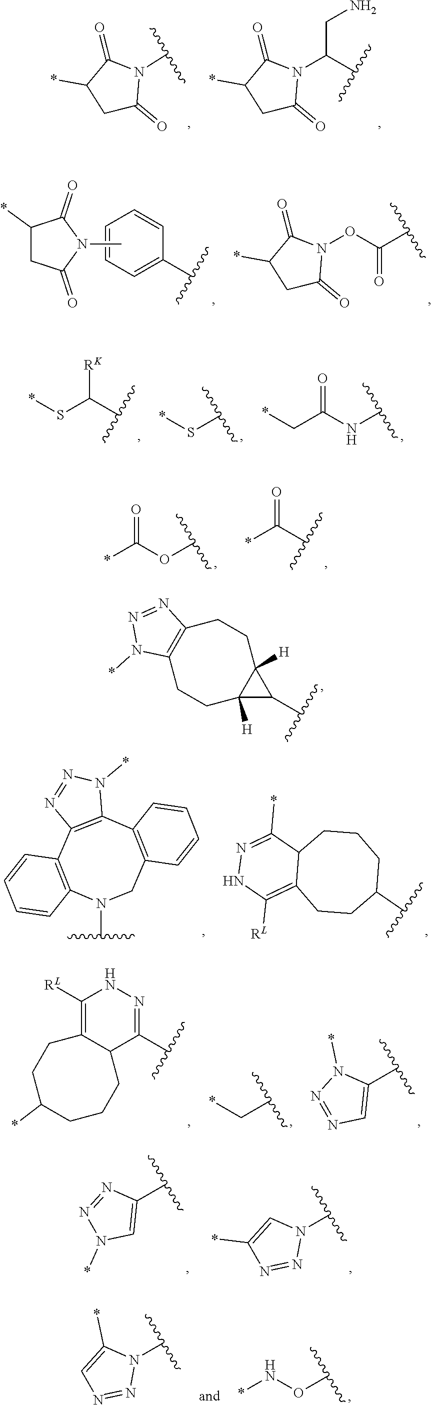

wherein R^{K} is H or CH₃, R^{L} is C₁₋₆ alkyl, and [symbol] indicates the point of attachment to the antibody or antigen-binding fragment thereof.

In some aspects of the antibody-drug conjugate G^{A} is:

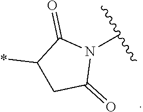 [SMILES: *C1CC(=O)N(*)C1=O]

In some aspects of the antibody-drug conjugate G^{A} is:

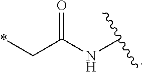 [SMILES: *CC(=O)N*]

In some aspects of the antibody-drug conjugate k is an integer from about 4 to about 8.

In some aspects of the antibody-drug conjugate k is about 4.

In some aspects of the antibody-drug conjugate k is about 8.

In some aspects of the antibody-drug conjugate each D^{C} is:

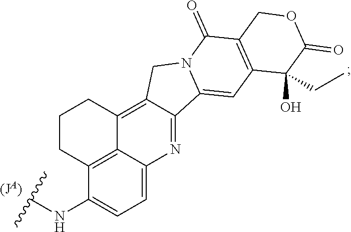 [SMILES: *Nc1ccc2nc3c(c4c2c1CCC4)Cn1c-3cc2c(c1=O)COC(=O)[C@]2(O)CC]

and (J^{A}) indicates the point of attachment to J^{A}.

In some aspects of the antibody-drug conjugate each D^{C} is:

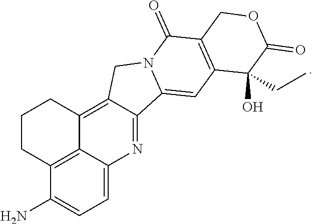

In some aspects of the antibody-drug conjugate G^{A}-J^{A}-D^{C} is:

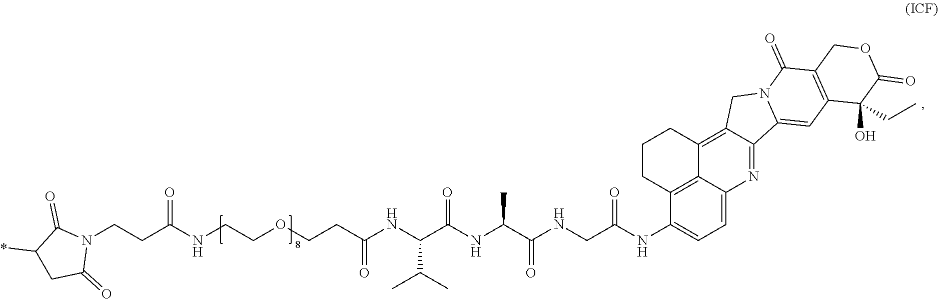

wherein [symbol] indicates the point of attachment to the antibody or antigen-binding fragment thereof.

In some aspects of the antibody-drug conjugate each J^{A} is a group of Formula (ICG):

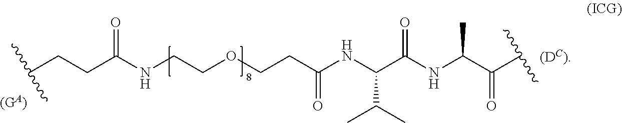

In some aspects of the antibody-drug conjugate G^{A} is selected from the group consisting of:

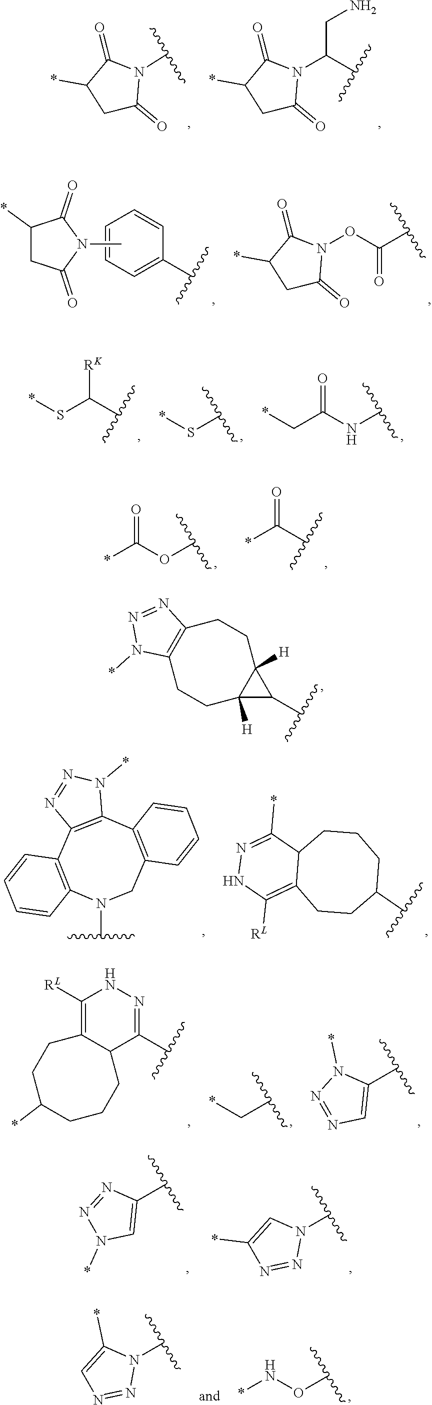

wherein R^{K} is H or CH₃, R^{L} is C₁₋₆ alkyl, and

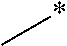 [SMILES: *C]

indicates the point of attachment to the antibody or antigen-binding fragment thereof.

In some aspects of the antibody-drug conjugate G^{A} is:

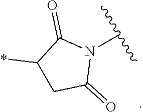 [SMILES: *C1CC(=O)N(*)C1=O]

In some aspects of the antibody-drug conjugate G^{A} is:

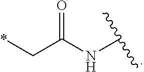 [SMILES: *CC(=O)N*]

In some aspects of the antibody-drug conjugate k is an integer from about 4 to about 8.

In some aspects of the antibody-drug conjugate k is about 4.

In some aspects of the antibody-drug conjugate k is about 8.

In some aspects of the antibody-drug conjugate each D^{C} is:

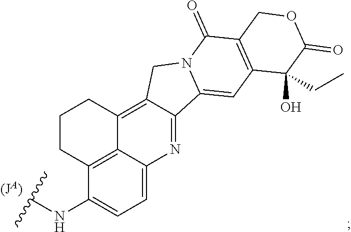 [SMILES: *Nc1ccc2nc3c(c4c2c1CCC4)Cn1c-3cc2c(c1=O)COC(=O)[C@]2(O)CC]

and (J^{A}) indicates the point of attachment to J^{A}.

In some aspects of the antibody-drug conjugate each D^{C} is:

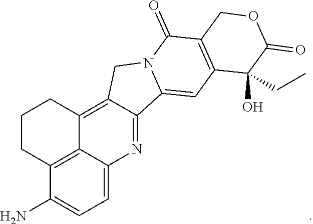

In some aspects of the antibody-drug conjugate G^{A}-J^{A}-D^{C} is:

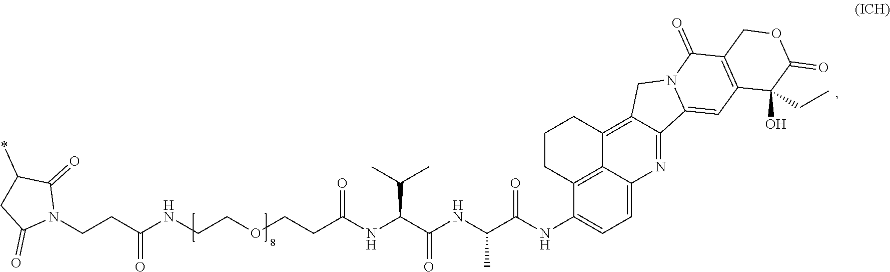

wherein [symbol] indicates the point of attachment to the antibody or antigen-binding fragment thereof.

In another aspect, this disclosure provides an antibody-drug conjugate comprising:
(i) an anti-CD22 antibody or antigen binding fragment thereof comprising: a heavy chain CDR1 (HCDR1) comprising the amino acid sequence of SEQ ID NO: 1, a heavy chain CDR2 (HCDR2) comprising the amino acid sequence of SEQ ID NO: 2, a heavy chain CDR3 (HCDR3) comprising the amino acid sequence of SEQ ID NO: 3, a light chain CDR1 (LCDR1) comprising the amino acid sequence of SEQ ID NO: 4, a light chain CDR2 (LCDR2) comprising the amino acid sequence of SEQ ID NO: 5, and a light chain CDR3 (LCDR3) comprising the amino acid sequence of SEQ ID NO: 6; and
(ii) a linker payload of Formula (ICC):

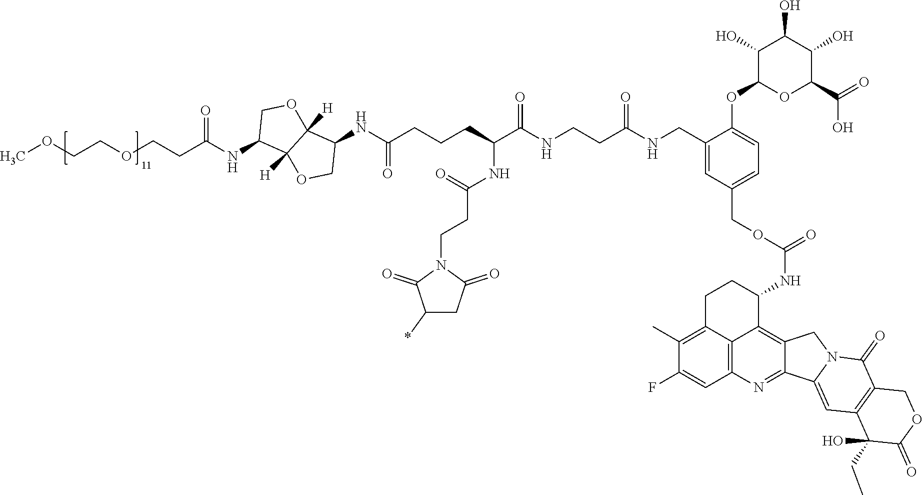

wherein [symbol] indicates the point of attachment to the antibody or antigen-binding fragment thereof; and
wherein the ADC has a drug to antibody ratio (DAR) ranging from about 4 to about 8.

In another aspect, this disclosure provides an antibody-drug conjugate comprising:
(i) an anti-CD22 antibody or antigen binding fragment thereof comprising: a heavy chain CDR1 (HCDR1) comprising the amino acid sequence of SEQ ID NO: 1, a heavy chain CDR2 (HCDR2) comprising the amino acid sequence of SEQ ID NO: 2, a heavy chain CDR3 (HCDR3) comprising the amino acid sequence of SEQ ID NO: 3, a light chain CDR1 (LCDR1) comprising the amino acid sequence of SEQ ID NO: 4, a light chain CDR2 (LCDR2) comprising the amino acid sequence of SEQ ID NO: 5, and a light chain CDR3 (LCDR3) comprising the amino acid sequence of SEQ ID NO: 6; and (ii) a linker payload of Formula (ICD):

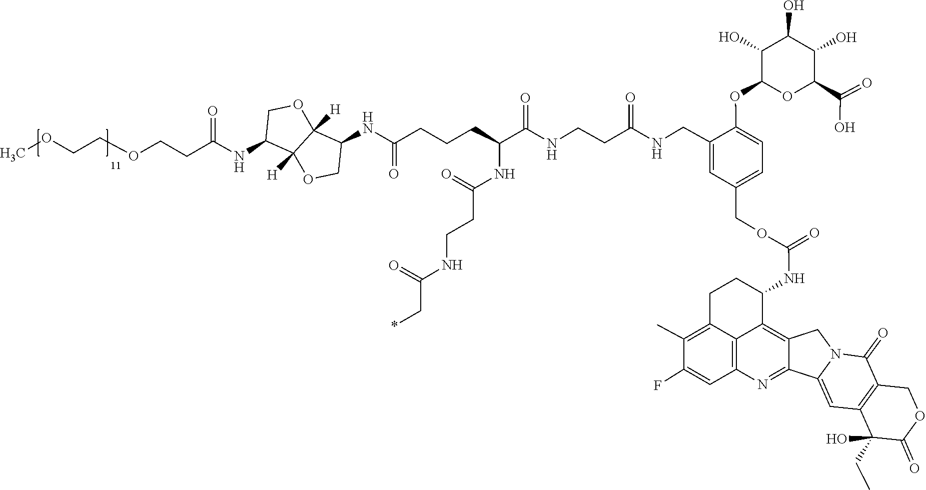

wherein [symbol] indicates the point of attachment to the antibody or antigen-binding fragment thereof; and wherein the ADC has a drug to antibody ratio (DAR) ranging from about 4 to about 8.

In another aspect, this disclosure provides an antibody-drug conjugate comprising:
(i) an anti-CD22 antibody or antigen binding fragment thereof comprising: a heavy chain CDR1 (HCDR1) comprising the amino acid sequence of SEQ ID NO: 1, a heavy chain CDR2 (HCDR2) comprising the amino acid sequence of SEQ ID NO: 2, a heavy chain CDR3 (HCDR3) comprising the amino acid sequence of SEQ ID NO: 3, a light chain CDR1 (LCDR1) comprising the amino acid sequence of SEQ ID NO: 4, a light chain CDR2 (LCDR2) comprising the amino acid sequence of SEQ ID NO: 5, and a light chain CDR3 (LCDR3) comprising the amino acid sequence of SEQ ID NO: 6; and
(ii) a linker payload of Formula (ICF):

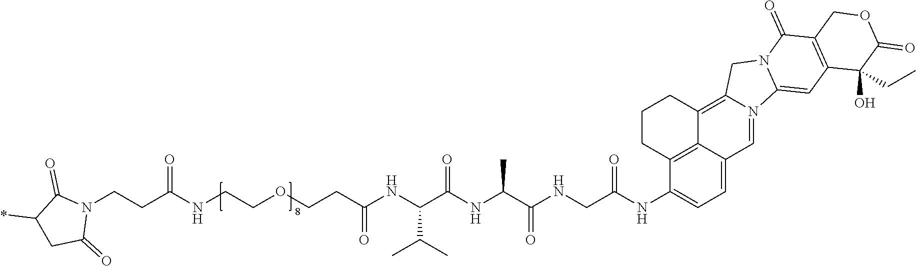

wherein [symbol] indicates the point of attachment to the antibody or antigen-binding fragment thereof; and wherein the ADC has a drug to antibody ratio (DAR) ranging from about 4 to about 8.

In another aspect, this disclosure provides an antibody-drug conjugate comprising:
(i) an anti-CD22 antibody or antigen binding fragment thereof comprising: a heavy chain CDR1 (HCDR1) comprising the amino acid sequence of SEQ ID NO: 1, a heavy chain CDR2 (HCDR2) comprising the amino acid sequence of SEQ ID NO: 2, a heavy chain CDR3 (HCDR3) comprising the amino acid sequence of SEQ ID NO: 3, a light chain CDR1 (LCDR1) comprising the amino acid sequence of SEQ ID NO: 4, a light chain CDR2 (LCDR2) comprising the amino acid sequence of SEQ ID NO: 5, and a light chain CDR3 (LCDR3) comprising the amino acid sequence of SEQ ID NO: 6; and
(ii) a linker payload of Formula (ICH):

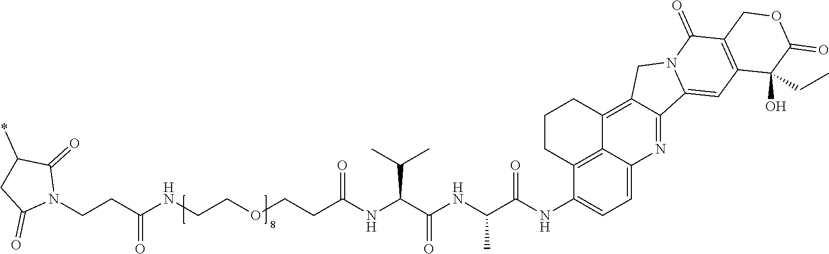

wherein [symbol] indicates the point of attachment to the antibody or antigen-binding fragment thereof; and wherein the ADC has a drug to antibody ratio (DAR) ranging from about 4 to about 8.

In some aspects of the antibody-drug conjugate as disclosed herein, the antibody or antigen binding fragment thereof comprises a VH chain comprising the amino acid sequence of SEQ ID NO: 7 and a VL chain comprising the amino acid sequence of SEQ ID NO: 8.

In some aspects of the antibody-drug conjugate as disclosed herein, the antibody or antigen binding fragment thereof comprises a heavy chain (HC) comprising the amino acid sequence of SEQ ID NO: 9, and light chain (LC) comprising the amino acid sequence of SEQ ID NO: 10.

In some aspects of the antibody-drug conjugate as disclosed herein, the antibody or antigen binding fragment thereof comprises a heavy chain (HC) comprising the amino acid sequence of SEQ ID NO: 13, and light chain (LC) comprising the amino acid sequence of SEQ ID NO: 10.

This disclosure also provides pharmaceutical compositions comprising the antibody-drug conjugates as disclosed herein.

This disclosure also provides the antibody-drug conjugate as disclosed herein or the pharmaceutical composition as disclosed herein, for use as a medicament.

This disclosure also provides the antibody-drug conjugate as disclosed herein or the pharmaceutical composition as disclosed herein, for use in the treatment of cancer. In some embodiments, the cancer expresses CD22.

In some aspects of the method of treating cancer, the method comprises administering to a subject the antibody-drug conjugate as disclosed herein, or the pharmaceutical composition as disclosed herein. In some embodiments, the cancer expresses CD22.

In some aspects, the subject is a human.

In some aspects of the methods disclosed herein, said cancer is a B-cell malignancy.

In some aspects of the pharmaceutical composition as disclosed herein, said cancer is a B-cell malignancy.

In some aspects of the methods disclosed herein or the pharmaceutical compositions as disclosed herein, said B-cell malignancy is selected from the group consisting of non-Hodgkin lymphoma, acute lymphoblastic leukemia (ALL), Burkitt lymphoma, chronic lymphocytic leukemia (CLL), small lymphocytic lymphoma (SLL), diffuse large B-cell lymphoma (DLBCL), follicular lymphoma (FL), mantle cell lymphoma (MCL), lymphoplasmacytic lymphoma, and Hairy cell leukemia.

In some aspects of the methods disclosed herein or the pharmaceutical compositions as disclosed herein, said B-cell malignancy is refractory [B-cell] acute lymphoblastic leukemia (ALL) or relapsed [B-cell] acute lymphoblastic leukemia (ALL).

This disclosure also provides the anti-CD22 antibody or antigen binding fragment as disclosed herein or the pharmaceutical compositions as disclosed herein, for use as a medicament.

This disclosure also provides the pharmaceutical compositions as disclosed herein, for use in the treatment of cancer. In some embodiments, said cancer expresses CD22.

This disclosure also provides a method of treating cancer, the method comprising administering to a subject an anti-CD22 antibody or antigen binding fragment thereof as disclosed herein or a pharmaceutical composition as disclosed herein. In some embodiments, said cancer expresses CD22. In some embodiments, the subject is a human.

In some aspects, said cancer is a B-cell malignancy.

In some aspects, said cancer is a B-cell malignancy is selected from the group consisting of non-Hodgkin lymphoma, acute lymphoblastic leukemia (ALL), Burkitt lymphoma, chronic lymphocytic leukemia (CLL), small lymphocytic lymphoma (SLL), diffuse large B-cell lymphoma (DLBCL), follicular lymphoma (FL), mantle cell lymphoma (MCL), lymphoplasmacytic lymphoma, and Hairy cell leukemia.

In some aspects, said B-cell malignancy is refractory [B-cell] acute lymphoblastic leukemia (ALL) or relapsed [B-cell] acute lymphoblastic leukemia (ALL).

In another aspect, this disclosure provides a method for detecting the presence or absence of a CD22 polypeptide in a sample, comprising:
(i) contacting a sample with an antibody or antigen binding fragment thereof as disclosed herein, or a pharmaceutical composition as disclosed herein, to provide an antibody-antigen complex; and
(ii) detecting the presence or absence of said antibody-antigen complex; wherein the presence of the antibody-antigen complex confirms the presence of a CD22 polypeptide; and wherein the absence of the antibody-antigen complex confirms the absence of CD22 polypeptide.

In some aspects, the presence of said antibody-antigen complex is indicative of the presence of a CD22 expressing cancer cell, and wherein the absence of said antibody-antigen complex is indicative of the absence of a CD22 expressing cancer cell.

In some aspects, the sample is an isolated sample obtainable from a subject.

In some aspects, the kit of parts comprises at least one of (i) a variable heavy chain, and (ii) a variable light chain of the (a) an anti-CD22 antibody or antigen binding fragment thereof as disclosed herein, (b) an antibody drug conjugate as disclosed herein, or (c) a pharmaceutical composition as disclosed herein.

### BRIEF DESCRIPTION OF THE DRAWINGS

Aspects of the disclosure will now be described, by way of example only, with reference to the following Figures and Examples.

FIG. 1 shows CD22 displays distinct cytoplasmic staining in non-Hodgkin lymphomas and remaining cancer tissues were negative. Data queried from Human Protein Atlas (HPA) database.

FIG. 2 shows mAb23-TM binding kinetics to human (top graph) and cynomolgus monkey (bottom graph) CD22 as measured by surface plasmon resonance.

FIG. 3A shows that mAb23-TM exhibits binding to human CD22 (HuCD22) and Cynomolgus CD22 (Cy), but does not show binding to murine CD22 (Mu) expressing AD293 cells.

FIG. 3B shows mAb23-TM and its conjugated ADCs, mAb23-TM β-glu exatecan and mAb23-TM Val-Ala TOP1i ('0132), exhibit similar binding to DLBCL cell line WSU-DLCL2 and OCILY19, with high and low endogenous CD22 expressing levels, respectively.

FIG. 4 shows the internalization kinetics of mAb23-TM and its conjugated ADCs, mAb23-TM β-glu exatecan and mAb23-TM Val-Ala TOP1i ('0132), at 0.5 μg/mL in DLBCL cell lines WSU-DLCL2 and OCILY19, with high and low endogenous CD22 expressing levels, respectively. No internalization signal was observed with the isotype control antibody (Nip228-TM).

FIG. 5 shows an exemplary schematic of the DAR8 ADC structures, and the synthesis used to procure the ADC. RT=room temperature.

FIG. 6A shows cytotoxic effect of mAb23-TM Val-Ala TOP1i ('0132) ADC in DLBCL cell lines and DLBCL CD22 knock out cell lines. CD22-specific killing of the ADC was confirmed by the dose-dependent decrease in cell viability of treated WSU-DLCL2 and OCILY19 cells and the lack thereof in the isogenic CD22 knock-out cell lines or with the isotype control ADC treatment.

FIG. 6B shows cytotoxic effect of mAb23-TM 3-glu exatecan ADC in DLBCL cell lines and DLBCL CD22 knock out cell lines. CD22-specific killing of the ADC was confirmed by the dose-dependent decrease in cell viability of treated WSU-DLCL2 and OCILY19 cells and the lack thereof in the isogenic CD22 knock-out cell lines or with the isotype control ADC treatment.

FIG. 7A shows cytotoxic effect of mAb23-TM Val-Ala TOP1i ('0132) ADC in CD22 expressing MCL, FL and B-ALL cell lines. CD22-specific killing of the ADC was confirmed by the dose-dependent decrease in cell viability of treated DOHH2 (FL), Granta519 (MCL), REH (B-ALL) and MUTZ5 (B-ALL) cells.

FIG. 7B shows cytotoxic effect of mAb23-TM 3-glu exatecan ADC in CD22 expressing MCL, FL and B-ALL cell lines. CD22-specific killing of the ADC was confirmed by the dose-dependent decrease in cell viability of treated DOHH2 (FL), Granta519 (MCL), REH (B-ALL) and MUTZ5 (B-ALL) cells.

FIG. 8A shows the plasma concentrations of either total mAb or total ADC following a single intravenous (IV) dose of 2 mg/kg of mAb23-TM 3-glu exatecan ADC or mAb23-TM Val-Ala TOP1i ('0132) ADC. Noncompartmental analysis (NCA) was conducted to calculate clearance, half-life, and volume of distribution and over a 14-day duration. There is minimal difference in clearance between total ADC or total mAb for both ADCs in CB17 mice.

FIG. 8B shows the tumor concentrations of free payload (exatecan or TOP1i ('0132) following a single intravenous (IV) dose of 2 mg/kg of mAb23-TM 3-glu exatecan ADC or mAb23-TM Val-Ala TOP1i ('0132) ADC in CB17 mice. The free payload concentration reached its peak value on day 3 after dosing. Exatecan exhibited approximately 2.5 times higher concentration than TOP1i ('0132) at the peak value.

FIG. 9A shows the efficacy of mAb23-TM Val-Ala TOP1i ('0132) ADC or isotype control ADC after a single intravenous (IV) dose ranging from 0.25-2 mg/kg in CB17 mice bearing 2 DLBCL cell-derived xenografts (CDX) subcutaneous models: WSU-DLCL2 and OCILY19, with relatively high and low CD22 expression levels, respectively. Notably, mAb23-TM Val-Ala TOP1i ('0132) demonstrated dose-dependent decreases in tumor volume in both models over a 50-day duration. The presented values are mean±SEM tumor volumes (n=3), with the dotted line representing the average baseline tumor volume at the time of dosing.

FIG. 9B shows the efficacy of mAb23-TM 3-glu exatecan ADC or isotype control ADC after a single intravenous (IV) dose at 0.25, 0.5 or 1 mg/kg in CB17 mice bearing 2 DLBCL cell-derived xenografts (CDX) subcutaneous models: WSU-DLCL2 and OCILY19, with relatively high and low CD22 expression levels, respectively. Notably, mAb23-TM 3-glu exatecan demonstrated dose-dependent decreases in tumor volume in both models over a 50-day duration. The presented values are mean±SEM tumor volumes (n=3), with the dotted line representing the average baseline tumor volume at the time of dosing.

FIG. 10A shows the survival benefit of mAb23-TM Val-Ala TOP1i ('0132) ADC after intravenous (IV) dosing weekly for total two doses at 1.2, 2.4 or 3.6 mg/kg in CB17 mice bearing DLBCL cell-derived xenograft (CDX) disseminated model SUDHL6. mAb23-TM Val-Ala TOP1i ('0132) provided survival benefit at dose-dependent manner following the treatment at each dose level compared to the untreated group (n=10).

FIG. 10B shows the survival benefit of mAb23-TM 3-glu exatecan ADC after intravenous (IV) dosing weekly for total two doses at 1.2, 2.4 or 3.6 mg/kg in CB17 mice bearing DLBCL cell-derived xenograft (CDX) disseminated model SUDHL6. mAb23-TM β-glu exatecan provided survival benefit at dose-dependent manner following the treatment at each dose level compared to the untreated group (n=10).

FIG. 11A shows the median best response of 6 different DLBCL PDX models following a single dose intravenous (IV) administration of mAb23-TM Val-Ala TOP1i ('0132) ADC or isotype control ADC at 0.8, 1.6 or 2.4 mg/kg. Models were considered to be responsive to the test agents if the percent tumor volume from baseline was −30% to −100%, inclusive.

FIG. 11B shows the median best response of 6 different DLBCL PDX models following a single dose intravenous (IV) administration of mAb23-TM β-glu exatecan ADC or isotype control ADC at 0.8, 1.6 or 2.4 mg/kg. Models were considered to be responsive to the test agents if the percent tumor volume from baseline was −30% to −100%, inclusive.

FIG. 12 shows the median best response of 6 different MCL/FL models following a single dose intravenous (IV) administration of mAb23-TM 3-glu exatecan ADC or isotype control ADC at 0.8, 1.6 or 2.4 mg/kg. Models were considered to be responsive to the test agents if the percent tumor volume from baseline was −30% to −100%, inclusive.

FIG. 13 shows the efficacy of mAb23-TM 3-glu exatecan ADC in reducing tumor burden (CD45+ cell population) in peripheral blood (PB) at day 7 after the first intravenous dose at 1 or 2.4 mg/kg in CB17 mice bearing B-ALL cell-derived xenograft (CDX) disseminated model REH (n=5, Top). Consequently, mAb23-TM β-glu exatecan provided survival benefit at dose-dependent manner following the treatment (administered every 3 weeks, with a total of two doses for the study through intravenous administration) at each dose level compared to the untreated group (n=10, Bottom).

FIG. 14 shows the efficacy of mAb23-TM β-glu exatecan ADC or isotype control ADC in B-ALL patient derived xenografts after administration of the relevant antibody at 1 mg/kg or 2.4 mg/kg intravenously once every three weeks for a total of two doses (DO and D21). The percentage of CD45+ population (tumor cells) was plotted and shown ±SEM (n=10).

FIG. 15 shows the survival benefit of mAb23-TM β-glu exatecan ADC or isotype control ADC in B-ALL patient derived xenografts after administration of the relevant antibody at 1 mg/kg or 2.4 mg/kg intravenously once every three weeks for a total of two doses (D0 and D21).

FIG. 16 shows cytotoxic effect of mAb23-TM β-glu exatecan ADC and an anti-CD22 calicheamicin ADC on primary B cells which express CD22, and primary T cells which do not express CD22.

FIG. 17 shows the binding kinetics of mAb23-TM and a benchmark anti-CD22 antibody to human DLBCL cell lines WSU-DLCL2 (high CD22) and OCI-LY19 (Low CD22) assessed by flow cytometry. Cells were incubated with antibodies across a titration range, and fluorescence was quantified to determine binding characteristics. Both mAb23-TM and a benchmark anti-CD22 antibody demonstrated similar binding affinities, with apparent Kd values ranging from 1.55-1.93 nM for WSU-DLCL2 and 1.92-3.77 nM for OCI-LY19.

FIG. 18 shows the internalization of mAb23-TM and a benchmark anti-CD22 antibody in human DLBCL cell lines WSU-DLCL2 and OCI-LY19. Internalization rates of mAb23-TM and a benchmark anti-CD22 antibody in CD22-expressing DLBCL cell lines were measured using a live-cell fluorescence assay. Antibodies were conjugated to a pH-sensitive fluorophore and applied to cells plated in poly-L-lysine-coated wells. Hourly fluorescence imaging over 24 hours revealed that mAb23-TM exhibited significantly greater internalization compared to a benchmark anti-CD22 antibody at concentrations of 1 μg/ml and 0.5 μg/ml, indicating more efficient cellular uptake.

FIG. 19 shows the cytotoxicity analysis of mAb23-TM and a benchmark anti-CD22 antibody conjugated with β-Glu-exatecan linker payload at DAR8 in DLBCL cell lines. WSU-DLCL2 and OCI-LY19 DLBCL cells expressing CD22 were treated with the ADCs using a serial dilution range. After 6 days of incubation, cell viability was assessed by luminescence (CellTiter-Glo assay), and IC₅₀ values were determined. mAb23-TM conjugate demonstrated significantly higher cytotoxicity, with IC₅₀ fold changes of 4.6 (WSU-DLCL2) and 3.1 (OCI-LY19) relative to a benchmark anti-CD22 antibody conjugate (Table 9), indicating superior intracellular payload release.

### DETAILED DESCRIPTION

The human B-lymphocyte-restricted antigen CD22 (also referred to as Sialic Acid-Binding Ig-Like Lectin 2; SIGLEC-2) is expressed early in B-cell development in pro-B cells, as a cytoplasmic protein, and later in B-cell development, at the late pre-B-cell stage, as a cell surface protein. Once expressed as a membrane protein, CD22 persists on B cells until they differentiate into plasma cells. The presence of cytoplasmic CD22 is a useful marker for B-cell malignancies, including B-cell acute lymphocytic leukemia. CD22 is also expressed in normal tonsil, spleen, appendix, lymph nodes, and ovary.

CD22 is over-expressed in naïve B-cells, memory B-cells, and B-cell malignancies. This over-expression is consistent with the role of a cancer antigen. Therefore, this disclosure includes successfully generated antibodies that show high binding to CD22 expressing cells. Advantageously, the antibodies can target multiple different cancer cell types expressing CD22, exemplifying the broad utility of the antibodies as anti-cancer therapies. Furthermore, the antibodies can advantageously be linked/conjugated to suitable drugs/cytotoxins (e.g., to provide Antibody-drug conjugates (ADC)), thus increasing the potency of the antibodies as a therapy by allowing for targeted toxin delivery to cancer cells (e.g., B-cell acute lymphoblastic leukemia; B-cell lymphoblastic leukemia; B-cell non-Hodgkin's leukemia).

The term “about” is used herein to mean approximately, roughly, around, or in the regions of. When the term “about” is used in conjunction with a numerical range, it modifies that range by extending the boundaries above and below the numerical values set forth. In general, the term “about” is used herein to modify a numerical value above and below the stated value by a variance of 10 percent, up or down (higher or lower).

Throughout this disclosure, various aspects of this disclosure are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6, etc., as well as individual numbers within that range, for example, 1, 2, 3, 4, 5, and 6. This applies regardless of the breadth of the range. Numeric ranges recited are inclusive of the numbers defining the range and include each integer within the defined range.

Units, prefixes, and symbols are denoted in their Systeme International de Unites (SI) accepted form. Numeric ranges are inclusive of the numbers defining the range. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the disclosure. Thus, ranges recited herein are understood to be shorthand for all of the values within the range, inclusive of the recited endpoints. For example, a range of 1 to 10 is understood to include any number, combination of numbers, or sub-range from the group consisting of 1, 2, 3, 4, 5, 6, 7, 8, 9, and 10.

Where a value is explicitly recited, it is to be understood that values which are about the same quantity or amount as the recited value are also within the scope of the disclosure. Thus, any value recited herein is inclusive of a precise value, as well as values which are about the same as the precise value. Where a combination is disclosed, each subcombination of the elements of that combination is also specifically disclosed and is within the scope of the disclosure. Conversely, where different elements or groups of elements are individually disclosed, combinations thereof are also disclosed. Where any element of a disclosure is disclosed as having a plurality of alternatives, examples of that disclosure in which each alternative is excluded singly or in any combination with the other alternatives are also hereby disclosed; more than one element of a disclosure can have such exclusions, and all combinations of elements having such exclusions are hereby disclosed.

The term “pharmaceutical composition” refers to a preparation that is in such form as to permit the biological activity of the active ingredient to be effective, and which contains no additional components which are unacceptably toxic to a subject to which the composition would be administered. Such composition can be sterile, and can comprise a pharmaceutically acceptable carrier, such as physiological saline.

Furthermore, the antibody or antigen binding fragment thereof of the disclosure has been demonstrated to target and have a cytotoxic effect on malignant B-cell lines (e.g., diffuse large B-cell lymphoma (DLBCL), follicular lymphoma (FL), mantle cell lymphoma (MCL) and B-cell acute lymphoblastic lymphoma (B-ALL) cell lines) exhibiting potent cell-killing activity across these malignant B-cell lines in vivo. Thus, the disclosure embraces the above defined antibody or antigen binding fragment thereof and the above defined pharmaceutical composition for use in a method of treating cancer. In certain aspects, the cancer comprises a cancer cell which expresses CD22.

“Cancer” as used herein can encompass all types of oncogenic processes and/or cancerous growths. In this disclosure, cancer can include, but is not limited to primary tumors as well as metastatic tissues or malignantly transformed cells, tissues, or organs. Cancer can encompass histopathologies and stages, e.g., stages of invasiveness/severity, of a cancer. Cancer can include relapsed and/or resistant cancer. The terms “cancer” and “tumor” can be used interchangeably. For example, both terms encompass solid and liquid tumors. As used herein, the term “cancer” or “tumor” includes premalignant, as well as malignant cancers and tumors.

In one aspect there is provided an anti-CD22 antibody or antigen binding fragment thereof, comprising a heavy chain CDR1 (HCDR1) comprising the amino acid sequence of SEQ ID NO: 1, a heavy chain CDR2 (HCDR2) comprising the amino acid sequence of SEQ ID NO: 2, a heavy chain CDR3 (HCDR3) comprising the amino acid sequence of SEQ ID NO: 3, a light chain CDR1 (LCDR1) comprising the amino acid sequence of SEQ ID NO: 4, a light chain CDR2 (LCDR2) comprising the amino acid sequence of SEQ ID NO: 5, and a light chain CDR3 (LCDR3) comprising the amino acid sequence of SEQ ID NO: 6.

In some aspects, the anti-CD22 antibody or antigen binding fragment thereof comprises a variable heavy (VH) chain at least 90%, at least 91% at least 92%, at least 93%, at least 94%, at least 95%, at least 96%, at least 97%, at least 98%, or at least 99% identical to the amino acid sequence of SEQ ID NO: 7, and a variable light (VL) chain at least 90%, at least 91% at least 92%, at least 93%, at least 94%, at least 95%, at least 96%, at least 97%, at least 98%, or at least 99% identical to the amino acid sequence of SEQ ID NO: 8.

In some aspects, the anti-CD22 antibody or antigen binding fragment thereof comprises a VH chain comprising the amino acid sequence of SEQ ID NO: 7 and a VL chain comprising the amino acid sequence of SEQ ID NO: 8.

In some aspects, anti-CD22 the antibody or binding fragment thereof comprises an Fc region.

In some aspects, anti-CD22 antibody or antigen binding fragment thereof comprises an Fc region comprising one or more mutations, which has reduced antibody dependent cellular cytotoxicity (ADCC) compared to an antibody having a wild-type Fc region.

In some aspects, the Fc region comprises a L234F/L235E/P331S (EU Index numbering) triple mutation (TM).

In some aspects, the anti-CD22 antibody or antigen binding fragment thereof comprises a constant heavy chain comprising the amino acid sequence of SEQ ID NO: 11 and a constant light chain amino comprising the acid sequence of SEQ ID NO: 12.

In some aspects, the anti-CD22 antibody or antigen binding fragment thereof comprises a heavy chain region comprising the amino acid sequence of SEQ ID NO: 9.

In some aspects, the anti-CD22 antibody or antigen binding fragment thereof comprises a light chain region comprising the amino acid sequence of SEQ ID NO: 10.

In some aspects, the anti-CD22 antibody or antigen binding fragment thereof comprises a heavy chain comprising the amino acid sequence of SEQ ID NO: 9; and a light chain comprising the amino acid sequence of SEQ ID NO: 10.

In some aspects, the anti-CD22 antibody or antigen binding fragment thereof comprises a heavy chain comprising the amino acid sequence of SEQ ID NO: 13; and a light chain comprising the amino acid sequence of SEQ ID NO: 10.

In some aspects, the anti-CD22 antigen binding fragment is a single chain Fv, Fab fragment, a Fab′ fragment, or a F(ab′)2 fragment.

In some aspects, the anti-CD22 antibody or antigen-binding fragment thereof is humanized, chimeric, or fully human.

In some aspects, the anti-CD22 antibody or antigen-binding fragment thereof is fully human.

In some aspects, the anti-CD22 antibody or antigen-binding fragment thereof is a monoclonal antibody.

In some aspects, the anti-CD22 antibody or antigen-binding fragment thereof is an IgG1.

In some aspects, the anti-CD22 antibody or antigen binding fragment thereof binds a B-cell derived cell line.

Certain definitions and aspects will now be outlined. It should be understood that the following definitions and aspects may pertain to any aspect described herein, e.g., any method, composition, and/or composition for use in therapy described herein.

The term “epitope” refers to a target protein region (e.g., polypeptide) capable of binding to (e.g., being bound by) an antibody or antigen binding fragment of the disclosure.

CD22 (CD22 antigen or SIGLEC-2) can act as a stimulatory and an inhibitory receptor due to the ITAM-like regions and ITIMS motifs located in its cytoplasmic tail. Upon BCR engagement, CD22 is able to hinder the resulting calcium signaling by recruiting the phosphatase SHP2. However, engagement of CD22 triggers B-cell proliferation in human B-cells and this proliferation is even greater when the BCR is stimulated. The human B-lymphocyte-restricted antigen CD22 is expressed early in B-cell development in pro-B cells, as a cytoplasmic protein, and later in B-cell development, at the late pre-B-cell stage, as a cell surface protein. Once expressed as a membrane protein, CD22 persists on B-cells until they differentiate into plasma cells. The presence of cytoplasmic CD22 is a useful marker for B-cell malignancies, including B-cell precursor acute lymphocytic leukemia. CD22 appears to be a heterodimer consisting of 130- and 140-kD glycoproteins with protein cores of 80 and 100 kD, respectively. Consistent with the structural similarities to the adhesion molecules, CD22 participates in adhesion between B-cells and other cell types. CD22 is also known as B-cell receptor CD22, B-lymphocyte cell adhesion molecule (BL-CAM), CD22 antigen, T-cell surface antigen Leu-14, and sialic acid-binding Ig-like lectin 2 (SIGLEC-2).

Without wishing to be bound by theory, CD22 is understood to be expressed on cells of various B-cell malignancies. As such, the ability of the claimed antibody to target, and optionally deliver a cytotoxin to a CD22 expressing cell renders said antibody particularly suitable for use in cancer therapy. Furthermore, CD22 expression is not limited to a particular cancer type, such that it can represent a target antigen for treating a broad spectrum of cancer types.

The RNA, DNA, and amino acid sequences of CD22 are known to those skilled in the art and can be found in many databases, for example, in the databases of the National Center for Biotechnology Information (NCBI) and UniProt. Examples of these sequences found at UniProt are at P20273 for human CD22; NCBI Gene ID 933; NCBI RefSeq NM_001771.

In one aspect, the anti-CD22 antibody or antigen binding fragment thereof, comprises a heavy chain CDR1 (HCDR1) comprising the amino acid sequence of SEQ ID NO: 1, a heavy chain CDR2 (HCDR2) comprising the amino acid sequence of SEQ ID NO: 2, a heavy chain CDR3 (HCDR3) comprising the amino acid sequence of SEQ ID NO: 3, a light chain CDR1 (LCDR1) comprising the amino acid sequence of SEQ ID NO: 4, a light chain CDR2 (LCDR2) comprising the amino acid sequence of SEQ ID NO: 5, and a light chain CDR3 (LCDR3) comprising the amino acid sequence of SEQ ID NO: 6.

Additionally or alternatively, an antibody or antigen binding fragment thereof described herein may be described by means of a variable heavy (VH) chain and a variable light (VL) chain thereof.

In some aspects, the anti-CD22 antibody or antigen binding fragment thereof comprises a VH chain comprising the amino acid sequence of SEQ ID NO: 7 and a VL chain comprising the amino acid sequence of SEQ ID NO: 8.

A variable heavy (VH) chain sequence (which the antibody or antigen binding fragment thereof may comprise) can comprise an amino acid sequence of SEQ ID NO: 7 (or a functional variant thereof).

A variable light (VL) chain sequence (which the antibody or antigen binding fragment thereof may comprise) can comprise an amino acid sequence of SEQ ID NO: 8 (or a functional variant thereof).

For example, in one aspect, the anti-CD22 antibody or antigen binding fragment thereof comprises a variable heavy (VH) chain at least 90%, at least 91% at least 92%, at least 93%, at least 94%, at least 95%, at least 96%, at least 97%, at least 98%, or at least 99% identical to the amino acid sequence of SEQ ID NO: 7, and a variable light (VL) chain at least 90%, at least 91% at least 92%, at least 93%, at least 94%, at least 95%, at least 96%, at least 97%, at least 98%, or at least 99% identical to the amino acid sequence of SEQ ID NO: 8.

Additionally or alternatively, an antibody or antigen binding fragment thereof described herein may be described by means of a heavy chain and/or light chain thereof.

In one aspect, the anti-CD22 antibody or antigen binding fragment thereof comprises a constant heavy chain comprising the amino acid sequence of SEQ ID NO: 11 and a constant light chain comprising the amino acid sequence of SEQ ID NO: 12.

In one aspect, the anti-CD22 antibody or antigen binding fragment thereof comprises a heavy chain region comprising the amino acid sequence of SEQ ID NO: 9.

In one aspect, the anti-CD22 antibody or antigen binding fragment thereof comprises a light chain region comprising the amino acid sequence of SEQ ID NO: 10.

In one aspect, the anti-CD22 antibody or antigen binding fragment thereof comprises a heavy chain comprising the amino acid sequence of SEQ ID NO: 9; and a light chain comprising the amino acid sequence of SEQ ID NO: 10.

In one aspect, the anti-CD22 antibody or antigen binding fragment thereof comprises a heavy chain comprising the amino acid sequence of SEQ ID NO: 13; and a light chain comprising the amino acid sequence of SEQ ID NO: 10.

In one aspect, the antibody or antigen binding fragment thereof comprises a heavy chain (e.g., comprising a VH and constant heavy chain) comprising an amino acid sequence having at least 70%, 75%, 80%, 90%, 95%, or 100% sequence identity to a reference amino acid sequence of SEQ ID NO: 9. For example, the antibody or antigen binding fragment thereof may comprise a heavy chain (e.g., comprising a VH and constant heavy chain) comprising the amino acid sequence of SEQ ID NO: 9.

In one aspect, the antibody or antigen binding fragment thereof comprises a heavy chain (e.g., comprising a VH and constant heavy chain) comprising an amino acid sequence having at least 70%, 75%, 80%, 90%, 95%, or 100% sequence identity to a reference amino acid sequence of SEQ ID NO: 13. For example, the antibody or antigen binding fragment thereof may comprise a heavy chain (e.g., comprising a VH and constant heavy chain) comprising the amino acid sequence of SEQ ID NO: 13.

In one aspect, the antibody or antigen binding fragment thereof comprises a light chain (e.g., comprising a VL and constant light chain) comprising an amino acid sequence having at least 70%, 75%, 80%, 90%, 95%, or 100% sequence identity to the amino acid sequence of SEQ ID NO: 10. In one aspect, the antibody or antigen binding fragment thereof comprises a light chain (e.g., comprising a VL and constant light chain) comprising the amino acid sequence of SEQ ID NO: 10.

Disclosed herein, is an antibody (or antigen binding fragment) thereof having affinity and specificity for a clinically relevant target and has demonstrated a unique advantage (e.g., unexpected technical effect) associated therewith.

An antibody or antigen binding fragment thereof described herein is capable of binding to CD22 as an integral component of a cancer cell (for example, CD22 as an integral component of a cell membrane of a cancer cell).

The antibody binding affinity can be measured by any suitable method of measuring binding affinity described herein or known to a person of ordinary skill in the arts.

Suitably, the antibody or antigen binding fragment of the disclosure binds to CD22 molecule with sufficient affinity such that the antibody is useful as a therapeutic agent or a diagnostic reagent in targeting CD22.

In one aspect, the antibody or antigen binding fragment thereof binds to CD22 (e.g., human CD22) with a dissociation constant (KD) of ≤500 nM, ≤300 nM or ≤280 nM. In one aspect, the antibody or antigen binding fragment thereof binds to CD22 (e.g., human CD22) with a KD of between about 200 nM to about 300 nM, between about 250 nM to about 300 nM, or between about 260 nM to about 280 nM.

The KD measurements (binding affinity) may be carried out by any suitable assay known in the art. Suitable assays include an affinity assay performable via a KinExA system (e.g., KinExA 3100, KinExA 3200, or KinExA 4000) (Sapidyne Instruments, Idaho), or ForteBio Octet system.

In one aspect, the extent of binding of an antibody or antigen binding fragment thereof of the disclosure to an unrelated, non-CD22 protein is less than about 10%, 5%, 2%, or 1% (e.g., less than about 10%) of the binding of the antibody (or antigen binding fragment thereof) to CD22 (e.g., human CD22). Said binding may be measured, e.g., by a radioimmunoassay (RIA), BIACORE (using recombinant CD22 as the analyte and antibody as the ligand, or vice versa), KINEXA, ForteBio Octet system, or other binding assays known in the art.

In one aspect, the CD22 polypeptide is comprised within a CD22 polypeptide sequence, or a fragment thereof.

A “CD22 polypeptide” may comprise the full length polypeptide sequence of CD22 (e.g., SEQ ID NO: 17), or may comprise a fragment of CD22 of any length of the full length polypeptide sequence of CD22 (e.g., comprising a polypeptide sequence of 5%, 15%, 25%, 35%, 45%, 55%, 65%, 75%, 85%, or 95% of the full length polypeptide sequence of CD22) which comprises an epitope which can bind (e.g., be bound by) an antibody or antigen binding fragment of the disclosure. The CD22 polypeptide may comprise a sequence having 75%, 80%, 85%, 90%, or 90% sequence identity to the sequence of SEQ ID NO: 17. The CD22 polypeptide can comprise the sequence of SEQ ID NO: 17.

The antibody or antigen binding fragment has high affinity for CD22 both in vitro an in vivo, and thus may advantageously be used in methods for detecting a CD22 epitope, and associated methods of diagnosis.

As described above, an antibody or antigen binding fragment thereof of the disclosure may be comprised within a pharmaceutical composition. The pharmaceutical composition may comprise one or more pharmaceutically acceptable excipient(s). In one aspect, a pharmaceutical composition of the disclosure can comprise a pharmaceutically acceptable, non-toxic, sterile carrier such as physiological saline, non-toxic buffers, preservatives and the like. Suitable formulations for use in the therapeutic methods disclosed herein are described in Remington's Pharmaceutical Sciences, 22nd ed., Ed. Lloyd V. Allen, Jr. (2012).

In one aspect, a pharmaceutical composition of the disclosure may be comprised within one or more formulations that is a capsule, a tablet, an aqueous suspension, a solution, a nasal aerosol, or a combination thereof.

In one aspect, the pharmaceutical composition comprises more than one type of antibody or antigen binding fragment of the disclosure. For example, a pharmaceutical composition may comprise two or more of an antibody, an antigen-binding fragment, an antibody or antigen binding fragment thereof conjugated to a cytotoxin, or a combination thereof.

The term “a pharmaceutically effective amount” of an antibody or antigen-binding fragment means an amount sufficient to achieve effective binding to a target and to achieve a benefit, e.g., to ameliorate symptoms of a disease or condition or to detect a substance or a cell.

In one aspect, a pharmaceutical composition may comprise a buffer (e.g., acetate, phosphate or citrate buffer), a surfactant (e.g., polysorbate), optionally a stabilizer agent (e.g., human albumin), etc.

To “treat” refers to therapeutic measures that cure, slow down, alleviate symptoms of, and/or halt progression of a diagnosed pathologic condition or disorder. Thus, those in need of treatment include those already with the disorder. In one aspect, a subject is successfully “treated” for a disease or disorder (e.g., cancer), according to the methods provided herein if the patient shows, e.g., total, partial, or transient alleviation or elimination of symptoms associated with the disease or disorder (e.g., cancer).

In one aspect, a method of the disclosure may be used to prevent the onset of a cancer comprising a cancer cell which expresses CD22. To “prevent” refers to prophylactic or preventative measures that prevent and/or slow the development of a targeted pathologic condition or disorder. Thus, those in need of prevention include those prone to have or susceptible to the disorder. In one aspect, a disease or disorder (e.g., cancer) is successfully prevented according to the methods provided herein if the patient develops, transiently or permanently, e.g., fewer or less severe symptoms associated with the disease or disorder, or a later onset of symptoms associated with the disease or disorder, than a patient who has not been subject to the methods of the disclosure.

The terms “subject”, “individual”, and “patient” are used interchangeably herein to refer to a mammalian subject. In one aspect the “subject” is a human, domestic animals, farm animals, sports animals, and zoo animals, e.g., humans, non-human primates, dogs, cats, guinea pigs, rabbits, rats, mice, horses, cattle, etc. In one aspect, the subject is a cynomolgus monkey (Macaca fascicularis). In one aspect, the subject is a human. In methods of the disclosure, the subject may not have been previously diagnosed as having cancer. Alternatively, the subject may have been previously diagnosed as having cancer. The subject may also be one who exhibits disease risk factors, or one who is asymptomatic for cancer. The subject may also be one who is suffering from or is at risk of developing cancer. Thus, in one aspect, a method of the disclosure may be used to confirm the presence of cancer in a subject. For example, the subject may previously have been diagnosed with cancer by alternative means. In one aspect, the subject has been previously administered a cancer therapy.

In one aspect, methods of treatment of the disclosure comprise one or more administration steps that is intravenous, intraarterial, intramuscular, subcutaneous, or a combination thereof. In one aspect, the administration is intravenous or intraarterial (e.g., by injection or drip), or a combination thereof. In another aspect the administration is subcutaneous.

In one aspect, a cancer referred to herein is a cancer characterized by the expression (e.g., overexpression) of a CD22 molecule. In other words, a cancer referred to herein may comprise a cancerous cell that expresses CD22. Said cancerous cell may be comprised within a tumor or a B-cell malignancy.

In one aspect, the cancer is a B-cell malignancy. As used herein, the term “B-cell malignancy” refers to a type of cancer that forms in B-cells. For example, a B-cell malignancy can be a non-Hodgkin lymphoma. For example, a B-cell malignancy can be Burkitt lymphoma, chronic lymphocytic leukemia (CLL), small lymphocytic lymphoma (SLL), diffuse large B-cell lymphoma (DLBCL), follicular lymphoma (FL), mantle cell lymphoma (MCL), lymphoplasmacytic lymphoma, or Hairy cell leukemia.

In one aspect, the B-cell malignancy is selected from the group consisting of non-Hodgkin lymphoma, acute lymphoblastic leukemia (ALL), Burkitt lymphoma, chronic lymphocytic leukemia (CLL), small lymphocytic lymphoma (SLL), diffuse large B-cell lymphoma (DLBCL), follicular lymphoma (FL), mantle cell lymphoma (MCL), lymphoplasmacytic lymphoma, and Hairy cell leukemia.

In one aspect, the B-cell malignancy is non-Hodgkin lymphoma. In another aspect, the B-cell malignancy is acute lymphoblastic leukemia (ALL). In another aspect, the B-cell malignancy is Burkitt lymphoma. In another aspect, the B-cell malignancy is chronic lymphocytic leukemia (CLL). In another aspect, the B-cell malignancy is small lymphocytic lymphoma (SLL). In another aspect, the B-cell malignancy is diffuse large B-cell lymphoma (DLBCL). In another aspect, the B-cell malignancy is follicular lymphoma (FL). In another aspect, the B-cell malignancy is mantle cell lymphoma (MCL). In another aspect, the B-cell malignancy is lymphoplasmacytic lymphoma. In a further aspect, the B-cell malignancy is Hairy cell leukemia.

In one aspect, the B-cell malignancy is selected from the group consisting of non-Hodgkin lymphoma (NHL), acute lymphoblastic leukemia (ALL), diffuse large B cell lymphoma (DLBCL), follicular lymphoma (FL), and chronic lymphocytic leukemia (CLL).

In one aspect, the B-cell malignancy is refractory B-cell acute lymphoblastic leukemia (ALL) or relapsed B-cell acute lymphoblastic leukemia (ALL). The B-cell malignancy may be refractory B-cell acute lymphoblastic leukemia (ALL). The B-cell malignancy may be relapsed B-cell acute lymphoblastic leukemia (ALL).

An antibody or antigen binding fragment thereof also finds utility in detecting a cancer cell, for example as part of a diagnostic method.

In a further aspect, there is provided a method for detecting the presence or absence of a CD22 polypeptide (e.g., a CD22 polypeptide epitope) in a sample, comprising:
(a) contacting a sample with an antibody or antigen binding fragment thereof, or a pharmaceutical composition comprising an antibody or antigen binding fragment thereof, to provide an antibody-antigen complex; wherein said antibody or antigen binding fragment thereof comprises a heavy chain CDR1 (HCDR1), a heavy chain CDR2 (HCDR2), a heavy chain CDR3 (HCDR3), a light chain CDR1 (LCDR1), a light chain CDR2 (LCDR2), and a light chain CDR3 (LCDR3) comprising the amino acid sequence of SEQ ID NO: 1, SEQ ID NO: 2, SEQ ID NO: 3, SEQ ID NO: 4, SEQ ID NO: 5, and SEQ ID NO: 6, respectively, or a functional variant thereof;
(b) detecting the presence or absence of said antibody-antigen complex; and
(c) wherein the presence of the antibody-antigen complex confirms the presence of a CD22 polypeptide (e.g., CD22 polypeptide epitope); or
(d) wherein the absence of the antibody-antigen complex confirms the absence of CD22 polypeptide (e.g., CD22 polypeptide epitope).

In a related aspect, there is provided a method for detecting the presence or absence of a cancer cell expressing a CD22 polypeptide (e.g., CD22 polypeptide epitope) in a sample, comprising:
(a) contacting a sample with an antibody or antigen binding fragment thereof, or a pharmaceutical composition comprising an antibody or antigen binding fragment thereof, to provide an antibody-antigen complex; wherein said antibody or antigen binding fragment thereof comprises a heavy chain CDR1 (HCDR1), a heavy chain CDR2 (HCDR2), a heavy chain CDR3 (HCDR3), a light chain CDR1 (LCDR1), a light chain CDR2 (LCDR2), and a light chain CDR3 (LCDR3) comprising the amino acid sequence of SEQ ID NO: 1, SEQ ID NO: 2, SEQ ID NO: 3, SEQ ID NO: 4, SEQ ID NO: 5, and SEQ ID NO: 6, respectively, or a functional variant thereof;
(b) detecting the presence or absence of said antibody-antigen complex; and
(c) wherein the presence of the antibody-antigen complex confirms the presence of a CD22 polypeptide (e.g., CD22 polypeptide epitope); or
(d) wherein the absence of the antibody-antigen complex confirms the absence of CD22 polypeptide (e.g., CD22 polypeptide epitope).

The disclosure embraces a corresponding use of the antibody or antigen binding fragment thereof of the disclosure for detecting a CD22 polypeptide (e.g., CD22 polypeptide epitope).

In one aspect, the presence of antibody-antigen complex is indicative of the presence of a cancer cell, and the absence of the antibody-antigen complex is indicative of the absence of a cancer cell. For example, the method may comprise confirming the presence of cancer where an antibody-antigen complex is detected, or not confirming the presence of cancer where an antibody-antigen complex is not detected.

In one aspect, the cancer cell is a cancer cell expressing a CD22 polypeptide (e.g., CD22 polypeptide epitope).

Thus, the present disclosure embraces corresponding use of the method steps described herein in methods of diagnosing a subject with a cancer, wherein said cancer comprises a CD22 expressing cancer cell.

In one aspect, a method of detection or method of diagnosis may comprise measuring the expression level of CD22 on a cell (or tissue) obtainable from a subject, and comparing the measured expression level with a standard CD22 expression in a control cell (or tissue), wherein an increase in the expression level compared to the control is indicative of the presence of cancer. Said control sample can comprise a non-cancer (e.g., normal) cell.

An “antibody-antigen complex” means a complex (e.g., macromolecular complex) comprising a CD22 antigen which has become bound to an antibody. The term “antibody-antigen complex” may be used synonymously with the terms “bound CD22-antibody complex” and “antibody bound to a CD22”.

An antibody-antigen complex may be detected by any means known to the skilled person. In one aspect, the antibody (or antigen binding fragment thereof) is labelled with a detectable label. Said label may be an epi-fluorescent label.

In one aspect, an antibody-antigen complex is detected by means of a secondary (e.g., detection) antibody which binds the antibody and/or antibody-antigen complex.

Suitably, said secondary antibody comprises a detection means, such as a tag/label to aid detection. Said detection means is conjugated to the secondary antibody. Examples of suitable labels include detectable labels such as radiolabels or fluorescent or colored molecules, enzymatic markers or chromogenic markers—e.g., dyes that provide a visible color change upon binding of the detection antibody to an antigen. By way of example, the label may be fluorescein-isothiocyanate (FITC), R-phycoerythrin, Alexa 532, CY3 or digoxigenin. The label may be a reporter molecule, which is detected directly, such as by detecting its fluorescent signal, or by exposure of the label to photographic or X-ray film. Alternatively, the label is not directly detectable, but may be detected, for example, in a two-phase system. An example of indirect label detection is binding of an antibody to the label.

In another aspect, said secondary antibody comprises a fluorescent tag, and an antibody-antigen complex is detected by the florescence emitted from an antibody-antigen-secondary antibody complex. An “antibody-antigen-secondary antibody complex” means a complex comprising an antigen (e.g., CD22) which has become bound to an antibody, wherein said complex has further become bound by a secondary antibody which binds said antibody and/or antibody-antigen complex.

Suitably, an antibody-antigen complex is detected when the signal (e.g., fluorescence) emitted from the detection label is greater than the signal detected in a control comprising no antibody (e.g., no antibody which binds a CD22). Said control may alternatively comprise a CD22, but the sample is not applied to said control.

Suitably, a “sample” is a sample obtained from a subject (e.g., biopsy), cell line, tissue culture, or other source of cells potentially expressing CD22. In one aspect, a sample is a biopsy from a subject. Said biopsy may be taken from a tumor, or a site at risk of developing a tumor.

In one aspect, the sample is an isolated sample obtainable (e.g., obtained) from a subject.

In another aspect, the CD22 polypeptide (e.g., CD22 polypeptide epitope) is an integral component of a cancer cell, for example, an integral component of the cell membrane of a cancer cell.

### Antibody-Drug Conjugates (ADCs)

An antibody or antigen binding fragment thereof of the disclosure may comprise a heterologous agent. In one aspect, an antibody or antigen binding fragment of the disclosure is linked to a heterologous agent. In another aspect, the antibody or antigen binding fragment is conjugated to a heterologous agent. Suitably, “conjugated” means linked via a covalent or ionic bond. In some aspects, said heterologous agent is a cytotoxin.

The heterologous agent may simply be referred to as an “agent” or “active agent”. For example, in alternative language, an antibody or antigen binding fragment thereof of the disclosure may comprise an active agent. In one aspect, an antibody or antigen binding fragment of the disclosure is linked to an active agent. In another aspect, the antibody or antigen binding fragment is conjugated to an active agent.

The heterologous/active agent can be a drug. In some aspects, the heterologous/active is a cytotoxin or a cytotoxic drug or a cytotoxic agent.

In some aspects, the anti-CD22 antibody or antigen binding fragment thereof of the disclosure is linked (e.g., conjugated) to a heterologous/active agent in methods of treatment, as described below.

In one aspect, the anti-CD22 antibody or antigen binding fragment thereof is conjugated to one or more drugs selected from the group consisting of a cytotoxin, a tubulysin derivative, an antimicrobial agent, a therapeutic agent, a prodrug, a peptide, a protein, an enzyme, a lipid, a biological response modifier, a pharmaceutical agent, a lymphokine, a heterologous antibody, a fragment of a heterologous antibody, a detectable label, a polyethylene glycol (PEG), a radioisotope, a degrader, and a combination thereof.

An agent and/or cytotoxin of the disclosure may be conjugated to the antibody or antigen binding fragment thereof by means of a spacer (e.g., at least one spacer). In one aspect, the spacer is a peptide spacer. In one aspect, the spacer is a non-peptide (e.g., chemical) spacer.

The cytotoxic agent or cytotoxin can be any molecule known in the art that inhibits or prevents the function of cells and/or causes destruction of cells (cell death), and/or exerts anti-neoplastic/anti-proliferative effects. A number of classes of cytotoxic agents are known to have potential utility in ADC molecules. These include, but are not limited to, topoisomerase I inhibitors, amanitins, auristatins, daunomycins, doxorubicins, duocarmycins, dolastatins, enediynes, lexitropsins, taxanes, puromycins, maytansinoids, vinca alkaloids, and tubulysins. Examples of such cytotoxic agents are AFP, MMAF, MMAE, AEB, AEVB, auristatin E, paclitaxel, docetaxel, CC-1065, SN-38, topotecan, morpholino-doxorubicin, rhizoxin, cyanomorpholino-doxorubicin, dolastatin-10, echinomycin, combretatstatin, chalicheamicin, maytansine, DM-1, vinblastine, methotrexate, and netropsin, and derivatives and analogs thereof. Additional disclosure regarding cytotoxins suitable for use in ADCs can be found, for example, in International Publication Nos. WO 2015/155345 and WO 2015/157592, incorporated by reference herein in their entirety.

In some aspects, the anti-CD22 antibody or antigen binding fragment thereof is conjugated to one or more drugs that is a cytotoxin selected from the group consisting of a DNA scission agent, a DNA binding agent, a DNA intercalating agent, a topoisomerase I inhibitor, a topoisomerase II inhibitor, a tubulysin derivative, and an antimitotic agent. In some aspects, the one or more drugs is a cytotoxin which is a topoisomerase I inhibitor.

In some aspects, the one or more drugs is a cytotoxin selected from the group consisting of calicheamicins, pyrrolobenzodiazepines, camptothecins, daunorubicins, doxorubicins, auristatins, and maytansinoids.

For example, the anti-CD22 antibody or antigen binding fragment may be conjugated to such heterologous agent or cytotoxic agent to provide an “antibody-drug conjugate” (ADC). In some aspects, the heterologous agent or cytotoxic agent is exatecan.

“Exatecan” as used herein, refers to the following structure:

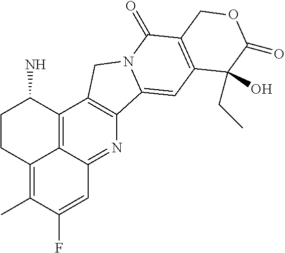 [SMILES: CC[C@@]1(O)C(=O)OCc2c1cc1n(c2=O)Cc2c-1nc1cc(F)c(C)c3c1c2[C@@H](N)CC3]

In some aspects the heterologous agent or cytotoxic agent is exatecan and the linker is a β-glu linker “β-Glu exatecan” refers to the following conjugated structure of Formula (ICC):

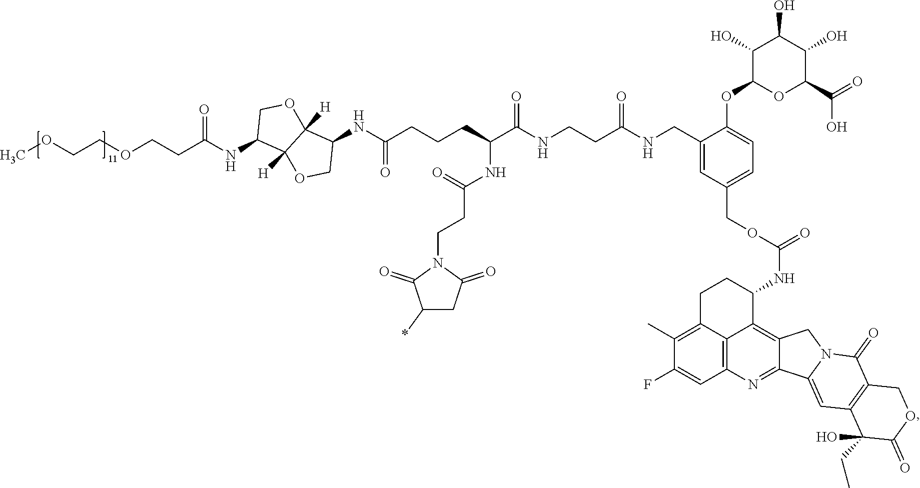

wherein [symbol] indicates the point of attachment to the antibody or antigen-binding fragment thereof.

Alternatively, “unconjugated β-Glu exatecan” refers to the following structure:

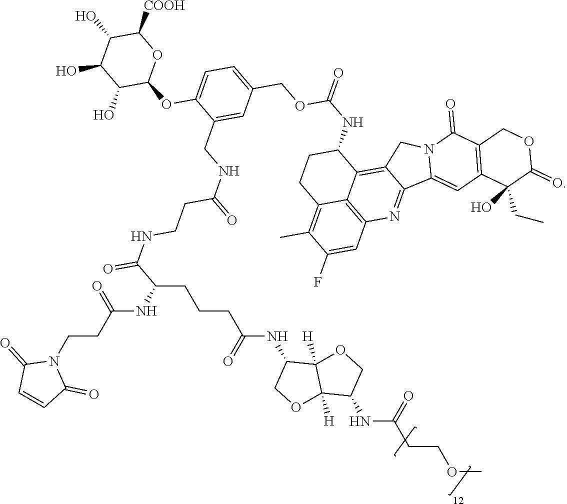

For example, the anti-CD22 antibody or antigen binding fragment may be conjugated to such heterologous agent or cytotoxic agent to provide an “antibody-drug conjugate” (ADC). In some aspects, the heterologous agent or cytotoxic agent is exatecan.

“TOP1i ('0132)” as used herein, refers to the following structure:

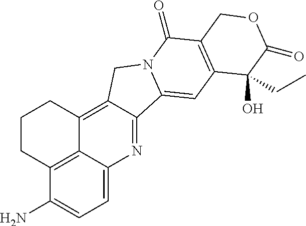 [SMILES: CC[C@@]1(O)C(=O)OCc2c1cc1n(c2=O)Cc2c-1nc1ccc(N)c3c1c2CCC3]

In some aspects, the heterologous agent or cytotoxic agent is TOP1i ('0132) and the linker is a Val-Ala linker “Val-Ala-TOP1i ('0132)” refers to the following conjugated structure of Formula (ICG):

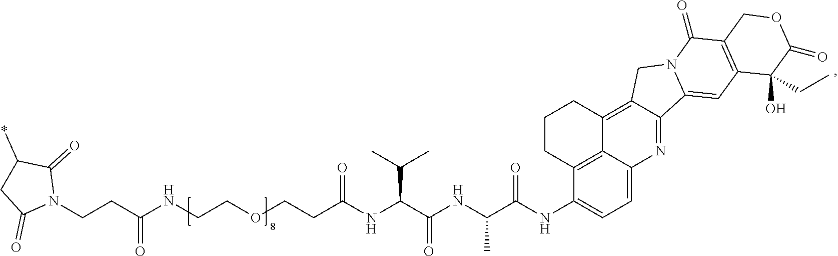

wherein [symbol] indicates the point of attachment to the antibody or antigen-binding fragment thereof.

Alternatively, “unconjugated Val-Ala-TOP1i ('0132)” refers to the following structure:

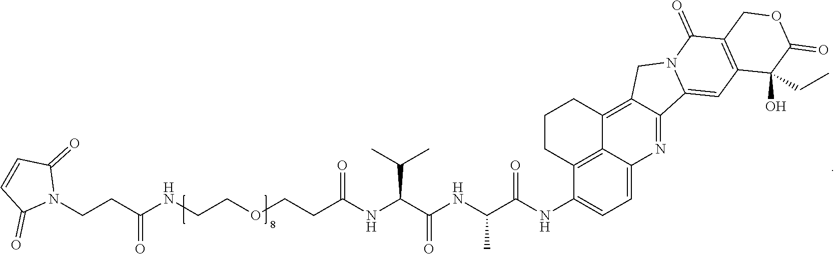

The agent is typically linked to, or “loaded onto” the antibody or antigen-binding fragment. The agent loading (p) is the average number of agent(s) per antibody or antigen-binding fragment (e.g., the Ligand unit).

The average number of agents per antibody (or antigen-binding fragment) in preparations of ADCs from conjugation reactions may be characterized by conventional means such as UV, reverse phase HPLC, HIC, mass spectroscopy, ELISA assay, and electrophoresis. The quantitative distribution of ADC in terms of p may also be determined. By ELISA, the averaged value of p in a particular preparation of ADC may be determined (Hamblett et al., Clin. Cancer Res. 10: 7063-70 (2004); Sanderson et al., Clin. Cancer Res. 11: 843-52 (2005)). In some instances, separation, purification, and characterization of homogeneous ADC, where p is a certain value from ADC with other drug loadings, may be achieved by means such as reverse phase HPLC or electrophoresis. Such techniques are also applicable to other types of conjugates.

Cysteine amino acids may be engineered at reactive sites in an antibody (or antigen-binding fragment thereof) and which do not form intrachain or intermolecular disulfide linkages (Junutula et al., Nature Biotech., 26(8): 925-32 (2008b); Dornan et al., Blood 114(13): 2721-29 (2009); U.S. Pat. Nos. 7,521,541 and 7,723,485; International Publication No. WO 2009/052249). The engineered cysteine thiols may react with a linker within an agent (e.g., of formula I below) which may have thiol-reactive, electrophilic groups such as maleimide or alpha-halo amides to form ADC with cysteine engineered antibodies. The location of the drug unit can thus be designed, controlled, and known. The drug loading can be controlled since the engineered cysteine thiol groups typically react with drug-linker reagents in high yield. Engineering an IgG antibody to introduce a cysteine amino acid by substitution at a single site on the heavy or light chain gives two new cysteines on the symmetrical antibody. A drug loading near 2 can be achieved with near homogeneity of the conjugation product ADC.

Where more than one nucleophilic or electrophilic group of the antibody or antigen binding fragment thereof reacts with an agent, then the resulting product may be a mixture of ADC compounds with a distribution of agent units attached to an antibody, e.g., 1, 2, 3, etc. Liquid chromatography methods such as polymeric reverse phase (PLRP) and hydrophobic interaction (HIC) may separate compounds in the mixture by agent loading value. Preparations of ADC with a single agent loading value (p) may be isolated.

Thus, the antibody-drug conjugate compositions of the disclosure may include mixtures of antibody-drug conjugates where the antibody or antigen binding fragment thereof has one or more agent moieties and where the agent moieties may be attached to the antibody or antigen binding fragment thereof at various amino acid residues.

In one aspect, the average number of agents per antibody (or antigen-binding fragment thereof) is in the range 1 to 20. In some aspects, the range is selected from 1 to 10, 2 to 10, 2 to 8, 2 to 6, and 4 to 10. In some aspects, there is one agent per antibody (or antigen-binding fragment thereof). In some aspects, the number of agents per antibody (or antigen-binding fragment thereof) can be expressed as a ratio of agent (i.e., drug) to antibody. This ratio is referred to as the Drug to Antibody Ratio (DAR). The DAR is the average number of drugs (i.e., agents/cytotoxins) linked to each antibody. In one aspect of the present disclosure, the DAR is in the range 1 to 20. In some aspects, the range of DAR is selected from 1 to 10, 2 to 10, 2 to 8, 2 to 6, and 4 to 10. In a particular aspect of the present disclosure, the DAR is about 8. In a particular aspect of the present disclosure, the DAR is 8. In a particular aspect of the present disclosure, the DAR is about 4. In a particular aspect of the present disclosure, the DAR is 4.

In one aspect, the antibody or antigen-binding fragment is conjugated to one or more heterologous agent selected from the group consisting of a topoisomerase I inhibitor, a tubulysin derivative, an antimicrobial agent, a therapeutic agent, a prodrug, a peptide, a protein, an enzyme, a lipid, a biological response modifier, a pharmaceutical agent, a lymphokine, a heterologous antibody, a fragment of a heterologous antibody, a detectable label, a polyethylene glycol (PEG), a radioisotope, and a combination thereof.

In one aspect, the antibody antigen binding fragment is conjugated to one or more cytotoxin selected from the group consisting of a topoisomerase I inhibitor, tubulysin derivative, and a combination thereof. For example, the antibody or antigen binding fragment thereof is conjugated to one or more cytotoxins selected from the group consisting of TOP1i ('0132), exatecan, and a combination thereof. In some aspects, the cytotoxin is exatecan.

The antibody or antigen binding fragment thereof may be conjugated to a topoisomerase I inhibitor. Topoisomerase inhibitors are chemical compounds that block the action of topoisomerase (topoisomerase I and II), which is a type of enzyme that controls the changes in DNA structure by catalyzing the breaking and rejoining of the phosphodiester backbone of DNA strands during the normal cell cycle.

### Further Aspects

Certain features of the topoisomerase I inhibitor linkers are described below. By way of example, an aspect of feature Q^{X} (e.g., within the linker of Formula (ICI) below) will be outlined.

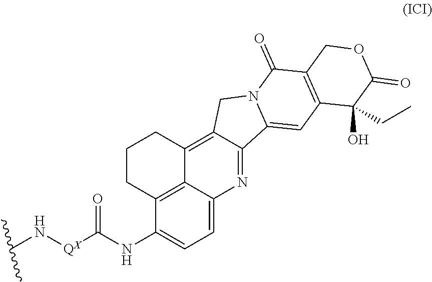

[symbol]

indicates the point of attachment to the antibody or to further aspects of the linker.

In one aspect, Q^{X} is an amino acid residue. The amino acid may be a natural amino acid or a non-natural amino acid. For example, Q^{X} may be selected from the group consisting of: Phe, Lys, Val, Ala, Cit, Leu, Ile, Arg, and Trp, where Cit is citrulline.

In one aspect, Q^{X} comprises a dipeptide residue. The amino acids in the dipeptide may be any combination of natural amino acids and non-natural amino acids. In some aspects, the dipeptide comprises natural amino acids. Where the linker is a cathepsin labile linker, the dipeptide is the site of action for cathepsin-mediated cleavage. The dipeptide then is a recognition site for cathepsin.

In one aspect, Q^{X} is selected from the group consisting of:

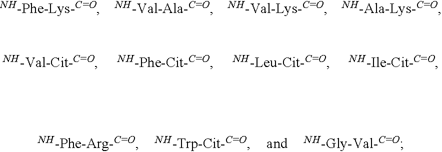

where Cit is citrulline.

In another aspect, Q is selected from the group consisting of:

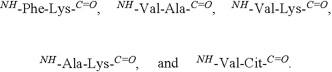

In another aspect, Q is selected from the group consisting of ^{NH}-Phe-Lys-^{C═O}, ^{NH}-Val-Cit-^{C═O} and ^{NH}-Val-Ala-^{C═O}

Other suitable dipeptide combinations include:

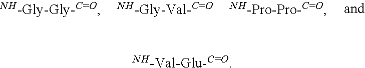

Other dipeptide combinations may be used, including those described by Dubowchik et al., Bioconjugate Chemistry 13: 855-69 (2002), which is incorporated herein by reference.

In some aspects, Q^{X} is a tripeptide residue. The amino acids in the tripeptide may be any combination of natural amino acids and non-natural amino acids. In some aspects, the tripeptide comprises natural amino acids. Where the linker is a cathepsin labile linker, the tripeptide is the site of action for cathepsin-mediated cleavage. The tripeptide then is a recognition site for cathepsin. Tripeptide linkers of particular interest are:

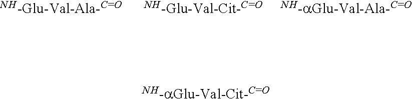

In some aspects, Q^{X} is a tetrapeptide residue. The amino acids in the tetrapeptide may be any combination of natural amino acids and non-natural amino acids. In some aspects, the tetrapeptide comprises natural amino acids. Where the linker is a cathepsin labile linker, the tetrapeptide is the site of action for cathepsin-mediated cleavage. The tetrapeptide then is a recognition site for cathepsin. Tetrapeptide linkers of particular interest are:

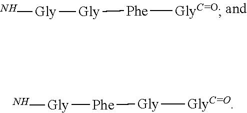

In some aspects, the tetrapeptide is:

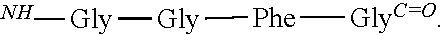

In the above representations of peptide residues, ^{NH}—represents the N-terminus, and —^{C═O} represents the C-terminus of the residue. The C-terminus binds to the NH of drug.

Glu, when used in the context of an amino acid, represents the residue of glutamic acid, i.e.:

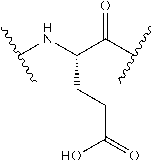 [SMILES: *N[C@@H](CCC(=O)O)C(*)=O]

αGlu, when used in the context of an amino acid, represents the residue of glutamic acid when bound via the α-chain, i.e.:

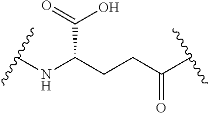 [SMILES: *N[C@@H](CCC(*)=O)C(=O)O]

In one aspect, the amino acid side chain is chemically protected, where appropriate. The side chain protecting group may be a group as discussed above. Protected amino acid sequences are cleavable by enzymes. For example, a dipeptide sequence comprising a Boc side chain-protected Lys residue is cleavable by cathepsin.

Protecting groups for the side chains of amino acids are well known in the art and are described in the Novabiochem Catalog, and as described above.

Illustrative examples of the synthesis of Topoisomerase I inhibitors and key intermediates are well-known in the art, and disclosed, for example, in International Publication No. WO 2020/200880, incorporated herein by reference.

Amine protecting groups are well-known to those skilled in the art. Particular reference is made to the disclosure of suitable protecting groups in Greene's Protecting Groups in Organic Synthesis, Fourth Edition, John Wiley & Sons, 2007 (ISBN 978-0-471-69754-1), pages 696-871.

The antibody or antigen fragment thereof of the disclosure may be conjugated to heterologous agents (such as a cytotoxin) using site-specific or non-site specific methods of conjugation. In one aspect, the antibodies and antigen fragment thereof comprise one, two, three, four, or more therapeutic moieties. In one aspect, all therapeutic moieties are the same.

Conventional conjugation strategies for antibodies or antigen-binding fragments thereof rely on randomly conjugating the payload to the antibody or fragment through lysines or cysteines. In one aspect, the antibody or antigen-binding fragment thereof is randomly conjugated to a heterologous agent (such as a cytotoxin), for example, by partial reduction of the antibody or fragment, followed by reaction with a desired agent, with or without a linker moiety attached. The antibody or fragment may be reduced using DTT or similar reducing agent. The agent with or without a linker moiety attached can then be added at a molar excess to the reduced antibody or fragment in the presence of DMSO. After conjugation, excess free cysteine may be added to quench unreacted agent. The reaction mixture may then be purified and buffer-exchanged into PBS.

Through transcriptomic and proteomic profiling of various solid tumour cell lines, β-glucuronidase expression has been identified as being upregulated and typically localised to the lysosome. International Publication Nos. WO 2007/011968 and WO 2015/182984 disclose certain antibody drug conjugates comprising β-glucuronidase-cleavable linkers. There remains a need for conjugates having β-glucuronidase-cleavable linkers that can selectively deliver a drug to a biological target and that have favourable physicochemical properties, including solubility and lipophilicity. The conjugates of the disclosure may be used for the treatment of diseases such as cancer.

In other aspects there is provided a conjugate of Formula (IA):

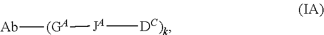

or a pharmaceutically acceptable salt thereof, wherein Ab is an anti-CD22 antibody or antigen binding fragment thereof comprising: a heavy chain CDR1 (HCDR1) comprising the amino acid sequence of SEQ ID NO: 1, a heavy chain CDR2 (HCDR2) comprising the amino acid sequence of SEQ ID NO: 2, a heavy chain CDR3 (HCDR3) comprising the amino acid sequence of SEQ ID NO: 3, a light chain CDR1 (LCDR1) comprising the amino acid sequence of SEQ ID NO: 4, a light chain CDR2 (LCDR2) comprising the amino acid sequence of SEQ ID NO: 5, and a light chain CDR3 (LCDR3) comprising the amino acid sequence of SEQ ID NO: 6;
k is an integer from 1 to 10, each G^{A} is independently a conjugation group conjugated to the antibody or antigen-binding fragment thereof, each D^{C} is:

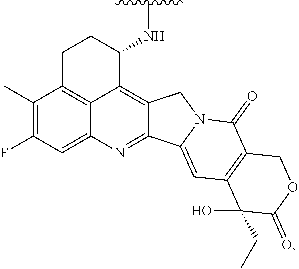 [SMILES: *N[C@H]1CCc2c(C)c(F)cc3nc4c(c1c23)Cn1c-4cc2c(c1=O)COC(=O)[C@]2(O)CC]

each J^{A} is independently a group of Formula (ICJ):

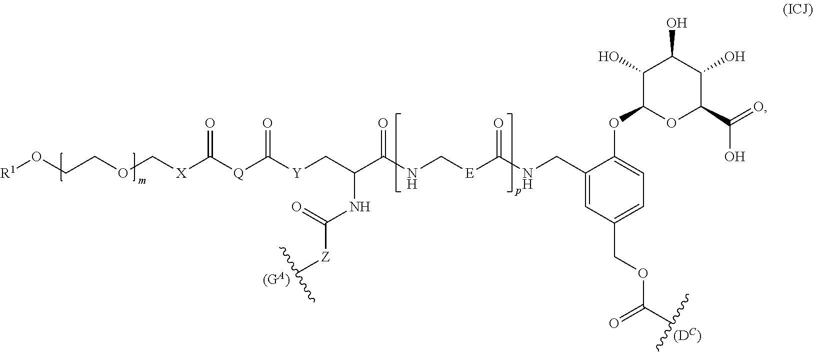

E is (CH₂)ₙ₁, wherein n1 is 0, 1, 2, or 3,
Q is

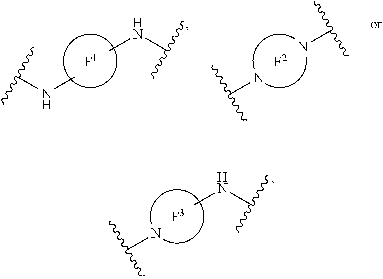

Wherein Ring F¹ is a saturated bicyclic ring having 6, 7, or 8 carbon atoms and optionally 1 or 2 oxygen atoms, Ring F² is a saturated bicyclic ring having the 2 nitrogen atoms shown, 4, 5, 6, 7, or 8 carbon atoms and optionally 1 oxygen atom, and Ring F³ is a saturated bicyclic ring having the 1 nitrogen atom shown, 5, 6, 7, or 8 carbon atoms and optionally 1 oxygen atom,
R¹ is C₁₋₄ alkyl,
X is (CH₂)ₙ₂, wherein n2 is 0, 1, 2, or 3,
Y is (CH₂)ₙ₃, wherein n3 is 0, 1, 2, 3, or 4,
Z is (CH₂)ₙ₄, wherein n4 is 1, 2, 3, 4, or 5,
m is an integer from 5 to 17,
p is 1 or 0,
(G^{A}) indicates the point of attachment to G^{A}, and
(D^{C}) indicates the point of attachment to D^{C}.

In a further aspect there is provided a pharmaceutical composition comprising a conjugate of Formula (IA), or a pharmaceutically acceptable salt thereof and a pharmaceutically acceptable excipient.

In a further aspect there is provided a conjugate of Formula (IA), or a pharmaceutically acceptable salt thereof, for use in therapy.

In a further aspect there is provided a conjugate of Formula (IA), or a pharmaceutically acceptable salt thereof, for use in the treatment of cancer.

In a further aspect there is provided the use of a conjugate of Formula (IA), or a pharmaceutically acceptable salt thereof, in the manufacture of a medicament.

In a further aspect there is provided the use of a conjugate of Formula (IA), or a pharmaceutically acceptable salt thereof, in the manufacture of a medicament for the treatment of cancer.

In a further aspect there is provided a method of treating cancer in a patient comprising administering to the patient an effective amount of a conjugate of Formula (IA), or a pharmaceutically acceptable salt thereof.

A conjugate of Formula (IA), or a pharmaceutically acceptable salt thereof, may undergo enzymatic cleavage to release a free drug (exatecan). Conjugates of Formula (IC) may exhibit improved efficacy and/or advantageous physical properties (for example, higher stability, lower lipophilicity, higher aqueous solubility, higher permeability and/or lower plasma protein binding), and/or favourable toxicity profiles (for example reduced off target toxicity), and/or favourable metabolic or pharmacokinetic profiles, in comparison with other conjugates. As such, conjugates of Formula (IA) may especially be suitable for use in therapy, such as the treatment of cancer.

So that the present specification may be more readily understood, certain terms are explicitly defined below. In addition, definitions are set forth as appropriate throughout the detailed description. Where examples are provided for a definition, they are not limiting.

The prefix C_{x-y}, where x and y are integers, indicates the numerical range of carbon atoms that are present in a group.

As used herein the term “alkyl” refers to a saturated, linear or branched hydrocarbon radical having the specified number of carbon atoms. Examples of C₁₋₄ alkyl groups include methyl (Me), ethyl (Et), n-propyl (ⁿPr), i-propyl (¹Pr), n-butyl (ⁿBu), i-butyl (ⁱBu), s-butyl (^{s}Bu), and t-butyl (^{t}Bu). Examples of C₁₋₆ alkyl groups include methyl, ethyl, n-propyl, i-propyl, n-butyl, i-butyl, s-butyl, t-butyl, n-pentyl, and n-hexyl.

As used herein the term “bicyclic ring” refers to a fused, bridged or spirocyclic bicyclic ring.

As used herein the term “conjugation group for conjugation to an antibody, or antigen-binding fragment thereof” refers to an atom or group of atoms capable of forming a covalent bond to an antibody, or antigen-binding fragment thereof, through a chemical reaction.

The use of “ [symbol] ” in formulas of this specification indicates the point of attachment to the antibody or antigen-binding fragment thereof. By way of illustration

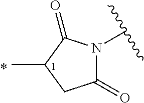 [SMILES: *N1C(=O)CC(C)C1=O]

indicates that that there is a covalent bond connecting the antibody, or antigen-binding fragment thereof, to the carbon atom marked 1.

For the avoidance of doubt, the use of

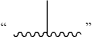

in formulas of this specification denotes the point of covalent attachment to a group, where the group is other than the antibody or antigen-binding fragment thereof.

Certain embodiments of this specification include a group which is said to be “optionally substituted”. In further embodiments said group is unsubstituted.

As used herein

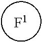

is ring F¹,

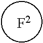

is ring F², and

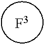

is ring F₃.

Units, prefixes, and symbols are denoted in their International System of Units (SI) accepted form. Numeric ranges are inclusive of the numbers defining the range.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary of Biochemistry and Molecular Biology, Revised, 2000, Oxford University Press, provide one of skill with a general dictionary of many of the terms used in this disclosure.

In aspects there is provided a conjugate of Formula (IA), or a pharmaceutically acceptable salt thereof, wherein Q is

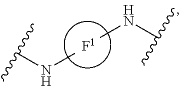

and wherein ring F¹ is a saturated bicyclic ring having 6, 7, or 8 carbon atoms and optionally 1 or 2 oxygen atoms. In further aspects the bicyclic ring is a fused bicyclic ring.

In aspects there is provided a conjugate of Formula (IA), or a pharmaceutically acceptable salt thereof, wherein Q is

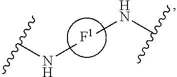

and ring F¹ is a saturated bicyclic ring having 6, 7, or 8 carbon atoms and 1 or 2 oxygen atoms. In further aspects the bicyclic ring is a fused bicyclic ring.

In aspects there is provided a conjugate of Formula (IA), or a pharmaceutically acceptable salt thereof, wherein Q is

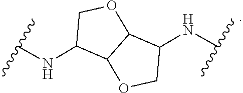 [SMILES: *NC1COC2C(N*)COC12]

In aspects there is provided a conjugate of Formula (IA), or a pharmaceutically acceptable salt thereof, wherein Q is

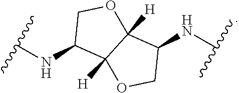 [SMILES: *N[C@H]1CO[C@H]2[C@@H]1OC[C@@H]2N*]

In aspects there is provided a conjugate of Formula (IA), or a pharmaceutically acceptable salt thereof, wherein Q is

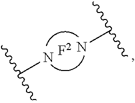

and wherein Ring F² is a saturated bicyclic ring having the 2 nitrogen atoms shown, 4, 5, 6, 7, or 8 carbon atoms and optionally 1 oxygen atom. In further aspects the bicyclic ring is a spirocyclic bicyclic ring. In further aspects the bicyclic ring is a bridged bicyclic ring.

In aspects there is provided a conjugate of Formula (IA), or a pharmaceutically acceptable salt thereof, wherein Q is

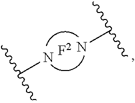

and wherein Ring F² is a saturated bicyclic ring having the 2 nitrogen atoms shown and 4, 5, 6, 7, or 8 carbon atoms. In further aspects, the bicyclic ring is a spirocyclic bicyclic ring. In further aspects, the bicyclic ring is a bridged bicyclic ring.

In aspects there is provided a conjugate of Formula (IA), or a pharmaceutically acceptable salt thereof, wherein Q is

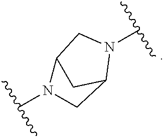 [SMILES: *N1CC2CC1CN2* ; "."]

In aspects there is provided a conjugate of Formula (IA), or a pharmaceutically acceptable salt thereof, wherein Q is

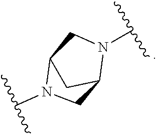 [SMILES: *N1C[C@H]2C[C@@H]1CN2*]

In aspects there is provided a conjugate of Formula (IA), or a pharmaceutically acceptable salt thereof, wherein Q is

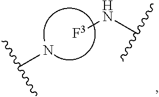

wherein Ring F³ is a saturated bicyclic ring having the 1 nitrogen atom shown, 5, 6, 7, or 8 carbon atoms and optionally 1 oxygen atom. In further aspects the bicyclic ring is a spirocyclic bicyclic ring. In further aspects the bicyclic ring is a bridged bicyclic ring.

In aspects there is provided a conjugate of Formula (IA), or a pharmaceutically acceptable salt thereof, wherein Q is

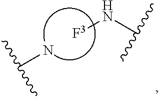

wherein Ring F³ is a saturated bicyclic ring having the 1 nitrogen atom shown and 5, 6, 7, or 8 carbon atoms. In further aspects the bicyclic ring is a spirocyclic bicyclic ring. In further aspects the bicyclic ring is a bridged bicyclic ring.

In aspects there is provided a conjugate of Formula (IA), or a pharmaceutically acceptable salt thereof, wherein E is (CH₂)ₙ₁, wherein n1 is 0, 1, 2, or 3. In further aspects E is a covalent bond. In further aspects E is CH₂. In further aspects E is (CH₂)₂. In further aspects E is (CH₂)₃.

In aspects there is provided a conjugate of Formula (IA), or a pharmaceutically acceptable salt thereof, wherein X is (CH₂)ₙ₂, wherein n2 is 0, 1, 2, or 3. In further aspects X is a covalent bond. In further aspects X is CH₂. In further aspects X is (CH₂)₂. In further aspects X is (CH₂)₃.

In aspects there is provided a conjugate of Formula (IA), or a pharmaceutically acceptable salt thereof, wherein Y is (CH₂)ₙ₃, wherein n3 is 0, 1, 2, 3, or 4. In further aspects Y is a covalent bond. In further aspects Y is CH₂. In further aspects Y is (CH₂)₂. In further aspects Y is (CH₂)₃. In further aspects Y is (CH₂)₄.

In aspects there is provided a conjugate of Formula (IA), or a pharmaceutically acceptable salt thereof, wherein Z is (CH₂)ₙ₄, wherein n4 is 1, 2, 3, 4, or 5. In further aspects Z is CH₂. In further aspects Z is (CH₂)₂. In further aspects Z is (CH₂)₃. In further aspects Z is (CH₂)₄. In further aspects Z is (CH₂)₅.

In aspects there is provided a conjugate of Formula (IA), or a pharmaceutically acceptable salt thereof, wherein m is 5, 6, 7, 8, 9, 10, 11, 12, 13, 14, 15, 16, or 17. In further aspects m is an integer from 6 to 16. In further aspects m is an integer from 7 to 15. In further aspects m is an integer from 8 to 14. In further aspects m is an integer from 9 to 13. In further aspects m is an integer from 10 to 12. In further aspects m is 11.

In aspects there is provided a conjugate of Formula (IA), or a pharmaceutically acceptable salt thereof, wherein R¹ is C₁₋₄ alkyl. In further aspects R¹ is CH₃.

In aspects there is provided a conjugate of Formula (IA), or a pharmaceutically acceptable salt thereof, wherein each J^{A} is a group of Formula (ICB2):

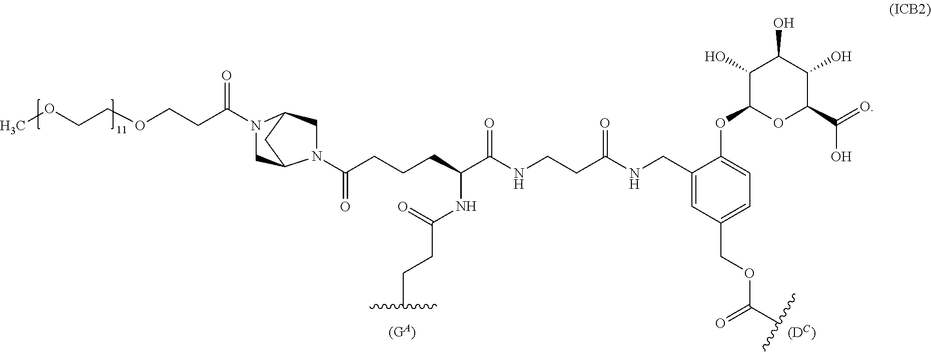

The present specification is intended to include all isotopes of atoms occurring in the present compounds and conjugates. Isotopes will be understood to include those atoms having the same atomic number but different mass numbers. For example, isotopes of hydrogen include tritium and deuterium. Isotopes of carbon include ¹³C and ¹⁴C. Isotopes of nitrogen include ¹⁵N.

The compounds disclosed herein may contain one or more chiral centers. Accordingly, if desired, such compounds can be prepared or isolated as pure stereoisomers, i.e., as individual enantiomers, diastereoisomers, or as a stereoisomerically enriched mixture. All such stereoisomer (and enriched) mixtures are included within the scope of the aspects, unless otherwise stated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents and the like.

Unless stereochemistry is explicitly indicated in a chemical structure or chemical name, the chemical structure or chemical name is intended to embrace all possible stereoisomers, diastereoisomers, conformers, rotamers and tautomers of the compound depicted. For example, a compound containing a chiral carbon atom is intended to embrace both the (R) enantiomer and the (S) enantiomer, as well as mixtures of the enantiomers, including racemic mixtures; and a compound containing two chiral carbons is intended to embrace all enantiomers and diastereoisomers including (R,R), (S,S), (R,S), and (S,R).

In one aspect, an agent (e.g., cytotoxin) is conjugated to an antibody or antigen binding fragment thereof by site-specific conjugation. In one aspect, site-specific conjugation of therapeutic moieties to antibodies using reactive amino acid residues at specific positions yields homogeneous ADC preparations with uniform stoichiometry.

The site specific conjugation can be through a cysteine, residue or a non-natural amino acid. In one aspect, the heterologous agent (such as a cytotoxin) is conjugated to the antibody or antigen binding fragment thereof through at least one cysteine residue.

In one aspect, the heterologous agent (such as a cytotoxin) is chemically conjugated to the side chain of an amino acid (e.g., at a specific Kabat position in the Fc region). In one aspect, the agent (e.g., the cytotoxic or imaging agent) is conjugated to the antibody or antigen binding fragment thereof through a cysteine substitution of at least one of positions 239, 248, 254, 273, 279, 282, 284, 286, 287, 289, 297, 298, 312, 324, 326, 330, 335, 337, 339, 350, 355, 356, 359, 360, 361, 375, 383, 384, 389, 398, 400, 413, 415, 418, 422, 440, 441, 442, 443, and 446, wherein the numbering corresponds to the EU index in Kabat. In one aspect, the specific Kabat positions are 239, 442, or both. In one aspect, the specific positions are Kabat position 442, an amino acid insertion between Kabat positions 239 and 240, or both. In one aspect, the heterologous agent (such as a cytotoxin) is conjugated to the antibody or antigen binding fragment thereof through a thiol-maleimide linkage. In some aspects, the amino acid side chain is a sulfhydryl side chain.

Reference herein to an antibody or antigen-binding fragment conjugated to a cytotoxin is synonymous with the term “antibody drug conjugate (ADC)”, or “anti-CD22 ADC”.

In one aspect, the antibody or antigen binding fragment thereof (e.g., anti-CD22 ADC) delivers a cytotoxic payload to a cell (such as a CD22-expressing cell) and inhibits or suppresses proliferation (e.g., of a tumor) by at least 10%, or at least 20%, or at least 30%, or at least 40%, or at least 50%, or at least 60%, or at least 70%, or at least 80%, or at least 90%, or about 100% (at least 40%) relative to a level of inhibition or suppression in the absence of the antibody or antigen binding fragment thereof (e.g., anti-CD22 ADC). Cellular proliferation can be assayed using art recognized techniques which measure rate of cell division, and/or the fraction of cells within a cell population undergoing cell division, and/or rate of cell loss from a cell population due to terminal differentiation or cell death (e.g., thymidine incorporation).

For the avoidance of doubt, reference to a “conjugate” herein means an antibody or antigen binding fragment conjugated to a heterologous agent (such as a cytotoxin) including any such agent described above.

In addition to the therapeutic applications of an antibody or antigen binding fragment of the disclosure described above, the “conjugates” of the present disclosure may be also used in a method of therapy. Thus, also provided is a method of treatment, comprising administering to a subject in need of treatment a therapeutically effective amount of a conjugate described herein (e.g., conjugate of formula IV). The term “therapeutically effective amount” is an amount sufficient to show benefit to a patient. Such benefit may be at least amelioration of at least one symptom. The actual amount administered, and rate and time-course of administration, will depend on the nature and severity of what is being treated. Prescription of treatment, e.g., decisions on dosage, is within the responsibility of general practitioners and other medical doctors.

A conjugate may be administered alone or in combination with other treatments, either simultaneously or sequentially dependent upon the condition to be treated. Examples of treatments and therapies include, but are not limited to, chemotherapy (the administration of active agents, including, e.g., drugs), surgery, and radiation therapy.

In another aspect, there is provided a polynucleotide comprising a nucleic acid sequence encoding an antibody or antigen binding fragment thereof of the disclosure.

In one aspect, the polynucleotide may be an isolated polynucleotide.

The sequence(s) (e.g., polynucleotide sequence(s)) of the present disclosure include sequences that have been removed from their naturally occurring environment, recombinant or cloned (e.g., DNA) isolates, and chemically synthesized analogues or analogues biologically synthesized by heterologous systems.

The sequence(s) (e.g., polynucleotide sequence(s)) of the present disclosure may be prepared by any means known in the art. For example, large amounts of the sequence(s) may be produced by replication and/or expression in a suitable host cell. The natural or synthetic DNA fragments coding for a desired fragment will typically be incorporated into recombinant nucleic acid constructs, typically DNA constructs, capable of introduction into and replication in a prokaryotic or eukaryotic cell. Usually the DNA constructs will be suitable for autonomous replication in a unicellular host, such as yeast or bacteria, but may also be intended for introduction to and integration within the genome of a cultured bacterial, insect, mammalian, plant or other eukaryotic cell lines.

The sequence(s) (e.g., polynucleotide sequence(s)) of the present disclosure may also be produced by chemical synthesis, e.g., a polynucleotide by the phosphoramidite method or the tri-ester method and may be performed on commercial automated oligonucleotide synthesizers. A double-stranded (e.g., DNA) fragment may be obtained from the single stranded product of chemical synthesis either by synthesizing the complementary strand and annealing the strand together under appropriate conditions or by adding the complementary strand using DNA polymerase with an appropriate primer sequence.

When applied to a sequence (e.g., polynucleotide sequence) of the disclosure, the term “isolated” denotes that the sequence has been removed from its natural genetic milieu and is thus free of other extraneous or unwanted coding sequences (but may include naturally occurring 5′ and 3′ untranslated regions such as promoters and terminators), and is in a form suitable for use within genetically engineered protein production systems. Such isolated molecules are those that are separated from their natural environment.

Another aspect provided herein is a host cell comprising a polynucleotide, said polynucleotide comprising a nucleic acid sequence encoding an antibody or antigen binding fragment thereof of the disclosure.

In one aspect, the polynucleotide encodes a VH chain of an antibody or antigen binding fragment thereof. In one aspect, a polynucleotide of the disclosure may encode a VL chain of an antibody or antigen binding fragment thereof. In one aspect, the polynucleotide may encode a VH and a VL chain of an antibody or antigen binding fragment thereof. In one aspect, the polynucleotide may further encode a leader sequence (e.g., which functions as a secretory sequence for controlling transport of a polypeptide from the cell).

In another aspect there is provided a vector (e.g., plasmid) comprising the polynucleotide of the disclosure.

Variants of a polynucleotide described above are embraced by the disclosure. Polynucleotide variants can contain alterations in the coding regions, non-coding regions, or both. In one aspect, a polynucleotide variant comprises an alteration that produces silent substitutions, additions, or deletions, but do not alter the properties or activities of the encoded polypeptide. In one aspect, a polynucleotide variant is produced by a silent substitution due to the degeneracy of the genetic code. A polynucleotide variant can be produced for a variety of reasons, e.g., to optimize codon expression for a particular host (change codons in the human mRNA to those preferred by a bacterial host such as E. coli). Vectors and cells comprising said polynucleotide variant are also provided.

The present disclosure embraces CD22 for producing an antibody or antigen binding fragment thereof that binds to a CD22 polypeptide (e.g., CD22 polypeptide epitope), comprising expressing a polynucleotide in a host cell, said polynucleotide comprising a nucleic acid sequence encoding an antibody or antigen binding fragment thereof of the disclosure.

The present disclosure further embraces an antibody or antigen binding fragment thereof obtainable by said methods for producing an antibody or antigen binding fragment thereof that binds to a CD22 polypeptide (e.g., CD22 polypeptide epitope).

In one aspect, the method for producing an antibody or antigen binding fragment thereof comprises (a) culturing the host cell, and (b) isolating the antibody or antigen binding fragment thereof expressed from the cell.

Suitable host cells for expression of an antibody or antigen binding fragment thereof of the disclosure include a prokaryote, yeast, insect, or higher eukaryotic cells (e.g., wherein the polynucleotide is under the control of appropriate promoters). Prokaryotes include gram negative or gram-positive organisms, for example E. coli or bacilli. Higher eukaryotic cells include established cell lines of mammalian origin as described herein. Cell-free translation systems can also be employed.

In one aspect, there is provided a kit comprising an antigen or antibody binding fragment described herein. There is further embraced use of said kit in the methods of the present disclosure.

In one aspect, a kit comprises an isolated (e.g., purified) antigen or antibody binding fragment of the disclosure. In one aspect, a kit comprises an isolated (e.g., purified) antigen or antibody binding fragment of the disclosure, wherein the antigen or antibody binding fragment comprises an agent (e.g., conjugated cytotoxin) described herein. In one aspect, the kit comprises one or more container. The kit may provide the antigen or antibody binding fragment and the agent individually (e.g., the agent is not conjugated to the antigen or antibody binding fragment, but is in a form suitable for conjugation thereto); optionally wherein the kit is further provided with instructions and/or reagents for conjugating the agent to the antigen or antibody binding fragment. In one aspect, the kit comprises all of the components necessary and/or sufficient to perform a detection assay, including all controls, directions for performing assays, and any necessary software for analysis and presentation of results.

An antibody or antigen binding fragment thereof of the disclosure can be used in assays for immunospecific binding by any method known in the art. The immunoassays that can be used include, but are not limited to, competitive and non-competitive assay systems using techniques such as Western blot, RIA, ELISA, ELISPOT, “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, and protein A immunoassays.

An antibody or antigen binding fragment thereof of the disclosure can be employed histologically, as in immunofluorescence, immunoelectron microscopy, or non-immunological assays, for example, for in situ detection of CD22 or conserved variants or peptide fragments thereof. In situ detection can be accomplished by removing a histological specimen from a patient, and applying thereto a labelled an antibody or antigen binding fragment thereof of the disclosure, e.g., applied by overlaying the labelled antibody or antigen binding fragment thereof onto a biological sample. Through the use of such a procedure, it is possible to determine not only the presence of CD22, or conserved variants or peptide fragments, but also its distribution in the examined tissue. Using the present disclosure, those of ordinary skill will readily perceive that any of a wide variety of histological methods (such as staining procedures) can be modified in order to achieve such in situ detection.

### Antibodies

The term “antibody” covers monoclonal antibodies and fragments thereof (e.g., exhibiting the desired biological activity). In one aspect, an antibody of the present disclosure is a monoclonal antibody. In another aspect, the antibody is a fully human monoclonal antibody. In one aspect, methods of the disclosure may employ polyclonal antibodies.

In one aspect, the antibody or antigen binding fragment is one or more of a murine antibody, a humanized antibody, a chimeric antibody, a monoclonal antibody, a polyclonal antibody, a recombinant antibody, a multispecific antibody, or a combination thereof.

In one aspect, the antigen-binding fragment is one or more of a Fv fragment, a Fab fragment, an F(ab′)2 fragment, a Fab′ fragment, a dsFv fragment, an scFv fragment, an sc(Fv)2 fragment, or a combination thereof.

In one aspect, the antibody or antigen binding fragment thereof is a monoclonal antibody (mAb).

In one aspect, the antibody or antigen binding fragment thereof (e.g., mAb) of the disclosure is a scFV.

In one aspect, the antibody is mAb23, which comprises a heavy chain amino acid sequence of SEQ ID NO: 9 and a light chain amino acid sequence of SEQ ID NO: 10.

In one aspect, the antibody or antigen binding fragment thereof can bind to CD22 molecules across species, e.g., the antibody or fragment can bind to mouse CD22, rat CD22, rabbit, human CD22, and/or cynomolgus monkey CD22. In one aspect, the antibody or fragment can bind to human CD22 and cynomolgus monkey CD22.

In one aspect, the antibody or antigen binding fragment thereof can specifically bind to CD22, e.g., human CD22 and cynomolgus monkey CD22.

In one aspect, the antibody or antigen-binding fragment thereof can include, in addition to a VH and a VL, a heavy chain constant region or fragment thereof. In one aspect, the heavy chain constant region is a human heavy chain constant region, e.g., a human IgG constant region, e.g., a human IgG1, IgG2, or IgG4 constant region. In one aspect (wherein the antibody or antigen-binding fragment thereof is conjugated to an agent, such as a cytotoxic agent), a cysteine residue is inserted between amino acid S239 and V240 in the CH2 region of IgG1. This cysteine is referred to as “a 239 insertion” or “239i.”

In one aspect, a heavy chain constant region or fragment thereof, e.g., a human IgG constant region or fragment thereof, can include one or more amino acid substitutions relative to a wild-type IgG constant domain wherein the modified IgG has an increased half-life compared to the half-life of an IgG having the wild-type IgG constant domain. For example, the IgG constant domain can contain one or more amino acid substitutions of amino acid residues at positions 234-257, 285-290, 308-331, 385-389, and 428-436, wherein the amino acid position numbering is according to the Eu index as set forth in Kabat. In one aspect, the IgG constant domain can contain one or more of a substitution of the amino acid at Kabat position 234 with Phenylalanine (F), a substitution of the amino acid at Kabat position 235 with Glutamic Acid (E), a substitution of the amino acid at Kabat position 252 with Tyrosine (Y), Phenylalanine (F), Tryptophan (W), or Threonine (T), a substitution of the amino acid at Kabat position 254 with Threonine (T), a substitution of the amino acid at Kabat position 256 with Serine (S), Arginine (R), Glutamine (Q), Glutamic acid (E), Aspartic acid (D), or Threonine (T), a substitution of the amino acid at Kabat position 257 with Leucine (L), a substitution of the amino acid at Kabat position 309 with Proline (P), a substitution of the amino acid at Kabat position 311 with Serine (S), a substitution of the amino acid at Kabat position 331 with Serine (S), a substitution of the amino acid at Kabat position 428 with Threonine (T), Leucine (L), Phenylalanine (F), or Serine (S), a substitution of the amino acid at Kabat position 433 with Arginine (R), Serine (S), Isoleucine (I), Proline (P), or Glutamine (Q), or a substitution of the amino acid at Kabat position 434 with Tryptophan (W), Methionine (M), Serine (S), Histidine (H), Phenylalanine (F), or Tyrosine. In one aspect, the IgG constant domain can contain amino acid substitutions relative to a wild-type human IgG constant domain including as substitution of the amino acid at Kabat position 252 with Tyrosine (Y), a substitution of the amino acid at Kabat position 254 with Threonine (T), and a substitution of the amino acid at Kabat position 256 with Glutamic acid (E). In one aspect, the antibody or antigen-binding fragment thereof comprises a heavy chain, wherein the heavy chain is a human IgG1 YTE mutant.

The affinity or avidity of an antibody or antigen binding fragment thereof for an antigen can be determined experimentally using any suitable method well known in the art, e.g., flow cytometry, enzyme-linked immunosorbent assay (ELISA), or radioimmunoassay (RIA), or kinetics (e.g., KINEXA® or BIACORE™ analysis). Direct binding assays as well as competitive binding assay formats can be readily employed. (See, e.g., Berzofsky et al., Antibody-Antigen Interactions, In Fundamental Immunology, Paul, W. E., Ed., Raven Press: New York, N.Y. (1984); Kuby, Immunology, W. H. Freeman and Company: New York, N.Y. (1992); and methods described herein.) The measured affinity of a particular antibody-antigen interaction can vary if measured under different conditions (e.g., salt concentration, pH, temperature). Thus, measurements of affinity and other antigen-binding parameters (e.g., KD or Kd, Kon, Koff) are made with standardized solutions of antibody and antigen, and a standardized buffer, as known in the art.

A “monoclonal antibody” (mAb) refers to a homogeneous antibody population involved in the highly specific recognition and binding of a single antigenic determinant, or epitope. This is in contrast to polyclonal antibodies that typically include different antibodies directed against different antigenic determinants. The term “monoclonal antibody” encompasses both intact and full-length monoclonal antibodies as well as antibody fragments (such as Fab, Fab′, F(ab′)2, Fv), single chain (scFv) mutants, fusion proteins comprising an antibody portion, and any other modified immunoglobulin molecule comprising an antigen recognition site. Furthermore, “monoclonal antibody” refers to such antibodies made in any number of ways including, but not limited to, hybridoma, phage selection, recombinant expression, and transgenic animals.

In another aspect, the antibody or antigen binding fragment thereof (e.g., mAb) of the disclosure is a humanized antibody or antigen binding fragment thereof. Suitably, said humanized the antibody or antigen binding fragment thereof is an IgG.

The term “humanized antibody” refers to an antibody derived from a non-human (e.g., murine) immunoglobulin, which has been engineered to contain minimal non-human (e.g., murine) sequences. Typically, humanized antibodies are human immunoglobulins in which residues from the complementary determining region (CDR) are replaced by residues from the CDR of a non-human species (e.g., mouse, rat, rabbit, or hamster) that have the desired specificity, affinity, and capability (Jones et al., Nature 321: 522-25 (1986); Riechmann et al., Nature 332: 323-27 (1988); Verhoeyen et al., Science 239: 1534-36 (1988)). In some instances, the Fv framework region (FW) residues of a human immunoglobulin are replaced with the corresponding residues in an antibody from a non-human species that has the desired specificity, affinity, and capability.

Humanized antibodies can be further modified by the substitution of additional residues either in the Fv framework region and/or within the replaced non-human residues to refine and optimize antibody specificity, affinity, and/or capability. In general, humanized antibodies will comprise substantially all of at least one, and typically two or three, variable domains containing all or substantially all of the CDR regions that correspond to the non-human immunoglobulin whereas all or substantially all of the FR regions are those of a human immunoglobulin consensus sequence. Humanized antibody can also comprise at least a portion of an immunoglobulin constant region or domain (Fc), typically that of a human immunoglobulin. Examples of methods used to generate humanized antibodies are described in U.S. Pat. Nos. 5,225,539 or 5,639,641.

A “variable region” of an antibody refers to the variable region of the antibody light chain or the variable region of the antibody heavy chain, either alone or in combination. The variable regions of the heavy and light chain each consist of four framework regions (FW) connected by three complementarity-determining regions (CDRs), also known as hypervariable regions. The CDRs in each chain are held together in close proximity by the FW regions and, with the CDRs from the other chain, contribute to the formation of the antigen-binding site of antibodies. There are at least two techniques for determining CDRs: (1) an approach based on cross-species sequence variability (i.e., Kabat et al., Sequences of Proteins of Immunological Interest, (5th ed., 1991, National Institutes of Health, Bethesda Md.)); and (2) an approach based on crystallographic studies of antigen-antibody complexes (Al-lazikani et al., J. Mol. Biol. 273: 927-48 (1997)). In addition, combinations of these two approaches are sometimes used in the art to determine CDRs.

The “Kabat numbering system” is generally used when referring to a residue in the variable domain (approximately residues 1-107 of the light chain and residues 1-113 of the heavy chain) (e.g., Kabat et al., Sequences of Immunological Interest, 5th Ed. Public Health Service, National Institutes of Health, Bethesda, Md. (1991)).

The amino acid position numbering as in Kabat, refers to the numbering system used for heavy chain variable domains or light chain variable domains of the compilation of antibodies in Kabat et al., Sequences of Proteins of Immunological Interest, 5th Ed. Public Health Service, National Institutes of Health, Bethesda, Md. (1991). Using this numbering system, the actual linear amino acid sequence can contain fewer or additional amino acids corresponding to a shortening of, or insertion into, a FW or CDR of the variable domain. For example, a heavy chain variable domain can include a single amino acid insert (residue 52a according to Kabat) after residue 52 of H2 and inserted residues (e.g., residues 82a, 82b, and 82c, etc. according to Kabat) after heavy chain FW residue 82.

The Kabat numbering of residues can be determined for a given antibody by alignment at regions of homology of the sequence of the antibody with a “standard” Kabat numbered sequence. Chothia refers instead to the location of the structural loops (Chothia and Lesk, J. Mol. Biol. 196: 901-17 (1987)). The end of the Chothia CDR-H1 loop, when numbered using the Kabat numbering convention, varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software. The table below lists the positions of the amino acids comprising the variable regions of the antibodies in each system.

| Region | Kabat | AbM | Chothia |
|---|---|---|---|
| LCDR1 | L24-L34 | L24-L34 | L24-L34 |
| LCDR2 | L50-L56 | L50-L56 | L50-L56 |
| LCDR3 | L89-L97 | L89-L97 | L89-L97 |
| HCDR1¹ | H31-H35B | H26-H35B | H26-H32 . . . 34 |
| HCDR1² | H31-H35 | H26-H35 | H26-H32 |
| HCDR2 | H50-H65 | H50-H58 | H52-H56 |
| HCDR3 | H95-H102 | H95-H102 | H95-H102 |

¹Kabat Numbering
²Chothia Numbering

ImMunoGeneTics (IMGT) also provides a numbering system for the immunoglobulin variable regions, including the CDRs. See, e.g., Lefranc et al., Dev. Comp. Immunol. 27: 55-77 (2003). The IMGT numbering system is based on an alignment of more than 5,000 sequences, structural data, and characterization of hypervariable loops and allows for easy comparison of the variable and CDR regions for all species. According to the IMGT numbering schema, VH-CDR1 is at positions 26 to 35, VH-CDR2 is at positions 51 to 57, VH-CDR3 is at positions 93 to 102, VL-CDR1 is at positions 27 to 32, VL-CDR2 is at positions 50 to 52, and VL-CDR3 is at positions 89 to 97.

As used throughout the specification the VH CDRs sequences described correspond to the classical Kabat numbering locations, namely Kabat VH-CDR1 is at positions 31-35, VH-CDR2 is at positions 50-65, and VH-CDR3 is at positions 95-102. VL-CDR1, VL-CDR2 and VL-CDR3 also correspond to classical Kabat numbering locations, namely at positions 24-34, 50-56, and 89-97, respectively.

In one aspect, an anti-CD22 antibody of the disclosure is a human antibody.

The term “human antibody” means an antibody produced in a human or an antibody having an amino acid sequence corresponding to an antibody produced in a human made using any technique known in the art. This definition of a human antibody includes intact or full-length antibodies, fragments thereof, and/or antibodies comprising at least one human heavy and/or light chain polypeptide such as, for example, an antibody comprising murine light chain and human heavy chain polypeptides.

In one aspect, an anti-CD22 antibody of the disclosure is a chimeric antibody.

The term “chimeric antibodies” refers to antibodies in which the amino acid sequence of the immunoglobulin molecule is derived from two or more species. Typically, the variable region of both light and heavy chains corresponds to the variable region of antibodies derived from one species of mammals (e.g., mouse, rat, rabbit, etc.) with the desired specificity, affinity, and capability while the constant regions are homologous to the sequences in antibodies derived from another (usually human) to avoid eliciting an immune response in that species.

The terms “YTE” or “YTE mutant” refer to a mutation in IgG1 Fc that results in an increase in the binding to human FcRn and improves the serum half-life of the antibody having the mutation. A YTE mutant comprises a combination of three mutations, M252Y/S254T/T256E (EU numbering Kabat et al., Sequences of Proteins of Immunological Interest, U.S. Public Health Service, National Institutes of Health, Washington, D.C. (1991)), introduced into the heavy chain of an IgG1. See U.S. Pat. No. 7,658,921, which is incorporated by reference herein. The YTE mutant has been shown to increase the serum half-life of antibodies approximately four-times as compared to wild-type versions of the same antibody (Dall'Acqua et al., J. Biol. Chem. 281: 23514-24 (2006); Robbie et al., Antimicrob. Agents Chemother. 57: 6147-53 (2013)). See also U.S. Pat. No. 7,083,784, which is hereby incorporated by reference in its entirety.

“Binding affinity” generally refers to the strength of the sum total of non-covalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (KD). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present disclosure.

Potency of the antibody or antigen binding fragment thereof is normally expressed as an IC50 value, in ng/mL unless otherwise stated. IC50 is the median inhibitory concentration of an antibody molecule. In functional assays, IC50 is the concentration that reduces a biological response by 50% of its maximum. In ligand-binding studies, IC50 is the concentration that reduces receptor binding by 50% of maximal specific binding level. IC50 can be calculated by any number of means known in the art.

The fold improvement in potency for the antibody or antigen binding fragment thereof of the disclosure as compared to a reference antibody can be at least about 2-fold, at least about 4-fold, at least about 6-fold, at least about 8-fold, at least about 10-fold, at least about 20-fold, at least about 30-fold, at least about 40-fold, at least about 50-fold, at least about 60-fold, at least about 70-fold, at least about 80-fold, at least about 90-fold, at least about 100-fold, at least about 110-fold, at least about 120-fold, at least about 130-fold, at least about 140-fold, at least about 150-fold, at least about 160-fold, at least about 170-fold, or at least about 180-fold or more.

Binding potency of an antibody is normally expressed as an EC50 value, in nM or pM unless otherwise stated. EC50 is the concentration of a drug that induces a median response between baseline and maximum after a specified exposure time. EC50 can be calculated by any number of means known in the art.

### Antibody Preparation

The antibodies of the present disclosure can be obtained using conventional techniques known to persons skilled in the art and their utility confirmed by conventional binding studies. By way of example, a simple binding assay is to incubate the cell expressing an antigen with the antibody. If the antibody is tagged with a fluorophore, the binding of the antibody to the antigen can be detected by FACS analysis.

### Isomers

Certain compounds/agents of the disclosure may exist in one or more particular geometric, optical, enantiomeric, diasteriomeric, epimeric, atropic, stereoisomeric, tautomeric, conformational, or anomeric forms, including but not limited to, cis- and trans-forms; E- and Z-forms; c-, t-, and r-forms; endo- and exo-forms; R-, S-, and meso-forms; D- and L-forms; d- and l-forms; (+) and (−) forms; keto-, enol-, and enolate-forms; syn- and anti-forms; synclinal- and anticlinal-forms; α- and β-forms; axial and equatorial forms; boat-, chair-, twist-, envelope-, and halfchair-forms; and combinations thereof, hereinafter collectively referred to as “isomers” (or “isomeric forms”).

The term “chiral” refers to molecules which have the property of non-superimposability of the mirror image partner, while the term “achiral” refers to molecules which are superimposable on their mirror image partner.

The term “stereoisomers” refers to compounds which have identical chemical constitution, but differ with regard to the arrangement of the atoms or groups in space.

“Diastereomer” refers to a stereoisomer with two or more centers of chirality and whose molecules are not mirror images of one another. Diastereomers have different physical properties, e.g., melting points, boiling points, spectral properties, and reactivities. Mixtures of diastereomers may separate under high resolution analytical procedures such as electrophoresis and chromatography.

“Enantiomers” refer to two stereoisomers of a compound which are non-superimposable mirror images of one another.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., “Stereochemistry of Organic Compounds”, John Wiley & Sons, Inc., New York, 1994. The compounds of the disclosure may contain asymmetric or chiral centers, and therefore exist in different stereoisomeric forms. It is intended that all stereoisomeric forms of the compounds of the disclosure, including but not limited to, diastereomers, enantiomers and atropisomers, as well as mixtures thereof such as racemic mixtures, form part of the present disclosure. Many organic compounds exist in optically active forms, i.e., they have the ability to rotate the plane of plane-polarized light. In describing an optically active compound, the prefixes D and L, or R and S, are used to denote the absolute configuration of the molecule about its chiral center(s). The prefixes d and l or (+) and (−) are employed to designate the sign of rotation of plane-polarized light by the compound, with (−) or l meaning that the compound is levorotatory. A compound prefixed with (+) or d is dextrorotatory. For a given chemical structure, these stereoisomers are identical except that they are mirror images of one another. A specific stereoisomer may also be referred to as an enantiomer, and a mixture of such isomers is often called an enantiomeric mixture. A 50:50 mixture of enantiomers is referred to as a racemic mixture or a racemate, which may occur where there has been no stereoselection or stereospecificity in a chemical reaction or process. The terms “racemic mixture” and “racemate” refer to an equimolar mixture of two enantiomeric species, devoid of optical activity.

“Enantiomerically enriched form” refers to a sample of a chiral substance whose enantiomeric ratio is greater than 50:50 but less than 100:0.

Note that, except as discussed below for tautomeric forms, specifically excluded from the term “isomers”, as used herein, are structural (or constitutional) isomers (i.e., isomers which differ in the connections between atoms rather than merely by the position of atoms in space). For example, a reference to a methoxy group, —OCH₃, is not to be construed as a reference to its structural isomer, a hydroxymethyl group, —CH₂OH. Similarly, a reference to ortho-chlorophenyl is not to be construed as a reference to its structural isomer, meta-chlorophenyl. However, a reference to a class of structures may well include structurally isomeric forms falling within that class (e.g., C₁₋₇ alkyl includes n-propyl and iso-propyl; butyl includes n-, iso-, sec-, and tert-butyl; methoxyphenyl includes ortho-, meta-, and para-methoxyphenyl).

The above exclusion does not pertain to tautomeric forms, for example, keto-, enol-, and enolate-forms, as in, for example, the following tautomeric pairs: keto/enol (illustrated below), imine/enamine, amide/imino alcohol, amidine/enediamine, nitroso/oxime, thioketone/enethiol, N-nitroso/hyroxyazo, and nitro/aci-nitro.

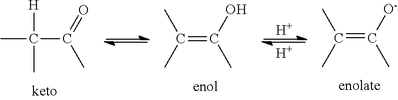

The term “tautomer” or “tautomeric form” refers to structural isomers of different energies which are interconvertible via a low energy barrier. For example, proton tautomers (also known as prototropic tautomers) include interconversions via migration of a proton, such as keto-enol and imine-enamine isomerizations. Valence tautomers include interconversions by reorganization of some of the bonding electrons.

Note that specifically included in the term “isomer” are compounds with one or more isotopic substitutions. For example, H may be in any isotopic form, including ¹H, ²H (D), and ³H (T); C may be in any isotopic form, including ¹²C, ¹³C, and ¹⁴C; O may be in any isotopic form, including ¹⁶O and ¹⁸O; and the like.

Examples of isotopes that can be incorporated into compounds of the disclosure include isotopes of hydrogen, carbon, nitrogen, oxygen, phosphorous, fluorine, chlorine and iodine, such as, but not limited to ²H (deuterium, D), ³H (tritium), ¹¹C, ¹³C, ¹⁴C, ¹⁵N, ¹⁸F, ³¹P, ³²p ³⁵S, ³⁶Cl, and ¹²⁵I. Various isotopically labeled compounds of the present disclosure, for example those into which radioactive isotopes such as 3H, 13C, and 14C are incorporated. Such isotopically labelled compounds may be useful in metabolic studies, reaction kinetic studies, detection or imaging techniques, such as positron emission tomography (PET) or single-photon emission computed tomography (SPECT) including drug or substrate tissue distribution assays, or in radioactive treatment of patients. Deuterium labelled or substituted therapeutic compounds of the disclosure may have improved DMPK (drug metabolism and pharmacokinetics) properties, relating to distribution, metabolism, and excretion (ADME). Substitution with heavier isotopes such as deuterium may afford certain therapeutic advantages resulting from greater metabolic stability, for example increased in vivo half-life or reduced dosage requirements. An 18F labeled compound may be useful for PET or SPECT studies. Isotopically labeled compounds of this disclosure and prodrugs thereof can generally be prepared by carrying out the procedures disclosed in the schemes or in the examples and preparations described below by substituting a readily available isotopically labeled reagent for a non-isotopically labeled reagent. Further, substitution with heavier isotopes, particularly deuterium (i.e., 2H or D) may afford certain therapeutic advantages resulting from greater metabolic stability, for example increased in vivo half-life or reduced dosage requirements or an improvement in therapeutic index. It is understood that deuterium in this context is regarded as a substituent. The concentration of such a heavier isotope, specifically deuterium, may be defined by an isotopic enrichment factor. In the compounds of this disclosure any atom not specifically designated as a particular isotope is meant to represent any stable isotope of that atom.

Unless otherwise specified, a reference to a particular compound includes all such isomeric forms, including (wholly or partially) racemic and other mixtures thereof. Methods for the preparation (e.g., asymmetric synthesis) and separation (e.g., fractional crystallization and chromatographic means) of such isomeric forms are either known in the art or are readily obtained by adapting the methods taught herein, or known methods, in a known manner.

### EMBODIMENTS

Embodiment 1. An anti-CD22 antibody or antigen binding fragment thereof, comprising a heavy chain CDR1 (HCDR1) comprising the amino acid sequence of SEQ ID NO: 1, a heavy chain CDR2 (HCDR2) comprising the amino acid sequence of SEQ ID NO: 2, a heavy chain CDR3 (HCDR3) comprising the amino acid sequence of SEQ ID NO: 3, a light chain CDR1 (LCDR1) comprising the amino acid sequence of SEQ ID NO: 4, a light chain CDR2 (LCDR2) comprising the amino acid sequence of SEQ ID NO: 5, and a light chain CDR3 (LCDR3) comprising the amino acid sequence of SEQ ID NO: 6.

Embodiment 2. The anti-CD22 antibody or antigen binding fragment thereof according to embodiment 1, wherein the antibody or antigen binding fragment thereof comprises a variable heavy (VH) chain at least 90%, at least 91% at least 92%, at least 93%, at least 94%, at least 95%, at least 96%, at least 97%, at least 98%, or at least 99% identical to the amino acid sequence of SEQ ID NO: 7, and a variable light (VL) chain at least 90%, at least 91% at least 92%, at least 93%, at least 94%, at least 95%, at least 96%, at least 97%, at least 98%, or at least 99% identical to the amino acid sequence of SEQ ID NO: 8.

Embodiment 3. The anti-CD22 antibody or antigen binding fragment thereof according to embodiment 2, wherein the antibody or antigen binding fragment thereof comprises a VH chain comprising the amino acid sequence of SEQ ID NO: 7 and a VL chain comprising the amino acid sequence of SEQ ID NO: 8.

Embodiment 4. The anti-CD22 antibody or antigen binding fragment thereof according to any one of embodiments 1 to 3, wherein the antibody or binding fragment thereof comprises an Fc region.

Embodiment 5. The anti-CD22 antibody or antigen binding fragment thereof according to embodiment 4, wherein the Fc region comprises one or more mutations, which has reduced antibody dependent cellular cytotoxicity (ADCC) compared to an antibody having a wild-type Fc region.

Embodiment 6. The anti-CD22 antibody or antigen binding fragment thereof according to embodiment 5, wherein the Fc region comprises a L234F/L235E/P331S (EU Index numbering) triple mutation (TM).

Embodiment 7. The anti-CD22 antibody or antigen binding fragment thereof according to any one of embodiments 1 to 6, wherein the anti-CD22 antibody or antigen binding fragment thereof comprises a constant heavy chain comprising the amino acid sequence of SEQ ID NO: 11 and a constant light chain comprising the amino acid sequence of SEQ ID NO: 12.

Embodiment 8. The anti-CD22 antibody or antigen binding fragment thereof according to any one of embodiments 1 to 7, wherein the antibody or antigen binding fragment thereof comprises a heavy chain comprising the amino acid sequence of SEQ ID NO: 9.

Embodiment 9. The anti-CD22 antibody or antigen binding fragment thereof according to any one of embodiments 1 to 8, wherein the antibody or antigen binding fragment thereof comprises a light chain comprising the amino acid sequence of SEQ ID NO: 10.

Embodiment 10. The anti-CD22 antibody or antigen binding fragment thereof according to any one of embodiments 1 to 9, wherein the antibody or antigen binding fragment thereof comprises a heavy chain comprising the amino acid sequence of SEQ ID NO: 9; and a light chain comprising the amino acid sequence of SEQ ID NO: 10.

Embodiment 11. The anti-CD22 antibody or antigen binding fragment thereof according to any one of embodiments 1 to 4, wherein the antibody or antigen binding fragment thereof comprises a heavy chain comprising the amino acid sequence of SEQ ID NO: 13; and a light chain comprising the amino acid sequence of SEQ ID NO: 10.

Embodiment 12. The anti-CD22 antigen binding fragment according to any one of embodiments 1 to 11, wherein the antigen-binding fragment is a single chain Fv, Fab fragment, a Fab′ fragment, or a F(ab′)2 fragment.

Embodiment 13. The anti-CD22 antibody or antigen-binding fragment thereof according to any one of embodiments 1 to 12, wherein the anti-CD22 antibody or antigen binding fragment thereof is humanized, chimeric, or fully human.

Embodiment 14. The anti-CD22 antibody or antigen-binding fragment thereof according to embodiment 13, wherein the anti-CD22 antibody or antigen binding fragment is fully human.

Embodiment 15. The anti-CD22 antibody or antigen-binding fragment thereof according to any one of embodiments 1 to 14, wherein the anti-CD22 antibody or antigen-binding fragment thereof is a monoclonal antibody.

Embodiment 16. The anti-CD22 antibody or antigen-binding fragment thereof according to any one of embodiments 1 to 15, wherein the anti-CD22 antibody or antigen-binding fragment thereof is an IgG1.

Embodiment 17. The anti-CD22 antibody or antigen binding fragment thereof according to any one of embodiments 1 to 16, wherein the antibody or antigen binding fragment thereof binds a B-cell derived cell line.

Embodiment 18. A pharmaceutical composition comprising an anti-CD22 antibody or antigen binding fragment thereof according to any one of embodiments 1 to 17.

Embodiment 19. A polynucleotide encoding the anti-CD22 antibody or antigen binding fragment thereof according to any one of embodiments 1 to 17.

Embodiment 20. The polynucleotide according to embodiment 19 comprising a heavy chain nucleic acid sequence of SEQ ID NO: 16 and a light chain nucleic acid sequence of SEQ ID NO: 15.

Embodiment 21. A host cell comprising the polynucleotide of either embodiment 19 or embodiment 20.

Embodiment 22. A method for producing an anti-CD22 antibody or antigen binding fragment thereof, comprising expressing the polynucleotide according to either embodiment 19 or embodiment 20 in a host cell.

Embodiment 23. An anti-CD22 antibody or antigen binding fragment thereof obtainable by the method of embodiment 22.

Embodiment 24. The anti-CD22 antibody or antigen binding fragment thereof according to any one of embodiments 1 to 17 or 23, wherein the antibody or antigen binding fragment thereof is conjugated to one or more drugs.

Embodiment 25. The anti-CD22 antibody or antigen binding fragment thereof according to embodiment 24, wherein the antibody or antigen binding fragment thereof is conjugated to one or more drugs selected from the group consisting of a cytotoxin, a tubulysin derivative, an antimicrobial agent, a therapeutic agent, a prodrug, a peptide, a protein, an enzyme, a lipid, a biological response modifier, a pharmaceutical agent, a lymphokine, a heterologous antibody, a fragment of a heterologous antibody, a detectable label, a polyethylene glycol (PEG), a radioisotope, and a combination thereof.

Embodiment 26. The anti-CD22 antibody or antigen binding fragment thereof according to embodiment 25, wherein the one or more drugs is a cytotoxin selected from the group consisting of a DNA scission agent, a DNA binding agent, a DNA intercalating agent, a topoisomerase I inhibitor, a topoisomerase II inhibitor, a tubulysin derivative, and an antimitotic agent.

Embodiment 27. The anti-CD22 antibody or antigen binding fragment thereof according to embodiment 25, wherein the one or more drugs is a cytotoxin selected from the group consisting of calicheamicins, pyrrolobenzodiazepines, camptothecins, daunorubicins, doxorubicins, auristatins, and maytansinoids.

Embodiment 28. The anti-CD22 antibody or antigen binding fragment thereof according to embodiment 25, wherein the one or more drugs is a cytotoxin which is a topoisomerase I inhibitor.

Embodiment 29. The anti-CD22 antibody or antigen binding fragment thereof according to embodiment 28, wherein the topoisomerase I inhibitor is exatecan:

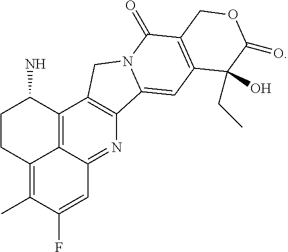 [SMILES: CC[C@@]1(O)C(=O)OCc2c1cc1n(c2=O)Cc2c-1nc1cc(F)c(C)c3c1c2[C@@H](N)CC3]

Embodiment 30. The anti-CD22 antibody or antigen binding fragment thereof according to embodiment 28, wherein the topoisomerase I inhibitor is:

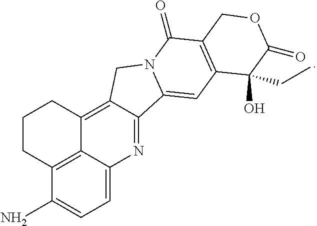 [SMILES: CC[C@@]1(O)C(=O)OCc2c1cc1n(c2=O)Cc2c-1nc1ccc(N)c3c1c2CCC3]

Embodiment 31. The anti-CD22 antibody or antigen binding fragment thereof according to any one of embodiments 24 to 30, wherein the antibody or antigen binding fragment is conjugated to a drug at a drug to antibody ratio (DAR) of about 4 to about 8.

Embodiment 32. The anti-CD22 antibody or antigen binding fragment thereof according to embodiment 31, wherein the antibody or antigen binding fragment is conjugated to a drug at a drug to antibody ratio (DAR) of about 8.

Embodiment 33. The anti-CD22 antibody or antigen binding fragment thereof according to embodiment 31, wherein the antibody or antigen binding fragment is conjugated to a drug at a drug to antibody ratio (DAR) of 4.

Embodiment 34. An antibody-drug conjugate (ADC) of Formula (I):

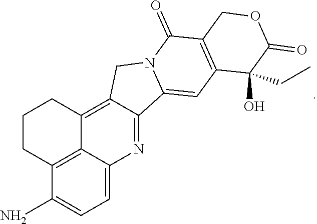

wherein
Ab is an antibody or antigen-binding fragment thereof according to any one of embodiments 1 to 17,
k is an integer from 1 to 10;
each G^{A} is independently a conjugation group conjugated to the antibody or antigen-binding fragment thereof;
each D^{C} is selected from the group consisting of a cytotoxin, a tubulysin derivative, an antimicrobial agent, a therapeutic agent, a prodrug, a peptide, a protein, an enzyme, a lipid, a biological response modifier, a pharmaceutical agent, a lymphokine, a heterologous antibody, a fragment of a heterologous antibody, a detectable label, a polyethylene glycol (PEG), and a radioisotope; and
each J^{A} is independently a linker.

Embodiment 35. The antibody-drug conjugate according to embodiment 34 wherein each J^{A} is independently a group of Formula (ICA):

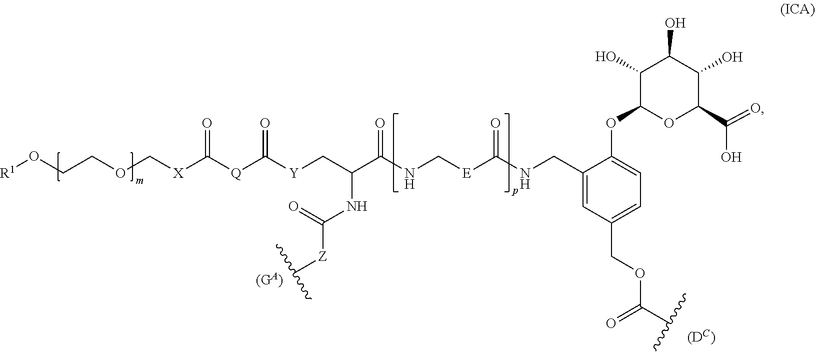

E is (CH₂)ₙ₁, wherein n1 is 0, 1, 2 or 3;
Q is

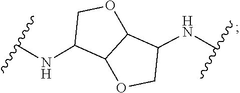 [SMILES: *NC1COC2C(N*)COC12]

R¹ is C₁₋₄ alkyl;
X is (CH₂)ₙ₂, wherein n2 is 0, 1, 2, or 3;
Y is (CH₂)ₙ₃, wherein n3 is 0, 1, 2, 3, or 4;
Z is (CH₂)ₙ₄, wherein n4 is 1, 2, 3, 4, or 5;
m is an integer from 5 to 17;
p is 1 or 0;
(G^{A}) indicates the point of attachment to G^{A}; and
(D^{C}) indicates the point of attachment to D^{C}.

Embodiment 36. The antibody-drug conjugate according to embodiment 35, wherein Q is:

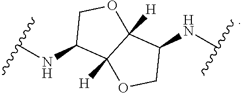 [SMILES: *N[C@H]1CO[C@H]2[C@@H]1OC[C@@H]2N*]

Embodiment 37. The antibody-drug conjugate according to either embodiment 35 or embodiment 36, wherein m is 9, 10, 11, 12, or 13.

Embodiment 38. The antibody-drug conjugate according to any one of embodiments 35 to 37, wherein R¹ is CH₃.

Embodiment 39. The antibody-drug conjugate according to any one of embodiments 35 to 38, wherein E is CH₂.

Embodiment 40. The antibody-drug conjugate according to any one of embodiments 35 to 39, wherein X is CH₂.

Embodiment 41. The antibody-drug conjugate according to any one of embodiments 35 to 40, wherein Y is (CH₂)₂.

Embodiment 42. The antibody-drug conjugate according to any one of embodiments 35 to 41, wherein Z is (CH₂)₂.

Embodiment 43. The antibody-drug conjugate according to any one of embodiments 35 to 41, wherein Z is CH₂.

Embodiment 44. The antibody-drug conjugate according to any one of embodiments 35 to 43, wherein p is 1.

Embodiment 45. The antibody-drug conjugate according to embodiment 35, wherein each J^{A} is a group of Formula (ICB):

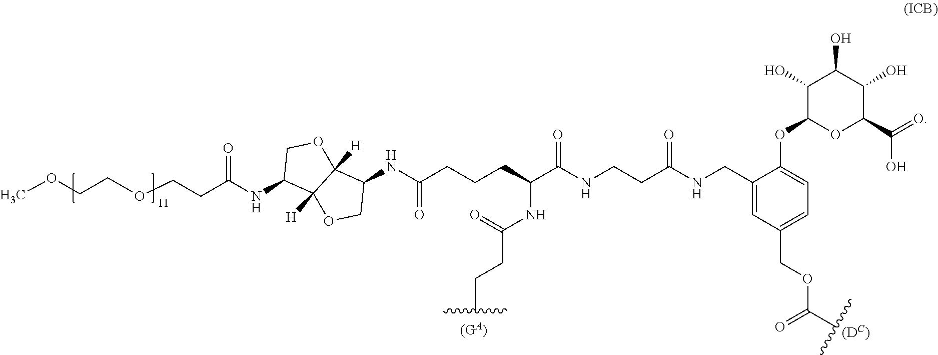

Embodiment 46. The antibody-drug conjugate according to any one of embodiments 34 to 45, wherein G^{A} is selected from the group consisting of:

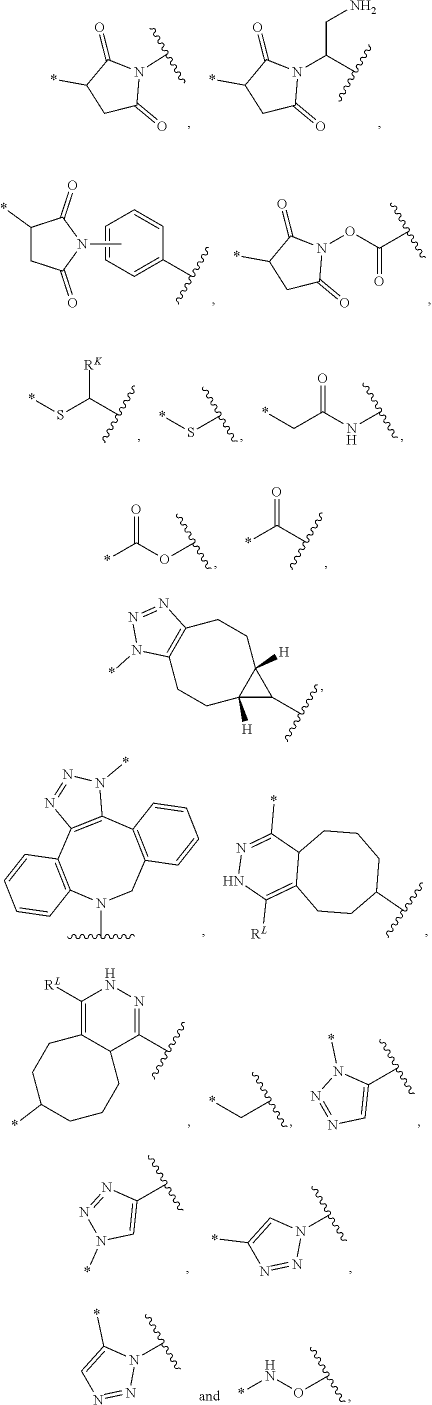

wherein R^{K} is H or CH₃, R^{L} is C₁₋₆ alkyl, and [symbol] indicates the point of attachment to the antibody or antigen-binding fragment thereof.

Embodiment 47. The antibody-drug conjugate according to embodiment 46, wherein G^{A} is:

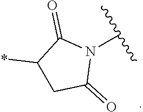 [SMILES: *C1CC(=O)N(*)C1=O]

Embodiment 48. The antibody-drug conjugate according to embodiment 46, wherein G^{A} is:

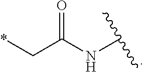 [SMILES: *CC(=O)N*]

Embodiment 49. The antibody-drug conjugate according to any one of embodiments 35 to 48, wherein k is an integer from about 4 to about 8.

Embodiment 50. The antibody-drug conjugate according to embodiment 49, wherein k is about 4.

Embodiment 51. The antibody-drug conjugate according to embodiment 49, wherein k is about 8.

Embodiment 52. The antibody-drug conjugate according to any one of embodiments 34 to 51, wherein each D^{C} is:

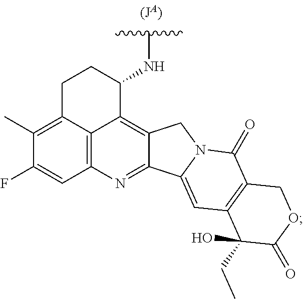 [SMILES: *N[C@H]1CCc2c(C)c(F)cc3nc4c(c1c23)Cn1c-4cc2c(c1=O)COC(=O)[C@]2(O)CC]

and (J^{A}) indicates the point of attachment to J^{A}.

Embodiment 53. The antibody-drug conjugate according to embodiment 52, wherein G^{A}-J^{A}-D^{C} is:

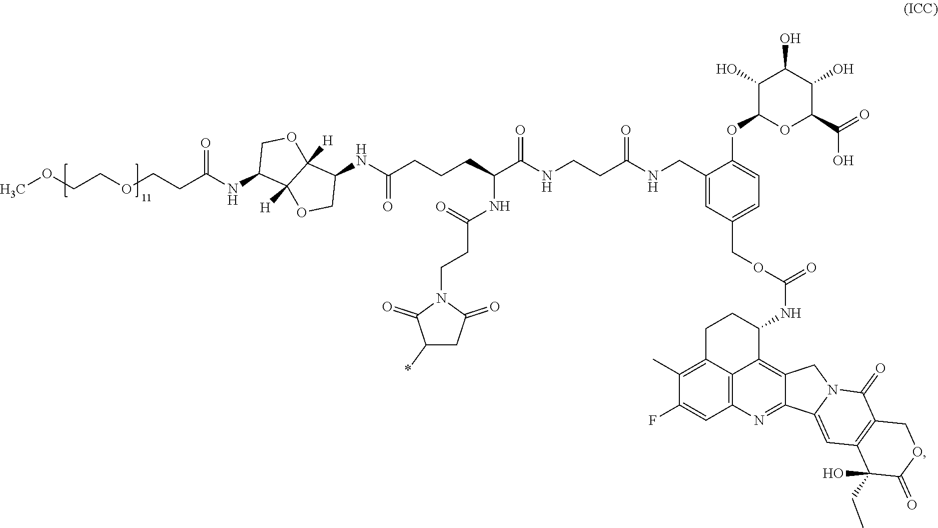

wherein [symbol] indicates the point of attachment to the antibody or antigen-binding fragment thereof.

Embodiment 54. The antibody-drug conjugate according to embodiment 52, wherein G^{A}-J^{A}-D^{C} is:

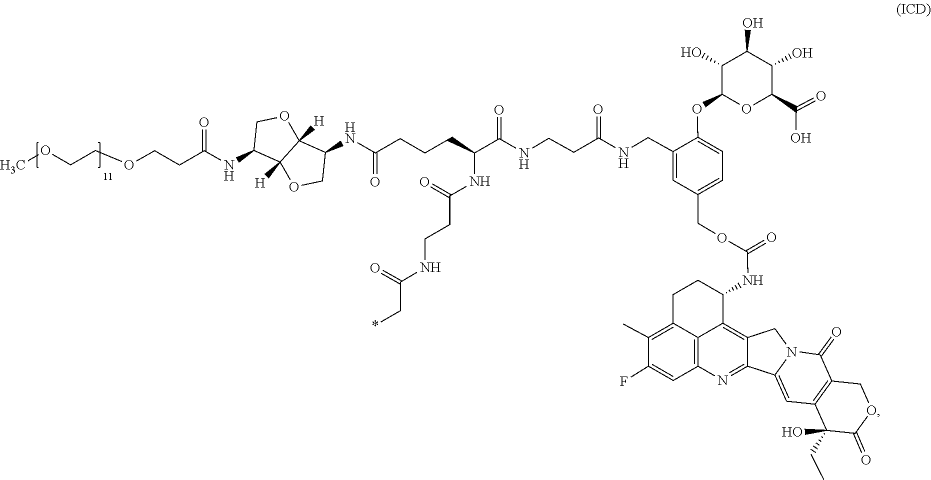

wherein [symbol] indicates the point of attachment to the antibody or antigen-binding fragment thereof.

Embodiment 55. The antibody-drug conjugate according to embodiment 34, wherein each J^{A} is a group of Formula (ICE):

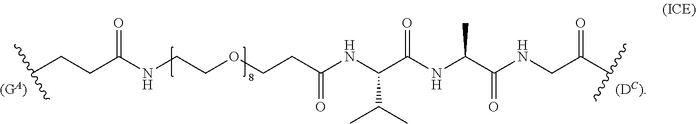

Embodiment 56. The antibody-drug conjugate according to embodiment 55, wherein G^{A} is selected from the group consisting of:

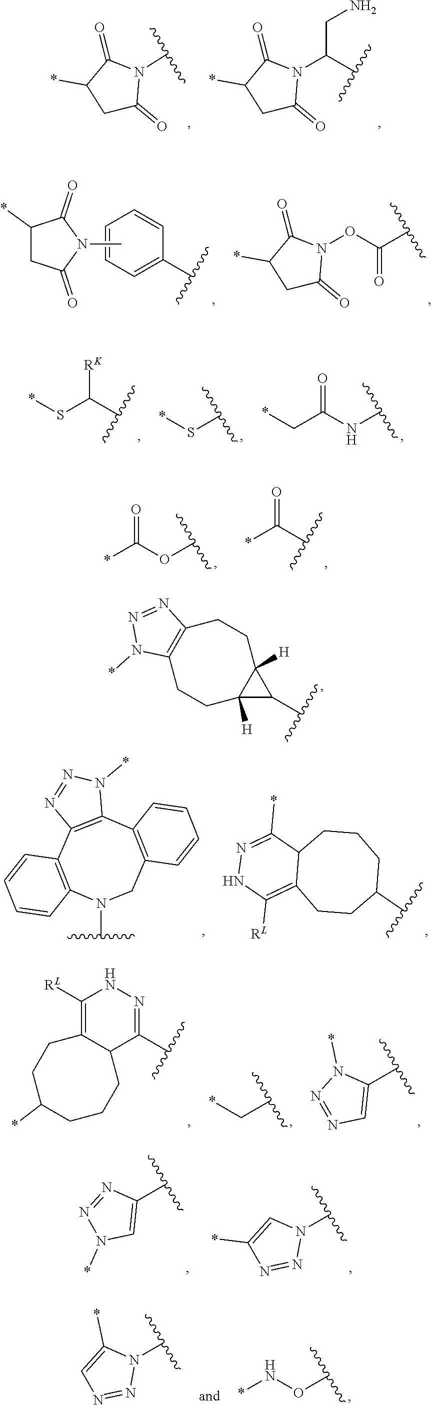

wherein R^{K} is H or CH₃, R^{L} is C₁₋₆ alkyl, and [symbol] indicates the point of attachment to the antibody or antigen-binding fragment thereof.

Embodiment 57. The antibody-drug conjugate according to embodiment 56, wherein G^{A} is:

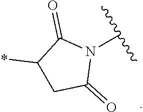 [SMILES: *C1CC(=O)N(*)C1=O]

Embodiment 58. The antibody-drug conjugate according to embodiment 56, wherein G^{A} is:

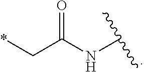 [SMILES: *CC(=O)N*]

Embodiment 59. The antibody-drug conjugate according to any one of embodiments 55 to 58, wherein k is an integer from about 4 to about 8.

Embodiment 60. The antibody-drug conjugate according to embodiment 59, wherein k is about 4.

Embodiment 61. The antibody-drug conjugate according to embodiment 59, wherein k is about 8.

Embodiment 62. The antibody-drug conjugate according to any one of embodiments 55 to 61, wherein each D^{C} is:

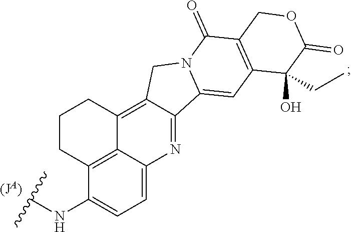 [SMILES: *Nc1ccc2nc3c(c4c2c1CCC4)Cn1c-3cc2c(c1=O)COC(=O)[C@]2(O)CC]

and (J^{A}) indicates the point of attachment to J^{A}.

Embodiment 63. The antibody-drug conjugate according to embodiment 62, wherein G^{A}-J^{A}-D^{C} is:

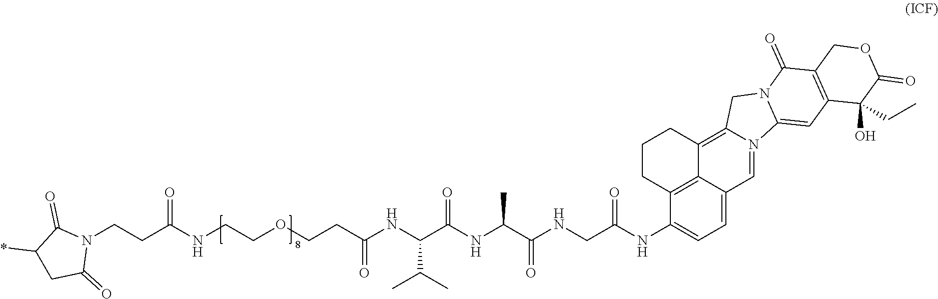

wherein [symbol] indicates the point of attachment to the antibody or antigen-binding fragment thereof.

Embodiment 64. The antibody-drug conjugate according to embodiment 34, wherein each J^{A} is a group of Formula (ICG):

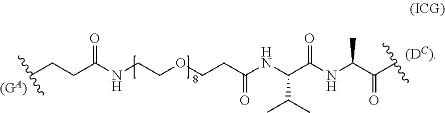

Embodiment 65. The antibody-drug conjugate according to embodiment 64, wherein G^{A} is selected from the group consisting of:

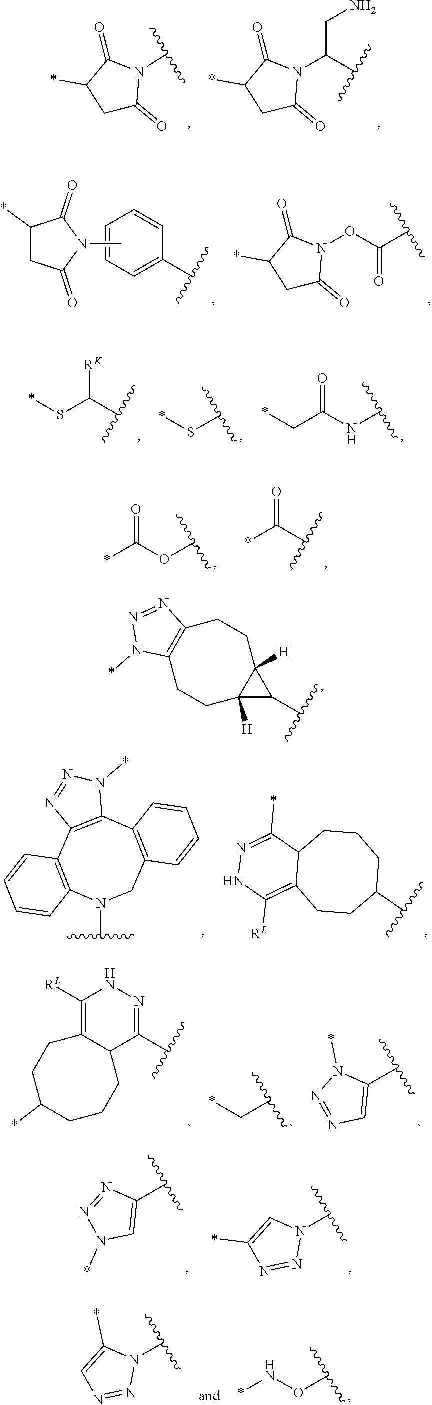

wherein R^{K} is H or CH₃, R^{L} is C₁₋₆ alkyl, and [symbol] indicates the point of attachment to the antibody or antigen-binding fragment thereof.

Embodiment 66. The antibody-drug conjugate according to embodiment 65, wherein G^{A} is:

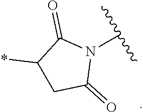 [SMILES: *C1CC(=O)N(*)C1=O]

Embodiment 67. The antibody-drug conjugate according to embodiment 65, wherein G^{A} is:

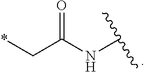 [SMILES: *CC(=O)N*]

Embodiment 68. The antibody-drug conjugate according to any one of embodiments 64 to 67, wherein k is an integer from about 4 to about 8.

Embodiment 69. The antibody-drug conjugate according to embodiment 68, wherein k is about 4.

Embodiment 70. The antibody-drug conjugate according to embodiment 68, wherein k is about 8.

Embodiment 71. The antibody-drug conjugate according to any one of embodiments 64 to 70, wherein each D^{C} is:

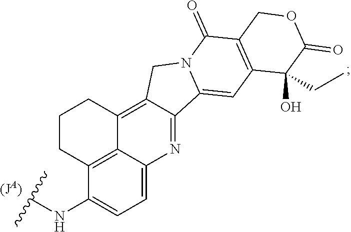 [SMILES: *Nc1ccc2nc3c(c4c2c1CCC4)Cn1c-3cc2c(c1=O)COC(=O)[C@]2(O)CC]

and (J^{A}) indicates the point of attachment to J^{A}.

Embodiment 72. The antibody-drug conjugate according to embodiment 71, wherein G^{A}-J^{A}-D^{C} is:

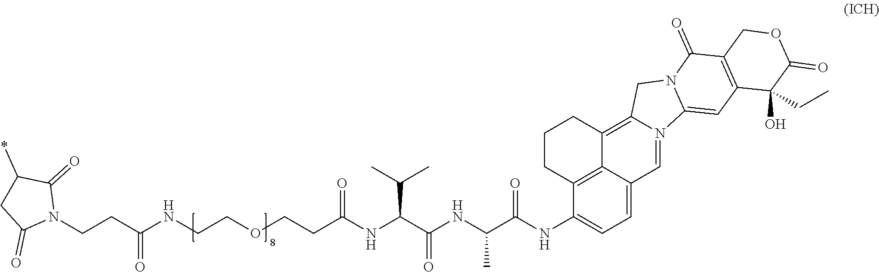

wherein [symbol] indicates the point of attachment to the antibody or antigen-binding fragment thereof.

Embodiment 73. An antibody-drug conjugate comprising:
(i) an anti-CD22 antibody or antigen binding fragment thereof comprising a heavy chain CDR1 (HCDR1) comprising the amino acid sequence of SEQ ID NO: 1, a heavy chain CDR2 (HCDR2) comprising the amino acid sequence of SEQ ID NO: 2, a heavy chain CDR3 (HCDR3) comprising the amino acid sequence of SEQ ID NO: 3, a light chain CDR1 (LCDR1) comprising the amino acid sequence of SEQ ID NO: 4, a light chain CDR2 (LCDR2) comprising the amino acid sequence of SEQ ID NO: 5, and a light chain CDR3 (LCDR3) comprising the amino acid sequence of SEQ ID NO: 6; and
(ii) a linker payload of Formula (ICC):

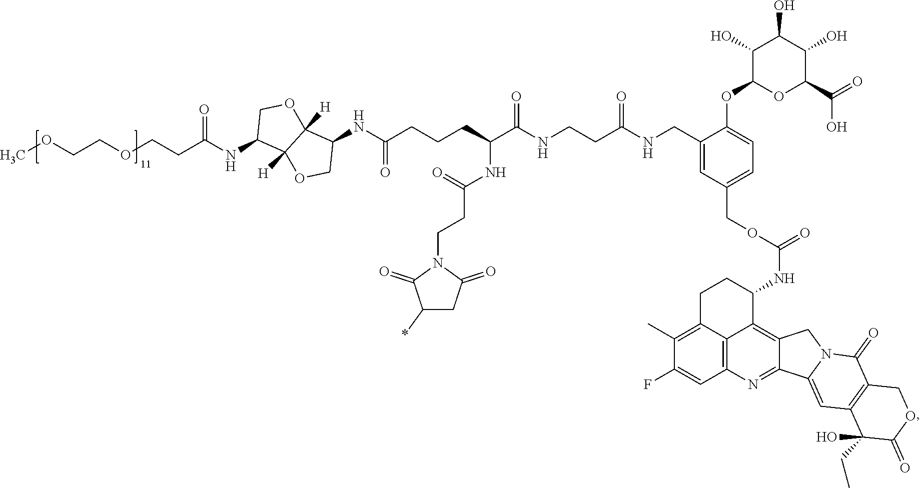

wherein [symbol] indicates the point of attachment to the antibody or antigen-binding fragment thereof; and
wherein the ADC has a drug to antibody ratio (DAR) ranging from about 4 to about 8.

Embodiment 74. An antibody-drug conjugate comprising:
(i) an anti-CD22 antibody or antigen binding fragment thereof comprising a heavy chain CDR1 (HCDR1) comprising the amino acid sequence of SEQ ID NO: 1, a heavy chain CDR2 (HCDR2) comprising the amino acid sequence of SEQ ID NO: 2, a heavy chain CDR3 (HCDR3) comprising the amino acid sequence of SEQ ID NO: 3, a light chain CDR1 (LCDR1) comprising the amino acid sequence of SEQ ID NO: 4, a light chain CDR2 (LCDR2) comprising the amino acid sequence of SEQ ID NO: 5, and a light chain CDR3 (LCDR3) comprising the amino acid sequence of SEQ ID NO: 6; and
(ii) a linker payload of Formula (ICD):

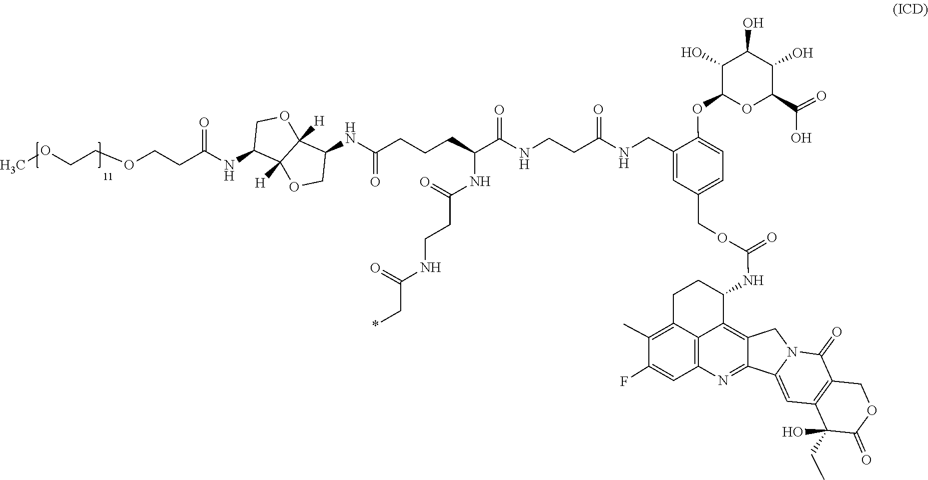

wherein [symbol] indicates the point of attachment to the antibody or antigen-binding fragment thereof; and
wherein the ADC has a drug to antibody ratio (DAR) ranging from about 4 to about 8.

Embodiment 75. An antibody-drug conjugate comprising:
(i) an anti-CD22 antibody or antigen binding fragment thereof comprising a heavy chain CDR1 (HCDR1) comprising the amino acid sequence of SEQ ID NO: 1, a heavy chain CDR2 (HCDR2) comprising the amino acid sequence of SEQ ID NO: 2, a heavy chain CDR3 (HCDR3) comprising the amino acid sequence of SEQ ID NO: 3, a light chain CDR1 (LCDR1) comprising the amino acid sequence of SEQ ID NO: 4, a light chain CDR2 (LCDR2) comprising the amino acid sequence of SEQ ID NO: 5, and a light chain CDR3 (LCDR3) comprising the amino acid sequence of SEQ ID NO: 6; and
(ii) a linker payload of Formula (ICF):

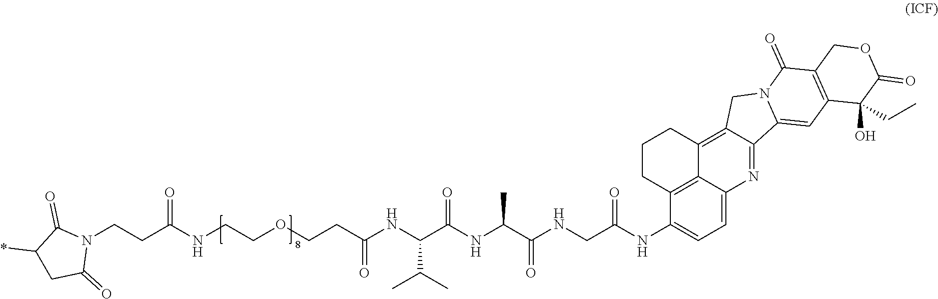

wherein [symbol] indicates the point of attachment to the antibody or antigen-binding fragment thereof; and
wherein the ADC has a drug to antibody ratio (DAR) ranging from about 4 to about 8.

Embodiment 76. An antibody-drug conjugate comprising:
(i) an anti-CD22 antibody or antigen binding fragment thereof comprising a heavy chain CDR1 (HCDR1) comprising the amino acid sequence of SEQ ID NO: 1, a heavy chain CDR2 (HCDR2) comprising the amino acid sequence of SEQ ID NO: 2, a heavy chain CDR3 (HCDR3) comprising the amino acid sequence of SEQ ID NO: 3, a light chain CDR1 (LCDR1) comprising the amino acid sequence of SEQ ID NO: 4, a light chain CDR2 (LCDR2) comprising the amino acid sequence of SEQ ID NO: 5, and a light chain CDR3 (LCDR3) comprising the amino acid sequence of SEQ ID NO: 6; and
(ii) a linker payload of Formula (ICH):

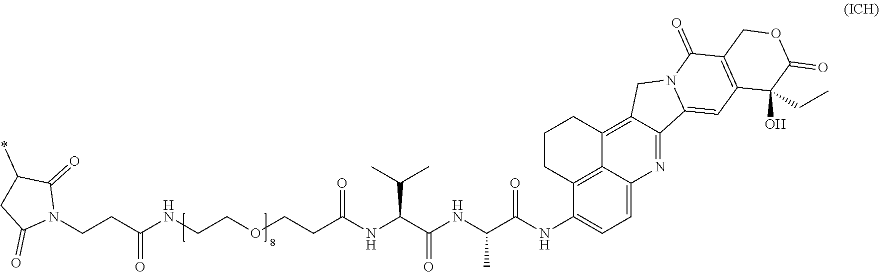

wherein [symbol] indicates the point of attachment to the antibody or antigen-binding fragment thereof; and
wherein the ADC has a drug to antibody ratio (DAR) ranging from about 4 to about 8.

Embodiment 77. The antibody-drug conjugate according to any one of embodiments 73 to 76, wherein the antibody or antigen binding fragment thereof comprises a VH chain comprising the amino acid sequence of SEQ ID NO: 7 and a VL chain comprising the amino acid sequence of SEQ ID NO: 8.

Embodiment 78. The antibody-drug conjugate according to any one of embodiments 73 to 77, wherein the antibody or antigen binding fragment thereof comprises a heavy chain (HC) comprising the amino acid sequence of SEQ ID NO: 9, and light chain (LC) comprising the amino acid sequence of SEQ ID NO: 10.

Embodiment 79. The antibody-drug conjugate according to any one of embodiments 73 to 77, wherein the antibody or antigen binding fragment thereof comprises a heavy chain (HC) comprising the amino acid sequence of SEQ ID NO: 13, and light chain (LC) comprising the amino acid sequence of SEQ ID NO: 10.

Embodiment 80. A pharmaceutical composition comprising an antibody-drug conjugate according to any one of embodiments 34 to 79.

Embodiment 81. The antibody-drug conjugate of any one of embodiments 34 to 79 or the pharmaceutical composition of embodiment 80, for use as a medicament.

Embodiment 82. The antibody-drug conjugate of any one of embodiments 34 to 79 or the pharmaceutical composition of embodiment 80, for use in the treatment of cancer.

Embodiment 83. The antibody-drug conjugate or the pharmaceutical composition for use in the treatment of cancer according to embodiment 82, wherein said cancer expresses CD22.

Embodiment 84. A method of treating cancer, the method comprising administering to a subject the antibody-drug conjugate of any one of embodiments 34 to 79 or the pharmaceutical composition of embodiment 80.

Embodiment 85. The method according to embodiment 84, wherein said cancer expresses CD22.

Embodiment 86. The method according to either embodiment 84 or embodiment 85, wherein the subject is a human.

Embodiment 87. The method according to any one of embodiments 84 to 86, or the antibody-drug conjugate or pharmaceutical composition for use according to any one of embodiments 71 to 73, wherein said cancer is a B-cell malignancy.

Embodiment 88. The method or antibody-drug conjugate or pharmaceutical composition for use according to embodiment 87, wherein said B-cell malignancy is selected from the group consisting of non-Hodgkin lymphoma, acute lymphoblastic leukemia (ALL), Burkitt lymphoma, chronic lymphocytic leukemia (CLL), small lymphocytic lymphoma (SLL), diffuse large B-cell lymphoma (DLBCL), follicular lymphoma (FL), mantle cell lymphoma (MCL), lymphoplasmacytic lymphoma, and Hairy cell leukemia.

Embodiment 89. The method or antibody-drug conjugate or pharmaceutical composition for use according to embodiment 88, wherein the B-cell malignancy is refractory [B-cell] acute lymphoblastic leukemia (ALL) or relapsed [B-cell] acute lymphoblastic leukemia (ALL).

Embodiment 90. The anti-CD22 antibody or antigen binding fragment thereof according to any one of embodiments 1 to 17 or the pharmaceutical composition of embodiment 18, for use as a medicament.

Embodiment 91. The anti-CD22 antibody or antigen binding fragment thereof according to any one of embodiments 1 to 17 or the pharmaceutical composition of embodiment 18, for use in the treatment of cancer.

Embodiment 92. The anti-CD22 antibody or antigen binding fragment thereof according to any one of embodiments 1 to 17 or the pharmaceutical composition of embodiment 18, wherein said cancer expresses CD22.

Embodiment 93. A method of treating cancer, the method comprising administering to a subject the anti-CD22 antibody or antigen binding fragment thereof according to any one of embodiments 1 to 17 or the pharmaceutical composition of embodiment 18.

Embodiment 94. The method according to embodiment 93, wherein said cancer expresses CD22.

Embodiment 95. The method according to either embodiment 93 or embodiment 94, wherein the subject is a human.

Embodiment 96. The method according to any one of embodiments 93 to 95, or the anti-CD22 antibody or antigen binding fragment thereof for use, or pharmaceutical composition for use according to either embodiment 81 or embodiment 82, wherein said cancer is a B-cell malignancy.

Embodiment 97. The method or the anti-CD22 antibody or antigen binding fragment thereof for use or pharmaceutical composition for use according to embodiment 96, wherein said B-cell malignancy is selected from the group consisting of non-Hodgkin lymphoma, acute lymphoblastic leukemia (ALL), Burkitt lymphoma, chronic lymphocytic leukemia (CLL), small lymphocytic lymphoma (SLL), diffuse large B-cell lymphoma (DLBCL), follicular lymphoma (FL), mantle cell lymphoma (MCL), lymphoplasmacytic lymphoma, and Hairy cell leukemia.

Embodiment 98. The method or antibody-drug conjugate or pharmaceutical composition for use according to embodiment 97, wherein the B-cell malignancy is refractory [B-cell] acute lymphoblastic leukemia (ALL) or relapsed [B-cell]acute lymphoblastic leukemia (ALL).

Embodiment 99. A method for detecting the presence or absence of a CD22 polypeptide in a sample, comprising:
(i) contacting a sample with an antibody or antigen binding fragment thereof according to any one of embodiments 1-17, or a pharmaceutical composition according to embodiment 18, to provide an antibody-antigen complex;
(ii) detecting the presence or absence of said antibody-antigen complex;
wherein the presence of the antibody-antigen complex confirms the presence of a CD22 polypeptide; and wherein the absence of the antibody-antigen complex confirms the absence of CD22 polypeptide.

Embodiment 100. The method according to embodiment 99, wherein the presence of said antibody-antigen complex is indicative of the presence of a CD22 expressing cancer cell, and wherein the absence of said antibody-antigen complex is indicative of the absence of a CD22 expressing cancer cell.

Embodiment 101. The method according to either embodiment 99 or embodiment 100, wherein the sample is an isolated sample obtainable from a subject.

Embodiment 102. A kit of parts comprising at least one of (i) a variable heavy chain, and (ii) a variable light chain of (a) the anti-CD22 antibody or antigen binding fragment thereof according to any one of embodiments 1 to 17, (b) the antibody drug conjugate according to any one of embodiments 34 to 79, or (c) the pharmaceutical composition according to either embodiment 18 or embodiment 80.

### EXAMPLES

### Example 1

### CD22 is Restrictedly Expressed in B Cell Malignancies

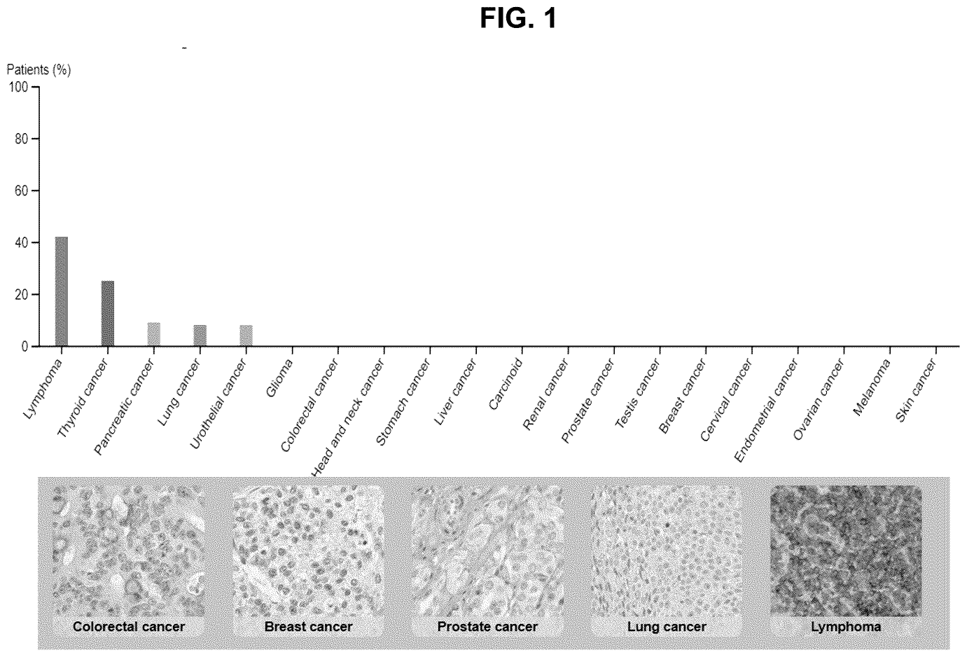

CD22, also known as Siglec-2, is a significant B-cell surface marker involved in the regulation of B-cell function. It acts as an inhibitory receptor for B-cell receptor (BCR) signaling through its interaction with sialic acid-containing ligands. CD22 is primarily found on the surface of mature B-cells, but is absent on the surface of pluripotent stem cells or other immune cells. Notably, it is highly expressed in B-cell malignancies, including various types of B-cell lymphomas and leukemias, with close to 90% positivity rates. FIG. 1 shows the % of patient samples expressing CD22 in specific cancers from Human Protein Atlas (HPA) database.

### Example 2

Generation of Anti-CD22 Antibody, mAb23-TM

A triple mutant (“TM”) version of the mAb23 (“mAb23-TM”) was modified from the lead anti-CD22 antibody (“mAb23”) to contain three mutations.

mAb23 was produced from hybridoma technology using ALIVAMAB Mouse that were immunized with recombinant human and cynomolgus CD22 as well as AD293 cells exogenously expressing human and cynomolgus CD22 extracellular domain. Lymphoid cells were harvested and mixed with P3X63Ag8.653 myeloma cells, and fusions were performed using polyethylene glycol (PEG) to generate hybridomas.

mAb23 was identified by binding to recombinant human and cynomolgus CD22 using enzyme-linked immunosorbent assays (ELISA). Hybridoma cell line supernatants were assayed for binding to either recombinant human, mouse or cynomolgus CD22. Bound antibodies were detected using anti-mouse antibodies conjugated to horse radish peroxidase (HRP). A known anti-CD22 antibody was used as a positive binding control and secondary antibody alone was used as a negative binding control. Wells showing binding to recombinant human/cynomolgus CD22 were assayed for binding to cells expressing CD22. Here AD293 exogenously expressing human CD22, cynomolgus CD22, or tumor cell lines expressing human CD22 (OCILY19 and HT) were incubated with the hybridoma supernatants and a fluorescently labelled secondary antibody. Cell surface binding was detected and quantified by fluorescence microscopy.

Hybridoma wells showing positive binding to recombinant human and cynomolgus monkey CD22 as well as to cells expressing human and cynomolgus CD22, were screened for internalization and cytotoxicity in CD22 expressing cells. AD293 cells expressing human or cynomolgus monkey CD22 were incubated with hybridoma supernatants and anti-mouse Fab conjugated with a pH sensitive fluorophore (pHRodo) for internalization or a Topoisomerase inhibitor cytotoxin for cytotoxicity. Cells showing intracellular fluorescence were detected and quantified by fluorescence microscopy. Cytotoxicity was assessed by measuring the viability of the cells using a CellTiter-Glo viability assay after a 3-day incubation.

Cells from hybridoma supernatants that demonstrated specific binding to human and cynomolgus monkey CD22 and supported effective internalization and cytotoxicity were collected and heavy and light chain genes were sequenced using next generation sequencing technologies. cDNA prepared from well 431 35k-1B8 was sequenced and variable heavy (VH) and variable light (VL) sequences determined. Two unique VH and three unique VL sequences were identified. G-blocks encoding each of the full length VH and VL were cloned into a bi-cistronic mammalian expression vector to generate antibodies with all the combinations of VH and VL genes. Each VH sequence was cloned into a region containing the constant domains of human IgG1, and the VL genes were cloned into a region containing the IgG1κ light chain constant domains.

The IgG VL and VH genes were transiently transfected into CHO cells and the antibodies expressed and tested for CD22 binding. Of the six possible combinations of VH and VL genes, two showed CD22 binding by ELISA and biolayer interferometry and supported internalization. The antibody containing VH sequence 20_7VHGL and VL sequence Hy9_VL2 was selected for further development and identified as mAb23.

To minimise Fc receptor interactions, the VH-VL were cloned into an antibody expression cassette where the Fc sequence contained three mutations (L234F, L235E, and S331P; identified as the triple mutant or ‘TM’) shown to reduce effector function. mAb23 containing the three mutations is known as the mAb23-TM.

### Example 3

In-Vitro Characterization of Anti-CD22 Antibody, mAb23-TM

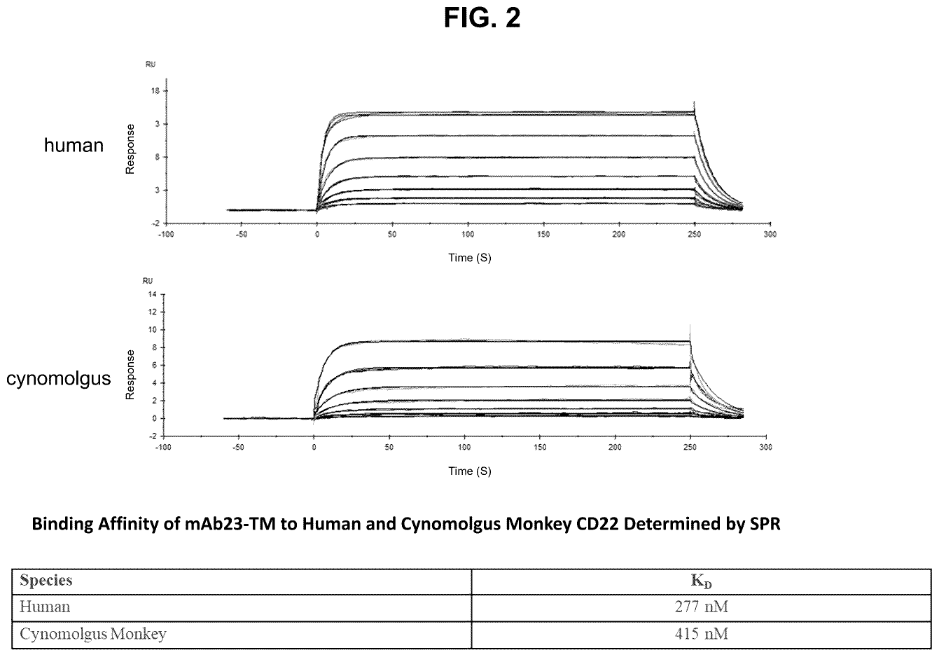

mAb23-TM binds similarly to human and cynomolgus monkey CD22 by Surface Plasmon Resonance (SPR). mAb23-TM binds to immobilized human and cynomolgus monkey CD22 with similar affinities (within 2-fold) with KD values of 277 nM and 415 nM for the human and cynomolgus monkey CD22, respectively (FIG. 2). The kinetic rate constants (kₒₙ and k_{off}) and equilibrium dissociation constants (KD) of anti-CD22 antibody mAb23-TM, for recombinant mammalian cell expressed human, and cynomolgus monkey (R&D Systems) CD22 variants, was determined at 25° C. by SPR on a Biacore T200 instrument (Cytiva, Marlborough, MA).

Binding affinity, cross-reactivity, and developability characteristics for mAb23-TM are summarized in Table 1 below:

TABLE 1

| | | Binding | | |
|---|---|---|---|---|
| | Binding | Affinity to | Heat Stress | Photo stress |
| Clone ID | Affinity to | cynomolgus | (% aggregate | (% aggregate |
| (hIgG1-TM) | human CD22 | monkey CD22 | increase) | increase) |
| mAb23-TM | 277 nM | 415 nM | 0.33% | 0.21% |

In Vitro Cell-Based Binding of mAb23-TM and its ADC Conjugates

Generation of AD293 Cell Lines Stably Expressing Human, Murine, or Cynomolgus Monkey CD22 Transgene: AD293 cell lines stably expressing human, cyno or murine CD22 were generated using a lentiviral system from System Biosciences (Palo Alto, CA). The human CD22 was obtained from cDNA using PCR. The cyno and murine CD22 transgene were generated by gene synthesis by Integrated DNA Technologies (IDT DNA, Coralville, IA). These genes were inserted into a lentiviral expression vector, pCDH1CMV-MCS-EF1puro (System Biosciences, Palo Alto, CA) using standard molecular biology techniques. The gene was placed under the control of the CMV promoter, followed by an EF1 promoter driving puromycin resistance. This expression vector was used to generate lentiviral particles from a 293X (Thermo Fisher Scientific, Waltham, MA) producer cell line using the pPACK H1 HIV Lentivector Packaging Kit (System Biosciences, Palo Alto, CA) as per the manufacturer's instructions. AD293 cells from Stratagene/Agilent Technologies (Santa Clara, CA) were transduced with these lentiviral particles and a stably-expressing transgene cell pool was selected using 1 μg/mL puromycin

Binding of mAb23-TM to AD293 Cell Lines Stably Expressing Human, Murine, and Cynomolgus Monkey CD22 Transgene

Flow cytometry was used to determine the binding of AF647 fluorophore conjugated unarmed antibody mAb23-TM to non-transduced AD293 cells and AD293 cells stably expressing human, murine, or cynomolgus monkey CD22. Cells were collected using TrypLE (ThermoFisher Scientific, Waltham, MA) and were transferred to a round-bottomed, 96-well plate at a density of 200,000 cells per well. Cells were pelleted by centrifugation at 500×g for 3 minutes at RT, treated with 2.5 μg/mL Fc block (BD Biosciences) 50 μL/well for 10 minutes and pelleted at 500×g for 3 minutes 4° C. A titration curve of 100 μg/mL to 0.78125 μg/mL was used for the antibody with 1:2 serial dilution. Cells were treated with total volume of 30 μL per well. Plates were incubated on ice for 30 minutes. Primary antibody was removed by centrifugation at 500×g for 3 minutes at 4° C. and were washed twice with 100 μL BD Stain buffer (BD Biosciences). Cells were resuspended in 100 μL BD Stain buffer and kept on ice. Fluorescence of single cells was measured using a Becton Dickinson Biosciences Fortessa cytometer and BD FACSDiva software (BD Biosciences, San Jose, CA). Data were analyzed using FLOWJO software (BD Biosciences, San Jose, CA).

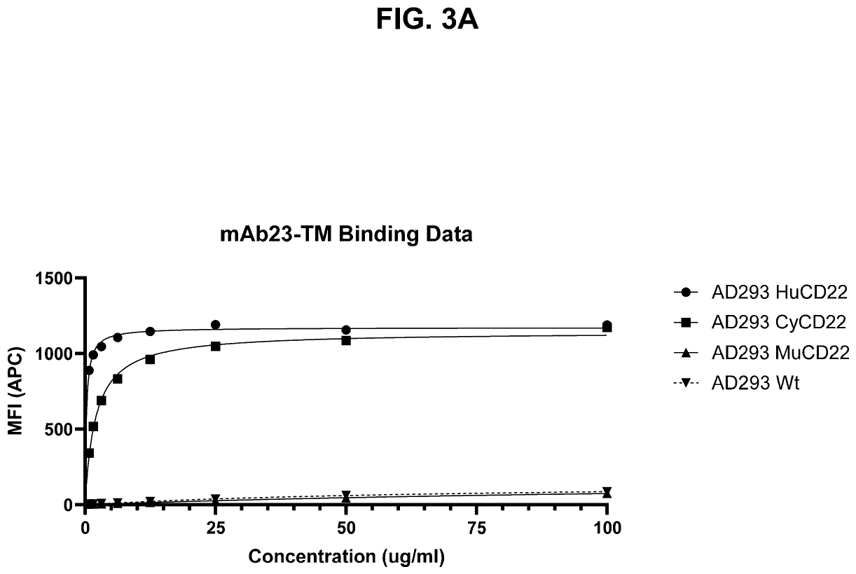

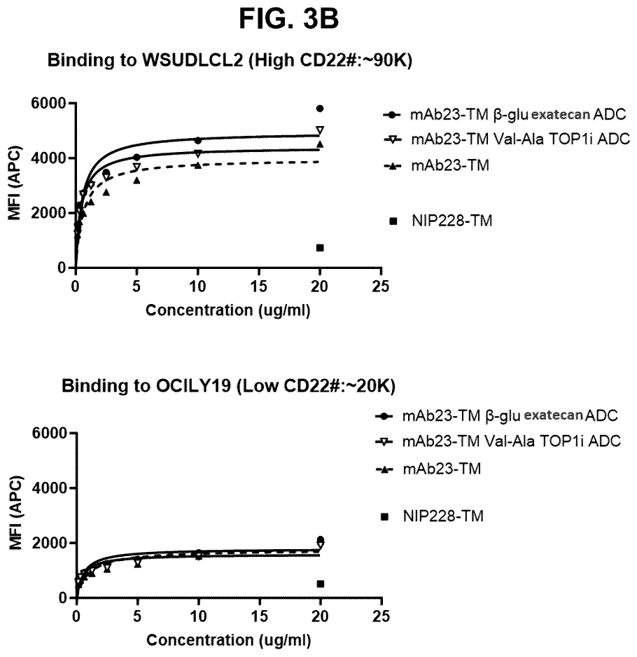

The result in FIG. 3A showed that mAb23-TM specifically bound to AD293 cells stably expressing human, cynomolgus monkey CD22, but did not bind to the murine CD22 or CD22-negative non-transduced AD293 cells.

Binding of Antibody mAb23-TM and its ADC Conjugates to Human Diffuse Large B-Cell Lymphoma (DLBCL) Cell Lines WSUDLCL2 and OCILY19.

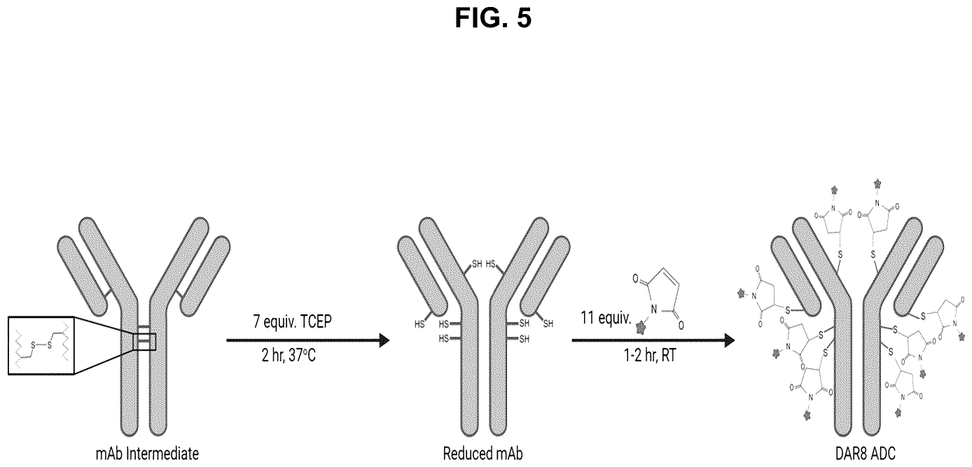

The TOP1i ('0132) and exatecan drugs were covalently bound to native cysteines in the antibody through a thiosuccinimide linkage, with about eight drugs bound per antibody (i.e., DAR of about 8). A schematic of CD22 ADCs is shown in FIG. 5. The anti-CD22 human IgG1κ monoclonal antibody mAb23-TM was conjugated via a cleavable maleimide-PEG8-valine-alanine linker to the TOP1i payload ('0132) for the mAb23-TM Val-Ala TOP1i ('0132), or via a β-glucuronidase-cleavable linker to exatecan payload ('4332) for the mAb23-TM β-glucuronidase exatecan ADC.

| | | |
|---|---|---|
| Generic name/ | mAb23-TM | mAb23-TM |
| chemical name | β-glu exatecan, | Val-Ala TOP1i, |
| | mAb23-TM-′4332 | mAb23-TM-′0132 |
| Isotype | Human IgG1 | Human IgG1 |

| | | | | |
|---|---|---|---|---|
| Relative molecular | 162 | kDa | 162 | kDa |
| weight | | | | |
| Concentration | 10.24 | mg/mL | 10 | mg/mL |

| | | |
|---|---|---|
| Purity (monomer | 97.5% | 98.8% |
| content) | | |
| DAR | 7.92 | 8 |

The payloads released from the ADCs referred to herein can be:

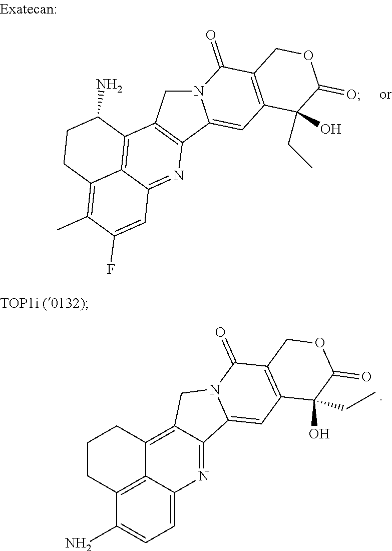 [SMILES: CC[C@@]1(O)C(=O)OCc2c1cc1n(c2=O)Cc2c-1nc1cc(F)c(C)c3c1c2[C@@H](N)CC3 ; CC[C@@]1(O)C(=O)OCc2c1cc1n(c2=O)Cc2c-1nc1ccc(N)c3c1c2CCC3]

Flow cytometry was used to determine the binding of AF647 fluorophore conjugated ADCs, antibody mAb23-TM and isotype NIP228-TM to WSU-DLCL2 and OCILY19 cell lines endogenously expressing human CD22. In culture cells were transferred to a round-bottomed, 96-well plate at a density of 200,000 cells per well. Cells were pelleted by centrifugation at 500×g for 3 minutes at RT, treated with 2.5 μg/mL Fc block (BD Biosciences) 50 μL/well for 5-10 minutes and pelleted at 500×g for 3 minutes 4° C. mAb23-TM and its ADC conjugates were prepared at twice the required concentration, 40 μg/mL in BD stain buffer (BD Biosciences). mAb23-TM, control antibody and the ADCs were serially diluted in the buffer 1:2. Viability stain, fixable live dead blue was prepared 2× and added at equal volume of 50 μL to 2× mAb23-TM and the ADCs to achieve 1× concentration. 40 μg/mL of unarmed antibody was prepared 2× and added with viability stain to get 1×. The cells were resuspended in 35 μL of BD Stain buffer containing concentrations from 20 μg/mL to 0.15625 μg/mL. Plates were incubated on ice for 30 minutes. The cells were centrifuged at 500×g for 3 minutes at 4° C. and washed twice with 100 μL/well FACS buffer. Cells were resuspended in 100 μL FACS buffer and kept on ice. Fluorescence of live, single cells was measured using a Becton Dickinson Biosciences Symphony cytometer and BD FACSDiva software (BD Biosciences, San Jose, CA). Data were analyzed using FLOWJO software (BD Biosciences, San Jose, CA).

As shown in FIG. 3B, the mAb23-TM and its ADC conjugates (mAb23-TM Val-Ala TOP1i ('0132) ADC and mAb23-TM β-glu exatecan ADCs), bound to OICLY19 and WSU-DLCL2 with low and high CD22 levels, respectively, demonstrating that both unarmed CD22 antibody and its conjugated ADCs recognize endogenously expressed CD22 in human cancer cell lines. Importantly, the binding of unarmed antibody mAb23-TM and its conjugated ADCs was comparable, showing that binding properties of the parental antibody are maintained after conjugation to the linker payloads.

### Example 4

Internalization Evaluation of mAb23-TM and its ADC Conjugates

Internalization assays were run to measure the rate and extent to which mAb23-TM and its ADC conjugates are internalized by cells after engaging with the target antigen. The assay relies on labeling the Fc-containing test antibodies with a Fab fragment-conjugated to a pH-sensitive fluorophore (Sartorius) and the ability of CD22 receptor to internalize when bound to antibody or ADCs. The internalization signal was detected with standard methods of live cell imaging using the Incucyte SX5.

Well flat bottom plates (96, Perkin Elmer, catalog #6055300) were coated with Cultrex Poly-L-Lysine (Fisher Scientific, PLL) 50 μL/well for 15 minutes at room temperature (RT) in BSC. PLL was aspirated, and plates were washed twice with 150 μL sterile DI water. The plates were dried in a 37° C. incubator overnight. Cells were counted and 7E6 cells of WSU-DLCL2 and OCILY19 were collected in a 15 mL sterile conical tube, centrifuged at 300×g for 10 minutes at RT and aspirated. Cell lines were resuspended in 5.6 mL of phenol red free RPMI160 with 20% filtered FBS medium. The cells were mixed and plated into the PLL coated plates at 80 μL/well in triplicates for all treatments. 20-fold diluted stocks of CD22 ADCs and mAbs were prepared in 1×DPBS. 2 μg/mL of each tested article was conjugated to Incucyte Fab Fluor Orange (Sartorius) at a 1:5 molar ratio of (mAb: fluorophore ration) in phenol red free medium for 15 minutes at RT prevented from light exposure. Final concentration of 0.5 μg/mL were prepared from conjugated ADCs and mAb in phenol red free medium. 20 μL of prepared treatments were added to cells in triplicates. The plate was mixed by tilting gently and placed in Sartorius Incucyte SX5 to collected hourly imaging over 24 hours. The raw data was normalized to isotype and analyzed data was plotted as [Total Orange Object Integrated Intensity (OCU×μm²/Image)/Confluence]×100.

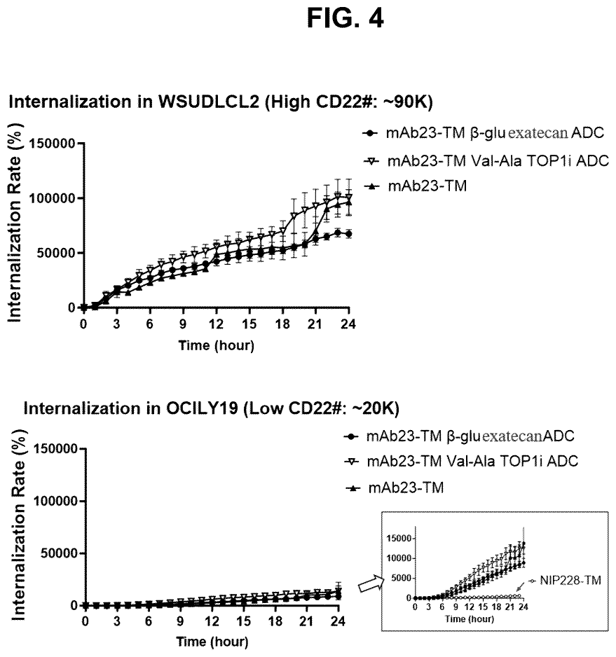

An increase in the normalized fluorescence intensity was observed with the unarmed antibody mAb23-TM and its conjugated ADC forms compared to the isotype antibody NIP228-TM in CD22-expressing DLBCL lines WSU-DLCL2 and OCILY19, which have relatively high (CD22 #: ˜90K) and low (CD22 #: ˜20K) CD22 levels, respectively (FIG. 4). The WSU-DLCL2 cell line demonstrated much stronger accumulation of internalized antibody or ADC compared to OCILY19. However, even though OCILY19 exhibited weaker fluorescence intensity with an average compared to WSU-DLCL2, it still internalized mAb23-TM and its ADC conjugates significantly more than the isotype control NIP228-TM as visualized by enlarged scale, thus demonstrating CD22-dependent internalization. Importantly, internalization of ADCs is comparable to mAb23-TM, suggesting no change in internalization kinetics after conjugation to the drug-linker payloads.

### Example 5

Potency of mAb23-TM Val-Ala TOP1i ('0132) and mAb23-TM β-Glu Exatecan ADCs on B-Cell Malignant Cell Lines

The cytotoxicity assays were designed to determine whether mAb23-TM Val-Ala TOP1i ('0132) and mAb23-TM β-glu exatecan ADCs could reduce the cell viability of CD22 expressing B-cell malignant cells including the Diffuse Large B-cell Lymphoma (DLBCL) cell lines WSU-DLCL2, OCILY19, their CD22 knockdown lines and additional malignant B-cell lines including B-cell Acute Lymphoblastic Leukemia (B-ALL), Follicular Lymphoma (FL), and Mantle Cell Lymphoma (MCL). The assay relies on the principle that dying cells will produce less adenosine triphosphate (ATP). By using a reagent that exhibits bioluminescence in the presence of ATP (CellTiter-Glo 2.0), dose-dependent decreases in cell viability are reflected in decreased bioluminescent signal. The bioluminescent signal was detected using standard plate-reading methods (SpectraMax M5).

Briefly, DLBCL, FL, MCL and B-ALL cells were seeded at a density of 2000 to 3000 cells/well in 30 μL of culture medium in 384 well ViewPlate™-384 TC (Corning Incorporated). Treatments were prepared as 4× stocks in culture medium containing 10 to 20% heat inactivated FBS and 10 μL was spiked into triplicate wells for a final volume of 40 μL per well, centrifuged till 300×g for treatment to settle. ADCs and isotype control were used with a titration curve of range from 60 μg/mL to 0.000014 μg/mL and 1:4 serial dilution. Cells were then incubated in a humidified atmosphere with 5% CO2 at 37° C. for 3 days. At the end of the experiment, 40 μL of CELLTITRE-GLO (Promega, Madison, WI USA) was added to each well. Plates were incubated at room temperature for 10 minutes to ensure complete cell lysis. Luminescence was measured using an EnVision 2104 Multilabel Reader (PerkinElmer, Waltham, MA). The potency for test article was determined by generating half-maximal inhibitory concentration (IC50) values using a nonlinear regression model [log inhibitor vs. response] in GraphPad Prism, version 8.1 (GraphPad, San Diego, CA) and presented as percent cell viability relative to mock-treated control cells (media alone) using the formula [(Treated cells)/(Mock−treated control cells)]×100.

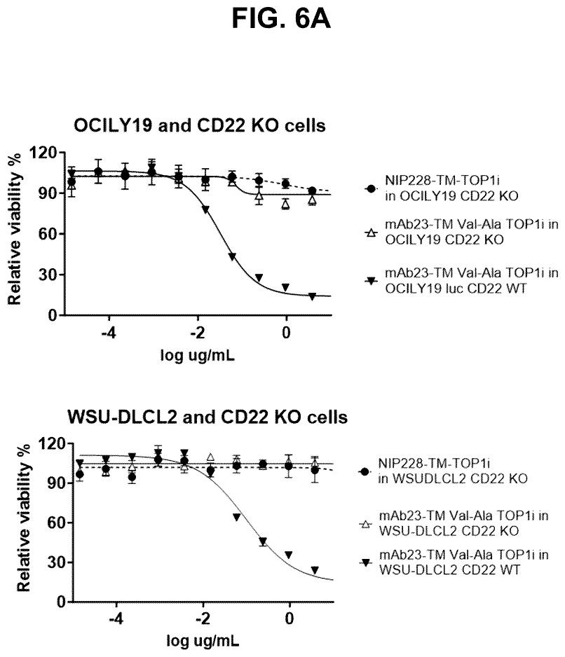

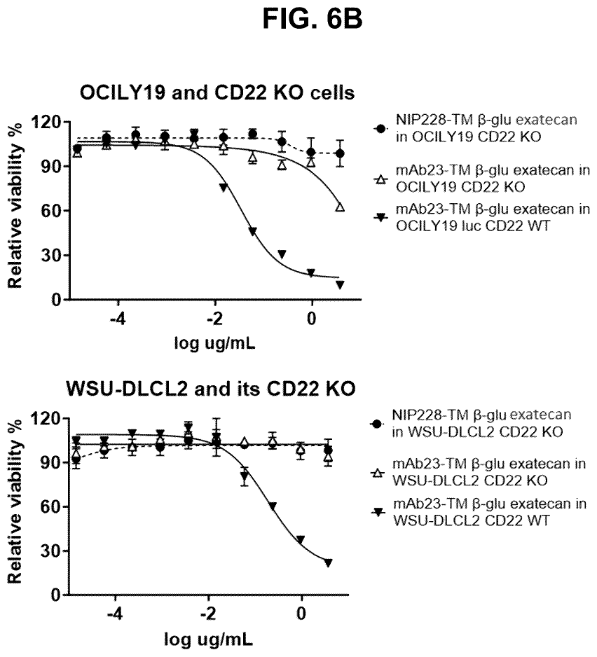

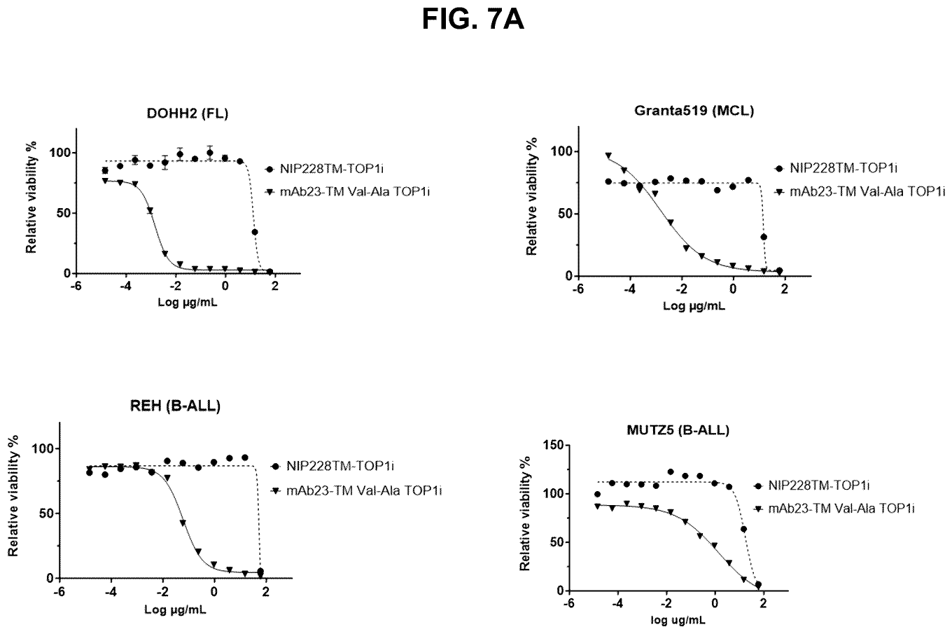

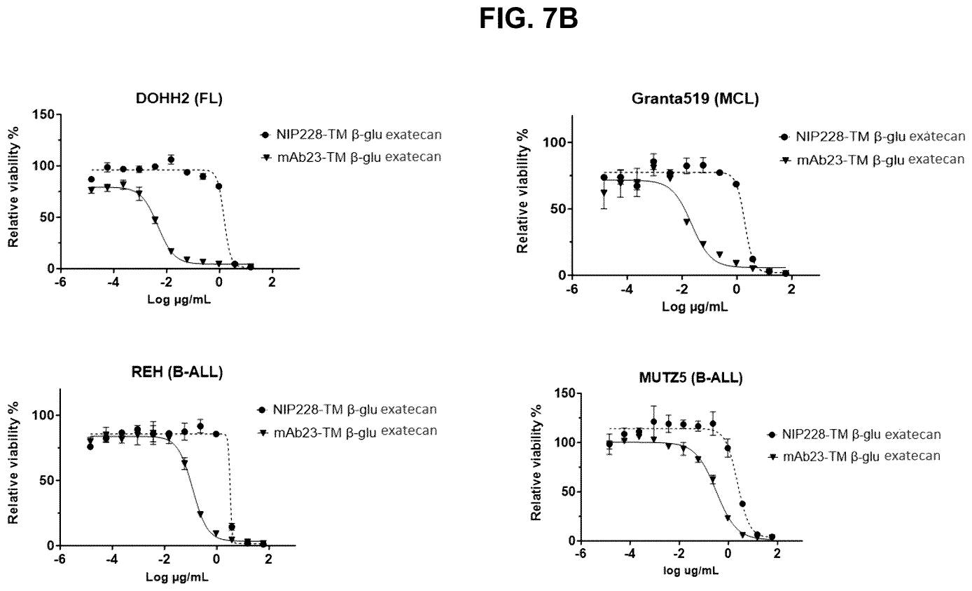

The results show that CD22 ADCs demonstrated strong cell killing with IC50 values of 187.8 and 31.8 ng/mL in WSU-DLCL2 and OCILY19, respectively (FIGS. 6A and 6B, Table 2). In contrast, there is no significant difference in activity observed between CD22 ADCs and isotype control ADCs in WSU-DLCL2 and OICLY19 CD22 knockdown cell lines, suggesting that CD22 ADCs can specifically kill cells expressing human CD22. The cytotoxicity effect of both ADCs on additional malignant B-cell lines, including FL, MCL, and B-ALL lines also exhibited potent cell-killing activity across these malignant B-cell lines, with IC50 values ranging from 1.4 to 1371 ng/mL (FIGS. 7A and 7B, Table 2).

TABLE 2
Cytotoxicity IC₅₀ values of CD22 ADCs and CD22
Levels in the B-cell Malignant Cell Lines

| | Estimated | mAb23-TM β- | mAb23-TM Val- |
|---|---|---|---|
| | CD22 Receptor | glu exatecan: | Ala TOP1i: |
| Cell lines | Density | IC₅₀ (ng/mL) | IC₅₀ (ng/mL) |
| OCILY19 luc (DLBCL) | 19048 | 34.1 | 31.8 |
| WSU-DLCL2 (DLBCL) | 89138 | 187.8 | 97.5 |
| OCILY19 KO (DLBCL) | 980 | N/A | N/A |
| WSU-DLCL2 KO (DLBCL) | 653 | N/A | N/A |
| DOHH2 (FL) | 44736 | 4.6 | 1.4 |
| Granta519 (MCL) | 17406 | 22.1 | 1.5 |
| REH (B-ALL) | 12641 | 118.6 | 338.2 |
| MUTZ5 (B-ALL) | 64144 | 316.5 | 1371 |

### Example 6

Pharmacokinetic Evaluation of mAb23-TM Val-Ala TOP1i ('0132) and mAb23-TM β-Glu Exatecan ADCs

A study was designed to characterize the pharmacokinetics (PK) properties of CD22 ADCs in tumor burdened mice, a DLBCL cell line derived xenograft model.

The xenograft model was established through subcutaneous implantation of 5×10⁶ WSU-DLCL2 cells in 50% Matrigel (Corning) in right flank of female CB17-SCID mice. When tumor volume reached approximately 500 mm³, mice were randomized and given a single IV injection of either mAb23-TM Val-Ala TOP1i or mAb23-TM β-glu exatecan at 2 mg/kg (n=3 per group). Plasma samples were collected at 1, 6, 24, 72, 168, and 336 hours post dose via terminal cardiac puncture. Samples were centrifuged to produce plasma which was stored frozen until analysis. Tumor PK tissue samples were harvested at 1, 6, 24, 72, and 168 hours post dose and snap frozen until analysis.

ADC and the total antibody (Total Ab) concentrations were measured with an immunocapture LC-MS/MS assay. A polyclonal anti-human antibody was conjugated to magnetic beads. Then 25 μL of plasma sample was diluted in TBS and incubated together with the magnetic beads. After capturing, the magnetic beads were washed multiple times before digestion with trypsin under the presence of an internal standard. An aliquot of the trypsin digested supernatant was quenched with the addition of an acid and analysed for the total Ab by LC-MS/MS. For the ADC assay, an aliquot of the trypsin digested supernatant was subjected to an enzymatic digestion overnight with Papain under the presence of an ADC internal standard. The digested samples were quenched with the addition of an acid before analysis by LC-MS/MS. The plasma concentration data was used to estimate PK parameters. ADC plasma concentration was quantified as ADC and the total antibody as divergence of these two profiles may indicate instability of the ADC. Parameters were estimated using noncompartmental analysis (NCA) in Phoenix WinNonlin version 8.4 (Certara, Princeton, USA).

Free payload concentrations in tumor tissue were measured using LC-MS analysis. Briefly, the collected tumor tissue at different time points were homogenized in a modified RIPA buffer (volume based on tumor weight or a uniform volume depending on the project). For the total ADC, immunocapture was used to pull down ADCs from the homogenate, the isolated ADCs are then sequentially digested: 1) cut up the antibody to gain access to the conjugated payloads then 2) cleave the linker to release the payload. For free payload, liquid/liquid extraction was used to isolate the unconjugated payload. For both workflows, further clean up or concentrating steps might be needed depending on sample qualities achieved. Both type of samples were analyzed via LC-MS using reverse phase chromatography and targeted MS analysis.

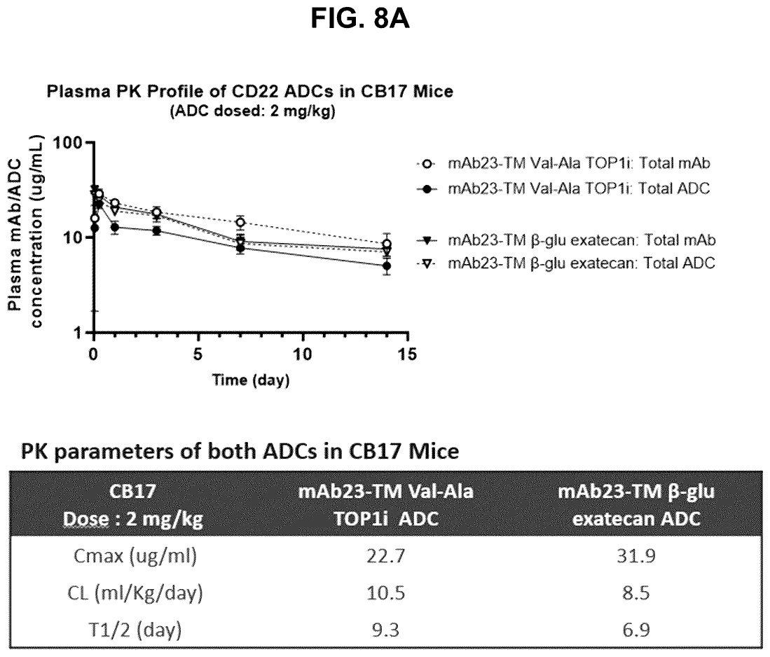

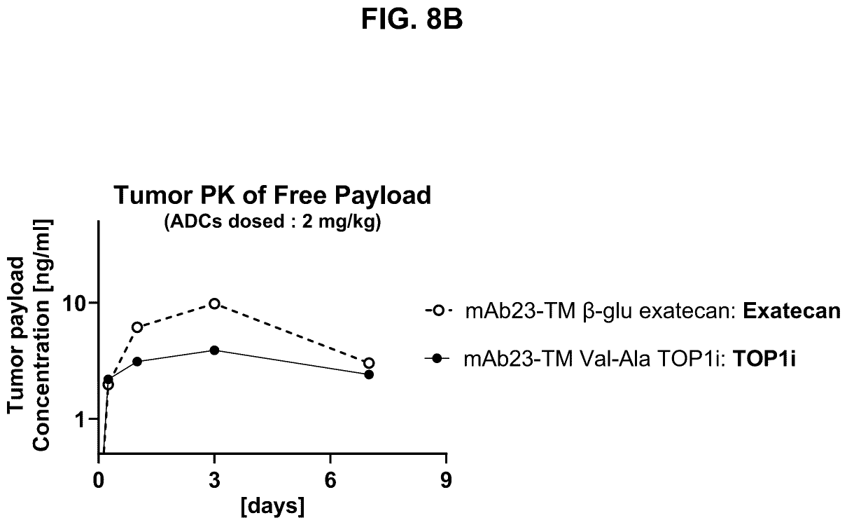

Plasma concentrations of total antibody and ADC following 2 mg/kg single IV administration of mAb23-TM Val-Ala TOP1i ('0132) or mAb23-TM β-glu exatecan ADCs to DLBCL tumor burdened female CB17 SCID mice and the PK parameters for both ADCs are shown in FIG. 8A. The results indicate minimal difference in clearance between total ADC and total mAb for both ADCs in CB17 mice, suggesting that both ADCs remain stable in mice. An apparent terminal elimination phase half-life (T½) of 9.3 and 6.9 days for mAb23-TM Val-Ala TOP1i ('0132) ADC and mAb23-TM β-glu exatecan ADC, respectively, were observed. Total ADC plasma clearance (CL) was 10.5 mL/kg/day for mAb23-TM Val-Ala TOP1i ('0132) and 8.5 mL/kg/day for mAb23-TM β-glu exatecan ADC.

Tumor tissue concentrations of free payloads, exatecan and TOP1i ('0132), following 2 mg/kg single IV administration of mAb23-TM Val-Ala TOP1i ('0132) or mAb23-TM β-glu exatecan ADCs up to day 7 post-treatment are shown in FIG. 8B. The free payload concentration reached its peak value on day 3 after dosing. Notably, exatecan exhibited approximately 2.5 times higher concentration than TOP1i ('0132) at the peak value, indicating that tumor tissue has higher free payload (exatecan) exposure from mAb23-TM β-glu exatecan ADC treatment group compared to the mAb23-TM Val-Ala TOP1i ('0132) ADC treatment group.

### Example 7

In Vivo Efficacy of mAb23-TM Val-Ala TOP1i ('0132) and mAb23-TM β-Glu Exatecan ADCs in DLBCL Tumor Models.

To determine whether mAb23-TM Val-Ala TOP1i ('0132) and mAb23-TM (3-glu exatecan ADCs showed dose-dependent anti-tumor effects in vivo, dose levels ranging from 0.25 mg/kg to 3.6 mg/kg were evaluated in B-cell malignant tumor models.

For DLBCL cell line derived xenograft (CDX) subcutaneous models, cell lines (OCILY19 and WSU-DLCL2) were implanted subcutaneously into female CB17 SCID mice between 6 to 8 weeks of age. When tumors reached the appropriate tumor volume range (150-200 mm³), animals were randomized into treatment and control groups (n=3 animals/group) and dosing was initiated. A single dose of test article was administered to the tumor-bearing mice via intravenous injection. Tumor dimensions and body weight were measured and recorded twice weekly. Tumor volumes were measured by digital caliper and the volumes of tumors were calculated using the following formula: tumor volume=V=(L×W×W)/2, where V is tumor volume, L is tumor length (the longest tumor dimension) and W is tumor width (the longest tumor dimension perpendicular to L).

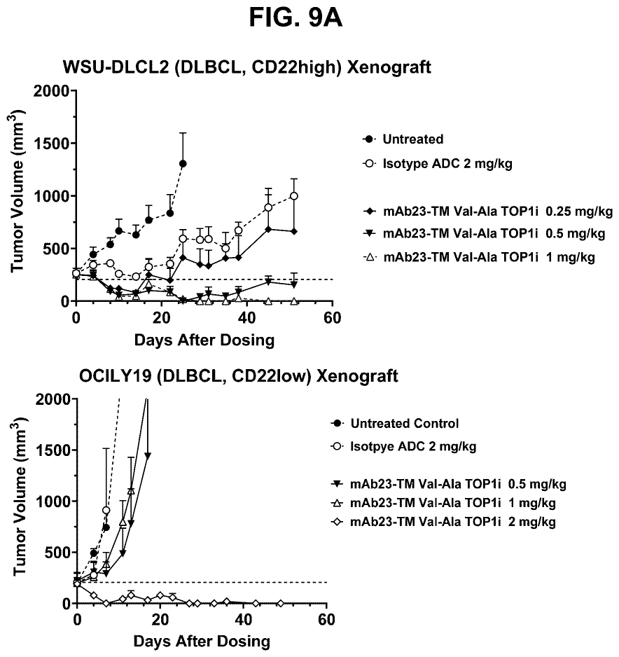

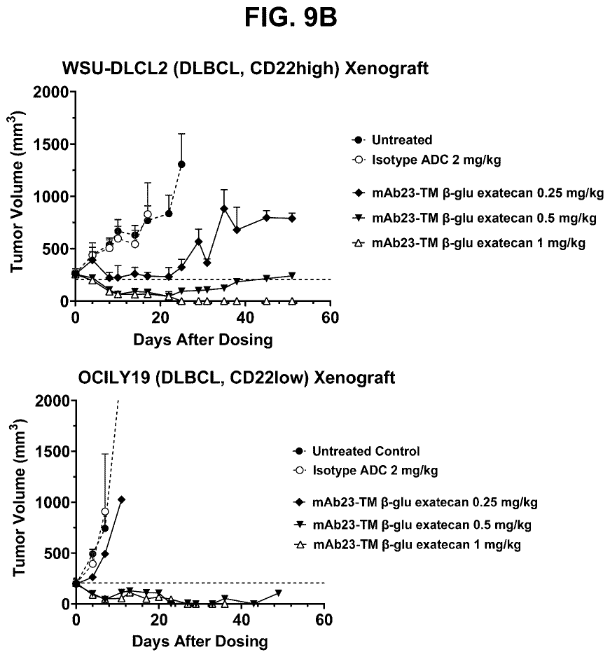

Results shown in FIGS. 9A and 9B demonstrate that treatment with either mAb23-TM Val-Ala TOP1i ('0132) or mAb23-TM β-glu exatecan results in dose-dependent growth inhibition of DLBCL tumor CDX xenografts. mAb23-TM Val-Ala TOP1i ('0132) (FIG. 9A) showed less potency compared to mAb23-TM β-glu exatecan (FIG. 9B) in low CD22 model OCILY19 at dose level of 0.5 mg/kg. Isotype control ADCs even at 2 mg/kg did not cause tumor regression in both models.

For a DLBCL cell line derived xenograft (CDX) disseminated model (SUDHL6 cell line), cells were intravenous (IV) implanted into female NSG mice by tail vein injection. Three to five satellite mice were terminated weekly after cell injection. Peripheral blood (PB) and bone marrow (BM) were collected for disease progression check using a FACS assay with a 2-color panel (Live/Dead and hCD45). When the tumor burden (measured by hCD45-positive percentage) in the BM reached ≥5%, body weight was used as a numeric parameter to randomize selected animals into specified groups, and dosing was initiated. The test articles were administered to the tumor-bearing mice via intravenous injection on a weekly basis, with a total dosage of 1.2, 2.4, or 3.6 mg/kg for two doses. The animals were checked for any effects of tumor growth and treatments on behavior such as mobility, food and water consumption, body weight gain/loss, eye/hair matting and any other abnormalities to determine the humane endpoint. Survival analysis was conducted using Kaplan-Meier survival curves in Prism.

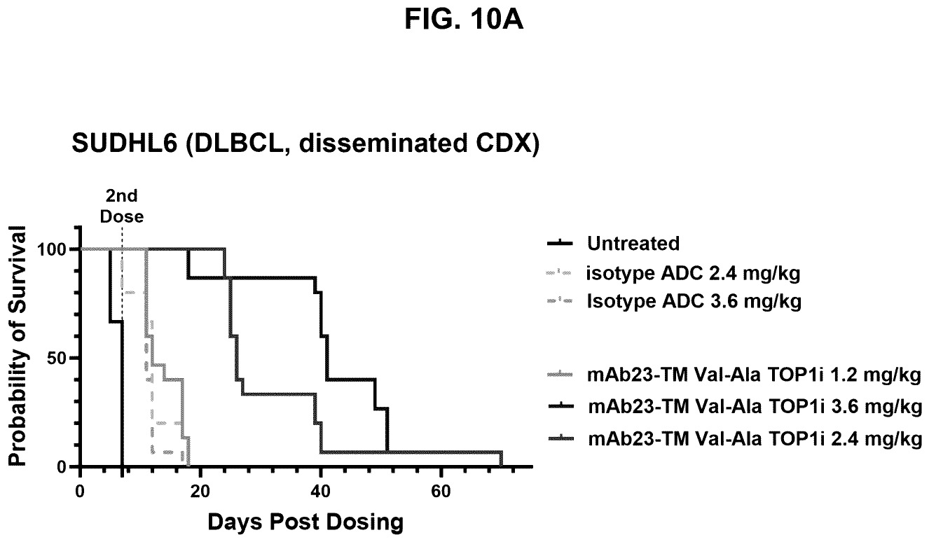

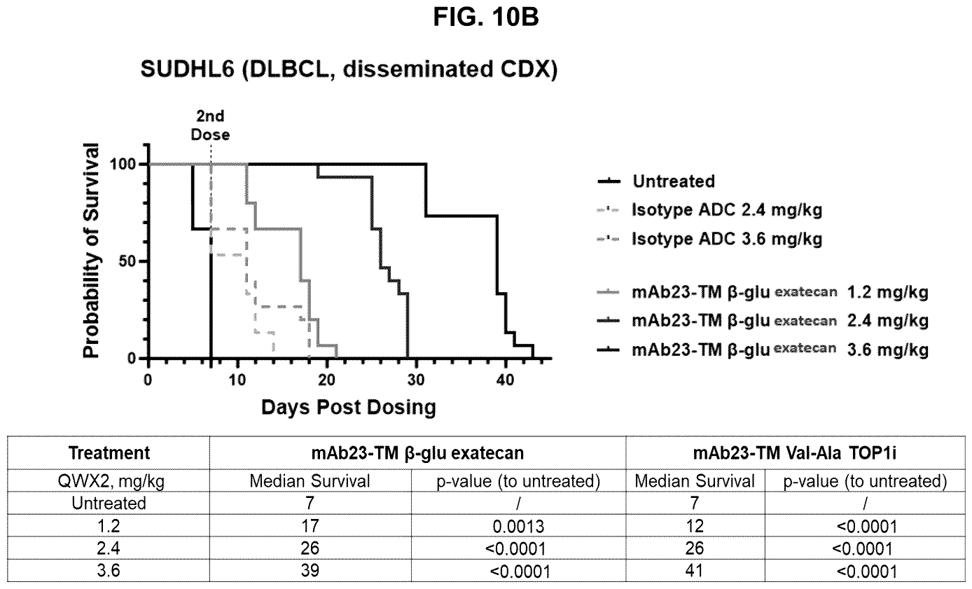

Results shown in FIGS. 10A and 10B demonstrate that treatment with either mAb23-TM Val-Ala TOP1i ('0132) or mAb23-TM β-glu exatecan leads to a significant dose-dependent survival benefit in a DLBCL disseminated CDX model.

For DLBCL patient derived xenograft (PDX) subcutaneous models, fresh tumor fragments from stock mice were harvested and used for inoculation into mice. Each mouse was inoculated subcutaneously in the right flank with DLBCL models tumor fragment (approximately 2-3 mm in diameter) for tumor development. The randomization was performed when the mean tumor size reached 100-200 mm³ with 3 mice per group and randomization was performed using tumor volumes and body weights. A single dose of test article was administered to the tumor-bearing mice via intravenous injection. Tumor dimensions and body weight were measured and recorded 3 times per week. Tumor volumes were measured three times per week after randomization in two dimensions using a caliper through to study end, and the volume was expressed in mm3 using the formula: V=(L×W×W)/2, where V is tumor volume, L is tumor length (the longest tumor dimension) and W is tumor width (the longest tumor dimension perpendicular to L).

Percent change in tumor volume was calculated for each individual tumor at the day of the greatest tumor regression from starting tumor volume. If the tumor does not regress over the course of the study, the best percent change in tumor volume is calculated at least one week after the study start (study day 6). For each individual tumor, the tumor volume at the analysis time point (ATV) was compared to the initial tumor volume from the start of dosing (ITV). Tumor Growth (%) was calculated as [(ATV−ITV)/ITV]×100. If the tumor volume increased between the ITV and the ATV, the Tumor Growth % will be a positive value. Models were considered to be responsive to the test agents if the percent tumor volume from baseline was −30% to −100%, inclusive. Reported and graphed Tumor Growth % are the median value of Tumor Growth % of the mice on study at the time point with the best response.

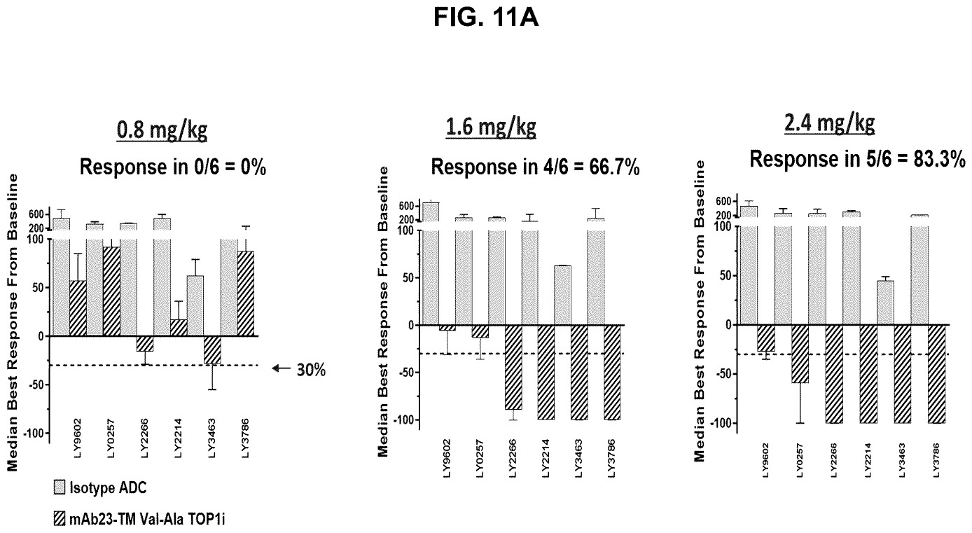

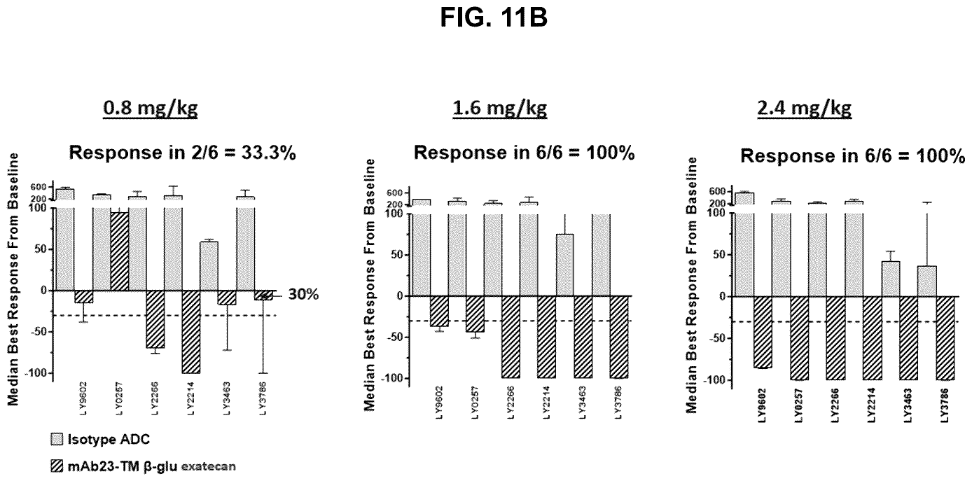

The results shown in FIGS. 11A and 11B demonstrated in vivo antitumor efficacy of mAb23-TM Val-Ala TOP1i ('0132) and mAb23-TM β-glu exatecan ADCs in a panel of 6 Diffuse Large B Cell Lymphoma (DLBCL) patient-derived xenograft (PDX) models. Tumor growth inhibition was observed following a single dose of 1.6 mg/kg of mAb23-TM Val-Ala TOP1i ('0132) ADC, with 66.7% of the models showing a reduction in tumor volume from baseline of 30% or greater. Furthermore, much greater antitumor activity was observed following a single dose of 2.4 mg/kg, with all models displaying a reduction in tumor volume from baseline of 30% or greater. No model achieved a response level at 0.8 mg/kg for mAb23-TM Val-Ala TOP1i ('0132) (FIG. 11A). Conversely, for mAb23-TM β-glu exatecan ADC, tumor growth inhibition was observed at 0.8 mg/kg, with 33.3% of the models displaying a reduction in tumor volume from the baseline of 30% or more. A 100% response was achieved following higher dose levels of 1.6 or 2.4 mg/kg (FIG. 11B). However, none of the models in the isotype control ADC-treated groups reached a 30% reduction in tumor volume at all three dose levels. Notably, mAb23-TM β-glu exatecan ADC demonstrated better efficacy than mAb23-TM Val-Ala TOP1i ('0132) ADC at low dose levels of 0.8 and 1.6 mg/kg in the DLBCL PDX models.

### Example 8

In Vivo Efficacy of mAb23-TM β-Glu Exatecan ADC in MCL/FL and B-ALL Tumor Models

To evaluate the antitumor efficacy of CD22 ADC in additional B-cell malignancies, mAb23-TM β-glu exatecan ADC was tested on 5 MCL/FL patient-derived xenografts (subcutaneous models) and one B-ALL cell line derived xenograft (disseminated model). The experimental procedures on for both the subcutaneous and disseminated models were the same as those applied for DLBCL models. Additionally, for B-ALL model, peripheral blood samples were collected after the 7^{th} day following the first dosing (administered every three weeks, totaling 2 doses) for flow cytometry analysis to determine the percentage of human CD45+ cells in the blood. The reduction of CD45+ cells (tumor cells) demonstrate the anti-tumor effectiveness of the treatment.

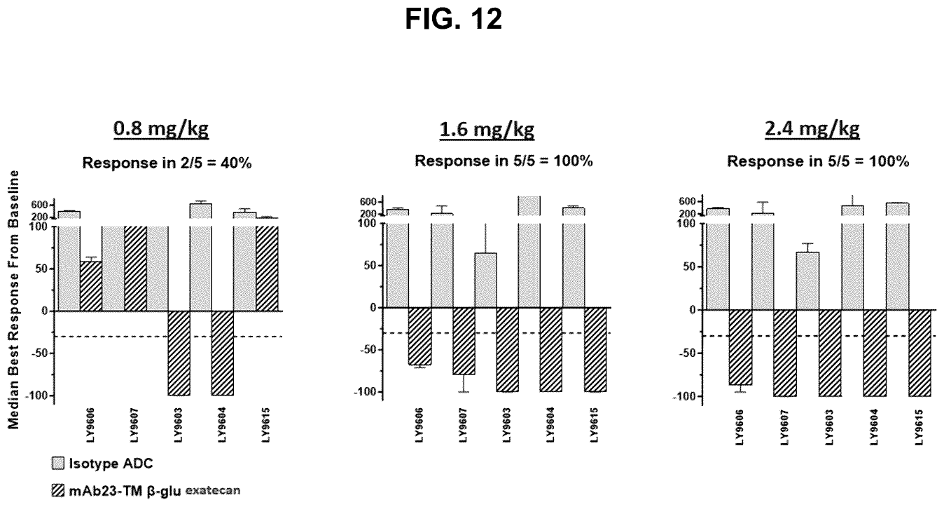

The results from the MCL/FL PDX models, as shown in FIG. 12, demonstrate the strong efficacy of mAb23-TM β-glu exatecan ADC treatment. The tumor growth inhibition was observed at 0.8 mg/kg, with 40% of the models displaying a reduction in tumor volume of 30% or more from the baseline. A 100% response was achieved following higher dose levels of 1.6 or 2.4 mg/kg. Notably, none of the models in the isotype control ADC-treated groups reached a 30% reduction in tumor volume at all three dose levels.

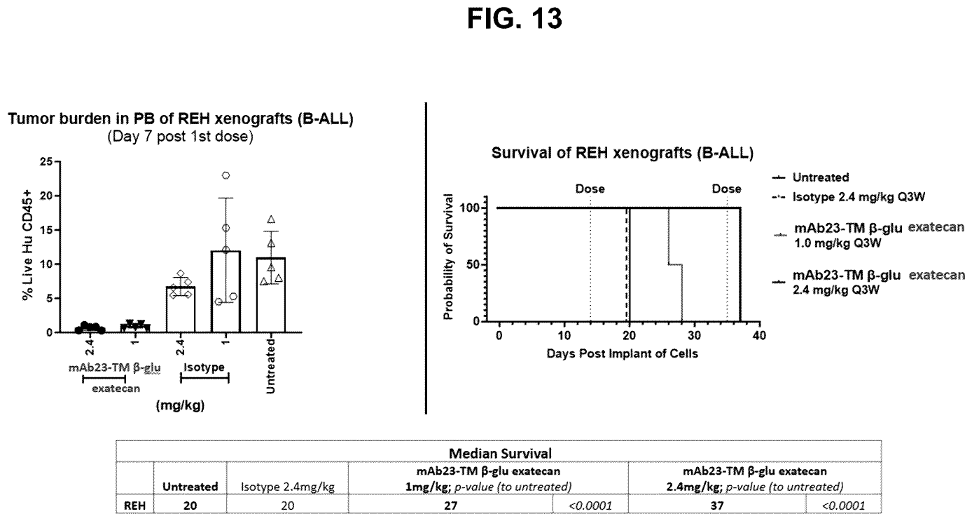

The results from the B-ALL REH cell line derived model are shown in FIG. 13. At day-7 post first dose treatment at 1 or 2.4 mg/kg, mAb23-TM β-glu exatecan ADC suppressed the proliferation of human CD45+ cells (B-ALL cells) in the blood (FIG. 13, top) compared to the untreated group. Consequently, mAb23-TM β-glu exatecan ADC provided significant survival benefit at each dose level compared to the untreated group (FIG. 13, bottom).

### Example 9

In Vivo Efficacy of mAb23-TM β-Glu Exatecan ADC in B-ALL PDX Tumor Models

The antitumor efficacy of the CD22 ADC was further assessed in B-ALL Patient-Derived Xenograft (PDX) disseminated models, representing both Philadelphia-positive (Ph+; 1 model) and Philadelphia-negative (Ph-; 3 models) subtypes, which also have varying levels of CD22 expression (Table 3). B-ALL patient derived xenografts were grown in NOD/SCID mice until the percentage of human CD45+ cells in the bone marrow of the surrogate mice reached a range of 5-17%. Mice were then randomly allocated to groups and left untreated or were given an isotype control ADC or mAb23-TM β-glu exatecan ADC at 1 mg/kg or 2.4 mg/kg intravenously once every three weeks for a total of two doses (D0 and D21). In-life peripheral blood (PB) was collected on DO, D7, D14, D21, D28 and D35 following the administration of the first dose in all groups. PB flow cytometry analysis (2 color panel: Live/Dead and hCD45), was employed to track treatment efficacy by assessing disease burden reduction. The percentage of CD45+ population (tumor cells) was plotted and shown ±SEM. Throughout the study, the animals were closely monitored daily for signs indicating they had reached humane endpoints. Upon reaching these endpoints, the animals were promptly euthanized, and their survival days were recorded. Kaplan-Meier survival curves were then generated based on this data.

TABLE 3
Model Characterization: 4 B-ALL Patient-Derived
Xenograft (PDX) Models Used in the Study

| | | | CD22 level (Estimated receptor density, | |
|---|---|---|---|---|
| | | | Surface Copy Number per cell) | |
| PDX | | Treatment | | Relative to B-ALL patients |
| model | Gene fusion | history | PDX model | (Average: ~4700, n = 14) * |
| AL7015 | BCR-ABL1 (Ph+) | R/R | 1455 | Low-medium |
| AL7447 | Ph− | R/R | 2657 | Medium |
| AL5514 | AFF1-KMT2A (Ph−) | NA | 304 | Low |
| AL7443 | AFF1-KMT2A (Ph−) | R/R | 111 | Extremely low |

* (Jasper et al 2011)
NA: not available;
R/R: refractory/relapsed;
Ph+: Philadelphia-positive;
Ph−: Philadelphia-negative

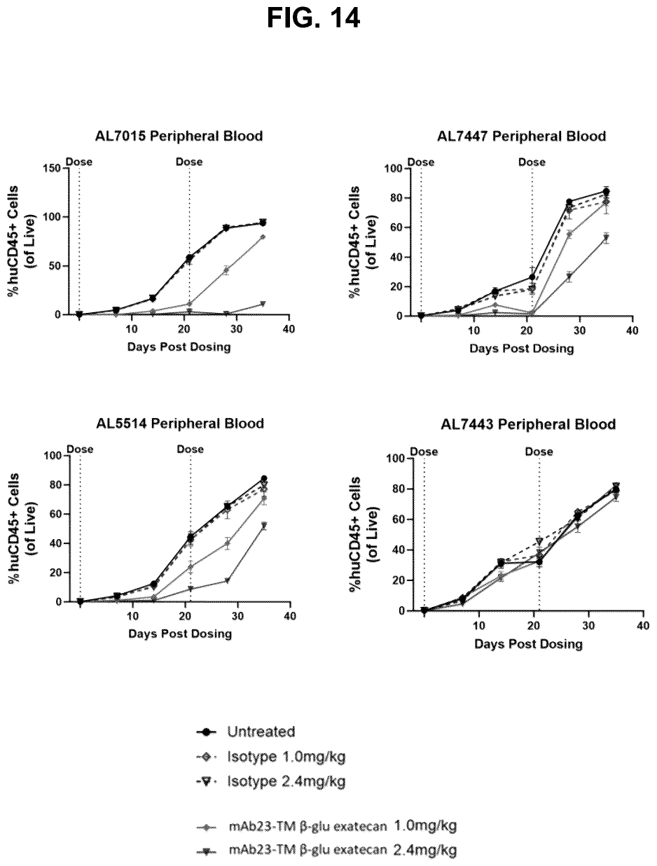

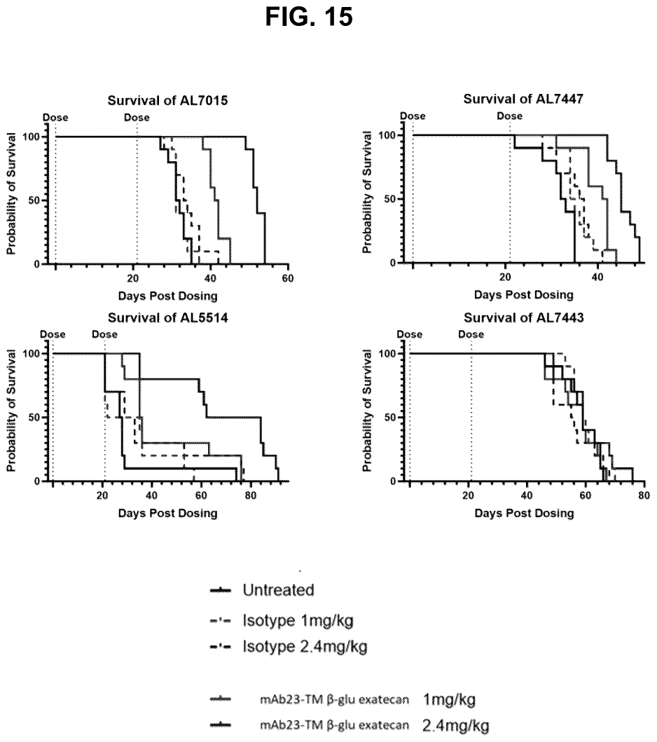

From this antitumor activity study of mAb23-TM β-glu exatecan ADC in the B-ALL PDX models, survival benefit was observed in 3 out of the 4 models, including the one model that is Ph+, after IV dosing Q3W, totaling 2 doses of mAb23-TM β-glu exatecan at 1 mg/kg or 2.4 mg/kg, compared to the untreated group (FIG. 14 and Table 4). The survival time was defined as the duration from the day of dosing initiation until the day of animal death or ethical endpoint, and median survival days were presented in the table. For mAb23-TM β-glu exatecan, the median survival ranged from 35 to 41 days at 1 mg/kg and from 45 to 73 days at 2.4 mg/kg across the three responsive models. In contrast, the untreated groups exhibited median survival days ranging from 27 to 32. All data were analyzed using GraphPad Prism 5.0, with statistical significance denoted by p<0.05. The analysis revealed that 3 out of the 4 models exhibited a statistically significant survival benefit from mAb23-TM β-glu exatecan treatment at both 1 mg/kg and 2.4 mg/kg dose levels. Additionally, in-life antitumor efficacy was assessed weekly through flow analysis of human CD45+ percentage in PB following the initial treatment for up to 5 weeks. mAb23-TM β-glu exatecan demonstrated consistently significant disease burden reduction in 3 out of 4 models, compared to the untreated group, at the majority of time points and at both dose levels (FIG. 15).

TABLE 4
Statistical Analysis of mAb23-TM β-glu exatecan Survival Benefit in B-ALL PDX Model

| | | | Median Survival Time (day) / p-value (to untreated) | | | | | |
|---|---|---|---|---|---|---|---|---|
| | | | | | mAb23-TM β-glu | | mAb23-TM β-glu | |
| PDX Model | | | | Isotype | exatecan | | exatecan | |
| Model ID | Gene fusion | CD22^{a} | Untreated | 2.4 mg/kg | 1 mg/kg | | 2.4 mg/kg | |
| AL 7015 | BCR-ABL1 | 1455 | 31.5 | 33.5 | 41.5 | <0.0001 | 52 | <0.0001 |
| | (Ph+) | | | | | | | |
| AL 7447 | Ph− | 2657 | 32.5 | 36.5 | 41.5 | 0.0002 | 45 | <0.0001 |
| AL 5514 | AFF1-KMT2A | 304 | 27.5 | 31 | 35.5 | 0.0059 | 73 | 0.0002 |
| | (Ph−) | | | | | | | |
| AL 7443 | AFF1-KMT2A | 111 | 59 | 55.5 | 59 | 0.9824 | 59 | 0.3064 |
| | (Ph−) | | | | | | | |

^{a}CD22 level (Estimated receptor density, Copy number/cell)

### Example 10

The mAb23-TM β-Glu Exatecan ADC Shows Better Target Selectivity Compared to an Anti-CD22-Calicheamicin ADC in a Cytotoxicity Assay on Primary B Cells and T Cells.

Briefly, human primary T cells and B cells, obtained and isolated from the same healthy donor, were seeded at a density of 5,000 cells per well in 100 μL of culture medium in a 96-well round-bottom TC plate (Corning Incorporated), using B cell/T cell basic culture medium, respectively. B cell and T cell culture activator cocktails and treatments were prepared as 4× stocks in basic culture medium. For the experimental setup, 50 μL of the culture activator cocktails was added to triplicate wells, except for unstimulated wells. This was followed by adding 50 μL of treatments to achieve a final volume of 200 μL per well. ADCs (mAb23-TM β-glu exatecan and a BESPONSA® biosimilar: anti-CD22-calicheamicin ADC) were used with a titration curve ranging from 60 μg/mL to 0.0006 μg/mL, employing a 1:10 serial dilution. Cells were incubated in a humidified atmosphere with 5% CO₂ at 37° C. for 4 days. At the end of the experiment, 100 μL of culture medium from each well was gently removed and replaced with 100 μL of CELLTITER-GLO reagent (Promega, Madison, WI, USA). The plate was shaken for 5 minutes, then mixed well, and 170 μL of the cell lysate was transferred into a 96-well clear-bottom white-wall plate. Luminescence was then measured using an EnVision 2104 Multilabel Reader (PerkinElmer, Waltham, MA, USA).

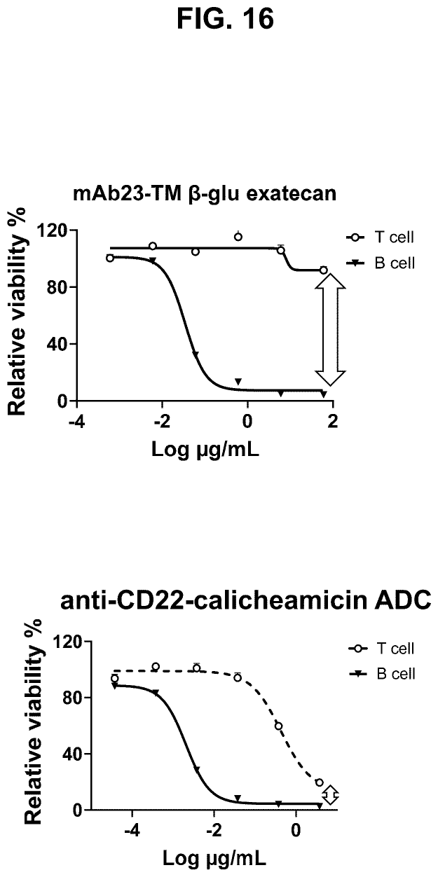

As primary B cells express CD22, the cytotoxicity observed from the CD22 ADC treatment reflects targeted activity. However, since T cells do not express CD22, any cell killing observed with them is due to off-target effects. The results indicate that the mAb23-TM β-glu exatecan ADC shows better target selectivity compared to the anti-CD22-calicheamicin ADC. This is demonstrated by a fold change in IC₅₀s between T cells and B cells, with mAb23-TM β-glu exatecan ADC having more than 1,700-fold selectivity compared to 215-fold selectivity for the anti-CD22-calicheamicin ADC (FIG. 16 and Table 5).

TABLE 5
Cytotoxicity IC₅₀ Values of the mAb23-TM β-glu
exatecan ADC and anti-CD22-calicheamicin ADC and
the Fold Changes between T cells and B cells

| | IC₅₀ (ng/mL) | |
|---|---|---|
| Primary cells | mAb23-TM β-glu exatecan | CD22-calicheamicin |
| B cells (On target) | 0.034 | 0.002 |
| T cells (Off target) | >60 | 0.43 |
| T cells/B cells | >1764 | 215 |

### Example 11

In Vivo Toxicity of mAb23-TM β-Glu Exatecan ADC in Non-Human Primates

Nonclinical toxicity studies conducted in non-human primates with mAb23-TM B-glu exatecan ADC indicate target organs of toxicity of the gastrointestinal tract and bone marrow following two intravenous doses (>20 mg/kg). For BESPONSA®, target organs of toxicity identified in repeat dose toxicity studies up to 6 months in duration in cynomolgus monkeys and rats included the liver, reproductive tissues (uterus, ovary, and testes), bone marrow, and lymphoid tissues (BESPONSA®BLA 2016) (see Table 6).

TABLE 6
mAb23-TM β-glu exatecan ADC has fewer nonclinical toxicity
target organs compared to BESPONSA ®

| | | mAb23-TM β-glu |
|---|---|---|
| Finding | BESPONSA ® | exatecan ADC |
| Lung | — | — |
| Skin | — | — |
| Kidney | — | — |
| Liver | Yes | — |
| Gonads | Yes | — |
| BM or Lymphoid tissue | Yes | Yes |
| GIT | — | Yes |

Nonclinical findings in the indicated tissues are shown for mAb23-TM β-glu exatecan ADC and the ADC, BESPONSA ®, which is approved for B-ALL.

### Example 12

mAb23-TM Demonstrates Superior Properties Relative to an Anti-CD22 Benchmark Antibody

In this study, head-to-head functional assays, including binding, internalization, and cytotoxicity assessments, were performed to compare mAb23-TM with an inotuzumab biosimilar (a benchmark anti-CD22 antibody). The results show that mAb23-TM exhibits distinct functional attributes relative to the benchmark anti-CD22 antibody, which may lead to improved clinical responses for mAb23-TM β-glu exatecan ADC compared to BESPONSA®.

Binding of Antibody mAb23-TM and a Benchmark Anti-CD22 Antibody to Human Diffuse Large B-Cell Lymphoma (DLBCL) Cell Lines WSU-DLCL2 and OCILY19.

Flow cytometry was performed to evaluate the binding of AF647-conjugated antibodies mAb23-TM, a benchmark anti-CD22 antibody, and the isotype control NIP228-TM to CD22-expressing DLBCL cell lines, WSU-DLCL2 (high CD22) and OCI-LY19 (low CD22). Cells were counted and transferred to round-bottom 96-well plates at a density of 200,000 cells per well. Following centrifugation at 500×g for 3 minutes at room temperature, cells were treated with 2.5 μg/mL Fc block (BD Biosciences), 50 μL per well, for 10 minutes and then pelleted again at 500×g for 3 minutes at 4° C. Antibody treatments were prepared as a titration curve ranging from 20 μg/mL to 0.15625 μg/mL with 1:2 serial dilutions, applied at 50 μL per well. Plates were incubated on ice for 30 minutes. Primary antibody was removed by centrifugation at 500×g for 3 minutes at 4° C., followed by two washes with 100 μL FACS buffer (BD Stain Buffer, BD Biosciences). Cells were resuspended in 100 μL BD Stain Buffer and held on ice. Fluorescence of single cells was analyzed using a BD Fortessa cytometer and BD FACSDiva software (BD Biosciences, San Jose, CA, USA), and data were processed using FlowJo™ software (BD Biosciences, San Jose, CA, USA) and in GraphPad Prism, version 10.4.1 (GraphPad, San Diego, CA USA)

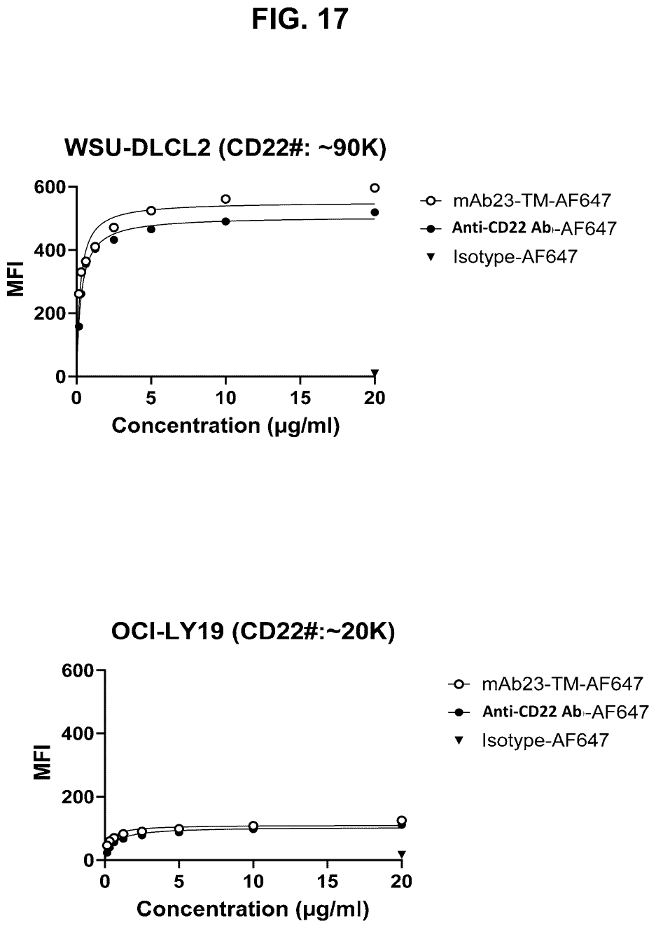

As shown in FIG. 17, both mAb23-TM and the benchmark anti-CD22 monoclonal antibodies demonstrated similar binding affinities to the DLBCL cell lines. The apparent Kd values were within a comparable range of 1.55 to 1.93 nM for WSU-DLCL2 and 1.92 to 3.77 nM for OCI-LY19.

Internalization of Antibody mAb23-TM and Benchmark Anti-CD22 Antibody in Human Diffuse Large B-Cell Lymphoma (DLBCL) Cell Lines WSU-DLCL2 and OCILY19.

Internalization assays were run to measure the rate and extent to which mAb23-TM and the benchmark anti-CD22 antibody (an inotuzumab biosimilar) are internalized by cells after engaging with the target antigen CD22. The assay relies on labeling the Fc-containing test antibodies with a Fab fragment-conjugated to a pH-sensitive fluorophore (Sartorius) and the ability of CD22 receptor to internalize when bound to antibodies. The internalization signal was detected with standard methods of live cell imaging using the Incucyte® SX5.

Ninety-six-well flat-bottom plates (Perkin Elmer, catalog #6055300) were coated with 50 μL/well Cultrex Poly-L-Lysine (PLL; Fisher Scientific) for 15 minutes at room temperature in a biological safety cabinet. After aspiration, plates were washed twice with 150 μL sterile deionized water, incubated at 37° C. for drying, and used for cell plating the following day. Cells were counted and seeded at a density of 100,000 cells per well in triplicate for mAb23-TM, the benchmark anti-CD22 antibody, and the isotype control, using 80 μL phenol red-free RPMI-160 medium supplemented with 20% filtered FBS. Antibody stocks (mAb23-TM, benchmark anti-CD22 antibody, and NIP228-TM isotype) were conjugated to Incucyte® Fab Fluor Orange (Sartorius) in phenol red-free medium, achieving a fluor-to-antibody molar ratio of 5. The antibody-dye mixtures were incubated for 15 minutes at room temperature, protected from light. Final working concentrations of conjugated antibody were prepared, and 20 μL of each treatment was added to cells in triplicate wells to achieve target dosing at 1 and 0.5 μg/mL. Plates were gently tilted to mix and transferred to the Sartorius Incucyte® SX5 system for hourly live-cell imaging over 24 hours. Raw image data were analyzed using Incucyte® 2023A software to quantify cell confluence and orange fluorescence intensity. Data analysis included normalization to the isotype control, and final values were calculated and visualized as [Total Orange Object Integrated Intensity (OCU×m²/Image)/Confluence]×100 using GraphPad Prism.

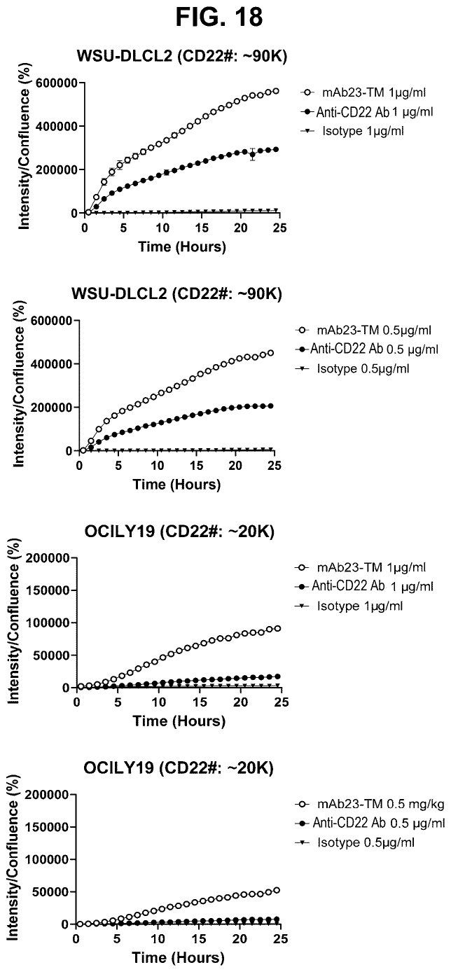

As shown in FIG. 18, mAb23-TM exhibits enhanced internalization compared to the benchmark anti-CD22 antibody in DLBCL cell lines. At concentrations of 1 μg/mL and 0.5 μg/mL, mAb23-TM demonstrated a significantly higher rate and intensity of internalization relative to the benchmark anti-CD22 antibody.

Cytotoxicity Assessment of mAb23-TM and Benchmark Anti-CD22 Antibody Conjugated with β-Glu-Exatecan Linker Payload at DAR8 in DLBCL Cell Lines

The cytotoxicity assays were designed to determine whether mAb23-TM, when conjugated to the same linker-payload, could be internalized more efficiently and release its payload more effectively, resulting in greater toxicity than the benchmark anti-CD22 antibody (inotuzumab biosimilar) under identical conjugation conditions.

WSU-DLCL2 and OCI-LY19 DLBCL cells were seeded at a density of 2,500 cells per well in 30 μL RPMI 1640 medium (Thermo Fisher Scientific) supplemented with 10% heat-inactivated FBS, using 384-well ViewPlate™-384 TC plates (Corning Incorporated). Treatment solutions were prepared as 4× stocks in RPMI 1640 containing 20% heat-inactivated FBS, and 10 μL was added per well (in triplicate) to yield a final volume of 40 μL per well. ADCs and isotype controls were tested over a titration curve ranging from 60 μg/mL to 0.000014 μg/mL using 1:4 serial dilutions. Following addition of treatments, cells were incubated at 37° C. in a humidified atmosphere with 5% CO₂ for six days. After incubation, 40 μL CellTiter-Glo Reagent (Promega) was added to each well and plates were incubated at room temperature for 10 minutes to ensure complete cell lysis. Luminescence was measured using an EnVision 2104 Multilabel Reader (PerkinElmer). Potency was determined by calculating half-maximal inhibitory concentration (IC₅₀) values using nonlinear regression modeling (log inhibitor vs. response) in GraphPad Prism version 10.4.1. Results were expressed as percent cell viability relative to mock-treated control cells using the formula:

$$[ ( Treated cells ) / ( Mock - treated control cells ) ] \times 100 .$$

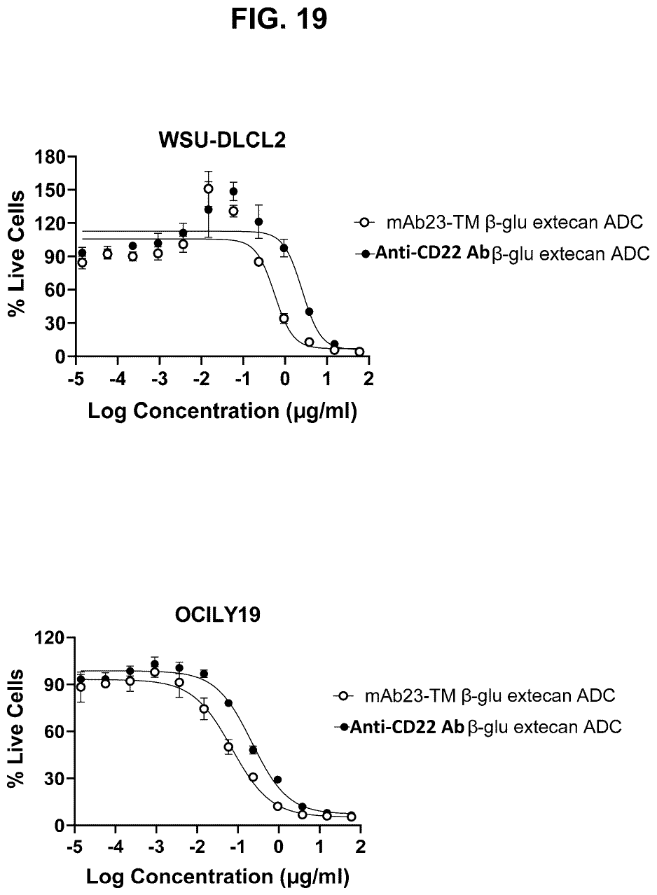

As shown in FIG. 19 and Table 7, mAb23-TM conjugated with β-Glu-Exatecan at DAR8 exhibited significantly greater cytotoxic activity compared to the benchmark anti-CD22 antibody -β-Glu-Exatecan in DLBCL cell lines. Pronounced increases in cytotoxicity were observed, with IC₅₀ fold reductions relative to the benchmark anti-CD22 antibody of 4.6 in WSU-DLCL2 cells and 3.1 in OCI-LY19 cells. These findings further support the potential of mAb23-TM as a superior CD22 ADC antibody backbone, enabling more effective and efficient intracellular payload release.

TABLE 7
Cytotoxicity IC₅₀ values (ng/ml)

| | mAb23- | Benchmark anti-CD22 | Efficacy |
|---|---|---|---|
| DLBCL Cell | TM β-glu | antibody β-glu | Fold Increase |
| line | exatecan (A) | exatecan (B) | (B/A) |
| WSU-DLCL2 | 554.7 | 2548 | 4.59 |
| OCI-LY19 | 70.12 | 218.8 | 3.12 |

### Example 13

### Linker-Payload Synthesis

### General Information

Flash chromatography was performed using a BIOTAGE ISOLERA and fractions checked for purity using thin-layer chromatography (TLC). TLC was performed using MERCK KIESELGEL 60 F254 silica gel, with fluorescent indicator on aluminium plates. Visualisation of TLC was achieved with UV light.

Extraction and chromatography solvents were bought and used without further purification from VWR U.K.

All fine chemicals were purchased from SIGMA-ALDRICH unless otherwise stated.

Pegylated reagents were obtained from QUANTA BIODESIGN US via STRATECH UK.

### LC/MS Conditions

Positive mode electrospray mass spectrometry was performed using a WATERS ACQUITY H-CLASS SQD2 using one of the following methods.

(a) The HPLC (WATERS ALLIANCE 2695) was run using a mobile phase of water (A) (formic acid 0.1%) and acetonitrile (B) (formic acid 0.1%).

LCMS 3 min: Initial composition 5% B held over 25 seconds, then increased from 5% B to 100% B over a 1 minute 35 seconds' period. The composition was held for 50 seconds at 100% B, then returned to 5% B in 5 seconds and held there for 5 seconds. The total duration of the gradient run was 3.0 minutes. Flow rate was 0.8 mL/minute. Wavelength detection range: 190 to 800 nm. Columns: WATERS ACQUITY UPLC BEH SHIELD RP18 1.7 μm 2.1×50 mm at 50° C. fitted with WATERS ACQUITY UPLC BEH SHIELD RP18 VANGUARD Pre-column, 130A, 1.7 μm, 2.1 mm×5 mm.

LCMS 15 min: initial composition 5% B held over 1 min, then increase from 5% B to 100% B over a 9 min period. The composition was held for 2 min at 100% B, then returned to 5% B in 0.10 minutes and hold there for 3 min. Total gradient run time equals 15 min. Flow rate 0.6 mL/min. Wavelength detection range: 190 to 800 nm. Oven temperature: 50° C. Column: WATERS ACQUITY UPLC CSH C18 1.7 μm 2.1×100 mm fitted with WATERS ACQUITY UPLC CSH C18 VANGUARD Pre-column, 1.7 μm, 2.1 mm×5 mm.

(b) The HPLC (Agilent 1290) was run using a mobile phase of water (A) (TFA 0.03%) and acetonitrile (B) (0.03% TFA), or water (A) (TFA 0.05%) and acetonitrile (B) (0.05% TFA). Initial composition was (a) 100% A held for 2-4 minutes then increased to 90% B over 2-5 minutes, or (b) 5%-20% B increased to 90%-98% B over 3-17 minutes. Flow rate was 0.3-1.5 mL/minute. Column was (1) ATLANTIS T3 3 m 4.6*150 mm at 40° C. (Detector ELSD or Wavelength detection range: 210 nm), (2) ACQUITY UPLC BEH C18 2.1*100 mm 1.7 μm at 40° C. (Wavelength detection range: 210 nm or 220 nm), (3) UPLC BEH C18 1.7 μm, 2.1*100 mm at 40° C. (Wavelength detection range: 223 nm), (4) XBRIDGE C18 (4.6*150, 3.5 μm) at 40° C., (5) ACQUITY UPLC HSS PFP 2.1*150 mm 1.8 μm at 40° C. (Wavelength detection range: 220 nm), (6) UPLC BEH Phenyl 1.7 μm, 2.1*150 mm at 40° C. (Wavelength detection range: 210 nm), (7) EC-C18 2.7 μm, 3.0*50 mm at 40° C. (Wavelength detection range: 210 nm), or (8) YMC-Triart C18 50*3.0 mm S-3 um, 12 nm at 45° C. (Detector ELSD). Injection volume 2 μL.

### HPLC Conditions

Reverse-phase ultra-fast high-performance liquid chromatography (UFLC) was carried out on a SHIMADZU PROMINENCE machine using a PHENOMENEX GEMINI NX 5μ C18 column (at 50° C.) dimensions: 150×21.2 mm. Eluents used were solvent A (H₂O with 0.1% formic acid) and solvent B (CH₃CN with 0.1% formic acid). All UFLC experiments were performed with gradient conditions: Initial composition 13% B increased to 30% B over a 3 minutes period, then increased to 45% B over 8 minutes and again to 100% over 6 minutes before returning to 13% over 2 min and hold for 1 min. The total duration of the gradient run was 20.0 minutes. Flow rate was 20.0 mL/minute and detection was at 254 and 223 nm.

### NMR Method

Proton NMR chemical shift values were measured on the delta scale at 400 MHz using a BRUKER AV400. The following abbreviations have been used: s, singlet; d, doublet; t, triplet; q, quartet; quin, quintet; m, multiplet; br, broad. Coupling constants are reported in Hz.

Abbreviations

| | |
|---|---|
| TLC | Thin layer chromatography |
| UV | Ultra violet |
| LCMS | Liquid chromatography mass spectrometry |
| CSH | Charged surface hybrid |
| UPLC | Ultra-performance liquid chromatography |
| RP | Reverse phase |
| NMR | Nuclear magnetic resonance |
| DBU | 1,8-Diazabicyclo[5.4.0]undec-7-ene |
| DMF | Dimethyl formamide |
| DCM | Dichloromethane |
| TBS | Tert-butyl dimethyl silane |
| MS | Molecular sieves |
| TFA | Trifluoro acetic acid |
| DMSO | Dimethyl sulfoxide |
| ESI | Electrospray ionisation |
| DIPEA | Di isopropyl ethylamine |
| THF | Tetra hydro furan |
| HATU | Hexafluorophosphate azabenzotriazole tetramethyl |
| | uronium |
| TEA | Triethylamine |
| HOPO | 1-Hydroxy-2-pyridone |
| RT | Retention time |
| ADC | Antibody-drug conjugate |
| UHPLC | Ultra-high performance liquid chromatography |
| mAb | Monoclonal antibody |
| SEC | Size exclusion chromatography |
| DAR | Drug to antibody ratio |
| RPMI | Roswell Park Memorial Institute |
| ND | Not detectable |
| IC₅₀ | Inhibitory concentration 50% |
| Fmoc | fluorenylmethoxycarbonyl |

### Intermediate 1

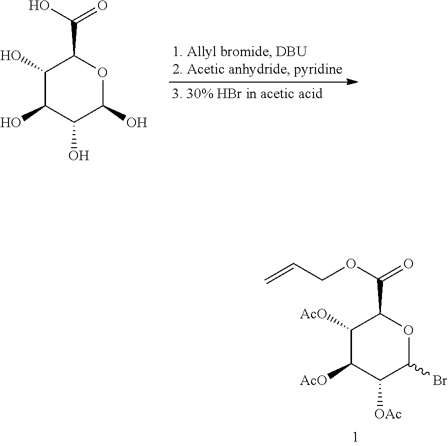 [SMILES: O=C(O)[C@H]1O[C@@H](O)[C@H](O)[C@@H](O)[C@@H]1O ; C=CCOC(=O)[C@H]1OC(Br)[C@H](OC(C)=O)[C@@H](OC(C)=O)[C@@H]1OC(C)=O]

2,3,4,6,7,8,9,10-octahydropyrimido[1,2-a]azepine (26.5 mL, 177.35 mmol) was added dropwise to a 1-L round bottom flask containing (2S,3S,4S,5R,6R)-3,4,5,6-tetrahydroxytetrahydro-2H-pyran-2-carboxylic acid (31.3 g, 161.22 mmol) in DMF (100 mL) at 21° C. Next, 3-bromoprop-1-ene (16.72 mL, 193.47 mmol) was added to the reaction mixture dropwise over 10 minutes and the reaction was stirred at 21° C. for 24 hours. Reaction mixture was cooled to 0° C. and treated with pyridine (104 mL, 1289.60 mmol). Acetic anhydride (244 mL, 2579.20 mmol) was next added to the reaction mixture. The reaction was warmed up to room temperature and run for 2 hours at 21° C. Reaction mixture concentrated under reduced vacuum and the remaining pyridine was azeotropically removed with toluene (1×100 mL). Crude material was diluted with DCM (65 mL) and cooled to 0° C. 30% Hydrobromic acid in acetic acid (175 mL, 3226.03 mmol) was next added to the reaction mixture at 0° C. The reaction was warmed up to room temperature and run for 2 hours 30 minutes at 21° C. Solvent was evaporated then the compound was purified by normal phase flash column chromatography to afford (2S,3S,4S,5R,6R)-2-((allyloxy)carbonyl)-6-bromotetrahydro-2H-pyran-3,4,5-triyl triacetate Intermediate 1 (33 g, 48% yield) as a beige translucent material. ¹H NMR (500 MHz, CDCl₃) δ 6.67 (d, J=4.0 Hz, 1H), 5.92 (ddt, J=16.6, 10.3, 6.0 Hz, 1H), 5.64 (t, J=9.7 Hz, 1H), 5.42-5.23 (m, 3H), 4.88 (dd, J=10.0, 4.0 Hz, 1H), 4.71-4.58 (m, 3H), 2.12 (s, 3H), 2.07 (s, 3H), 2.05 (s, 3H); LCMS (ESI) m/z 445.0 (M+Na)+.

### Alternative Synthesis of Intermediate 1

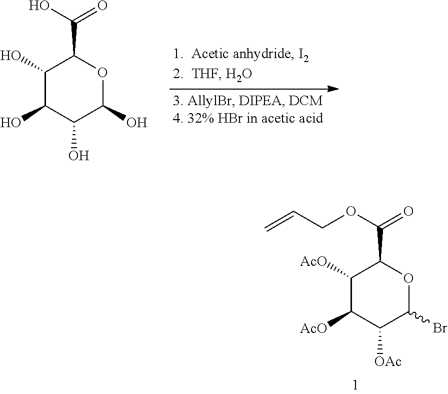 [SMILES: O=C(O)[C@H]1O[C@@H](O)[C@H](O)[C@@H](O)[C@@H]1O ; C=CCOC(=O)[C@H]1OC(Br)[C@H](OC(C)=O)[C@@H](OC(C)=O)[C@@H]1OC(C)=O]

Iodine (1.19 kg, 4.69 mol) was added to acetic anhydride (3500 mL) stirred at 0-10° C. under nitrogen. The resulting mixture was adjusted to 20-30° C. and Glucuronic acid (7 kg, 36.06 mol) was added portion wise, maintaining the temperature at 25-30° C. The reaction was stirred at this temperature for 1 hour under nitrogen and then cooled to 0° C. A solution of sodium thiosulfate pentahydrate (2.33 kg) in water (35.2 L) was added to the stirred mixture at 0-10° C., and then stirred to 2 hours at 20-30° C. Water (35.2 L) was added to the stirred mixture, extracted with isopropyl acetate (3×35.2 L), and the organic layer was concentrated to dryness to give crude 1,2,3,4-tetra-O-acetyl-β-D-glucuronic acid (16.08 kg, 61% w/w assay, 75%). LCMS m/z (ES+), [M+Na]⁺=384.6.

Crude 1,2,3,4-tetra-O-acetyl-β-D-glucuronic acid, 61% w/w (16 kg; 27.05 mol) was dissolved in isopropyl acetate (42.83 kg) and stirred at 20-30° C. N,N-diispropylethylamine (12.25 kg, 94.68 mol) was added to the reaction at 20-30° C. over 11 minutes followed by 3-bromopropene (9.8 kg, 81.15 mol) added dropwise at 20-30° C. over 5 minutes. The resulting mixture was stirred for 48 hours at 20-30° C. Isopropyl acetate (42.83 kg) and water (49 kg) were added to the stirred mixture. The organic layer separated and adjusted to pH 4-5 at 20-30° C. by addition of aqueous hydrochloric acid (0.6N, 43.71 kg). The separated organic layer was washed with brine (25% aqueous solution, 49 L), and concentrated to dryness to give crude 1,2,3,4-tetra-O-acetyl-β-D-glucuronic acid allyl ester as a brown solid (12.0 kg, 87.5% w/w assay, 86.6%). LCMS m/z (ES+), [M+Na]⁺=424.833% hydrobromic acid in acetic acid (534 mL, 2.982 mol) was added dropwise to a stirred mixture of crude 1,2,3,4-tetra-O-acetyl-β-D-glucuronic acid allyl ester (200 g, 0.497 mol) in isopropyl acetate (500 mL) at 0° C. The reaction was adjusted to 20-30° C. and stirred for 8 hours. The reaction mixture was extracted with isopropyl acetate (2400 mL), the extract washed with brine (25% aqueous, 3×2000 mL) and concentrated to dryness to give crude product as a black oil (231.3 g, 80.5%). LCMS (ES+), [M+Na]⁺=445.2 & 447, ¹H NMR (300 MHz, CDCl₃) δ 6.65 (d, J=4.2 Hz, 1H), 5.59-5.84 (m, 1H), 5.62 (t, J=9.6 Hz, 1H), 5.40-5.23 (m, 3H), 4.87 (dd, J=9.9, 3.9 Hz, 1H), 4.66-4.59 (m, 3H), 2.21-2.03 (m, 9H)

### Intermediate 2

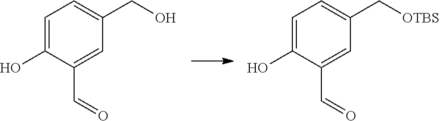 [SMILES: O=Cc1cc(CO)ccc1O ; CC(C)(C)[Si](C)(C)OCc1ccc(O)c(C=O)c1]

TBS-Cl (20.80 g, 138.02 mmol) in DCM (25 mL) was added dropwise to 1H-imidazole (17.90 g, 262.90 mmol) and 2-hydroxy-5-(hydroxymethyl)benzaldehyde (20 g, 131.45 mmol) in DCM (500 mL) at 0° C. over a period of 2 hours under nitrogen. The resulting mixture was stirred at 0° C. for 2 hours. The reaction mixture was quenched with water (500 mL), extracted with DCM (2×300 mL), the organic layer was dried over Na2SO4, filtered and evaporated to afford 5-(((tert-butyldimethylsilyl)oxy)methyl)-2-hydroxybenzaldehyde Intermediate 2 (35.0 g, 100%) as a colourless material. m/z (ES+), [M+Na]⁺=289; NH₄HCO₃, HPLC tR=1.505 min

### Intermediate 3

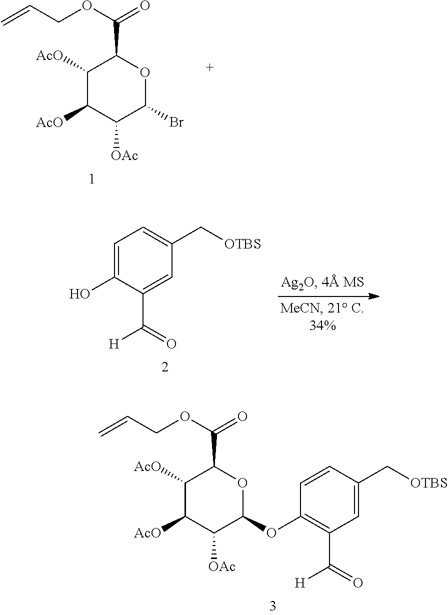 [SMILES: C=CCOC(=O)[C@H]1O[C@H](Br)[C@H](OC(C)=O)[C@@H](OC(C)=O)[C@@H]1OC(C)=O ; CC(C)(C)[Si](C)(C)OCc1ccc(O)c(C=O)c1 ; C=CCOC(=O)[C@H]1O[C@@H](Oc2ccc(CO[Si](C)(C)C(C)(C)C)cc2C=O)[C@H](OC(C)=O)[C@@H](OC(C)=O)[C@@H]1OC(C)=O]

To a vacuum-dried 500 mL round-bottom flask was added molecular sieves (4 Å beads, 5.0 g), silver oxide (29.2 g, 125.8 mmol) and acetonitrile (150 mL), producing a black slurry. To this slurry was added a solution of Intermediate 1 (10.7 g, 25.2 mmol) in acetonitrile (50 mL) over 20 min followed by the addition of 5-(((tert-butyldimethylsilyl)oxy)methyl)-2-hydroxybenzaldehyde (Intermediate 2, 13.6 g, 51.1 mmol) in acetonitrile (50 mL) in one portion. The resulting mixture was stirred vigorously at 20° C. for 16 h. After 16 h, the reaction mixture was filtered through a 5-cm pad of Celite and rinsed with dichloromethane (3×25 mL). Solvent was evaporated then the compound was purified by normal phase flash column chromatography to afford (2S,3S,4S,5R,6S)-2-((allyloxy)carbonyl)-6-(4-(((tert-butyldimethylsilyl)oxy)methyl)-2-formylphenoxy)tetrahydro-2H-pyran-3,4,5-triyl triacetate as a white material Intermediate 3 (5.2 g, 34% yield). ¹H NMR (400 MHz, CDCl₃) δ 10.34 (s, 1H), 7.77 (d, J=1.8 Hz, 1H), 7.58 (dd, J=8.6, 2.1 Hz, 1H), 7.14 (d, J=8.6 Hz, 1H), 5.81-5.92 (m, 1H), 5.39-5.35 (m, 4H), 5.28-5.22 (m, 2H), 4.71 (s, 2H), 4.58-4.67 (m, 2H), 4.20-4.28 (m, 1H), 2.073 (s, 3H), 2.069 (s, 3H), 2.04 (s, 3H), 0.94 (s, 9H), 0.11 (s, 6H); LCMS (ESI) m/z 626.3 (M+NH₄)+.

### Intermediate 4

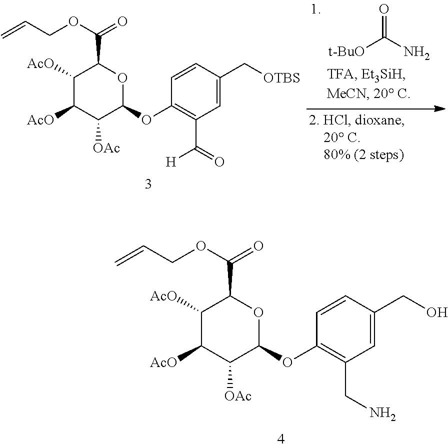 [SMILES: C=CCOC(=O)[C@H]1O[C@@H](Oc2ccc(CO[Si](C)(C)C(C)(C)C)cc2C=O)[C@H](OC(C)=O)[C@@H](OC(C)=O)[C@@H]1OC(C)=O ; CC(C)(C)OC(N)=O ; C=CCOC(=O)[C@H]1O[C@@H](Oc2ccc(CO)cc2CN)[C@H](OC(C)=O)[C@@H](OC(C)=O)[C@@H]1OC(C)=O]

To a solution of Intermediate 3 (5.2 g, 8.6 mmol) in acetonitrile (40 mL) was added tert-butyl carbamate (3.8 g, 32.3 mmol), trifluoroacetic acid (2.0 mL, 25.9 mmol), and triethylsilane (4.1 mL, 25.8 mmol). Stirred for 2 h at 20° C. then solvent was evaporated. To the resulting colorless oil was added 1,4-dioxane (8 mL) and HCl (4.0 M in 1,4-dioxane, 50 mL, 200 mmol). The mixture was stirred at 20° C. for 30 min the solvent was evaporated. The resulting white powder was dissolved in DMSO (3 mL) then passed through cation-exchange resin pre-treated with methanol (WATERS PORAPAK CX). The desired compound was eluted off the resin with methanol to afford (2S,3S,4S,5R,6S)-2-((allyloxy)carbonyl)-6-(2-(aminomethyl)-4-(hydroxymethyl)phenoxy)tetrahydro-2H-pyran-3,4,5-triyl triacetate as a white material Intermediate 4 (2.5 g, 80% over 2 steps). ¹H NMR (500 MHz, CDCl₃) δ 7.26 (d, J=2.2 Hz, 1H), 7.21 (dd, J=8.3, 2.2 Hz, 1H), 7.01 (d, J=8.3 Hz, 1H), 5.90-5.82 (m, 1H), 5.42-5.24 (m, 6H), 5.16 (d, J=7.1 Hz, 1H), 4.64-4.55 (m, 4H), 4.19 (d, J=9.3 Hz, 1H), 3.84 (d, J=14.0 Hz, 1H), 3.67 (d, J=14.0 Hz, 1H), 2.32 (s, 3H), 2.09 (s, 3H), 2.07 (s, 3H), 2.03 (s, 3H). LCMS (ESI) m/z 496.5 (M+H)+.

### Intermediate 5

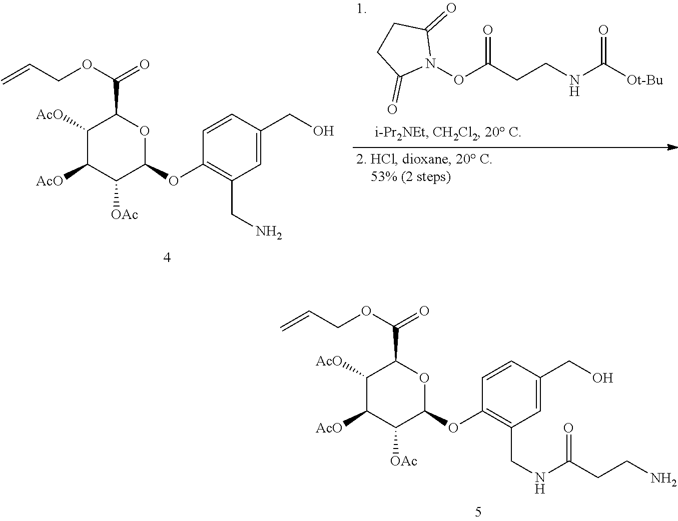 [SMILES: C=CCOC(=O)[C@H]1O[C@@H](Oc2ccc(CO)cc2CN)[C@H](OC(C)=O)[C@@H](OC(C)=O)[C@@H]1OC(C)=O ; CC(C)(C)OC(=O)NCCC(=O)ON1C(=O)CCC1=O ; C=CCOC(=O)[C@H]1O[C@@H](Oc2ccc(CO)cc2CNC(=O)CCN)[C@H](OC(C)=O)[C@@H](OC(C)=O)[C@@H]1OC(C)=O]

To a suspension of Intermediate 4 (2.5 g, 5.0 mmol) in dichloromethane (20 mL) was added N-ethyl-N-isopropylpropan-2-amine (1.8 mL, 10.1 mmol) and 2,5-dioxopyrrolidin-1-yl 3-((tert-butoxycarbonyl)amino)propanoate (1.3 g, 4.4 mmol). Stirred at 20° C. for 10 minutes then water (50 mL) was added. Organic layer was separated then the aqueous layer was extracted with dichloromethane (3×30 mL). Combined organic layers were dried over Na₂SO₄ the solvent was evaporated. To a solution of (2S,3S,4S,5R,6S)-2-((allyloxy)carbonyl)-6-(2-((3-((tert-butoxycarbonyl)amino)propanamido)methyl)-4-(hydroxymethyl)phenoxy)tetrahydro-2H-pyran-3,4,5-triyl triacetate (2.8 g, 4.2 mmol) in dichloromethane (20 mL) was added hydrochloric acid (4.0 M in 1,4-dioxane, 2.6 mL, 83.9 mmol). Stirred at 20° C. for 2 h then solvent was evaporated. The compound was purified by reverse phase flash column chromatography to afford (2S,3S,4S,5R,6S)-2-((allyloxy)carbonyl)-6-(2-((3-aminopropanamido)methyl)-4-(hydroxymethyl)phenoxy)tetrahydro-2H-pyran-3,4,5-triyl triacetate as a colorless material Intermediate 5 (1.3 g, 53% over 2 steps). ¹H NMR (500 MHz, D₂O) δ 7.22 (d, J=2.2 Hz, 1H), 7.16 (d, J=8.2 Hz, 1H), 7.09 (d, J=8.4 Hz, 1H), 5.84 (ddt, J=16.6, 10.5, 6.0 Hz, 1H), 5.44 (t, J=9.2 Hz, 1H), 5.38 (dd, J=7.6, 3.3 Hz, 1H), 5.35-5.23 (m, 4H), 4.62 (d, J=9.8 Hz, 1H), 4.56 (d, J=6.0 Hz, 2H), 4.51 (s, 2H), 4.26 (q, J=15.3 Hz, 2H), 3.23 (t, J=6.8 Hz, 2H), 2.68 (td, J=6.8, 1.9 Hz, 2H), 2.06 (d, J=10.4 Hz, 9H). LCMS (ESI) m/z 567.2 (M+H)+.

### Alternative Synthesis of Intermediate 5

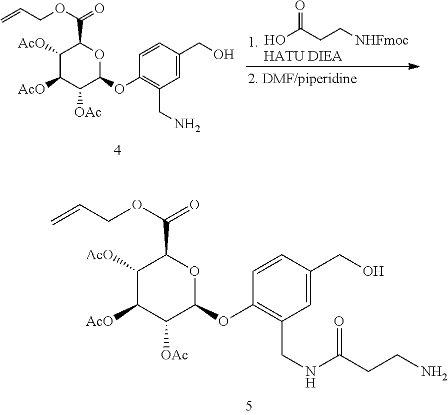

To a stirred reactor containing Intermediate 4 (2.1 kg, 90.5% w/w, 3.57 mol) and acetonitrile (19 L) was added Fmoc-β-alanine (1.11 kg, 3.57 mol). The stirred mixture was cooled to 0° C. To this was added hexafluorophosphate azabenzotriazole tetramethyl uronium (1.36 kg, 3.57 mol) and N,N-diispropylethylamine (0.92 kg, 7.14 mol), and stirred for 4 hours, maintaining the temperature at 0° C. Water (19 L) and ethyl acetate (19 L) was added to the stirred mixture. The organic phase was separated and concentrated to −19 L under vacuum. Ethyl acetate (28.5 L) was added to the concentrated solution and stirred at 20-25° C. for 18 hours. The resulting suspension was filtered, the cake washed with ethyl acetate (3.87 L), and dried under vacuum to (2S,3R,4S,5S,6S)-2-(2-((3-((((9H-fluoren-9-yl)methoxy)carbonyl)amino)propanamido)methyl)-4-(hydroxymethyl)phenoxy)-6-((allyloxy)carbonyl)tetrahydro-2H-pyran-3,4,5-triyl triacetate (1.6 kg, 99% w/w, 56%). LCMS m/z 789 [M+H]⁺

To a stirred reactor containing (2S,3R,4S,5S,6S)-2-(2-((3-((((9H-fluoren-9-yl)methoxy)carbonyl)amino)propanamido)methyl)-4-(hydroxymethyl)phenoxy)-6-((allyloxy)carbonyl)tetrahydro-2H-pyran-3,4,5-triyl triacetate (1 kg, 1.27 mol) and tetrahydrofuran (10 L) at −45° C. under nitrogen was added 2,3,4,6,7,8,9,10-octahydropyrimido[1,2-a]azepine (385.98 g, 2.54 mol). The mixture was stirred at −45° C. for four hours then diluted with acetonitrile (5 L) and quenched by the addition of hydrogen chloride in tert-butyl methyl ether solution (2.54 L, 2.0 M, 5.07 mol). The mixture was concentrated to ˜5 L under vacuum, and diluted with n-heptane (5 L). The acetonitrile layer was collected containing Intermediate 8 (3.88 kg of MeCN solution, 89.97% area, assumed 100%). LCMS m/z 566.6 [M+H]+

### Intermediate 7

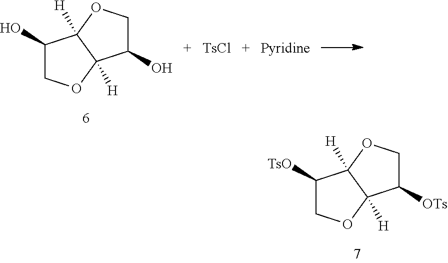 [SMILES: O[C@@H]1CO[C@H]2[C@@H]1OC[C@H]2O ; "TsCl" ; "Pyridine" ; Cc1ccc(S(=O)(=O)O[C@@H]2CO[C@H]3[C@@H]2OC[C@H]3OS(=O)(=O)c2ccc(C)cc2)cc1]

To a 250 mL round bottom flask was added Intermediate 6 (5.0 g, 34.21 mmol)) in dry DCM (100 mL) under nitrogen gas. To the solution was added pyridine (13.84 mL, 171.07 mmol) followed by tosyl-Cl (16.31 g, 85.53 mmol). The reaction mixture was stirred at 20° C. for 16 h. LC-MS analysis showed formation of desired product and completion of reaction. The reaction mixture was diluted with dichloromethane (200). Organic layer was separated, and compound was extracted in 200 mL dichloromethane. Combined organic layer was washed with HCl solution (1M-300 mL), brine (200 mL) and dried over magnesium sulfate. Solvent was removed under reduced pressure to get crude products. The compound was purified via silica gel column to give (3R,3aS,6R,6aS)-hexahydrofuro[3,2-b]furan-3,6-diyl bis(4-methylbenzenesulfonate) Intermediate 7 (14.90 g, 96%). 1H NMR (500 MHz, CDCl3) δ 7.90-7.76 (m, 4H), 7.45-7.33 (m, 4H), 4.94-4.80 (m, 2H), 4.55-4.44 (m, 2H), 3.94 (dd, J=9.6, 6.7 Hz, 2H), 3.75 (dd, J=9.6, 7.6 Hz, 2H), 2.48 (s, 6H). LCMS (ESI) m/z 455.21 (M +H)⁺.

### Intermediate 8

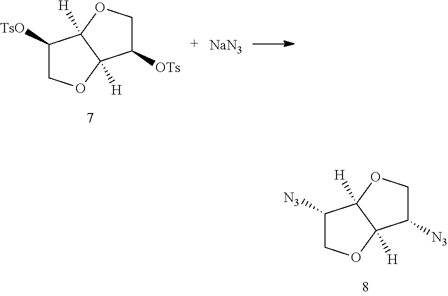

To a 50 mL round bottom flask was added Intermediate 7 (6.0 g, 13.20 mmol) in dry DMF (15 mL) under nitrogen gas. To the solution was added sodium azide (2.146 g, 33.00 mmol). The reaction mixture was at 140° C. for 3 hrs. LC-MS analysis showed formation of desired product and completion of reaction. The reaction mixture was diluted with dichloromethane (200×2 mL), and organic layer was separated, washed with water (200 mL), brine (200 mL) and dried over magnesium sulfate. Solvent was removed under reduced pressure to get (3S,3aR,6S,6aR)-3,6-diazidohexahydrofuro[3,2-b]furan Intermediate 8 (2.050 g, 79%). ¹H NMR (500 MHz, CDCl₃) δ 4.61 (d, J=1.9 Hz, 2H), 4.05 (d, J=4.0 Hz, 2H), 3.97-3.82 (m, 4H). LCMS (ESI) m/z 197.1 (M+H)⁺.

### Intermediate 9

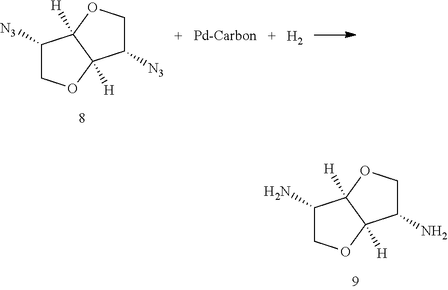

To a 250 mL round bottom flask was added Intermediate 8 (1 g, 5.10 mmol) in dry THF (20 mL) under nitrogen gas. To the solution was added barium palladium(II) carbonate (0.618 g, 0.51 mmol). The reaction mixture was flushed with hydrogen (1.028 g, 509.76 mmol) gas and stirred at 23° C. for 3 hrs. under H₂ gas. LC-MS analysis showed formation of desired product and completion of reaction. The reaction mixture was diluted with methanol (20 mL) filtered through celite pad. Celite pad was washed with methanol (50 mL). Filtrate was dried over magnesium sulfate. Solvent was removed under reduced pressure to get (3S,3aR,6S,6aR)-hexahydrofuro[3,2-b]furan-3,6-diamine Intermediate 9 (0.590 g, 80%). ¹H NMR (500 MHz, DMSO) δ 4.23 (s, 2H), 3.68 (dd, J=8.7, 4.5 Hz, 2H), 3.41 (dd, J=8.7, 1.9 Hz, 2H), 3.23 (dd, J=4.5, 1.9 Hz, 2H), 1.54 (s, 4H). LCMS (ESI) m/z 145.2 (M+H)⁺.

### Alternative Synthesis of Intermediate 9

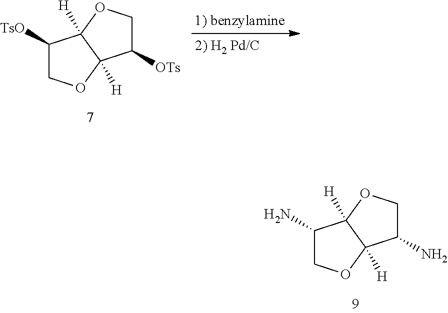 [SMILES: Cc1ccc(S(=O)(=O)O[C@@H]2CO[C@H]3[C@@H]2OC[C@H]3OS(=O)(=O)c2ccc(C)cc2)cc1 ; N[C@H]1CO[C@H]2[C@@H]1OC[C@@H]2N]

To reactor was added intermediate 6 (4 kg, 8.8 mol) and benzylamine (12 L) under nitrogen. The stirred mixture was heated to 160° C. for 24 hours then cooled to 20-25° C., diluted with tert-butyl methyl ether (80 L) and cooled further to 10° C. To this was added para-toluene sulfonic acid (12.11 kg, 70.44 mol), the mixture stirred for 2.5 hours at 20-25° C. and then filtered. The cake was washed with tert-butyl methyl ether (8 L) and the combined filtrates washed with saturated aqueous sodium hydrogen carbonate solution (20 L). The organic phase was evaporated to dryness, dissolved in ethanol (20 L) and evaporated to dryness to give crude intermediate 7 (3.05 kg, 80.5% w/w, 85.9%) LCMS m/z (ES+), [M+H]⁺=325.1

To a reactor was added Intermediate 7 (1.5 kg, 3.08 mol) and ethanol (12.12 L) under a dry under nitrogen atmosphere. To the solution was added 10% wt palladium on carbon (120.8, 10% w/w). The reaction mixture was flushed with hydrogen gas and stirred at 80° C. for 16 hours under hydrogen. Mixture cooled to 20-25° C. and filtered through cellulose (2.42 kg). The cake was washed with ethanol (2.44 L) and combined filtrates were concentrated to dryness. The residue was dissolved in acetonitrile (6.04 L) and concentrated to dryness to give Intermediate 9 (509 g, 84% w/w, 80.2%). LCMS m/z (ES+), [M+H]⁺=145

### Intermediate 11

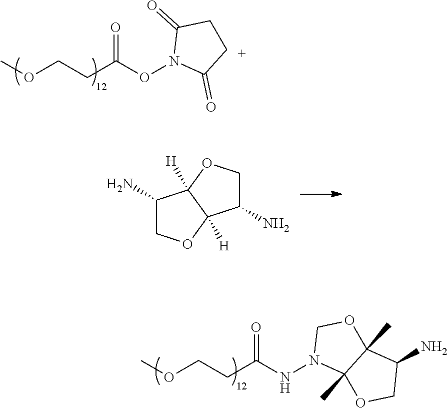

To a 250 mL round bottom flask was added Intermediate 9 (1.50 g, 10.40 mmol) in dry THF (25 mL) under nitrogen gas. To the solution was added sodium hydrogen carbonate (1.748 g, 20.81 mmol) and 2,5-dioxopyrrolidin-1-yl 2,5,8,11,14,17,20,23,26,29,32,35-dodecaoxaoctatriacontan-38-oate Intermediate 10 (7.13 g, 10.40 mmol) in portions under nitrogen gas and stirred at 20° C. for 6 hrs. LC-MS analysis showed formation of desired product and completion of reaction. The reaction mixture was quenched by addition of methanol (10 mL). The reaction mixture was diluted with methanol (20 mL) filtered through celite pad. Celite pad was washed with methanol (50 mL). Filtrate was dried over magnesium sulfate. Solvent was removed under reduced pressure to get crude product. the crud product was purified via silica gel column to give N-((3S,3aR,6S,6aR)-6-aminohexahydrofuro[3,2-b]furan-3-yl)-2,5,8,11,14,17,20,23,26,29,32,35-dodecaoxaoctatriacontan-38-amide Intermediate 11 (4.00 g, 53.8%). ¹H NMR (500 MHz, MeOD) δ 4.64-4.59 (m, 1H), 4.45 (dd, J=4.1, 1.3 Hz, 1H), 4.29 (dt, J=4.1, 1.9 Hz, 1H), 3.96 (ddd, J=10.2, 9.3, 4.9 Hz, 2H), 3.81-3.74 (m, 3H), 3.73-3.62 (m, 45H), 3.61-3.56 (m, 2H), 3.45 (dt, J=3.8, 1.8 Hz, 1H), 3.40 (s, 3H), 2.52-2.47 (m, 2H). LCMS (ESI) m/z 715.6 (M+H)⁺.

### Intermediate 13

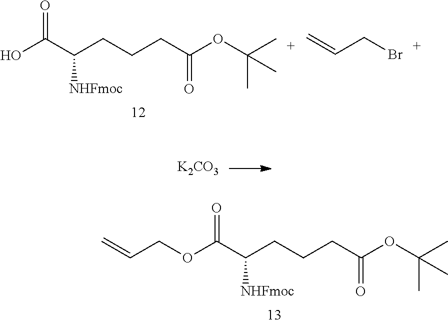

To a 250 mL round bottom flask was added (S)-2-((((9H-fluoren-9-yl)methoxy)carbonyl)amino)-6-(tert-butoxy)-6-oxohexanoic acid Intermediate 12 (5 g, 11.38 mmol) in dry DMF (20 mL) under nitrogen gas. To the solution was added potassium carbonate (3.14 g, 22.75 mmol) and 3-bromoprop-1-ene (1.485 mL, 17.06 mmol) in portions under nitrogen gas and stirred at 20° C. for 16 hrs. LC-MS analysis showed formation of desired product and completion of reaction. The reaction mixture was diluted with water (500 mL) and organic layer was extracted with ethyl acetate (2×300 mL), washed with water (300 mL), brine (200 mL) and dried over sodium sulfate (20 g). The solvent was removed to get crude product. The crude product was purified via silica gel column to give 1-allyl 6-(tert-butyl) (S)-2-((((9H-fluoren-9-yl)methoxy)carbonyl)amino)hexanedioate Intermediate 13 (5.10 g, 93%). ¹H NMR (500 MHz, CDCl₃) δ 7.79-7.73 (m, 2H), 7.61 (q, J=3.9 Hz, 2H), 7.40 (t, J=7.5 Hz, 2H), 7.32 (tt, J=7.4, 1.2 Hz, 2H), 5.91 (ddt, J=16.5, 10.9, 5.8 Hz, 1H), 5.42-5.23 (m, 3H), 4.66 (d, J=5.8 Hz, 2H), 4.40 (q, J=4.8 Hz, 3H), 4.23 (t, J=7.1 Hz, 1H), 2.26 (t, J=7.2 Hz, 2H), 1.96-1.82 (m, 1H), 1.72 (dq, J=13.5, 6.1 Hz, 3H), 1.60-1.47 (m, 1H), 1.45 (s, 9H). LCMS (ESI) m/z 480.2 (M+H)⁺.

### Intermediate 14

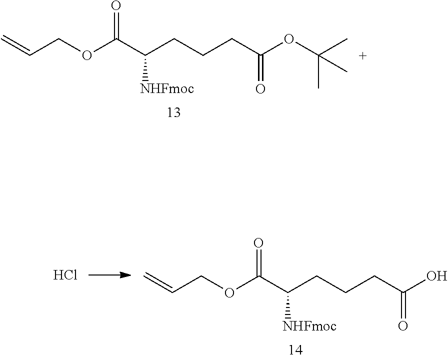

To a 100 mL round bottom flask was added Intermediate 13 (5 g, 10.43 mmol) in dry THF (20 mL) under nitrogen gas. To the solution was added HCl (13.03 mL, 52.13 mmol), 4 molar in dioxane under nitrogen gas and stirred at 20° C. for 6 hrs. LC-MS analysis showed formation of desired product and completion of reaction. The reaction mixture was diluted with water (200 mL) and organic layer was extracted with dichloromethane (2×300 mL), washed with brine (200 mL) and dried over sodium sulfate (20 g). The solvent was removed to get (S)-5-((((9H-fluoren-9-yl)methoxy)carbonyl)amino)-6-(allyloxy)-6-oxohexanoic acid Intermediate 14 (4.20 g, 95%). ¹H NMR (500 MHz, CDCl₃) δ 7.75 (dq, J =7.6, 1.0 Hz, 2H), 7.62-7.52 (m, 2H), 7.42-7.35 (m, 2H), 7.30 (tt, J=7.4, 1.2 Hz, 2H), 5.90 (ddt, J=16.4, 10.8, 5.8 Hz, 1H), 5.48 (d, J=8.4 Hz, 1H), 5.37-5.20 (m, 2H), 4.64 (d, J=5.8 Hz, 2H), 4.40 (d, J=7.2 Hz, 3H), 4.22 (t, J=7.0 Hz, 1H), 2.45-2.24 (m, 2H), 1.93 (p, J=5.6 Hz, 1H), 1.72 (td, J=13.9, 6.8 Hz, 3H). (ESI) m/z 424.5 (M−H)⁻.

### Intermediate 15

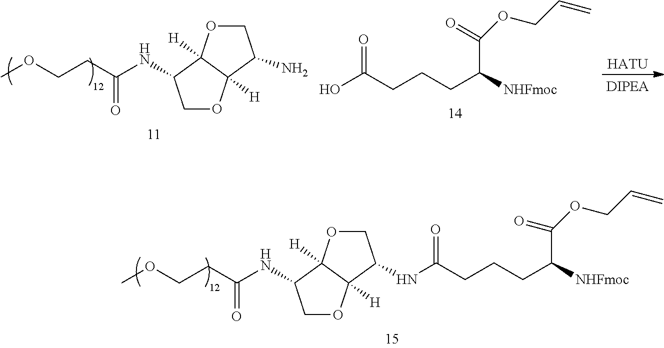

To a 100 mL round bottom flask was added Intermediate 14 (1.925 g, 4.55 mmol) under nitrogen gas. To the solution was added HATU (1.862 g, 4.90 mmol) followed by DIPEA (1.222 mL, 6.99 mmol). The reaction mixture was stirred at room temperature for 15 min then Intermediate 11 (2.5 g, 3.50 mmol). was added and reaction mixture was stirred at 23° C. for 3 hrs. LC-MS analysis showed formation of desired product and completion of reaction. The reaction mixture was diluted with DCM (300 mL), washed with water (200 mL), organic layer was extracted (2×100 mL), washed with Brine (50 mL), dried over sodium sulfate (5 g). Solvent was removed under reduced pressure to get crude product. The crude product was purified via silica gel column to give allyl (S)-6-(((3S,3aR,6S,6aR)-6-(2,5,8,11,14,17,20,23,26,29,32,35-dodecaoxaoctatriacontan-38-amido)hexahydrofuro[3,2-b]furan-3-yl)amino)-2-((((9H-fluoren-9-yl)methoxy)carbonyl)amino)-6-oxohexanoate Intermediate 15 (3.40 g, 87%) ¹H NMR (500 MHz, MeOD) δ 7.86 (dd, J=7.6, 1.2 Hz, 2H), 7.74 (t, J=7.8 Hz, 2H), 7.46 (td, J=7.5, 1.4 Hz, 2H), 7.38 (tt, J=7.5, 1.3 Hz, 2H), 6.05-5.93 (m, 1H), 5.39 (dq, J=17.2, 1.6 Hz, 1H), 5.28 (dq, J=10.5, 1.4 Hz, 1H), 4.73-4.65 (m, 2H), 4.58 (qd, J=4.1, 1.0 Hz, 2H), 4.46 (dd, J=10.6, 7.0 Hz, 1H), 4.40 (dd, J=10.6, 7.0 Hz, 1H), 4.36-4.31 (m, 2H), 4.31-4.24 (m, 2H), 4.01 (ddd, J=9.6, 5.0, 1.1 Hz, 2H), 3.84-3.73 (m, 5H), 3.72-3.60 (m, 44H), 3.60-3.56 (m, 2H), 3.41 (s, 3H), 2.49 (td, J=6.0, 1.9 Hz, 2H), 2.31 (hept, J=7.2 Hz, 2H), 1.96-1.85 (m, 1H), 1.85-1.68 (m, 3H). LCMS (ESI) m/z 1121.3 (M+H)⁺.

### Intermediate 16

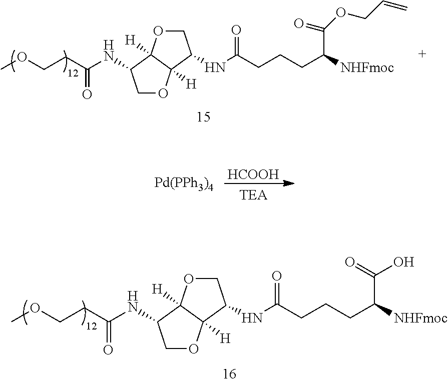

To a 50 mL round bottom flask was added Intermediate 15 (4.3 g, 3.84 mmol) in dry DCM (10 mL) under nitrogen gas. To the solution was added triethylamine (0.535 mL, 3.84 mmol) followed by triphenylphosphine (0.101 g, 0.38 mmol). To the reaction mixture was added Pd(PPh₃)₄ (0.444 g, 0.38 mmol) then formic acid (0.147 mL, 3.84 mmol) was added and reaction mixture was stirred at 23° C. for 6 hrs. LC-MS analysis showed formation of desired product and completion of reaction. Solvent was removed under reduced pressure to get crude products. The crude product was purified via silica gel column to give (S)-6-(((3S,3aR,6S,6aR)-6-(2,5,8,11,14,17,20,23,26,29,32,35-dodecaoxaoctatriacontan-38-amido)hexahydrofuro[3,2-b]furan-3-yl)amino)-2-((((9H-fluoren-9-yl)methoxy)carbonyl)amino)-6-oxohexanoic acid Intermediate 16 (3.50 g, 84%). ¹H NMR (500 MHz, DMSO) δ 8.12 (dd, J=10.2, 7.0 Hz, 2H), 7.90 (d, J=7.6 Hz, 2H), 7.74 (d, J=7.5 Hz, 2H), 7.63 (d, J=8.1 Hz, 1H), 7.46-7.38 (m, 2H), 7.34 (td, J=7.4, 1.2 Hz, 2H), 4.38 (s, 2H), 4.32-4.20 (m, 3H), 4.11 (ddt, J=7.2, 4.8, 2.1 Hz, 2H), 3.93 (td, J=8.4, 4.6 Hz, 1H), 3.85 (dd, J=9.3, 5.1 Hz, 2H), 3.63-3.57 (m, 4H), 3.54-3.45 (m, 42H), 3.45-3.40 (m, 2H), 3.24 (s, 3H), 2.33 (t, J=6.5 Hz, 2H), 2.09 (s, 3H), 1.68 (d, J=9.1 Hz, 1H), 1.64-1.48 (m, 3H). LCMS (ESI) m/z 1080.6 (M+H)⁺.

### Intermediate 17

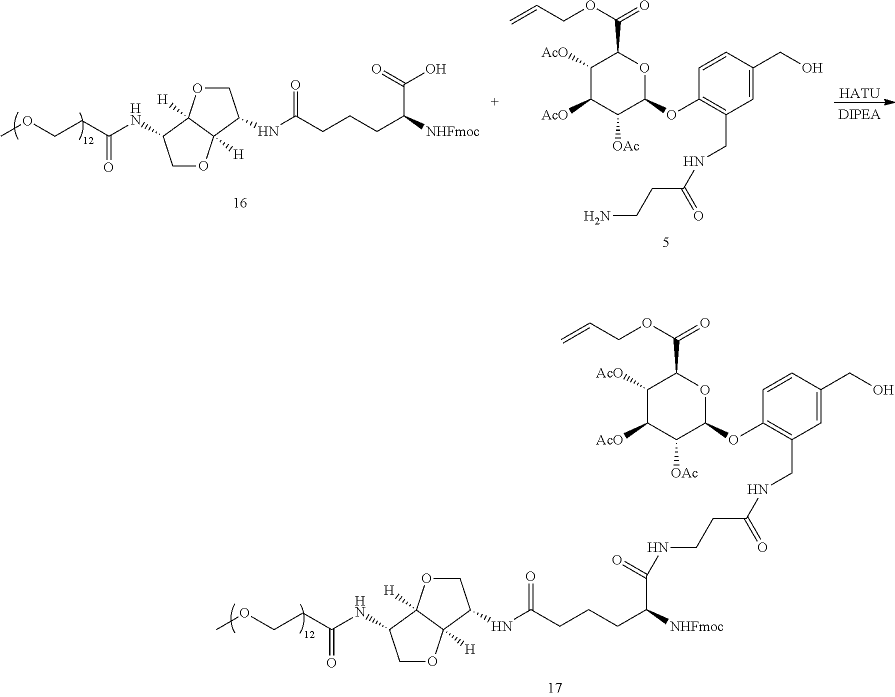

To a 100 mL round bottom flask was added Intermediate 16 (0.8 g, 0.74 mmol) under nitrogen gas. To the solution was added HATU (0.366 g, 0.96 mmol) followed by DIPEA (0.388 mL, 2.22 mmol). The reaction mixture was stirred at room temperature for 15 min then Intermediate 5 (0.670 g, 1.11 mmol). was added and reaction mixture was stirred at 23° C. for 3 hrs. LC-MS analysis showed formation of desired product and completion of reaction. The reaction mixture was diluted with DCM (100 mL), washed with water (100 mL), organic layer was extracted (2×100 mL), washed with Brine (100 mL), dried over sodium sulfate (15 g). Solvent was removed under reduced pressure and purified via silica gel column to give (2S,3R,4S,5S,6S)-2-(2-((S)-5-(4-(((3S,3aR,6S,6aR)-6-(2,5,8,11,14,17,20,23,26,29,32,35-dodecaoxaoctatriacontan-38-amido)hexahydrofuro[3,2-b]furan-3-yl)amino)-4-oxobutyl)-1-(9H-fluoren-9-yl)-3,6,10-trioxo-2-oxa-4,7,11-triazadodecan-12-yl)-4-(hydroxymethyl)phenoxy)-6-((allyloxy)carbonyl)tetrahydro-2H-pyran-3,4,5-triyl triacetate Intermediate 17 (0.640 g, 53.1%). 1H NMR (500 MHz, DMSO) δ 8.21 (t, J=6.0 Hz, 1H), 8.11 (dd, J=19.5, 6.9 Hz, 2H), 7.94 (t, J=5.8 Hz, 1H), 7.89 (d, J=7.5 Hz, 2H), 7.73 (t, J=7.0 Hz, 2H), 7.49-7.39 (m, 3H), 7.33 (td, J=7.5, 1.2 Hz, 2H), 7.17 (dd, J=8.4, 2.2 Hz, 1H), 7.13 (s, 1H), 7.01 (d, J=8.4 Hz, 1H), 5.89 (ddt, J=17.3, 10.5, 5.7 Hz, 1H), 5.56 (d, J=7.9 Hz, 1H), 5.48 (t, J=9.6 Hz, 1H), 5.33 (dq, J=17.2, 1.6 Hz, 1H), 5.26 (dq, J=10.5, 1.4 Hz, 1H), 5.19-5.07 (m, 3H), 4.76 (d, J=10.0 Hz, 1H), 4.62 (ddt, J=13.3, 5.6, 1.4 Hz, 1H), 4.54 (ddt, J=13.3, 5.8, 1.4 Hz, 1H), 4.44-4.36 (m, 4H), 4.31-4.18 (m, 4H), 4.11 (d, J=5.9 Hz, 3H), 3.93 (d, J=5.9 Hz, 1H), 3.88-3.81 (m, 2H), 3.66-3.56 (m, 5H), 3.56-3.45 (m, 42H), 3.45-3.39 (m, 3H), 3.29-3.27 (m, 1H), 3.24 (s, 3H), 3.18 (d, J=5.0 Hz, 1H), 2.33 (ddt, J=14.6, 10.0, 7.6 Hz, 4H), 2.10-2.05 (m, 2H), 2.04 (s, 3H), 1.99 (d, J=4.6 Hz, 6H), 1.54 (dt, J =43.7, 8.9 Hz, 4H). LCMS (ESI) m/z 1629.8 (M+H)⁺.

### Intermediate 18

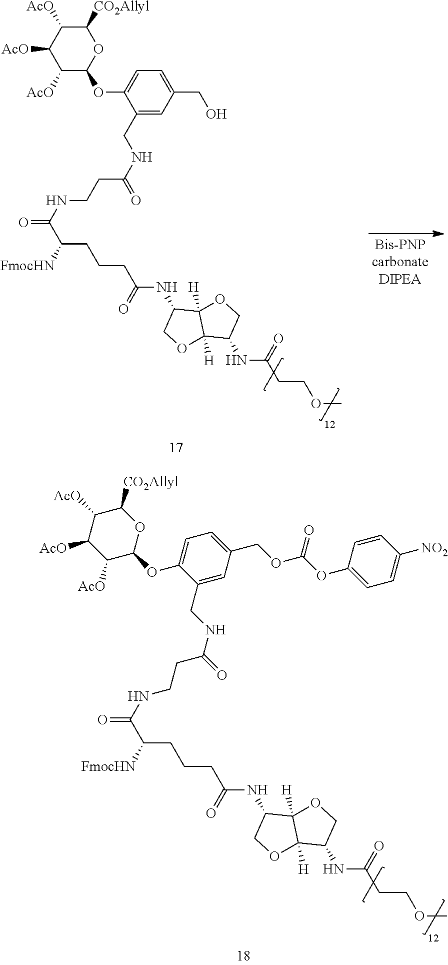

To a solution of Intermediate 17 (25.00 mg, 0.02 mmol, 1.0 eq) in DMF was added bis(4-nitrophenyl) carbonate (28.0 mg, 0.09 mmol, 4.5 eq) and DIPEA (0.013 mL, 0.08 mmol, 4.0 eq). The mixture was stirred at 23° C. for 2 hours. Following this time the mixture was concentrated in vacuo and the residue was sonicated in DCM (200 μL) and diethyl ether (2 mL). The resulting suspension was dried on vacuum filter and the process repeated. The residue was dried to give (2S,3R,4S,5S,6S)-2-(2-((S)-5-(4-(((3S,3aR,6S,6aR)-6-(2,5,8,11,14,17,20,23,26,29,32,35-dodecaoxaoctatriacontan-38-amido)hexahydrofuro [3,2-b]furan-3-yl)amino)-4-oxobutyl)-1-(9H-fluoren-9-yl)-3,6,10-trioxo-2-oxa-4,7,11-triazadodecan-12-yl)-4-((((4-nitrophenoxy)carbonyl)oxy)methyl)phenoxy)-6-((allyloxy)carbonyl)tetrahydro-2H-pyran-3,4,5-triyl triacetate Intermediate 18 (32 mg, 0.02 mmol, 91%) as a yellow material. RT 7.74 min. LCMS (ESI) m/z 1794.7 [M+H]⁺.

### Intermediate 19

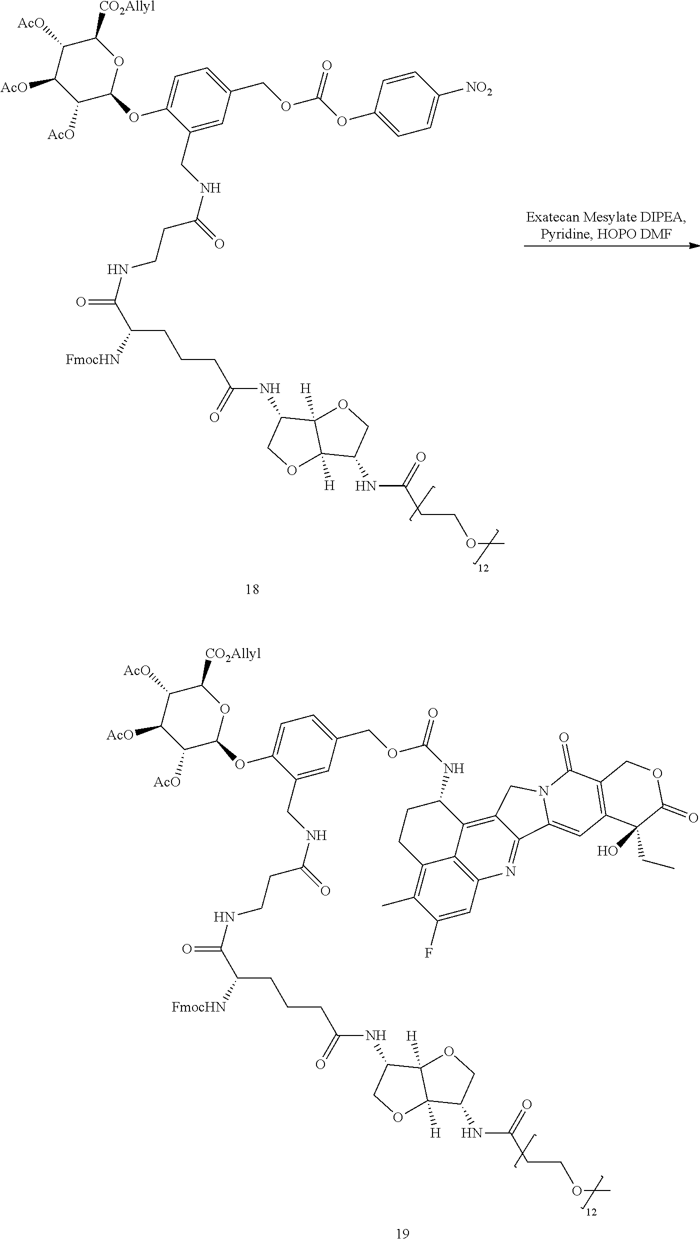

To a solution of exatecan mesylate (7.41 mg, 0.01 mmol, 1.0 eq) in DCM (1 mL) and DMF (1.000 mL) was added DIPEA (7.28 μl, 0.04 mmol, 4.0 eq), Intermediate 18 (25.00 mg, 0.01 mmol, 1.0 eq), and HOPO (1.703 mg, 0.02 mmol, 2.0 eq) and the resultant mixture was stirred at 23° C. for 18 hours. Following this time the mixture was concentrated in vacuo and the residue was purified by reverse phase flash column chromatography (C18 BIOTAGE prepacked column, 40-60% MeCN [0.1% formic acid]/water [0.1% formic acid]) to give (2S,3R,4S,5S,6S)-2-(2-((S)-5-(4-(((3S,3aR,6S,6aR)-6-(2,5,8,11,14,17,20,23,26,29,32,35-dodecaoxaoctatriacontan-38-amido)hexahydrofuro [3,2-b]furan-3-yl)amino)-4-oxobutyl)-1-(9H-fluoren-9-yl)-3,6,10-trioxo-2-oxa-4,7,11-triazadodecan-12-yl)-4-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)phenoxy)-6-((allyloxy)carbonyl)tetrahydro-2H-pyran-3,4,5-triyl triacetate Intermediate 19 (13 mg, 0.01 mmol, 63%) as a yellow material. RT 7.66 min. LCMS (ESI) 2090.3 [M+H]+.

### Intermediate 20

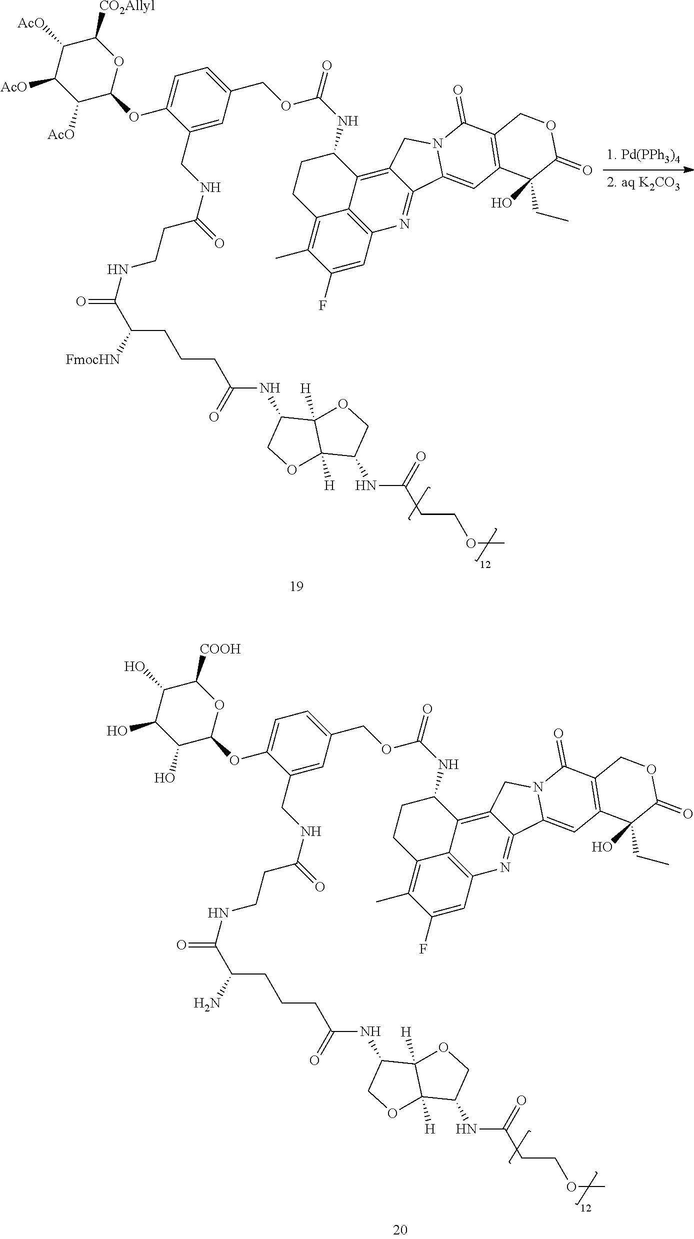

To a solution of Intermediate 19 (15.00 mg, 7.18 μmol, 1.0 eq) in DCM (1 mL) was added triethylamine (2.00 μl, 0.01 mmol, 1.4 eq), Pd(PPh₃)₄ (1.00 mg, 0.87 μmol, 12 mol %) and formic acid (0.541 μl, 0.01 mmol, 1.4 eq) and the mixture stirred at 23° C. for 18 hours the mixture. Following this time the reaction mixture was concentrated and the crude residue was dissolved in methanol (0.25 mL) and THF (0.25 mL). To this solution was added potassium carbonate (9.44 mg, 0.07 mmol, 10 eq) in water (0.5 mL) and the mixture was stirred at 23° C. for 3 hours. Following this time the mixture was concentrated in vacuo to remove organics. The remaining aqueous solution was acidified with citric acid (1 N) until pH 4 was reached and the mixture was stirred for 1 hour at 23° C. Following this time the mixture was filtered and the residue was purified by reverse phase flash column chromatography (C18 BIOTAGE prepacked column, 20-40% MeCN [0.1% formic acid]/water [0.1% formic acid]) to give (2S,3S,4S,5R,6S)-6-(2-((3-((S)-6-(((3S,3aR,6S,6aR)-6-(2,5,8,11,14,17,20,23,26,29,32,35-dodecaoxaoctatriacontan-38-amido)hexahydrofuro [3,2-b]furan-3-yl)amino)-2-amino-6-oxohexanamido)propanamido)methyl)-4-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)phenoxy)-3,4,5-trihydroxytetrahydro-2H-pyran-2-carboxylic acid Intermediate 20 (8.6 mg, 5.03 μmol, 70%) as a yellow material. RT 5.08 min. LCMS (ESI) 1702.6 [M+H]+.

### Linker-Payload β-Glu-Exatecan (LP-1)

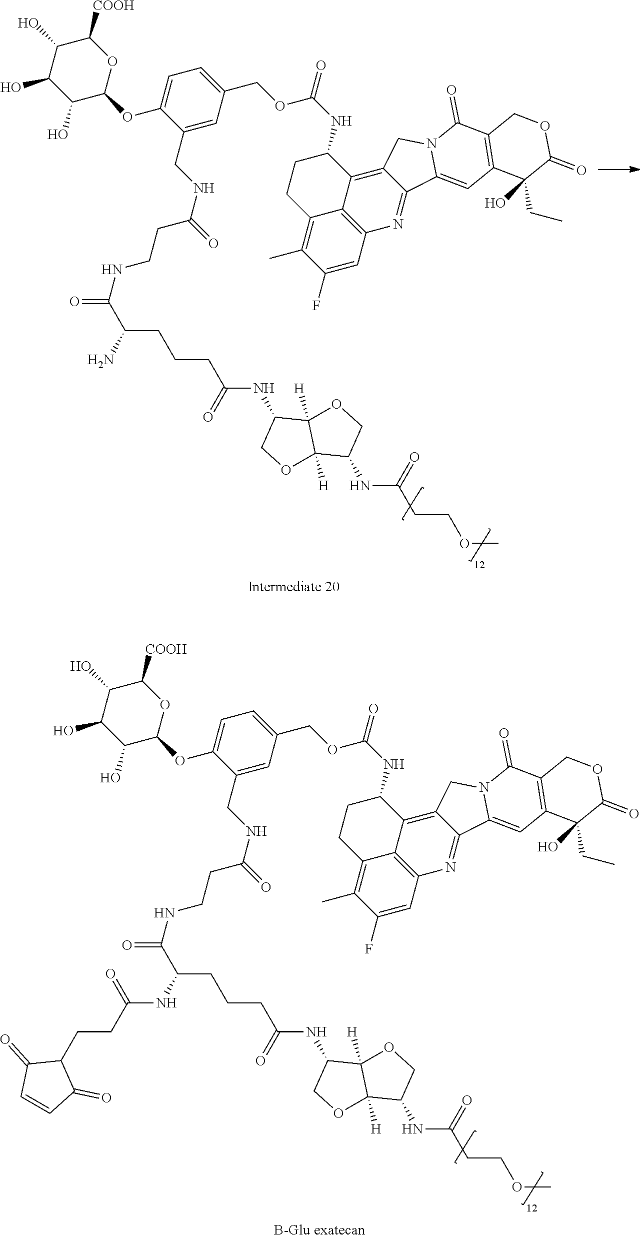

To a solution of Intermediate 20 (6.20 mg, 3.64 μmol, 1.0 eq) in DMF (0.5 mL) was added pyridine (0.7 μl, 5.5 μmol, 1.5 eq) and 2,5-dioxopyrrolidin-1-yl 3-(2,5-dioxo-2,5-dihydro-1H-pyrrol-1-yl)propanoate (0.970 mg, 3.64 μmol, 1.0 eq). The mixture was stirred at 23° C. for 3 hours. Following this time the reaction mixture was concentrated in vacuo and the residue purified by reverse phase HPLC (CSH 35% MeCN [0.1% formic acid]/water [0.1% formic acid] over 7 min) to give (2S,3S,4S,5R,6S)-6-(2-((3-((S)-6-(((3S,3aR,6S,6aR)-6-(2,5,8,11,14,17,20,23,26,29,32,35-dodecaoxaoctatriacontan-38-amido)hexahydrofuro [3,2-b]furan-3-yl)amino)-2-(3-(2,5-dioxo-2,5-dihydro-1H-pyrrol-1-yl)propanamido)-6-oxohexanamido)propanamido)methyl)-4-(((((1S,9S)-9-ethyl-5-fluoro-9-hydroxy-4-methyl-10,13-dioxo-2,3,9,10,13,15-hexahydro-1H,12H-benzo [de]pyrano [3′,4′:6,7]indolizino [1,2-b]quinolin-1-yl)carbamoyl)oxy)methyl)phenoxy)-3,4,5-trihydroxytetrahydro-2H-pyran-2-carboxylic acid LP-1 (2.7 mg, 1.46 μmol, 40%) as a white material. RT 5.87 min. LCMS (ESI) 1853.5 [M+H]+.

### Alternative Synthesis of LP-1

### Intermediate A

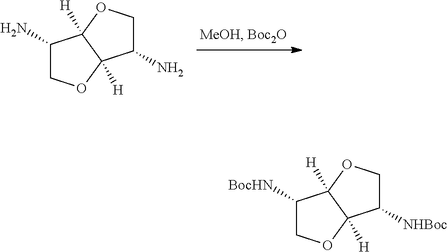 [SMILES: N[C@H]1CO[C@H]2[C@@H]1OC[C@@H]2N ; CC(C)(C)OC(=O)N[C@H]1CO[C@H]2[C@@H]1OC[C@@H]2NC(=O)OC(C)(C)C]

To intermediate 9 (22.0 g, 152.6 mmol) in methanol (440 mL) was added boc anhydride (83.3 g, 381.5 mmol, 2.5 eq) and the reaction was stirred for 2 hours at 20° C. ELSD analysis showed formation of the desired product and completion of the reaction. Solvent was removed under reduced pressure to get crude product. The crude product was slurried with MTBE (200 mL), filtered, washed with MTBE (40 mL) and dried to give tert-butyl N-[(3S,3aR,6S,6aR)-6-(tert-butoxycarbonylamino)-2,3,3a,5,6,6a-hexahydrofuro[3,2-b]furan-3-yl]carbamate Intermediate A (44.0 g, 84.3%). LCMS m/z 366.8 [M+Na]+

### Intermediate B

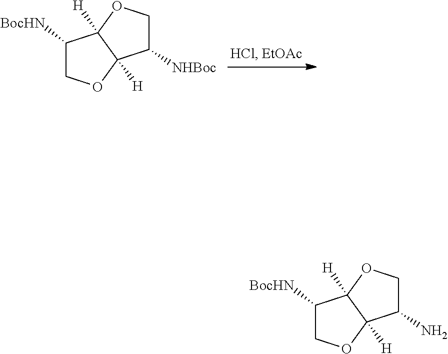 [SMILES: CC(C)(C)OC(=O)N[C@H]1CO[C@H]2[C@@H]1OC[C@@H]2NC(=O)OC(C)(C)C ; CC(C)(C)OC(=O)N[C@H]1CO[C@H]2[C@@H]1OC[C@@H]2N]

To intermediate A (2.0 g, 5.81 mmol, 1.0 eq) in ethyl acetate (40 mL) was added HCl in ethyl acetate (4M, 8.0 mL) and the reaction was stirred for 6 hours at 20° C. ELSD analysis showed formation of the desired product and completion of the reaction. The reaction was quenched with potassium phosphate solution (2M, 20 mL), the layers were separated and the organic layer retained. The aqueous layer was extracted with ethyl acetate (10 mL) and the organic layers combined. Solvent was removed under reduced pressure to give tert-butyl N-[(3S,3aR,6S,6aR)-3-amino-2,3,3a,5,6,6a-hexahydrofuro [3,2-b]furan-6-yl]carbamate Intermediate B (800 mg, 57.1%). ¹H NMR (400 MHz, DMSO-d6) δ 4.32 (m, 1H), 3.93 (m, 1H), 3.85 (m, 2H), 3.6 (m, 2H), 3.28 (m, 1H), 1.47 (s, 9H). LCMS m/z 145 [M+H-Boc]⁺

### Intermediate C

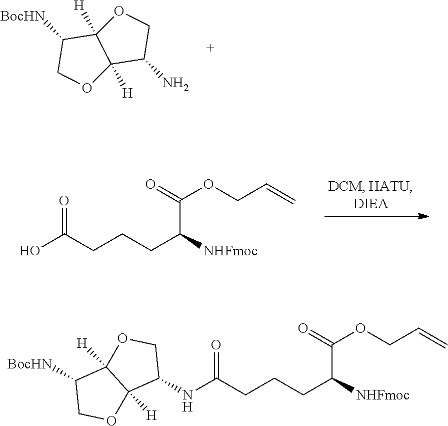

To a solution of Intermediate 14 (17.16 g, 40.5 mmol, 1.1 eq) in DCM (180 mL) at 25° C., was added HATU (16.8 g, 44.2 mmol, 1.2 eq) and DIPEA (10.9 ml, 62.6 mmol, 1.7 eq). The reaction mixture was stirred for 15 mins. To the reaction solution was added Intermediate B (9.0 g, 36.8 mmol, 1.0 eq) in DCM 90 mL)). The reaction mixture was stirred for 2 hours. LC-MS analysis showed formation of the desired product and completion of the reaction. The reaction mixture was washed twice with NaCl (15% aq, 90 mL) and the aqueous layers were discarded. Solvent was removed under reduced pressure to give allyl (2S)-6-[[(3S,3aR,6S,6aR)-6-(tert-butoxycarbonylamino)-2,3,3a,5,6,6a-hexahydrofuro[3,2-b]furan-3-yl]amino]-2-(9H-fluoren-9-ylmethoxycarbonylamino)-6-oxo-hexanoate Intermediate C (9.0 g, 65.2%). LCMS m/z 671.6 [M+Na]⁺

### Intermediate D

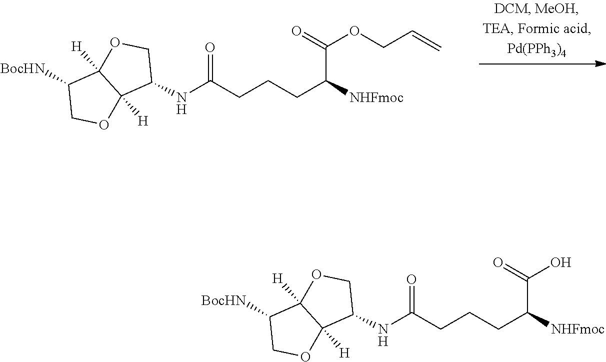

To a solution of Intermediate C (9.0 g, 13.9 mmol, 1.0 eq) in DCM/MeOH (20:1, 128.6 mL:6.4 mL) was added formic acid (1.05 mL, 27.7 mmol, 2.0 eq), triethylamine (5.79 mL, 41.6 mmol, 3.0 eq) and Pd(PPh₃)₄ (1.6 g, 1.39 mmol, 0.1 eq). The reaction mixture was stirred for 3 hours at 20-25° C. LC-MS analysis showed formation of the desired product and completion of the reaction. Solvent was removed under reduced pressure to get crude product. The crude product was purified via silica gel column (DCM to DCM:MeOH 4:1) to give (2S)-6-[[(3S,3aR,6S,6aR)-6-(tert-butoxycarbonylamino)-2,3,3a,5,6,6a-hexahydrofuro[3,2-b]furan-3-yl]amino]-2-(9H-fluoren-9-ylmethoxycarbonylamino)-6-oxo-hexanoic acid Intermediate D (7.0 g, 82.9%). LCMS m/z 610.1 [M+H]⁺

### Intermediate E

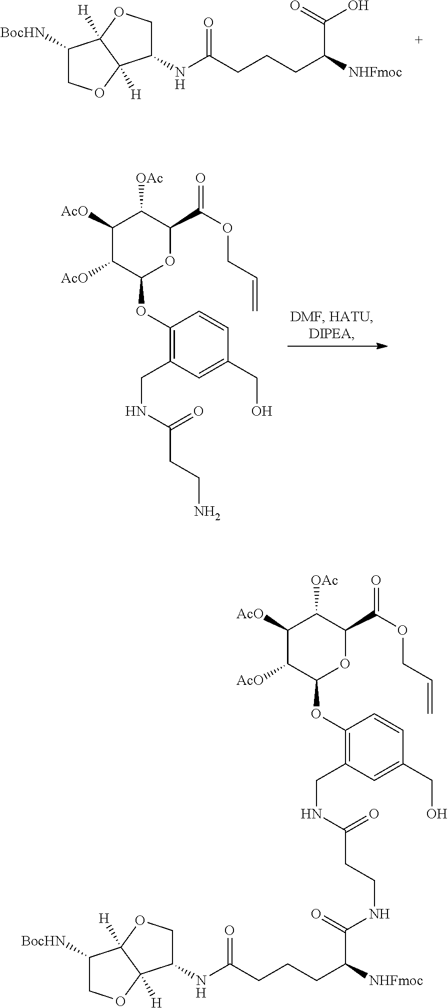

To a solution of Intermediate D (4.0 g, 6.56 mmol, 1.0 eq) in DMF (80 mL) at 0-5° C. was added Intermediate 5 (11.2 g, 19.7 mmol, 3.0 eq), HATU (3.74 g, 9.84 mmol, 1.5 eq), and DIPEA (3.43 mL, 19.7 mmol, 3.0 eq). The reaction mixture was stirred for 1 hours at 0-5° C. LC-MS analysis showed formation of the desired product and completion of the reaction. The reaction mixture was diluted with ethyl acetate (80 mL) and washed twice with NaCl (15% aq, 40 mL). The aqueous layers were discarded and the solvent was removed from the organic layer under reduced pressure to get crude product. The crude product was purified via silica gel column (EtOAC to 20% MeOH) to give allyl (2S,3S,4S,5R,6S)-6-[2-[[3-[[(2S)-6-[[(3S,3aR,6S,6aR)-6-(tert-butoxycarbonylamino)-2,3,3a,5,6,6a-hexahydrofuro[3,2-b]furan-3-yl]amino]-2-(9H-fluoren-9-ylmethoxycarbonylamino)-6-oxo-hexanoyl]amino]propanoylamino]methyl]-4-(hydroxymethyl)phenoxy]-3,4,5-triacetoxy-tetrahydropyran-2-carboxylate Intermediate E (3.6 g, 47.4%) LCMS m/z 1158.2 [M+H]⁺

### Intermediate F

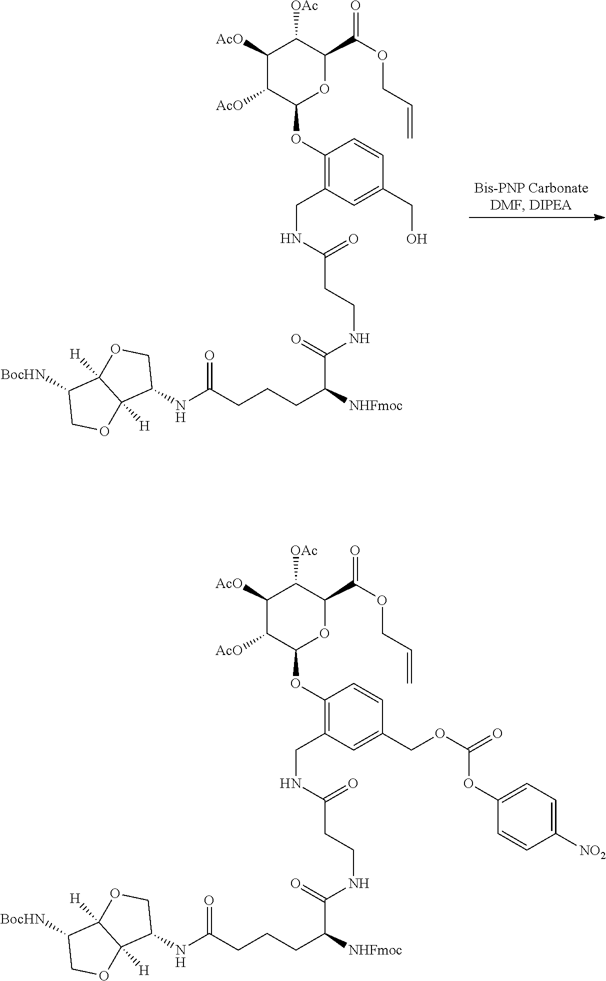

To a solution of Intermediate E (800 mg, 691 mol, 1.0 eq) in DMF (8 mL) was added bis(4-nitrophenyl) carbonate (840 mg, 2.76 mmol, 4.0 eq) and DIPEA (481 μL, 2.76 mmol, 4.0 eq). The mixture was stirred at 20-25° C. for 2 hours. LC-MS analysis showed formation of the desired product and completion of the reaction. Solvent was removed under reduced pressure to get crude product. The crude product was purified via silica gel column (5%-50% MeCN in water, 0.1% formic acid) to give allyl (2S,3S,4S,5R,6S)-6-[2-[[3-[[(2S)-6-[[(3S,3aR,6S,6aR)-6-(tert-butoxycarbonylamino)-2,3,3a,5,6,6a-hexahydrofuro[3,2-b]furan-3-yl]amino]-2-(9H-fluoren-9-ylmethoxycarbonylamino)-6-oxo-hexanoyl]amino]propanoylamino]methyl]-4-[(4-nitrophenoxy)carbonyloxymethyl]phenoxy]-3,4,5-triacetoxy-tetrahydropyran-2-carboxylate Intermediate F (400 mg, 43.8%) 1H NMR (300 MHz, DMSO-d6) δ ppm 1.14-1.29 (m, 3H) 1.38 (s, 9H) 1.53 (br dd, J=18.52, 8.25 Hz, 4H) 1.98-2.09 (m, 12H) 2.13 (d, J=2.93 Hz, 1H) 2.32-2.45 (m, 2H) 3.35 (s, 11H) 3.58 (br d, J=6.42 Hz, 2H) 3.71 (s, 12H) 3.78-3.88 (m, 3H) 3.90-4.17 (m, 4H) 4.18-4.42 (m, 6H) 4.50-4.67 (m, 2H) 4.80 (d, J=10.09 Hz, 1H) 5.09-5.38 (m, 6H) 5.47-5.55 (m, 1H) 5.65 (d, J=7.89 Hz, 1H) 5.82-5.96 (m, 1H) 6.10 (s, 4H) 7.11 (d, J=8.44 Hz, 1H) 7.21 (br d, J=5.50 Hz, 1H) 7.28-7.37 (m, 4H) 7.37-7.44 (m, 3H) 7.46-7.61 (m, 3H) 7.65-7.76 (m, 2H) 7.89 (d, J=7.34 Hz, 2H) 7.99 (br t, J=5.50 Hz, 1H) 8.09 (d, J=6.97 Hz, 1H) 8.27-8.34 (m, 3H) LCMS m/z 1323.1 [M+H]⁺

### Intermediate G

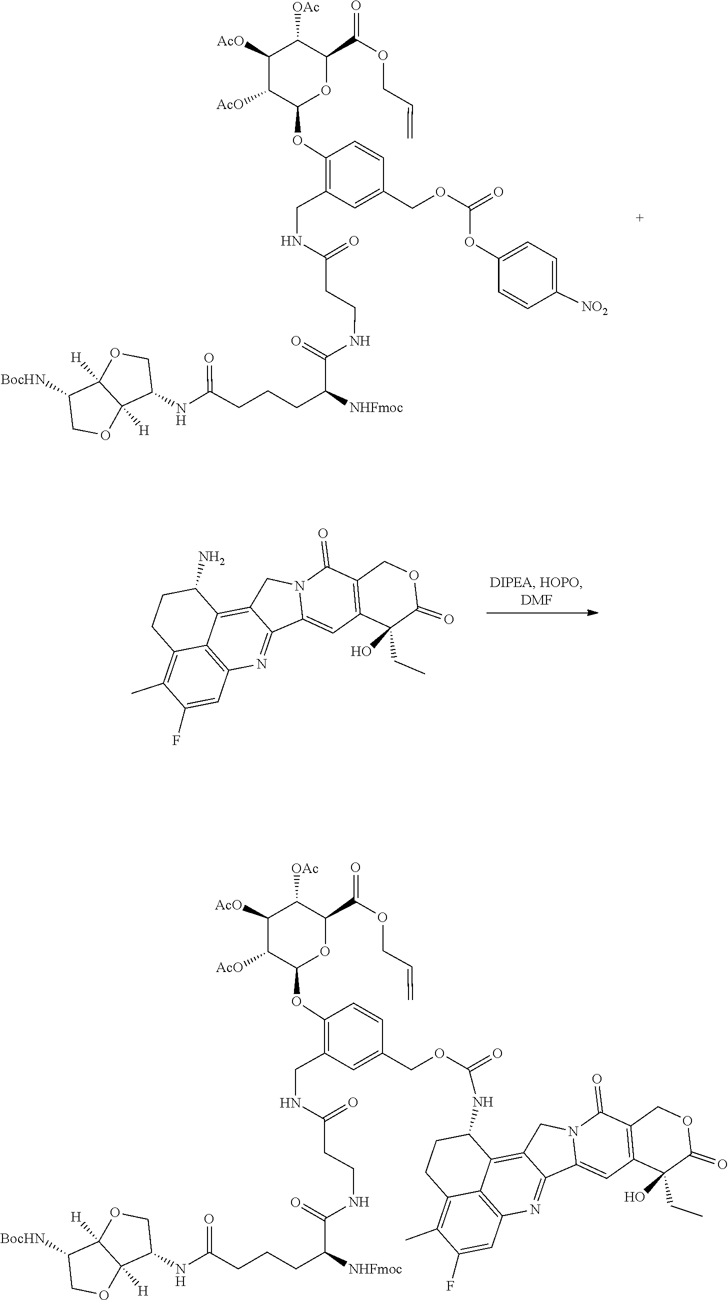

To a solution of exatecan mesylate (1.12 g, 2.12 mmol, 4.0 eq) in DMF (7 mL) was added DIPEA (369 μl, 2.12 mmol, 4.0 eq), Intermediate F (700 mg, 529 μmol, 1.0 eq), and HOPO (117.5 mg, 1.06 mmol, 2.0 eq) and the resultant mixture was stirred at 20-25° C. for 1 hour. LC-MS analysis showed formation of the desired product and completion of the reaction. The reaction mixture was diluted with ethyl acetate (9 mL) and washed with NaCl (15% aq, 12 mL). The solvent was removed under reduced pressure to get crude product. The crude product was slurried with ethyl acetate/MTBE (1:1, 5 mL), filtered and dried to give allyl (2S,3S,4S,5R,6S)-6-[2-[[3-[[(2S)-6-[[(3S,3aR,6S,6aR)-6-(tert-butoxycarbonylamino)-2,3,3a,5,6,6a-hexahydrofuro[3,2-b]furan-3-yl]amino]-2-(9H-fluoren-9-ylmethoxycarbonylamino)-6-oxo-hexanoyl]amino]propanoylamino]methyl]-4-[[(10S,23S)-10-ethyl-18-fluoro-10-hydroxy-19-methyl-5,9-dioxo-8-oxa-4,15-diazahexacyclo[14.7.1.02,14.04,13.06,11.020,24]tetracosa-1,6(11),12,14,16(24),17,19-heptaen-23-yl]carbamoyloxymethyl]phenoxy]-3,4,5-triacetoxy-tetrahydropyran-2-carboxylate Intermediate G (360 mg, 73.5%). 1H NMR (400 MHz, DMSO-d6) δ ppm 0.81-0.94 (m, 2H) 1.11-1.26 (m, 3H) 1.37 (s, 6H) 1.42-1.59 (m, 3H) 1.81-1.92 (m, 1H) 1.95-2.15 (m, 9H) 2.29-2.44 (m, 3H) 2.73 (s, 1H) 2.89 (s, 1H) 3.49-3.61 (m, 2H) 3.71 (s, 16H) 3.81 (br d, J=6.36 Hz, 2H) 3.85-3.95 (m, 1H) 3.99-4.12 (m, 2H) 4.12-4.28 (m, 2H) 4.33 (br d, J=3.91 Hz, 1H) 4.39 (br d, J=4.16 Hz, 1H) 4.48-4.64 (m, 1H) 4.78 (br d, J=10.27 Hz, 1H) 5.06-5.34 (m, 5H) 5.41-5.53 (m, 2H) 5.60 (br d, J=7.58 Hz, 1H) 5.81-5.93 (m, 1H) 6.09 (s, 5H) 6.92 (d, J=8.26 Hz, 2H) 7.09 (br d, J=8.56 Hz, 1H) 7.17-7.42 (m, 5H) 7.64-7.79 (m, 2H) 7.80-7.90 (m, 1H) 7.93-7.99 (m, 1H) 8.00-8.14 (m, 3H) LCMS m/z 810.3 [M+2H]²⁺

### Intermediate H

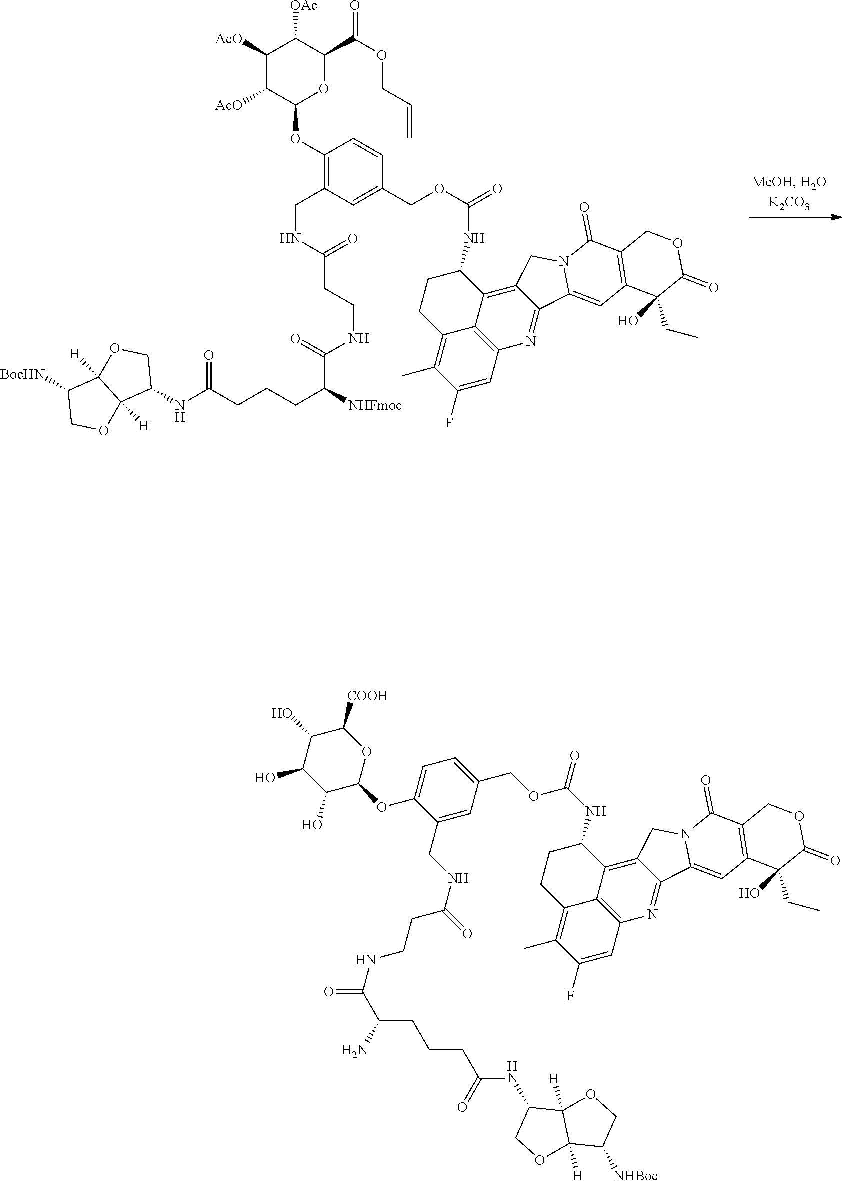

To a solution of Intermediate G (1.5 g 80%, 741 mol, 1.0 eq) in MeOH (2.6 mL) was added K₂CO₃ (1.02 g, 7.41 mmol, 10.0 eq) and water (260 μL). The resultant mixture was stirred at 20-25° C. for 1 hour. LC-MS analysis showed formation of the desired product and completion of the reaction. Solvent was removed under reduced pressure to get crude product. The crude product was purified via silica gel column (5%-50% MeCN in water, 0.1% formic acid) to give (2S,3S,4S,5R,6S)-6-[2-[[3-[[(2S)-6-[[(3S,3aR,6S,6aR)-6-(tert-butoxycarbonylamino)-2,3,3a,5,6,6a-hexahydrofuro[3,2-b]furan-3-yl]amino]-2-amino-6-oxo-hexanoyl]amino]propanoylamino]methyl]-4-[[[(10S,23S)-10-ethyl-18-fluoro-10-hydroxy-19-methyl-5,9-dioxo-8-oxa-4,15-diazahexacyclo[14.7.1.02,14.04,13.06,11.020,24]tetracosa-1,6(11),12,14,16(24),17,19-heptaen-23-yl]carbamoyloxymethyl]phenoxy]-3,4,5-trihydroxy-tetrahydropyran-2-carboxylic acid Intermediate H (450 mg, 67%) 1H NMR (500 MHz, DMSO-d6) δ ppm 0.84-0.92 (m, 3H) 1.24 (br s, 4H) 1.29 (br d, J=7.32 Hz, 1H) 1.38 (s, 8H) 1.43-1.53 (m, 2H) 1.60 (br s, 2H) 1.81-2.11 (m, 5H) 2.14-2.24 (m, 2H) 2.29-2.36 (m, 1H) 2.39 (s, 3H) 2.40-2.44 (m, 1H) 3.13-3.23 (m, 7H) 3.23-3.32 (m, 14H) 3.35 (br s, 12H) 3.50-3.60 (m, 8H) 3.77-3.85 (m, 3H) 4.05 (br s, 1H) 4.17 (br dd, J=13.43, 3.97 Hz, 1H) 4.34 (br d, J=3.36 Hz, 1H) 4.39 (d, J=3.97 Hz, 1H) 4.49 (br dd, J=13.28, 7.78 Hz, 1H) 4.60 (br d, J=6.71 Hz, 1H) 5.03 (br d, J=12.21 Hz, 1H) 5.10 (br d, J=12.21 Hz, 1H) 5.28 (br s, 3H) 5.45 (s, 2H) 6.52 (br s, 1H) 7.12 (d, J=8.24 Hz, 1H) 7.20 (br s, 1H) 7.32 (s, 1H) 7.32-7.34 (m, 1H) 7.40 (s, 1H) 7.78 (d, J=10.68 Hz, 1H) 8.03-8.15 (m, 1H) 8.17 (s, 2H) 8.24 (br d, J=6.10 Hz, 1H) 8.47 (br s, 1H) 9.30 (br s, 1H). LCMS m/z 616.2 [M+2H]²⁺

### Intermediate I

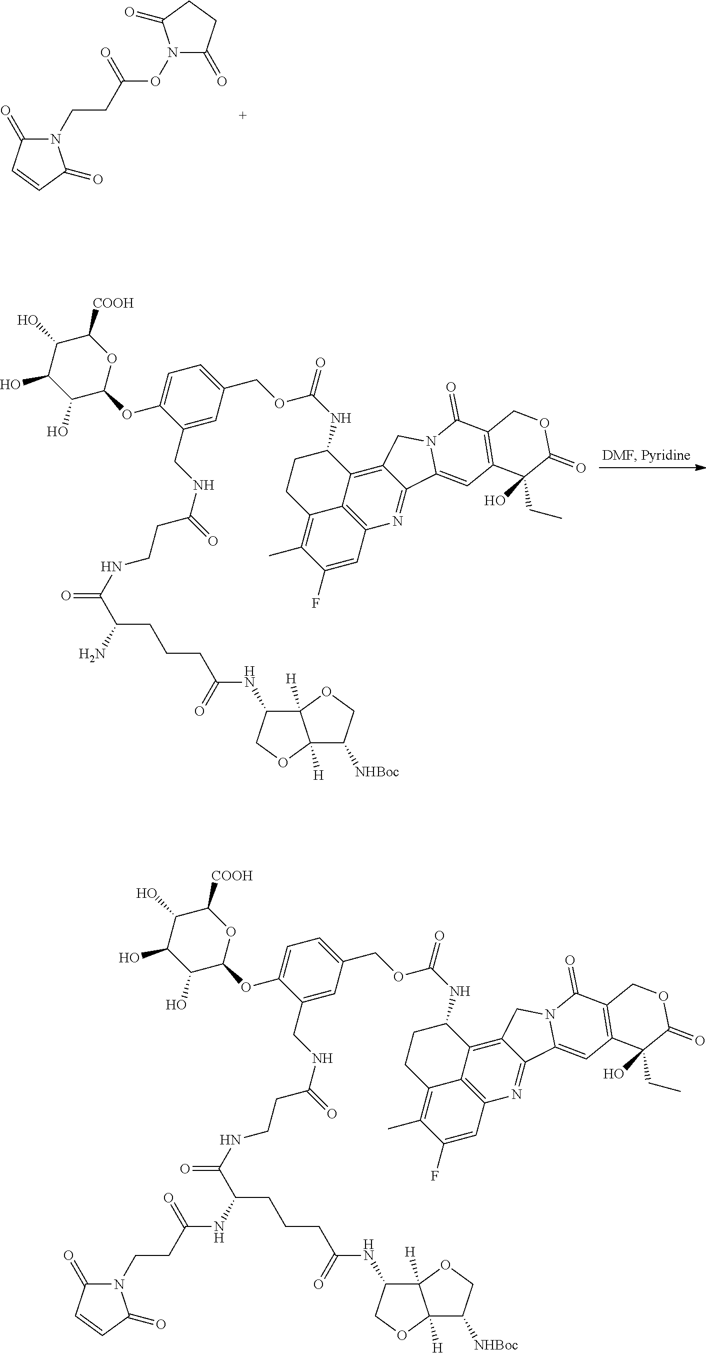 [SMILES: O=C(CCN1C(=O)C=CC1=O)ON1C(=O)CCC1=O ; CC[C@@]1(O)C(=O)OCc2c1cc1n(c2=O)Cc2c-1nc1cc(F)c(C)c3c1c2[C@@H](NC(=O)OCc1ccc(O[C@@H]2O[C@H](C(=O)O)[C@@H](O)[C@H](O)[C@H]2O)c(CNC(=O)CCNC(=O)[C@@H](N)CCCC(=O)N[C@H]2CO[C@H]4[C@@H]2OC[C@@H]4NC(=O)OC(C)(C)C)c1)CC3 ; CC[C@@]1(O)C(=O)OCc2c1cc1n(c2=O)Cc2c-1nc1cc(F)c(C)c3c1c2[C@@H](NC(=O)OCc1ccc(O[C@@H]2O[C@H](C(=O)O)[C@@H](O)[C@H](O)[C@H]2O)c(CNC(=O)CCNC(=O)[C@H](CCCC(=O)N[C@H]2CO[C@H]4[C@@H]2OC[C@@H]4NC(=O)OC(C)(C)C)NC(=O)CCN2C(=O)C=CC2=O)c1)CC3]

To a solution of Intermediate H (450 mg, 365 mol, 1.0 eq) in DMF (4.5 mL) at 20-25° C. was added pyridine (225 μL) and N-succinimidyl 3-maleimidiopropionate (195 mg, 731 mol, 2.0 eq) and the reaction mixture was stirred for 1 hour. LC-MS analysis showed formation of the desired product and completion of the reaction. Solvent was removed under reduced pressure to get crude product. The crude product was purified via silica gel column (5%-50% MeCN in water, 0.1% formic acid) to give (2S,3S,4S,5R,6S)-6-(2-((3-((S)-6-(((3S,3aR,6S,6aR)-6-((tert-butoxycarbonyl)amino)hexahydrofuro[3,2-b]furan-3-yl)amino)-2-(3-(2,5-dioxo-2,5-dihydro-1H-pyrrol-1-yl)propanamido)-6-oxohexanamido)propanamido)methyl)-4-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)phenoxy)-3,4,5-trihydroxytetrahydro-2H-pyran-2-carboxylic acid Intermediate I (230 mg, 52.4%) 1H NMR (500 MHz, DMSO-d6) δ ppm 0.84-0.91 (m, 2H) 1.23 (br d, J=9.77 Hz, 5H) 1.38 (s, 6H) 1.81-1.94 (m, 1H) 1.96-2.07 (m, 2H) 2.13-2.24 (m, 1H) 2.25-2.36 (m, 1H) 2.50-2.52 (m, 7H) 3.05-3.18 (m, 1H) 3.24 (br d, J=6.10 Hz, 2H) 3.29 (br s, 3H) 3.34 (br s, 24H) 3.51-3.63 (m, 4H) 3.71 (s, 9H) 3.75-3.88 (m, 3H) 4.01-4.15 (m, 2H) 4.21 (br d, J=9.46 Hz, 1H) 4.34 (d, J=3.66 Hz, 1H) 4.36-4.46 (m, 1H) 4.72 (br s, 1H) 5.06-5.15 (m, 1H) 5.27 (br s, 2H) 5.45 (s, 2H) 6.09 (s, 3H) 6.51 (s, 1H) 6.97 (s, 1H) 7.10 (d, J=8.24 Hz, 1H) 7.19 (br d, J=5.80 Hz, 1H) 7.31 (s, 1H) 7.33 (s, 1H) 7.77 (d, J=10.99 Hz, 1H) 7.94 (br t, J=5.34 Hz, 1H) 8.06 (br d, J=8.55 Hz, 1H) 8.12 (br d, J=6.71 Hz, 1H) 8.20 (br d, J=8.24 Hz, 1H) LCMS m/z 1382.1 [M+H]⁺

### Intermediate J

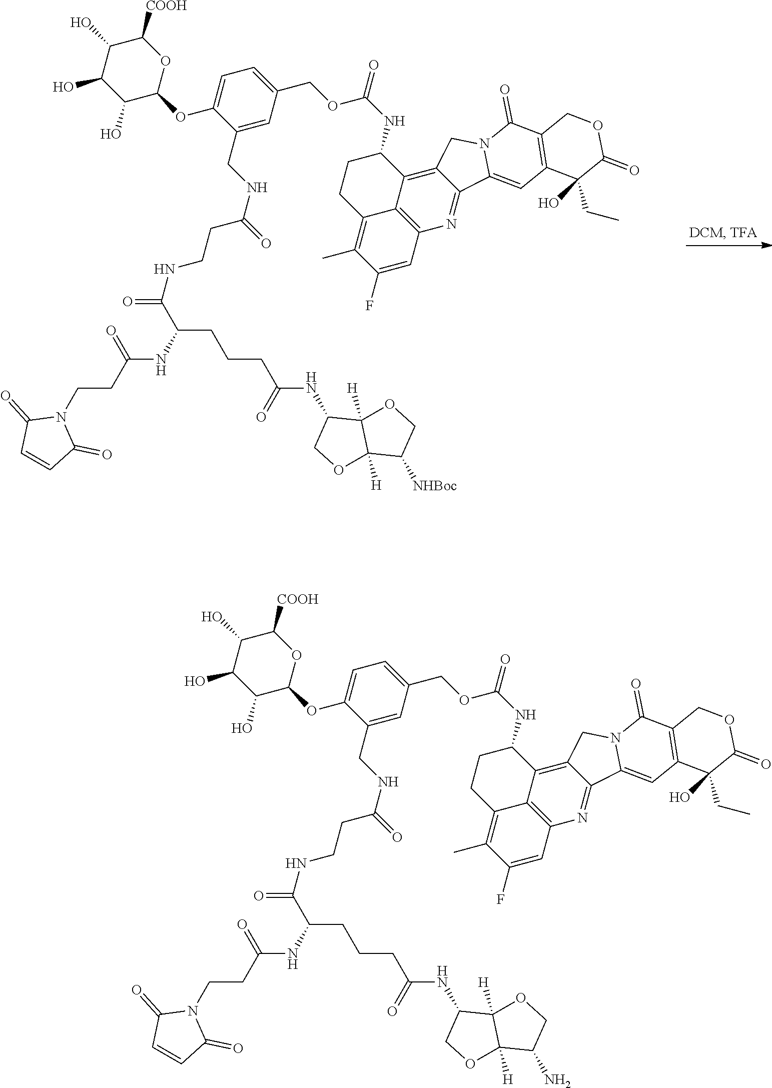 [SMILES: CC[C@@]1(O)C(=O)OCc2c1cc1n(c2=O)Cc2c-1nc1cc(F)c(C)c3c1c2[C@@H](NC(=O)OCc1ccc(O[C@@H]2O[C@H](C(=O)O)[C@@H](O)[C@H](O)[C@H]2O)c(CNC(=O)CCNC(=O)[C@H](CCCC(=O)N[C@H]2CO[C@H]4[C@@H]2OC[C@@H]4NC(=O)OC(C)(C)C)NC(=O)CCN2C(=O)C=CC2=O)c1)CC3 ; CC[C@@]1(O)C(=O)OCc2c1cc1n(c2=O)Cc2c-1nc1cc(F)c(C)c3c1c2[C@@H](NC(=O)OCc1ccc(O[C@@H]2O[C@H](C(=O)O)[C@@H](O)[C@H](O)[C@H]2O)c(CNC(=O)CCNC(=O)[C@H](CCCC(=O)N[C@H]2CO[C@H]4[C@@H]2OC[C@@H]4N)NC(=O)CCN2C(=O)C=CC2=O)c1)CC3]

To a solution of Intermediate I (230 mg, 166 μmol, 1.0 eq) in DCM (2.6 mL) was added TFA (1.3 mL), and the reaction mixture was stirred for 1 hour at 25° C. LC-MS analysis showed formation of the desired product and completion of the reaction. Solvent was removed under reduced pressure to get crude product. The crude product was purified via silica gel column (5%-50% MeCN in water, 0.1% formic acid) to give (2S,3S,4S,5R,6S)-6-[2-[[3-[[(2S)-6-[[(3S,3aR,6S,6aR)-3-amino-2,3,3a,5,6,6a-hexahydrofuro[3,2-b]furan-6-yl]amino]-2-[3-(2,5-dioxopyrrol-1-yl)propanoylamino]-6-oxo-hexanoyl]amino]propanoylamino]methyl]-4-[[(10S,23S)-10-ethyl-18-fluoro-10-hydroxy-19-methyl-5,9-dioxo-8-oxa-4,15-diazahexacyclo[14.7.1.02,14.04,13.06,11.020,24]tetracosa-1,6(11),12,14,16(24),17,19-heptaen-23-yl]carbamoyloxymethyl]phenoxy]-3,4,5-trihydroxy-tetrahydropyran-2-carboxylic acid Intermediate J (120 mg, 54.5%). LCMS m/z 641.5 [M+H]⁺

Linker-Payload β-Glu-exatecan (LP-1)

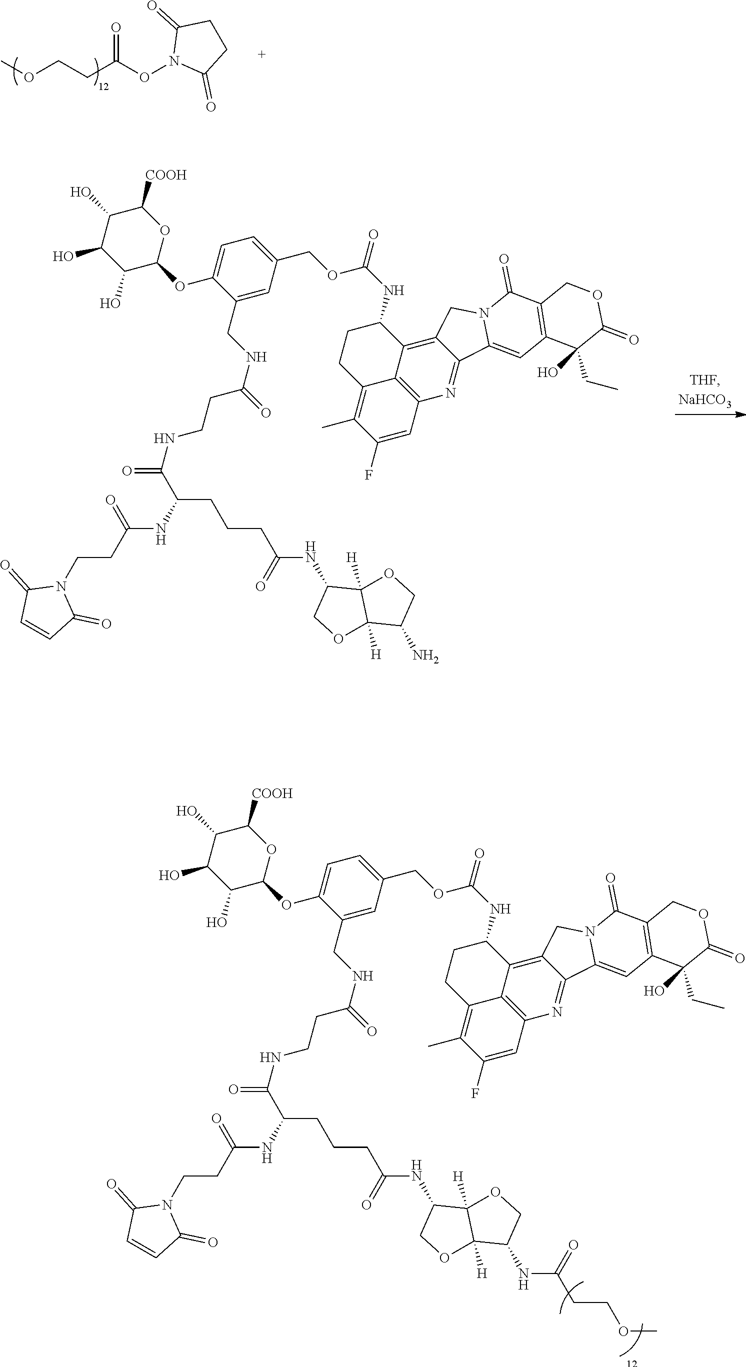

To a solution of Intermediate J (100 mg, 78.0 mol, 1.0 eq) in THE (1 mL) was added NaHCO₃ (13.1 mg, 156 mol, 2.0 eq) and 2,5-dioxopyrrolidin-1-yl 2,5,8,11,14,17,20,23,26,29,32,35-dodecaoxaoctatriacontan-38-oate Intermediate 10 (64.2 mg, 93.6 mol, 1.2 eq). The reaction mixture was stirred at 20-25° C. for 16 h. LC-MS analysis showed formation of the desired product and completion of the reaction. Solvent was removed under reduced pressure to get crude product. Solvent was removed under reduced pressure to get crude product. The crude product was purified via silica gel column (5%-50% MeCN in water, 0.1% formic acid) to give give (2S,3S,4S,5R,6S)-6-(2-((3-((S)-6-(((3S,3aR,6S,6aR)-6-(2,5,8,11,14,17,20,23,26,29,32,35-dodecaoxaoctatriacontan-38-amido)hexahydrofuro [3,2-b]furan-3-yl)amino)-2-(3-(2,5-dioxo-2,5-dihydro-1H-pyrrol-1-yl)propanamido)-6-oxohexanamido)propanamido)methyl)-4-(((((1S,9S)-9-ethyl-5-fluoro-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)carbamoyl)oxy)methyl)phenoxy)-3,4,5-trihydroxytetrahydro-2H-pyran-2-carboxylic acid LP-1 (60 mg, 40%). LCMS m/z 927.2 [M+2H]²⁺

### Example 14

### Antibody-Drug Conjugation

mAb23-TM-β-Glu-Exatecan (DAR 8)

A 50 mM solution of Tris(2-carboxyethyl)phosphine hydrochloride (TCEP) in phosphate-buffered saline pH 7.4 (PBS) was added (10 molar equivalent/antibody, 3.3 micromoles, 6.7 μL at 50 mM) to a 9.1 mL solution of mAb23-TM antibody (50 mg, 0.33 micromoles) in reduction buffer containing PBS and 1 mM ethylenediaminetetraacetic acid (EDTA) and a final antibody concentration of 5.5 mg/mL. The resultant mixture was incubated at +37° C. for 2h (or until full reduction observed by UHPLC) in an orbital shaker with gentle (60 rpm) shaking. LP-1 was added as a DMSO solution (14 molar equivalent/antibody, 4.6 μmole, in 0.78 mL DMSO) to reduced mAb23-TM-antibody in PBS, 1 mM EDTA, pH 7.4 to a 10% (v/v) final DMSO concentration. The solution left to react at room temperature for 1 hours with gentle shaking. Then the conjugation was quenched by addition of N-acetyl cysteine (23.0 micromoles, 0.23 mL at 100 mM) and purified by SEC-FPLC using PBS pH 7.4 as elution buffer. Fractions with monomer >95% pooled and concentrated through spin filtration using a 15 mL AMICON ULTRACELL 30 kDa MWCO spin filter followed by sterile-filtration.

UHPLC analysis on a SHIMADZU PROMINENCE system using a THERMO SCIENTIFIC MAbPac 50 mm×2.1 mm column eluting with a gradient of water and acetonitrile on a reduced sample of ADC at 214 nm and 330 nm shows a mixture of light chain conjugated to 1 molecule of LP-1, and heavy chain conjugated to 3.0 molecules of LP-1, consistent with a drug-per-antibody ratio (DAR) of 8.0 molecules of LP-1 per antibody.

UHPLC analysis on a SHIMADZU PROMINENCE system using A TOSOH BIOSCIENCE TSKgel SuperSW mAb HTP 4 μm 4.6×150 mm column (with a 4 μm 3.0×20 mm guard column) eluting with 0.3 mL/minute sterile-filtered SEC buffer containing 200 mM potassium phosphate pH 6.95, 250 mM potassium chloride and 10% isopropanol (v/v) on a sample of ADC at 280 nm shows a monomer purity of 99.0%. UHPLC SEC analysis gives a concentration of final ADC at 4.9 mg/mL in 7.5 mL, obtained mass is 36.8 mg (73% yield).

LC-MS analysis on a EXACTIVE PLUS EMR mass spectrometer connected to DIONEX 3000 HPLC equipment using a THERMO SCIENTIFIC MAbPac 50 mm×2.1 mm column eluting with a gradient of water and acetonitrile on a de-glycosylated and reduced sample of ADC at 214 nm shows a mixture of light chain conjugated to 1 molecule LP-1, and heavy chain conjugated to 3.0 molecules of LP-1, consistent with a drug-per-antibody ratio (DAR) of 8.0 molecules of LP-1 per antibody.

UHPLC analysis on a SHIMADZU PROMINENCE system using A PROTEOMIX HIC Butyl-NP5, 5 um, non-porous, 4.6×35 mm (SEPAX) column eluting with a gradient of 1.5M ammonium sulphate, 25 mM sodium acetate, pH 7.4 and 25 mM sodium acetate, pH 7.4 with 20% acetonitrile (v/v) on a neat sample of ADC at 214 nm shows singly conjugated LP-1, consistent with a drug-per-antibody ratio (DAR) of 8 molecules of LP-1 per antibody.

mAb23-TM-Val-Ala-Gly-TOP1i ('0132) (DAR 8)

A 50 mM solution of Tris(2-carboxyethyl)phosphine hydrochloride (TCEP) in phosphate-buffered saline pH 7.4 (PBS) was added (10 molar equivalent/antibody, 4.7 micromoles, 9.3 μL at 50 mM) to a 12.7 mL solution of mAb23-TM antibody (70 mg, 0.47 micromoles) in reduction buffer containing PBS and 1 mM ethylenediaminetetraacetic acid (EDTA) and a final antibody concentration of 5.5 mg/mL. The resultant mixture was incubated at +37° C. for 2h (or until full reduction observed by UHPLC) in an orbital shaker with gentle (60 rpm) shaking. LP-2

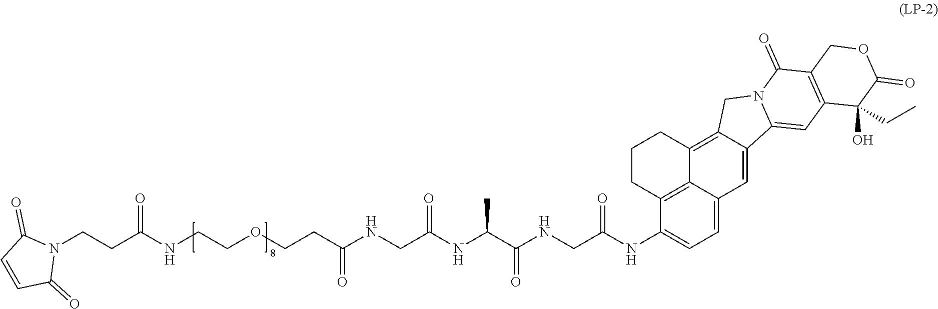

was added as a DMSO solution (15 molar equivalent/antibody, 7.05 μmole, in 1.1 mL DMSO) to reduced mAb23-TM-antibody in PBS, 1 mM EDTA, pH 7.4 to a 10% (v/v) final DMSO concentration. The solution left to react at room temperature for 1 hours with gentle shaking. Then the conjugation was quenched by addition of N-acetyl cysteine (35.0 micromoles, 0.35 mL at 100 mM) and purified by SEC-FPLC using PBS pH 7.4 as elution buffer. Fractions with monomer >95% pooled and concentrated through spin filtration using a 15 mL AMICON ULTRACELL 30 kDa MWCO spin filter followed by sterile-filtration.

UHPLC analysis on a SHIMADZU PROMINENCE system using a THERMO SCIENTIFIC MAbPac 50 mm×2.1 mm column eluting with a gradient of water and acetonitrile on a reduced sample of ADC at 214 nm and 330 nm shows a mixture of light chain conjugated to 1 molecule of LP-2, and heavy chain conjugated to 3.0 molecules of LP-2, consistent with a drug-per-antibody ratio (DAR) of 8.0 molecules of LP-2 per antibody.

UHPLC analysis on a SHIMADZU PROMINENCE system using A TOSOH BIOSCIENCE TSKgel SuperSW mAb HTP 4 μm 4.6×150 mm column (with a 4 μm 3.0×20 mm guard column) eluting with 0.3 mL/minute sterile-filtered SEC buffer containing 200 mM potassium phosphate pH 6.95, 250 mM potassium chloride and 10% isopropanol (v/v) on a sample of ADC at 280 nm shows a monomer purity of 99.7%. UHPLC SEC analysis gives a concentration of final ADC at 5.5 mg/mL in 12.7 mL, obtained mass of 61.0 mg (86% yield).

LC-MS analysis on a EXACTIVE PLUS EMR mass spectrometer connected to DIONEX 3000 HPLC equipment using a THERMO SCIENTIFIC MAbPac 50 mm×2.1 mm column eluting with a gradient of water and acetonitrile on a de-glycosylated and reduced sample of ADC at 214 nm shows a mixture of light chain conjugated to 1 molecule LP-1, and heavy chain conjugated to 3.0 molecules of LP-2, consistent with a drug-per-antibody ratio (DAR) of 8.0 molecules of LP-2 per antibody.

UHPLC analysis on a SHIMADZU PROMINENCE system using A PROTEOMIX HIC Butyl-NP5, 5 um, non-porous, 4.6×35 mm (SEPAX) column eluting with a gradient of 1.5M ammonium sulphate, 25 mM sodium acetate, pH 7.4 and 25 mM sodium acetate, pH 7.4 with 20% acetonitrile (v/v) on a neat sample of ADC at 214 nm shows singly conjugated LP-1, consistent with a drug-per-antibody ratio (DAR) of 8 molecules of LP-2 per antibody.

### Antibody-Drug Conjugation

ADC-1: mAb23-TM-Val-Ala-TOP1i ('0132) (DAR 8)

A 50 mM solution of Tris(2-carboxyethyl)phosphine hydrochloride (TCEP) in water was added (7.5 molar equivalent/antibody, 50.88 millimoles, 1.02 mL at 50 mM) to a 50 mL solution of mAb23-TM antibody (1 g, 6.78 millimoles) in reduction buffer of 20 mM Sodium Phosphate, 1 mM Ethylenediaminetetraacetic acid (EDTA), pH 7 and a final antibody concentration of 20 mg/mL. The resultant mixture was incubated at room temperature (+20° C. to +28° C.) for 3 hours in an orbital shaker with gentle (60 rpm) shaking. Val-Ala was added as a DMSO solution (10.5 molar equivalent/antibody, 70 millimoles, in 2.94 mL DMSO) to reduced mAb23-TM-antibody in the reduction buffer. DMSO is then added to the solution (2.61 mL) to a 10% (v/v) final DMSO concentration. The solution left to react at room temperature for 1 hour with gentle shaking. Then the conjugation was quenched by addition of N-acetyl cysteine in water (210 millimoles, 2.1 mL at 100 mM). After conjugation, the crude conjugate undergoes ultrafiltration/diafiltration in a diafiltration buffer of 20 mM histidine/histidine-HCl, 150 mM sodium chloride, pH 6.0 for 10 diavolumes on a Millipore Ultracel C membrane with 30 kDa molecular weight cut off. Then another diafiltration occurs on the same membrane with a new diafiltration buffer of 20 mM histidine/histidine-HCl, pH 6.0.

HPLC analysis on the Agilent 1260 system using a Waters BioResolve RP Mab polyphenyl, 2.1*150 mm, 2.7 μm, 450 Å, Part #176004158 column eluting with a gradient of water and acetonitrile on a reduced sample of ADC at 280 nm. Ref 450 nm shows a mixture of light chain conjugated to 1 molecule of LP-1, and heavy chain conjugated to 3.0 molecules of LP-1, consistent with an average drug-per-antibody ratio (DAR) of 8.0 molecules of LP-1 per antibody (DAR8).

HPLC analysis on the Agilent 1260 system using A Waters UPLC Protein BEH SEC Column, 200 Å, 1.7 m (4.6 mm×150 mm), eluting with 0.5 mL/minute sterile-filtered SEC buffer containing 50 mM Sodium Phosphate, 450 mM Arginine with 0.05% Sodium Azide, pH 7.0. The sample of ADC are monitored at 280 nm shows a monomer purity of ˜96-99%.

LC-MS analysis on a QTOF mass spectrometer connected to Waters Acquity UPLC HPLC equipment using a Waters BioResolve RP mAb polyphenyl, 2.1×150 mm, 2.7 μm, 450 Å column eluting with a gradient of water and acetonitrile on a de-glycosylated and reduced sample of ADC at 280 nm shows a mixture of light chain conjugated to 1 molecule LP-1, and heavy chain conjugated to 3.0 molecules of LP-1, consistent with a drug-per-antibody ratio (DAR) of 8.0 molecules of LP-1 per antibody (DAR8).

HPLC analysis on the Agilent 1260 system using Thermo MabPac HIC-10, 5 μm, 4.6*250 mm column eluting with a gradient of 1.5M ammonium sulfate, 20 mM sodium acetate, pH 5 and 20 mM sodium acetate 5% IPA, pH 5 on a neat sample of ADC at 214 nm shows singly conjugated LP-1, consistent with a drug-per-antibody ratio (DAR) of 8 molecules of LP-1 per antibody (DAR8).

SEQUENCES

| SEQ ID NO: | Identifier | Sequence |
|---|---|---|
| SEQ ID NO: 1 | mAb23 VH CDR 1 | SYAMT |
| SEQ ID NO: 2 | mAb23 VH CDR 2 | TISGSGVSTYSADSVKG |
| SEQ ID NO: 3 | mAb23 VH CDR 3 | HWELLDAFDI |
| SEQ ID NO: 4 | mAb23 VL CDR 1 | RASQSISYNYLA |
| SEQ ID NO: 5 | mAb23 VL CDR 2 | GASSRAS |
| SEQ ID NO: 6 | mAb23 VL CDR 3 | QQYGSSPLT |
| SEQ ID NO: 7 | mAb23 VH | EVQLLESGGGLVQPGGSLRLSCAASGFTFSSYAMTWVRQAPGKGLE |
| | | WVSTISGSGVSTYSADSVKGRFTISRDNSKNTLYLQMNSLRAEDTA |
| | | VYYCALHWELLDAFDIWGQGTMVTVSS |
| SEQ ID NO: 8 | mAb23 VL | EIVLTQSPGTLSLSPGERATLSCRASQSISYNYLAWYQQKPGQAPR |
| | | LLIYGASSRASGISDRFSGSGSGTDFTLTISRLEPEDFVVYYCQQY |
| | | GSSPLTFGGGTKVEVK |
| SEQ ID NO: 9 | mAb23 HC (TM) | EVQLLESGGGLVQPGGSLRLSCAASGFTFSSYAMTWVRQAPGKGLE |
| | | WVSTISGSGVSTYSADSVKGRFTISRDNSKNTLYLQMNSLRAEDTA |
| | | VYYCALHWELLDAFDIWGQGTMVTVSSASTKGPSVFPLAPSSKSTS |
| | | GGTAALGCLVKDYFPEPVTVSWNSGALTSGVHTFPAVLQSSGLYSL |
| | | SSVVTVPSSSLGTQTYICNVNHKPSNTKVDKRVEPKSCDKTHTCPP |
| | | CPAPEFEGGPSVFLFPPKPKDTLMISRTPEVTCVVVDVSHEDPEVK |
| | | FNWYVDGVEVHNAKTKPREEQYNSTYRVVSVLTVLHQDWLNGKEYK |
| | | CKVSNKALPASIEKTISKAKGQPREPQVYTLPPSREEMTKNQVSLT |
| | | CLVKGFYPSDIAVEWESNGQPENNYKTTPPVLDSDGSFFLYSKLTV |
| | | DKSRWQQGNVFSCSVMHEALHNHYTQKSLSLSPGK |
| SEQ ID NO: | mAb23 LC | EIVLTQSPGTLSLSPGERATLSCRASQSISYNYLAWYQQKPGQAPR |
| 10 | | LLIYGASSRASGISDRESGSGSGTDETLTISRLEPEDFVVYYCQQY |
| | | GSSPLTFGGGTKVEVKRTVAAPSVFIFPPSDEQLKSGTASVVCLLN |
| | | NFYPREAKVQWKVDNALQSGNSQESVTEQDSKDSTYSLSSTLTLSK |
| | | ADYEKHKVYACEVTHQGLSSPVTKSENRGEC |
| SEQ ID NO: | mAb23 HC-TM | ASTKGPSVFPLAPSSKSTSGGTAALGCLVKDYFPEPVTVSWNSGAL |
| 11 | constant region | TSGVHTFPAVLQSSGLYSLSSVVTVPSSSLGTQTYICNVNHKPSNT |
| | (CH1-hinge-CH2- | KVDKRVEPKSCDKTHTCPPCPAPEFEGGPSVFLFPPKPKDTLMISR |
| | CH3) | TPEVTCVVVDVSHEDPEVKENWYVDGVEVHNAKTKPREEQYNSTYR |
| | | VVSVLTVLHQDWLNGKEYKCKVSNKALPASIEKTISKAKGQPREPQ |
| | | VYTLPPSREEMTKNQVSLTCLVKGFYPSDIAVEWESNGQPENNYKT |
| | | TPPVLDSDGSFFLYSKLTVDKSRWQQGNVFSCSVMHEALHNHYTQK |
| | | SLSLSPGK |
| SEQ ID NO: | mAb23 LC | RTVAAPSVFIFPPSDEQLKSGTASVVCLLNNFYPREAKVQWKVDNA |
| 12 | constant region | LQSGNSQESVTEQDSKDSTYSLSSTLTLSKADYEKHKVYACEVTHQ |
| | (CH1) | GLSSPVTKSENRGEC |
| SEQ ID NO: | mAb23 HC (without | EVQLLESGGGLVQPGGSLRLSCAASGFTESSYAMTWVRQAPGKGLE |
| 13 | TM) | WVSTISGSGVSTYSADSVKGRFTISRDNSKNTLYLQMNSLRAEDTA |
| | | VYYCALHWELLDAFDIWGQGTMVTVSSASTKGPSVFPLAPSSKSTS |
| | | GGTAALGCLVKDYFPEPVTVSWNSGALTSGVHTFPAVLQSSGLYSL |
| | | SSVVTVPSSSLGTQTYICNVNHKPSNTKVDKRVEPKSCDKTHTCPP |
| | | CPAPELLGGPSVFLFPPKPKDTLMISRTPEVTCVVVDVSHEDPEVK |
| | | FNWYVDGVEVHNAKTKPREEQYNSTYRVVSVLTVLHQDWLNGKEYK |
| | | CKVSNKALPAPIEKTISKAKGQPREPQVYTLPPSREEMTKNQVSLT |
| | | CLVKGFYPSDIAVEWESNGQPENNYKTTPPVLDSDGSFFLYSKLTV |
| | | DKSRWQQGNVFSCSVMHEALHNHYTQKSLSLSPGK |
| SEQ ID NO: | mAb23 HC (without | ASTKGPSVFPLAPSSKSTSGGTAALGCLVKDYFPEPVTVSWNSGAL |
| 14 | TM) | TSGVHTFPAVLQSSGLYSLSSVVTVPSSSLGTQTYICNVNHKPSNT |
| | constant region | KVDKRVEPKSCDKTHTCPPCPAPELLGGPSVFLFPPKPKDTLMISR |
| | (CH1-hinge-CH2- | TPEVTCVVVDVSHEDPEVKENWYVDGVEVHNAKTKPREEQYNSTYR |
| | CH3) | VVSVLTVLHQDWLNGKEYKCKVSNKALPAPIEKTISKAKGQPREPQ |
| | | VYTLPPSREEMTKNQVSLTCLVKGFYPSDIAVEWESNGQPENNYKT |
| | | TPPVLDSDGSFFLYSKLTVDKSRWQQGNVFSCSVMHEALHNHYTQK |
| | | SLSLSPGK |
| SEQ ID NO: | mAb23 LC | GAAATCGTATTGACACAATCTCCTGGGACCCTCTCTCTCTCTCCCG |
| 15 | Nucleotide | GAGAGAGAGCAACCCTCAGTTGTCGCGCGTCTCAGAGCATTTCATA |
| | Sequence | CAACTACTTGGCGTGGTATCAGCAGAAACCTGGGCAGGCTCCCAGG |
| | | CTTCTGATTTACGGGGCGTCAAGTAGGGCCTCCGGCATTAGCGATA |
| | | GGTTTTCAGGTAGCGGCAGTGGTACAGATTTCACCTTGACGATAAG |
| | | TCGCCTCGAACCAGAGGATTTCGTGGTCTATTACTGCCAACAGTAT |
| | | GGGAGCTCCCCGCTTACATTTGGCGGCGGTACAAAGGTCGAGGTAA |
| | | AACGTACGGTGGCTGCACCATCTGTCTTCATCTTCCCGCCATCTGA |
| | | TGAGCAGTTGAAATCTGGAACTGCCTCTGTTGTGTGCCTGCTGAAT |
| | | AACTTCTATCCCAGAGAGGCCAAAGTACAGTGGAAGGTGGATAACG |
| | | CCCTCCAATCGGGTAACTCCCAGGAGAGTGTCACAGAGCAGGACAG |
| | | CAAGGACAGCACCTACAGCCTCAGCAGCACCCTGACGCTGAGCAAA |
| | | GCAGACTACGAGAAACACAAAGTCTACGCCTGCGAAGTCACCCATC |
| | | AGGGCCTGAGCTCGCCCGTCACAAAGAGCTTCAACAGGGGAGAGTG |
| | | TTAGT |
| SEQ ID NO: | mAb23 HC (TM) | GAGGTCCAGTTGCTTGAAAGCGGCGGGGGTCTTGTCCAGCCCGGTG |
| 16 | Nucleotide | GGAGTCTGCGACTCTCTTGTGCTGCGAGTGGTTTCACCTTTAGCTC |
| | Sequence | CTACGCTATGACTTGGGTTAGACAAGCCCCGGGCAAGGGACTTGAA |
| | | TGGGTATCCACTATTTCAGGCTCTGGCGTAAGCACTTATAGTGCGG |
| | | ATTCCGTTAAAGGGCGATTTACAATCAGTCGAGACAATTCAAAGAA |
| | | CACGCTTTACTTGCAGATGAACTCATTGCGAGCGGAGGATACTGCC |
| | | GTCTATTACTGTGCGTTGCATTGGGAACTTCTCGACGCGTTTGACA |
| | | TTTGGGGTCAGGGCACTATGGTCACTGTGAGTTCAGCGTCGACCAA |
| | | GGGCCCATCCGTCTTCCCCCTGGCACCCTCCTCCAAGAGCACCTCT |
| | | GGGGGCACAGCGGCCCTGGGCTGCCTGGTCAAGGACTACTTCCCCG |
| | | AACCGGTGACGGTGTCCTGGAACTCAGGCGCTCTGACCAGCGGCGT |
| | | GCACACCTTCCCGGCTGTCCTACAGTCCTCAGGACTCTACTCCCTC |
| | | AGCAGCGTGGTGACCGTGCCCTCCAGCAGCTTGGGCACCCAGACCT |
| | | ACATCTGCAACGTGAATCACAAGCCCAGCAACACCAAGGTGGACAA |
| | | GAGAGTTGAGCCCAAATCTTGTGACAAAACTCACACATGCCCACCG |
| | | TGCCCAGCACCTGAATTCGAGGGGGGACCGTCAGTCTTCCTCTTCC |
| | | CCCCAAAACCCAAGGACACCCTCATGATCTCCCGGACCCCTGAGGT |
| | | CACATGCGTGGTGGTGGACGTGAGCCACGAAGACCCTGAGGTCAAG |
| | | TTCAACTGGTACGTGGACGGCGTGGAGGTGCATAATGCCAAGACAA |
| | | AGCCGCGGGAGGAGCAGTACAACAGCACGTACCGTGTGGTCAGCGT |
| | | CCTCACCGTCCTGCACCAGGACTGGCTGAATGGCAAGGAGTACAAG |
| | | TGCAAGGTCTCCAACAAAGCCCTCCCAGCCTCCATCGAGAAAACCA |
| | | TCTCCAAAGCCAAAGGGCAGCCCCGAGAACCACAGGTCTACACCCT |
| | | GCCCCCATCCCGGGAGGAGATGACCAAGAACCAGGTCAGCCTGACC |
| | | TGCCTGGTCAAAGGCTTCTATCCCAGCGACATCGCCGTGGAGTGGG |
| | | AGAGCAATGGGCAGCCGGAGAACAACTACAAGACCACGCCTCCCGT |
| | | GCTGGACTCCGACGGCTCCTTCTTCCTCTATAGCAAGCTCACCGTG |
| | | GACAAGAGCAGGTGGCAGCAGGGGAACGTCTTCTCATGCTCCGTGA |
| | | TGCATGAGGCTCTGCACAACCACTACACGCAGAAGAGCCTCTCCCT |
| | | GTCTCCGGGTAAATGA |
| SEQ ID NO: | Human CD22 amino | MHLLGPWLLLLVLEYLAFSDSSKWVFEHPETLYAWEGACVWIPCTYRALD |
| 17 | acid sequence | GDLESFILFHNPEYNKNTSKFDGTRLYESTKDGKVPSEQKRVQFLGDKNK |
| | | NCTLSIHPVHLNDSGQLGLRMESKTEKWMERIHLNVSERPFPPHIQLPPE |
| | | IQESQEVTLTCLLNFSCYGYPIQLQWLLEGVPMRQAAVTSTSLTIKSVFT |
| | | RSELKFSPQWSHHGKIVTCQLQDADGKFLSNDTVQLNVKHTPKLEIKVTP |
| | | SDAIVREGDSVTMTCEVSSSNPEYTTVSWLKDGTSLKKQNTFTLNLREVT |
| | | KDQSGKYCCQVSNDVGPGRSEEVFLQVQYAPEPSTVQILHSPAVEGSQVE |
| | | FLCMSLANPLPTNYTWYHNGKEMQGRTEEKVHIPKILPWHAGTYSCVAEN |
| | | ILGTGQRGPGAELDVQYPPKKVTTVIQNPMPIREGDTVTLSCNYNSSNPS |
| | | VTRYEWKPHGAWEEPSLGVLKIQNVGWDNTTIACAACNSWCSWASPVALN |
| | | VQYAPRDVRVRKIKPLSEIHSGNSVSLQCDESSSHPKEVQFFWEKNGRLL |
| | | GKESQLNFDSISPEDAGSYSCWVNNSIGQTASKAWTLEVLYAPRRLRVSM |
| | | SPGDQVMEGKSATLTCESDANPPVSHYTWFDWNNQSLPYHSQKLRLEPVK |
| | | VQHSGAYWCQGTNSVGKGRSPLSTLTVYYSPETIGRRVAVGLGSCLAILI |
| | | LAICGLKLQRRWKRTQSQQGLQENSSGQSFFVRNKKVRRAPLSEGPHSLG |
| | | CYNPMMEDGISYTTLRFPEMNIPRTGDAESSEMQRPPPDCDDTVTYSALH |
| | | KRQVGDYENVIPDFPEDEGIHYSELIQFGVGERPQAQENVDYVILKH |

1. An anti-CD22 antibody or antigen binding fragment thereof, comprising a heavy chain CDR1 (HCDR1) comprising the amino acid sequence of SEQ ID NO: 1, a heavy chain CDR2 (HCDR2) comprising the amino acid sequence of SEQ ID NO: 2, a heavy chain CDR3 (HCDR3) comprising the amino acid sequence of SEQ ID NO: 3, a light chain CDR1 (LCDR1) comprising the amino acid sequence of SEQ ID NO: 4, a light chain CDR2 (LCDR2) comprising the amino acid sequence of SEQ ID NO: 5, and a light chain CDR3 (LCDR3) comprising the amino acid sequence of SEQ ID NO: 6.

2. The anti-CD22 antibody or antigen binding fragment thereof according to claim 1, wherein the antibody or antigen binding fragment thereof comprises a variable heavy (VH) chain at least 90%, at least 91% at least 92%, at least 93%, at least 94%, at least 95%, at least 96%, at least 97%, at least 98%, or at least 99% identical to the amino acid sequence of SEQ ID NO: 7, and a variable light (VL) chain at least 90%, at least 91% at least 92%, at least 93%, at least 94%, at least 95%, at least 96%, at least 97%, at least 98%, or at least 99% identical to the amino acid sequence of SEQ ID NO: 8.

3. The anti-CD22 antibody or antigen binding fragment thereof according to claim 2, wherein the antibody or antigen binding fragment thereof comprises a VH chain comprising the amino acid sequence of SEQ ID NO: 7 and a VL chain comprising the amino acid sequence of SEQ ID NO: 8.

4. The anti-CD22 antibody or antigen binding fragment thereof according to any one of claims 1 to 3, wherein the antibody or binding fragment thereof comprises an Fc region.

5. The anti-CD22 antibody or antigen binding fragment thereof according to claim 4, wherein the Fc region comprises one or more mutations, which has reduced antibody dependent cellular cytotoxicity (ADCC) compared to an antibody having a wild-type Fc region.

6. The anti-CD22 antibody or antigen binding fragment thereof according to claim 5, wherein the Fc region comprises a L234F/L235E/P331S (EU Index numbering) triple mutation (TM).

7. The anti-CD22 antibody or antigen binding fragment thereof according to any one of claims 1 to 6, wherein the anti-CD22 antibody or antigen binding fragment thereof comprises a constant heavy chain comprising the amino acid sequence of SEQ ID NO: 11 and a constant light chain comprising the amino acid sequence of SEQ ID NO: 12.

8. The anti-CD22 antibody or antigen binding fragment thereof according to any one of claims 1 to 7, wherein the antibody or antigen binding fragment thereof comprises a heavy chain comprising the amino acid sequence of SEQ ID NO: 9.

9. The anti-CD22 antibody or antigen binding fragment thereof according to any one of claims 1 to 8, wherein the antibody or antigen binding fragment thereof comprises a light chain comprising the amino acid sequence of SEQ ID NO: 10.

10. The anti-CD22 antibody or antigen binding fragment thereof according to any one of claims 1 to 9, wherein the antibody or antigen binding fragment thereof comprises a heavy chain comprising the amino acid sequence of SEQ ID NO: 9; and a light chain comprising the amino acid sequence of SEQ ID NO: 10.

11. The anti-CD22 antibody or antigen binding fragment thereof according to any one of claims 1 to 4, wherein the antibody or antigen binding fragment thereof comprises a heavy chain comprising the amino acid sequence of SEQ ID NO: 13; and a light chain comprising the amino acid sequence of SEQ ID NO: 10.

12. The anti-CD22 antigen binding fragment according to any one of claims 1 to 11, wherein the antigen-binding fragment is a single chain Fv, Fab fragment, a Fab′ fragment, or a F(ab′)2 fragment.

13. The anti-CD22 antibody or antigen-binding fragment thereof according to any one of claims 1 to 12, wherein the anti-CD22 antibody or antigen binding fragment thereof is humanized, chimeric, or fully human.

14. The anti-CD22 antibody or antigen-binding fragment thereof according to claim 13, wherein the anti-CD22 antibody or antigen binding fragment is fully human.

15. The anti-CD22 antibody or antigen-binding fragment thereof according to any one of claims 1 to 14, wherein the anti-CD22 antibody or antigen-binding fragment thereof is a monoclonal antibody.

16. The anti-CD22 antibody or antigen-binding fragment thereof according to any one of claims 1 to 15, wherein the anti-CD22 antibody or antigen-binding fragment thereof is an IgG1.

17. The anti-CD22 antibody or antigen binding fragment thereof according to any one of claims 1 to 16, wherein the antibody or antigen binding fragment thereof binds a B-cell derived cell line.

18. A pharmaceutical composition comprising an anti-CD22 antibody or antigen binding fragment thereof according to any one of claims 1 to 17.

19. A polynucleotide encoding the anti-CD22 antibody or antigen binding fragment thereof according to any one of claims 1 to 17.

20. The polynucleotide according to claim 19 comprising a heavy chain nucleic acid sequence of SEQ ID NO: 16 and a light chain nucleic acid sequence of SEQ ID NO: 15.

21. A host cell comprising the polynucleotide of either claim 19 or claim 20.

22. A method for producing an anti-CD22 antibody or antigen binding fragment thereof, comprising expressing the polynucleotide according to either claim 19 or claim 20 in a host cell.

23. An anti-CD22 antibody or antigen binding fragment thereof obtainable by the method of claim 22.

24. The anti-CD22 antibody or antigen binding fragment thereof according to any one of claims 1 to 17 or 23, wherein the antibody or antigen binding fragment thereof is conjugated to one or more drugs.

25. The anti-CD22 antibody or antigen binding fragment thereof according to claim 24, wherein the antibody or antigen binding fragment thereof is conjugated to one or more drugs selected from the group consisting of a cytotoxin, a tubulysin derivative, an antimicrobial agent, a therapeutic agent, a prodrug, a peptide, a protein, an enzyme, a lipid, a biological response modifier, a pharmaceutical agent, a lymphokine, a heterologous antibody, a fragment of a heterologous antibody, a detectable label, a polyethylene glycol (PEG), a radioisotope, and a combination thereof.

26. The anti-CD22 antibody or antigen binding fragment thereof according to claim 25, wherein the one or more drugs is a cytotoxin selected from the group consisting of a DNA scission agent, a DNA binding agent, a DNA intercalating agent, a topoisomerase I inhibitor, a topoisomerase II inhibitor, a tubulysin derivative, and an antimitotic agent.

27. The anti-CD22 antibody or antigen binding fragment thereof according to claim 25, wherein the one or more drugs is a cytotoxin selected from the group consisting of calicheamicins, pyrrolobenzodiazepines, camptothecins, daunorubicins, doxorubicins, auristatins, and maytansinoids.

28. The anti-CD22 antibody or antigen binding fragment thereof according to claim 25, wherein the one or more drugs is a cytotoxin which is a topoisomerase I inhibitor.

29. The anti-CD22 antibody or antigen binding fragment thereof according to claim 28, wherein the topoisomerase I inhibitor is exatecan:

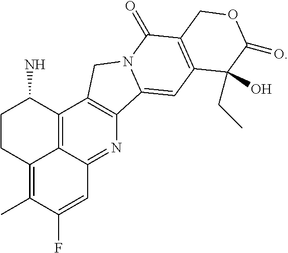 [SMILES: CC[C@@]1(O)C(=O)OCc2c1cc1n(c2=O)Cc2c-1nc1cc(F)c(C)c3c1c2[C@@H](N)CC3]

30. The anti-CD22 antibody or antigen binding fragment thereof according to claim 28, wherein the topoisomerase I inhibitor is:

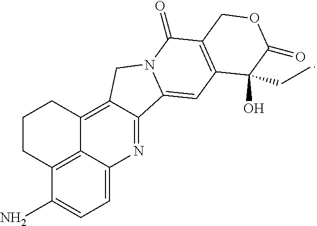 [SMILES: CC[C@@]1(O)C(=O)OCc2c1cc1n(c2=O)Cc2c-1nc1ccc(N)c3c1c2CCC3]

31. The anti-CD22 antibody or antigen binding fragment thereof according to any one of claims 24 to 30, wherein the antibody or antigen binding fragment is conjugated to a drug at a drug to antibody ratio (DAR) of about 4 to about 8.

32. The anti-CD22 antibody or antigen binding fragment thereof according to claim 31, wherein the antibody or antigen binding fragment is conjugated to a drug at a drug to antibody ratio (DAR) of about 8.

33. The anti-CD22 antibody or antigen binding fragment thereof according to claim 31, wherein the antibody or antigen binding fragment is conjugated to a drug at a drug to antibody ratio (DAR) of 4.

34. An antibody-drug conjugate (ADC) of Formula (I): 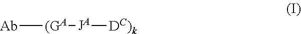
wherein
Ab is an antibody or antigen-binding fragment thereof according to any one of claims 1 to 17,
k is an integer from 1 to 10;
each G^{A} is independently a conjugation group conjugated to the antibody or antigen-binding fragment thereof;
each D^{C} is selected from the group consisting of a cytotoxin, a tubulysin derivative, an antimicrobial agent, a therapeutic agent, a prodrug, a peptide, a protein, an enzyme, a lipid, a biological response modifier, a pharmaceutical agent, a lymphokine, a heterologous antibody, a fragment of a heterologous antibody, a detectable label, a polyethylene glycol (PEG), and a radioisotope; and
each J^{A} is independently a linker.

35. The antibody-drug conjugate according to claim 34 wherein each J^{A} is independently a group of Formula (ICA): 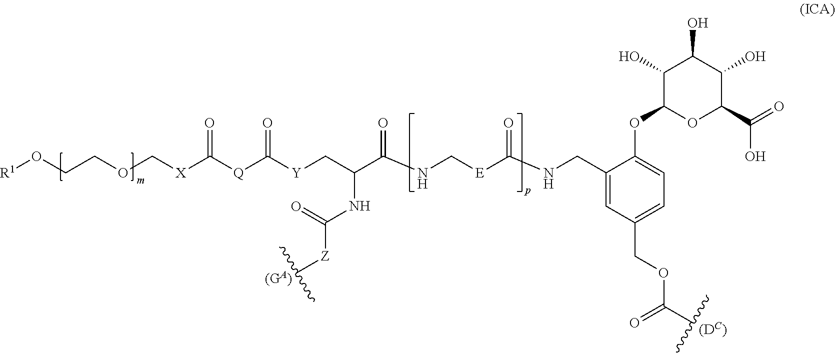
E is (CH₂)ₙ₁, wherein n1 is 0, 1, 2 or 3;
Q is 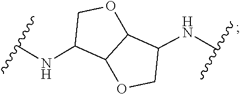 [SMILES: *NC1COC2C(N*)COC12]
R¹ is C₁₋₄ alkyl;
X is (CH₂)ₙ₂, wherein n2 is 0, 1, 2, or 3;
Y is (CH₂)ₙ₃, wherein n3 is 0, 1, 2, 3, or 4;
Z is (CH₂)ₙ₄, wherein n4 is 1, 2, 3, 4, or 5;
m is an integer from 5 to 17;
p is 1 or 0;
(G^{A}) indicates the point of attachment to G^{A}; and
(D^{C}) indicates the point of attachment to D^{C}.

36. The antibody-drug conjugate according to claim 35, wherein Q is:

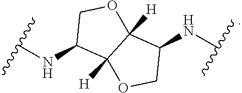 [SMILES: *N[C@H]1CO[C@H]2[C@@H]1OC[C@@H]2N*]

37. The antibody-drug conjugate according to either claim 35 or claim 36, wherein m is 9, 10, 11, 12, or 13.

38. The antibody-drug conjugate according to any one of claims 35 to 37, wherein R¹ is CH₃.

39. The antibody-drug conjugate according to any one of claims 35 to 38, wherein E is CH₂.

40. The antibody-drug conjugate according to any one of claims 35 to 39, wherein X is CH₂.

41. The antibody-drug conjugate according to any one of claims 35 to 40, wherein Y is (CH₂)₂.

42. The antibody-drug conjugate according to any one of claims 35 to 41, wherein Z is (CH₂)₂.

43. The antibody-drug conjugate according to any one of claims 35 to 41, wherein Z is CH₂.

44. The antibody-drug conjugate according to any one of claims 35 to 43, wherein p is 1.

45. The antibody-drug conjugate according to claim 35, wherein each J^{A} is a group of Formula (ICB):

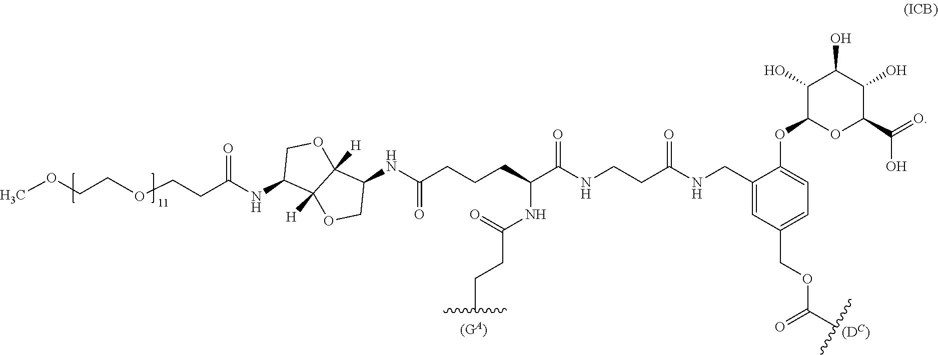

46. The antibody-drug conjugate according to any one of claims 34 to 45, wherein G^{A} is selected from the group consisting of: 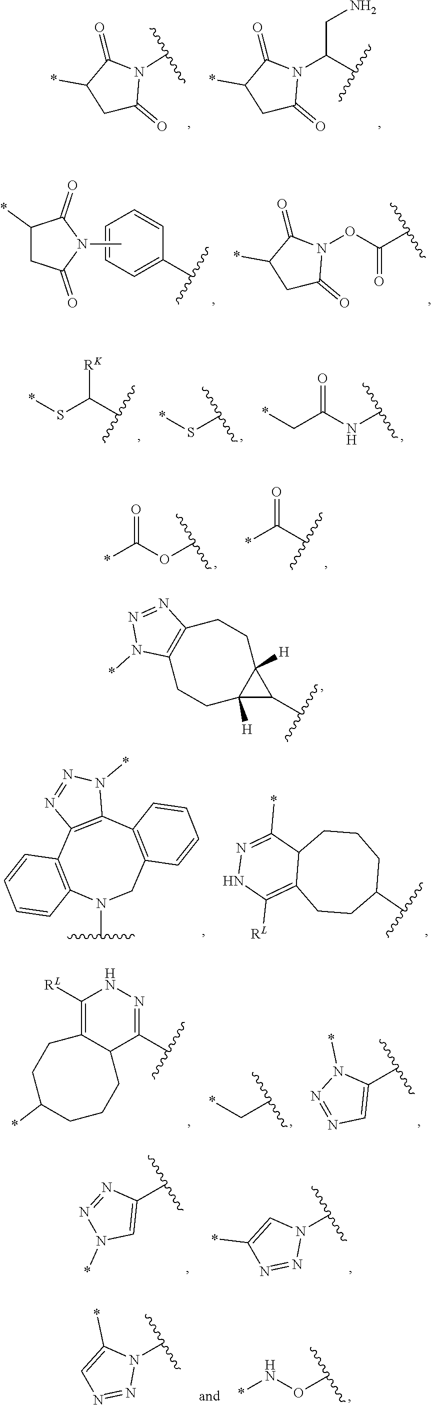
wherein R^{K} is H or CH₃, R^{L} is C₁₋₆ alkyl, and [symbol] indicates the point of attachment to the antibody or antigen-binding fragment thereof.

47. The antibody-drug conjugate according to claim 46, wherein G^{A} is:

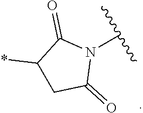 [SMILES: *C1CC(=O)N(*)C1=O]

48. The antibody-drug conjugate according to claim 46, wherein G^{A} is:

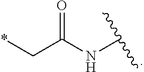 [SMILES: *CC(=O)N*]

49. The antibody-drug conjugate according to any one of claims 35 to 48, wherein k is an integer from about 4 to about 8.

50. The antibody-drug conjugate according to claim 49, wherein k is about 4.

51. The antibody-drug conjugate according to claim 49, wherein k is about 8.

52. The antibody-drug conjugate according to any one of claims 34 to 51, wherein each D^{C} is: 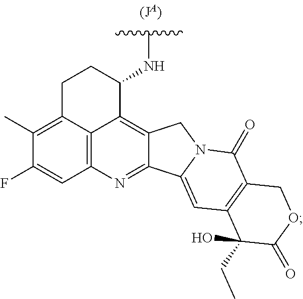 [SMILES: *N[C@H]1CCc2c(C)c(F)cc3nc4c(c1c23)Cn1c-4cc2c(c1=O)COC(=O)[C@]2(O)CC]
and (J^{A}) indicates the point of attachment to J^{A}.

53. The antibody-drug conjugate according to claim 52, wherein G^{A}-J^{A}-D^{C} is: 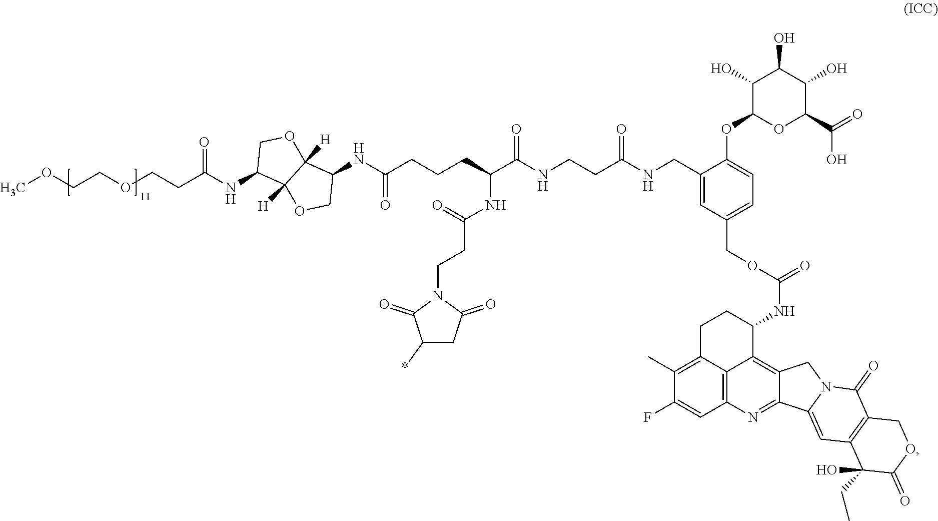
wherein [symbol] indicates the point of attachment to the antibody or antigen-binding fragment thereof.

54. The antibody-drug conjugate according to claim 52, wherein G^{A}-J^{A}-D^{C} is: 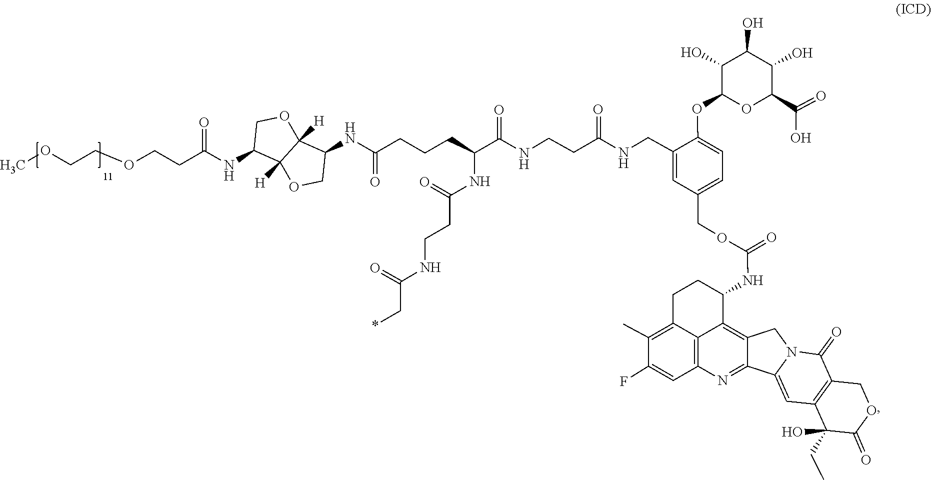
wherein [symbol] indicates the point of attachment to the antibody or antigen-binding fragment thereof.

55. The antibody-drug conjugate according to claim 34, wherein each J^{A} is a group of Formula (ICE):

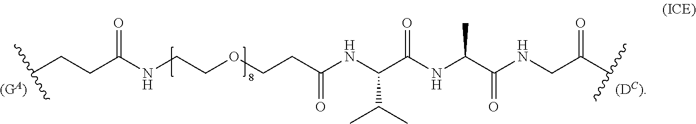

56. The antibody-drug conjugate according to claim 55, wherein G^{A} is selected from the group consisting of: 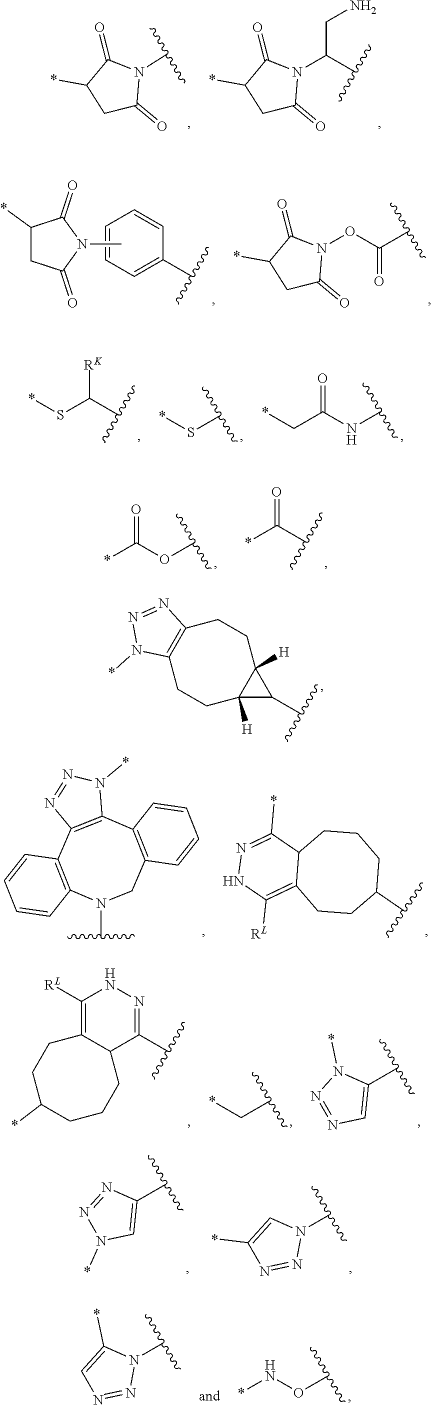
wherein R^{K} H or CH₃, R^{L} is C₁₋₆ alkyl, and [symbol] indicates the point of attachment to the antibody or antigen-binding fragment thereof.

57. The antibody-drug conjugate according to claim 56, wherein G^{A} is

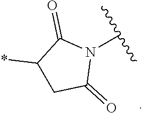 [SMILES: *C1CC(=O)N(*)C1=O]

58. The antibody-drug conjugate according to claim 56, wherein G^{A} is

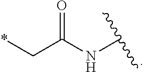 [SMILES: *CC(=O)N*]

59. The antibody-drug conjugate according to any one of claims 55 to 58, wherein k is n integer from about 4 to about 8.

60. The antibody-drug conjugate according to claim 59, wherein k is about 4.

61. The antibody-drug conjugate according to claim 59, wherein k is about 8.

62. The antibody-drug conjugate according to any one of claims 55 to 61, wherein each D^{C} is: 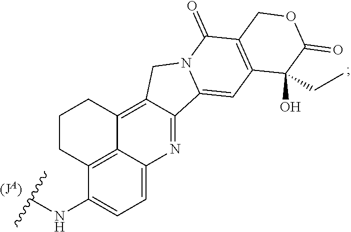 [SMILES: *Nc1ccc2nc3c(c4c2c1CCC4)Cn1c-3cc2c(c1=O)COC(=O)[C@]2(O)CC]
and (J^{A}) indicates the point of attachment to J^{A}.

63. The antibody-drug conjugate according to claim 62, wherein G^{A}-J^{A}-D^{C} is: 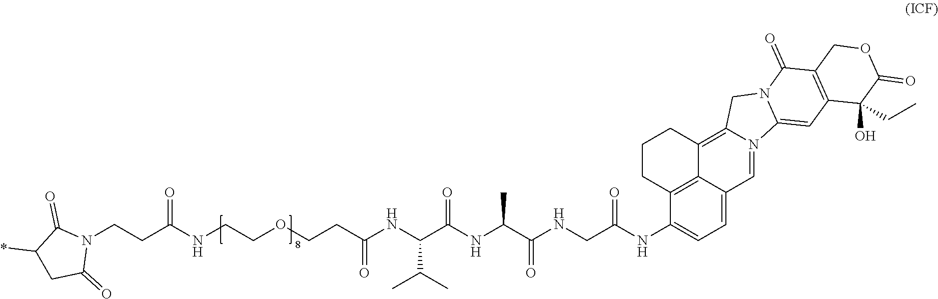
wherein [symbol] indicates the point of attachment to the antibody or antigen-binding fragment thereof.

64. The antibody-drug conjugate according to claim 34, wherein each J^{A} is a group of Formula (ICG):

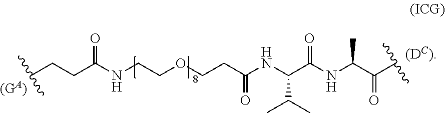

65. The antibody-drug conjugate according to claim 64, wherein G^{A} is selected from the group consisting of: 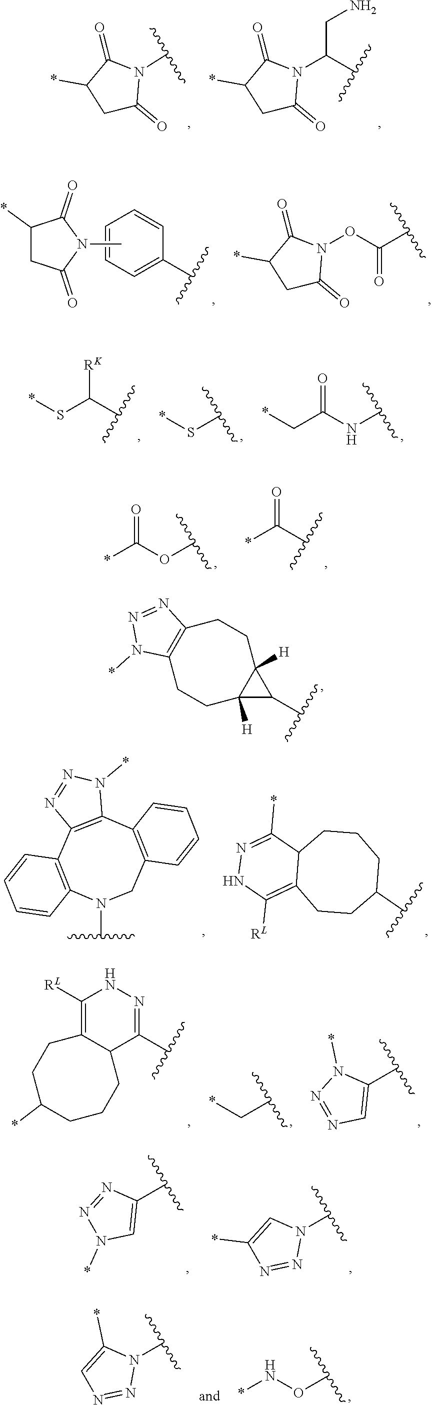
wherein R^{K} is H or CH₃, R^{L} is C₁₋₆ alkyl, and [symbol] indicates the point of attachment to the antibody or antigen-binding fragment thereof.

66. The antibody-drug conjugate according to claim 65, wherein G^{A} is:

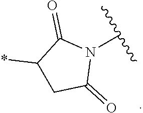 [SMILES: *C1CC(=O)N(*)C1=O]

67. The antibody-drug conjugate according to claim 65, wherein G^{A} is:

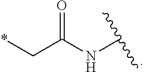 [SMILES: *CC(=O)N*]

68. The antibody-drug conjugate according to any one of claims 64 to 67, wherein k is an integer from about 4 to about 8.

69. The antibody-drug conjugate according to claim 68, wherein k is about 4.

70. The antibody-drug conjugate according to claim 68, wherein k is about 8.

71. The antibody-drug conjugate according to any one of claims 64 to 70, wherein each D^{C} is: 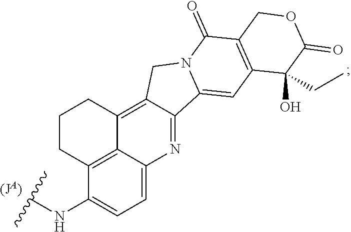 [SMILES: *Nc1ccc2nc3c(c4c2c1CCC4)Cn1c-3cc2c(c1=O)COC(=O)[C@]2(O)CC]
and (J^{A}) indicates the point of attachment to J^{A}.

72. The antibody-drug conjugate according to claim 71, wherein G^{A}-J^{A}-D^{C} is: 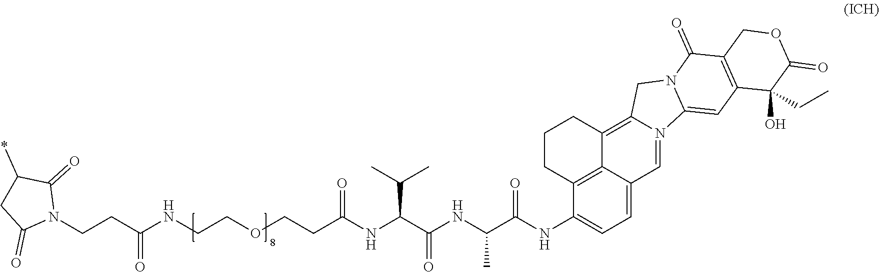
wherein [symbol] indicates the point of attachment to the antibody or antigen-binding fragment thereof.

73. An antibody-drug conjugate comprising:
(i) an anti-CD22 antibody or antigen binding fragment thereof comprising a heavy chain CDR1 (HCDR1) comprising the amino acid sequence of SEQ ID NO: 1, a heavy chain CDR2 (HCDR2) comprising the amino acid sequence of SEQ ID NO: 2, a heavy chain CDR3 (HCDR3) comprising the amino acid sequence of SEQ ID NO: 3, a light chain CDR1 (LCDR1) comprising the amino acid sequence of SEQ ID NO: 4, a light chain CDR2 (LCDR2) comprising the amino acid sequence of SEQ ID NO: 5, and a light chain CDR3 (LCDR3) comprising the amino acid sequence of SEQ ID NO: 6; and
(ii) a linker payload of Formula (ICC): 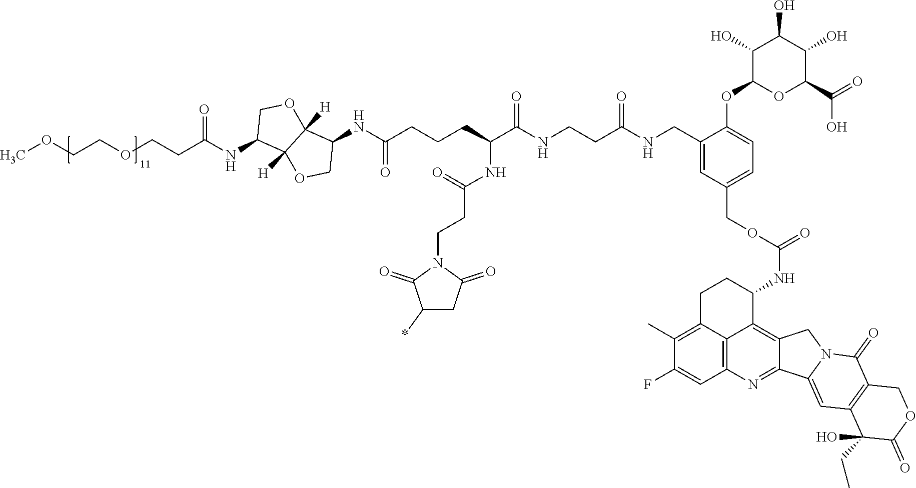
wherein [symbol] indicates the point of attachment to the antibody or antigen-binding fragment thereof; and
wherein the ADC has a drug to antibody ratio (DAR) ranging from about 4 to about 8.

74. An antibody-drug conjugate comprising:
(i) an anti-CD22 antibody or antigen binding fragment thereof comprising a heavy chain CDR1 (HCDR1) comprising the amino acid sequence of SEQ ID NO: 1, a heavy chain CDR2 (HCDR2) comprising the amino acid sequence of SEQ ID NO: 2, a heavy chain CDR3 (HCDR3) comprising the amino acid sequence of SEQ ID NO: 3, a light chain CDR1 (LCDR1) comprising the amino acid sequence of SEQ ID NO: 4, a light chain CDR2 (LCDR2) comprising the amino acid sequence of SEQ ID NO: 5, and a light chain CDR3 (LCDR3) comprising the amino acid sequence of SEQ ID NO: 6; and
(ii) a linker payload of Formula (ICD): 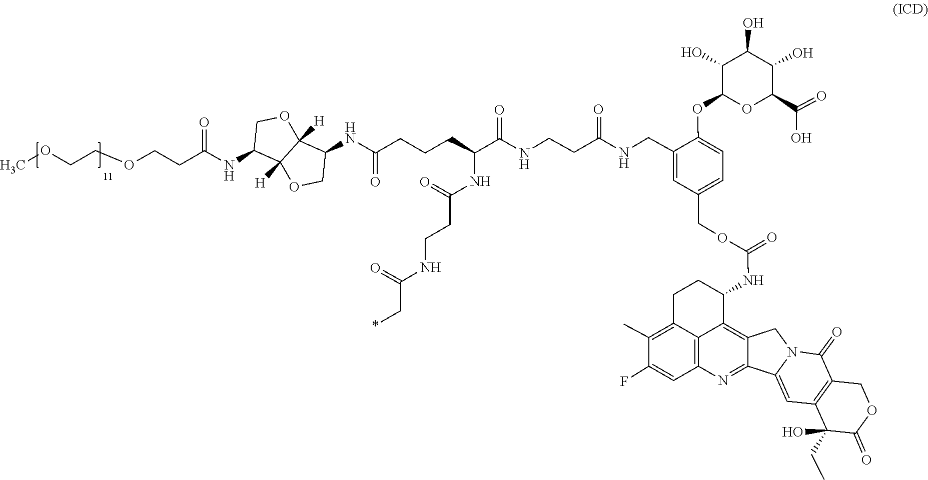
wherein [symbol] indicates the point of attachment to the antibody or antigen-binding fragment thereof; and
wherein the ADC has a drug to antibody ratio (DAR) ranging from about 4 to about 8.

75. An antibody-drug conjugate comprising:
(i) an anti-CD22 antibody or antigen binding fragment thereof comprising a heavy chain CDR1 (HCDR1) comprising the amino acid sequence of SEQ ID NO: 1, a heavy chain CDR2 (HCDR2) comprising the amino acid sequence of SEQ ID NO: 2, a heavy chain CDR3 (HCDR3) comprising the amino acid sequence of SEQ ID NO: 3, a light chain CDR1 (LCDR1) comprising the amino acid sequence of SEQ ID NO: 4, a light chain CDR2 (LCDR2) comprising the amino acid sequence of SEQ ID NO: 5, and a light chain CDR3 (LCDR3) comprising the amino acid sequence of SEQ ID NO: 6; and
(ii) a linker payload of Formula (ICF): 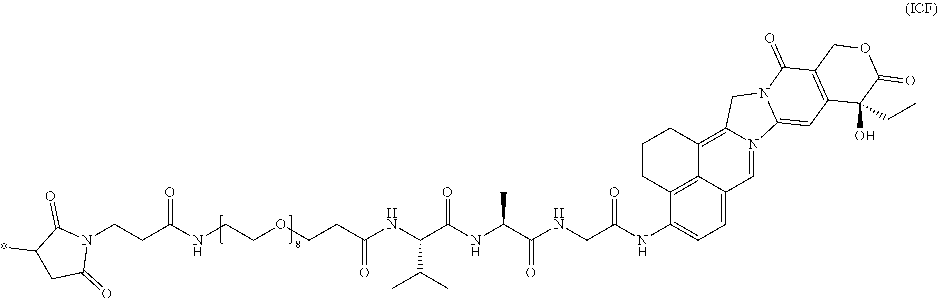
wherein [symbol] indicates the point of attachment to the antibody or antigen-binding fragment thereof; and
wherein the ADC has a drug to antibody ratio (DAR) ranging from about 4 to about 8.

76. An antibody-drug conjugate comprising:
(i) an anti-CD22 antibody or antigen binding fragment thereof comprising a heavy chain CDR1 (HCDR1) comprising the amino acid sequence of SEQ ID NO: 1, a heavy chain CDR2 (HCDR2) comprising the amino acid sequence of SEQ ID NO: 2, a heavy chain CDR3 (HCDR3) comprising the amino acid sequence of SEQ ID NO: 3, a light chain CDR1 (LCDR1) comprising the amino acid sequence of SEQ ID NO: 4, a light chain CDR2 (LCDR2) comprising the amino acid sequence of SEQ ID NO: 5, and a light chain CDR3 (LCDR3) comprising the amino acid sequence of SEQ ID NO: 6; and
(ii) a linker payload of Formula (ICH): 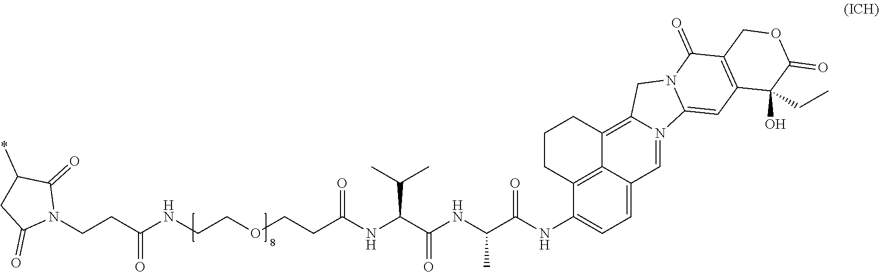
wherein [symbol] indicates the point of attachment to the antibody or antigen-binding fragment thereof; and
wherein the ADC has a drug to antibody ratio (DAR) ranging from about 4 to about 8.

77. The antibody-drug conjugate according to any one of claims 73 to 76, wherein the antibody or antigen binding fragment thereof comprises a VH chain comprising the amino acid sequence of SEQ ID NO: 7 and a VL chain comprising the amino acid sequence of SEQ ID NO: 8.

78. The antibody-drug conjugate according to any one of claims 73 to 77, wherein the antibody or antigen binding fragment thereof comprises a heavy chain (HC) comprising the amino acid sequence of SEQ ID NO: 9, and light chain (LC) comprising the amino acid sequence of SEQ ID NO: 10.

79. The antibody-drug conjugate according to any one of claims 73 to 77, wherein the antibody or antigen binding fragment thereof comprises a heavy chain (HC) comprising the amino acid sequence of SEQ ID NO: 13, and light chain (LC) comprising the amino acid sequence of SEQ ID NO: 10.

80. A pharmaceutical composition comprising an antibody-drug conjugate according to any one of claims 34 to 79.

81. The antibody-drug conjugate of any one of claims 34 to 79 or the pharmaceutical composition of claim 80, for use as a medicament.

82. The antibody-drug conjugate of any one of claims 34 to 79 or the pharmaceutical composition of claim 80, for use in the treatment of cancer.

83. The antibody-drug conjugate or the pharmaceutical composition for use in the treatment of cancer according to claim 82, wherein said cancer expresses CD22.

84. A method of treating cancer, the method comprising administering to a subject the antibody-drug conjugate of any one of claims 34 to 79 or the pharmaceutical composition of claim 80.

85. The method according to claim 84, wherein said cancer expresses CD22.

86. The method according to either claim 84 or claim 85, wherein the subject is a human.

87. The method according to any one of claims 84 to 86, or the antibody-drug conjugate or pharmaceutical composition for use according to any one of claims 71 to 73, wherein said cancer is a B-cell malignancy.

88. The method or antibody-drug conjugate or pharmaceutical composition for use according to claim 87, wherein said B-cell malignancy is selected from the group consisting of non-Hodgkin lymphoma, acute lymphoblastic leukemia (ALL), Burkitt lymphoma, chronic lymphocytic leukemia (CLL), small lymphocytic lymphoma (SLL), diffuse large B-cell lymphoma (DLBCL), follicular lymphoma (FL), mantle cell lymphoma (MCL), lymphoplasmacytic lymphoma, and Hairy cell leukemia.

89. The method or antibody-drug conjugate or pharmaceutical composition for use according to claim 88, wherein the B-cell malignancy is refractory [B-cell] acute lymphoblastic leukemia (ALL) or relapsed [B-cell] acute lymphoblastic leukemia (ALL).

90. The anti-CD22 antibody or antigen binding fragment thereof according to any one of claims 1 to 17 or the pharmaceutical composition of claim 18, for use as a medicament.

91. The anti-CD22 antibody or antigen binding fragment thereof according to any one of claims 1 to 17 or the pharmaceutical composition of claim 18, for use in the treatment of cancer.

92. The anti-CD22 antibody or antigen binding fragment thereof according to any one of claims 1 to 17 or the pharmaceutical composition of claim 18, wherein said cancer expresses CD22.

93. A method of treating cancer, the method comprising administering to a subject the anti-CD22 antibody or antigen binding fragment thereof according to any one of claims 1 to 17 or the pharmaceutical composition of claim 18.

94. The method according to claim 93, wherein said cancer expresses CD22.

95. The method according to either claim 93 or claim 94, wherein the subject is a human.

96. The method according to any one of claims 93 to 95, or the anti-CD22 antibody or antigen binding fragment thereof for use, or pharmaceutical composition for use according to either claim 81 or claim 82, wherein said cancer is a B-cell malignancy.

97. The method or the anti-CD22 antibody or antigen binding fragment thereof for use or pharmaceutical composition for use according to claim 96, wherein said B-cell malignancy is selected from the group consisting of non-Hodgkin lymphoma, acute lymphoblastic leukemia (ALL), Burkitt lymphoma, chronic lymphocytic leukemia (CLL), small lymphocytic lymphoma (SLL), diffuse large B-cell lymphoma (DLBCL), follicular lymphoma (FL), mantle cell lymphoma (MCL), lymphoplasmacytic lymphoma, and Hairy cell leukemia.

98. The method or antibody-drug conjugate or pharmaceutical composition for use according to claim 97, wherein the B-cell malignancy is refractory [B-cell] acute lymphoblastic leukemia (ALL) or relapsed [B-cell] acute lymphoblastic leukemia (ALL).

99. A method for detecting the presence or absence of a CD22 polypeptide in a sample, comprising:
(i) contacting a sample with an antibody or antigen binding fragment thereof according to any one of claims 1-17, or a pharmaceutical composition according to claim 18, to provide an antibody-antigen complex;
(ii) detecting the presence or absence of said antibody-antigen complex;
wherein the presence of the antibody-antigen complex confirms the presence of a CD22 polypeptide; and wherein the absence of the antibody-antigen complex confirms the absence of CD22 polypeptide.

100. The method according to claim 99, wherein the presence of said antibody-antigen complex is indicative of the presence of a CD22 expressing cancer cell, and wherein the absence of said antibody-antigen complex is indicative of the absence of a CD22 expressing cancer cell.

101. The method according to either claim 99 or claim 100, wherein the sample is an isolated sample obtainable from a subject.

102. A kit of parts comprising at least one of (i) a variable heavy chain, and (ii) a variable light chain of (a) the anti-CD22 antibody or antigen binding fragment thereof according to any one of claims 1 to 17, (b) the antibody drug conjugate according to any one of claims 34 to 79, or (c) the pharmaceutical composition according to either claim 18 or claim 80.


SEQUENCE LISTING (17 sequences)

SEQ ID NO: 1 - protein, 5 aa, synthetic construct
SYAMT

SEQ ID NO: 2 - protein, 17 aa, synthetic construct
TISGSGVSTYSADSVKG

SEQ ID NO: 3 - protein, 10 aa, synthetic construct
HWELLDAFDI

SEQ ID NO: 4 - protein, 12 aa, synthetic construct
RASQSISYNYLA

SEQ ID NO: 5 - protein, 7 aa, synthetic construct
GASSRAS

SEQ ID NO: 6 - protein, 9 aa, synthetic construct
QQYGSSPLT

SEQ ID NO: 7 - protein, 119 aa, synthetic construct
EVQLLESGGGLVQPGGSLRLSCAASGFTFSSYAMTWVRQAPGKGLEWVSTISGSGVSTYSADSVKGRFTISRDNSKNTLYLQMNSLRAEDTAVYYCALHWELLDAFDIWGQGTMVTVSS

SEQ ID NO: 8 - protein, 108 aa, synthetic construct
EIVLTQSPGTLSLSPGERATLSCRASQSISYNYLAWYQQKPGQAPRLLIYGASSRASGISDRFSGSGSGTDFTLTISRLEPEDFVVYYCQQYGSSPLTFGGGTKVEVK

SEQ ID NO: 9 - protein, 449 aa, synthetic construct
EVQLLESGGGLVQPGGSLRLSCAASGFTFSSYAMTWVRQAPGKGLEWVSTISGSGVSTYSADSVKGRFTISRDNSKNTLYLQMNSLRAEDTAVYYCALHWELLDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 215 aa, synthetic construct
EIVLTQSPGTLSLSPGERATLSCRASQSISYNYLAWYQQKPGQAPRLLIYGASSRASGISDRFSGSGSGTDFTLTISRLEPEDFVVYYCQQYGSSPLTFGGGTKVEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 107 aa, synthetic construct [sequence omitted]

SEQ ID NO: 13 - protein, 449 aa, synthetic construct
EVQLLESGGGLVQPGGSLRLSCAASGFTFSSYAMTWVRQAPGKGLEWVSTISGSGVSTYSADSVKGRFTISRDNSKNTLYLQMNSLRAEDTAVYYCALHWELLDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 649 aa, synthetic construct
GAAATCGTATTGACACAATCTCCTGGGACCCTCTCTCTCTCTCCCGGAGAGAGAGCAACCCTCAGTTGTCGCGCGTCTCAGAGCATTTCATACAACTACTTGGCGTGGTATCAGCAGAAACCTGGGCAGGCTCCCAGGCTTCTGATTTACGGGGCGTCAAGTAGGGCCTCCGGCATTAGCGATAGGTTTTCAGGTAGCGGCAGTGGTACAGATTTCACCTTGACGATAAGTCGCCTCGAACCAGAGGATTTCGTGGTCTATTACTGCCAACAGTATGGGAGCTCCCCGCTTACATTTGGCGGCGGTACAAAGGTCGAGGTAAAACGTACGGTGGCTGCACCATCTGTCTTCATCTTCCCGCCATCTGATGAGCAGTTGAAATCTGGAACTGCCTCTGTTGTGTGCCTGCTGAATAACTTCTATCCCAGAGAGGCCAAAGTACAGTGGAAGGTGGATAACGCCCTCCAATCGGGTAACTCCCAGGAGAGTGTCACAGAGCAGGACAGCAAGGACAGCACCTACAGCCTCAGCAGCACCCTGACGCTGAGCAAAGCAGACTACGAGAAACACAAAGTCTACGCCTGCGAAGTCACCCATCAGGGCCTGAGCTCGCCCGTCACAAAGAGCTTCAACAGGGGAGAGTGTTAGT

SEQ ID NO: 16 - protein, 1350 aa, synthetic construct
GAGGTCCAGTTGCTTGAAAGCGGCGGGGGTCTTGTCCAGCCCGGTGGGAGTCTGCGACTCTCTTGTGCTGCGAGTGGTTTCACCTTTAGCTCCTACGCTATGACTTGGGTTAGACAAGCCCCGGGCAAGGGACTTGAATGGGTATCCACTATTTCAGGCTCTGGCGTAAGCACTTATAGTGCGGATTCCGTTAAAGGGCGATTTACAATCAGTCGAGACAATTCAAAGAACACGCTTTACTTGCAGATGAACTCATTGCGAGCGGAGGATACTGCCGTCTATTACTGTGCGTTGCATTGGGAACTTCTCGACGCGTTTGACATTTGGGGTCAGGGCACTATGGTCACTGTGAGTTCAGCGTCGACCAAGGGCCCATCCGTCTTCCCCCTGGCACCCTCCTCCAAGAGCACCTCTGGGGGCACAGCGGCCCTGGGCTGCCTGGTCAAGGACTACTTCCCCGAACCGGTGACGGTGTCCTGGAACTCAGGCGCTCTGACCAGCGGCGTGCACACCTTCCCGGCTGTCCTACAGTCCTCAGGACTCTACTCCCTCAGCAGCGTGGTGACCGTGCCCTCCAGCAGCTTGGGCACCCAGACCTACATCTGCAACGTGAATCACAAGCCCAGCAACACCAAGGTGGACAAGAGAGTTGAGCCCAAATCTTGTGACAAAACTCACACATGCCCACCGTGCCCAGCACCTGAATTCGAGGGGGGACCGTCAGTCTTCCTCTTCCCCCCAAAACCCAAGGACACCCTCATGATCTCCCGGACCCCTGAGGTCACATGCGTGGTGGTGGACGTGAGCCACGAAGACCCTGAGGTCAAGTTCAACTGGTACGTGGACGGCGTGGAGGTGCATAATGCCAAGACAAAGCCGCGGGAGGAGCAGTACAACAGCACGTACCGTGTGGTCAGCGTCCTCACCGTCCTGCACCAGGACTGGCTGAATGGCAAGGAGTACAAGTGCAAGGTCTCCAACAAAGCCCTCCCAGCCTCCATCGAGAAAACCATCTCCAAAGCCAAAGGGCAGCCCCGAGAACCACAGGTCTACACCCTGCCCCCATCCCGGGAGGAGATGACCAAGAACCAGGTCAGCCTGACCTGCCTGGTCAAAGGCTTCTATCCCAGCGACATCGCCGTGGAGTGGGAGAGCAATGGGCAGCCGGAGAACAACTACAAGACCACGCCTCCCGTGCTGGACTCCGACGGCTCCTTCTTCCTCTATAGCAAGCTCACCGTGGACAAGAGCAGGTGGCAGCAGGGGAACGTCTTCTCATGCTCCGTGATGCATGAGGCTCTGCACAACCACTACACGCAGAAGAGCCTCTCCCTGTCTCCGGGTAAATGA

SEQ ID NO: 17 - protein, 847 aa, synthetic construct
MHLLGPWLLLLVLEYLAFSDSSKWVFEHPETLYAWEGACVWIPCTYRALDGDLESFILFHNPEYNKNTSKFDGTRLYESTKDGKVPSEQKRVQFLGDKNKNCTLSIHPVHLNDSGQLGLRMESKTEKWMERIHLNVSERPFPPHIQLPPEIQESQEVTLTCLLNFSCYGYPIQLQWLLEGVPMRQAAVTSTSLTIKSVFTRSELKFSPQWSHHGKIVTCQLQDADGKFLSNDTVQLNVKHTPKLEIKVTPSDAIVREGDSVTMTCEVSSSNPEYTTVSWLKDGTSLKKQNTFTLNLREVTKDQSGKYCCQVSNDVGPGRSEEVFLQVQYAPEPSTVQILHSPAVEGSQVEFLCMSLANPLPTNYTWYHNGKEMQGRTEEKVHIPKILPWHAGTYSCVAENILGTGQRGPGAELDVQYPPKKVTTVIQNPMPIREGDTVTLSCNYNSSNPSVTRYEWKPHGAWEEPSLGVLKIQNVGWDNTTIACAACNSWCSWASPVALNVQYAPRDVRVRKIKPLSEIHSGNSVSLQCDFSSSHPKEVQFFWEKNGRLLGKESQLNFDSISPEDAGSYSCWVNNSIGQTASKAWTLEVLYAPRRLRVSMSPGDQVMEGKSATLTCESDANPPVSHYTWFDWNNQSLPYHSQKLRLEPVKVQHSGAYWCQGTNSVGKGRSPLSTLTVYYSPETIGRRVAVGLGSCLAILILAICGLKLQRRWKRTQSQQGLQENSSGQSFFVRNKKVRRAPLSEGPHSLGCYNPMMEDGISYTTLRFPEMNIPRTGDAESSEMQRPPPDCDDTVTYSALHKRQVGDYENVIPDFPEDEGIHYSELIQFGVGERPQAQENVDYVILKH